(19) United States
(12) Patent Application Publication
(10) Pub. No.: US 2022/0275049 A1
(43) Pub. Date: Sep. 1, 2022

(54) ANTIGENIC POLYPEPTIDES AND METHODS OF USE THEREOF

(71) Applicant: AGENUS INC., Lexington, MA (US)

(72) Inventors: Benjamin Maxime MORIN, Lexington, MA (US); Mark Arthur FINDEIS, Lexington, MA (US); Bishnu JOSHI, Lexington, MA (US)

(21) Appl. No.: 17/582,445

(22) Filed: Jan. 24, 2022

Related U.S. Application Data
(63) Continuation of application No. PCT/US2020/043431, filed on Jul. 24, 2020.
(60) Provisional application No. 62/878,157, filed on Jul. 24, 2019.

(51) Int. Cl. C07K 14/74; A61P 35/02; A61K 45/06

(52) U.S. Cl. CPC C07K 14/70539; A61P 35/02; A61K 45/06; A61K 38/00

(57) ABSTRACT

Provided are novel antigenic polypeptides comprising tumor-associated peptides, and compositions comprising the same. Such antigenic polypeptides and compositions are particularly useful as immunotherapeutics (e.g., cancer vaccines). Also provided are methods of inducing a cellular immune response using such polypeptides and compositions, methods of treating a disease using such polypeptides and compositions, kits comprising such polypeptides and compositions, and methods of making such compositions.

### 1. RELATED APPLICATIONS

This application is a Continuation Application of International Patent Application No. PCT/US2020/043431, filed Jul. 24, 2020, which claims priority to U.S. Provisional Patent Application Ser. No. 62/878,157, entitled “Antigenic Polypeptides And Methods Of Use Thereof”, filed Jul. 24, 2019. The contents of the aforementioned application is hereby incorporated by reference herein in its entirety.

### 2. SEQUENCE LISTING

The sequence listing attached herewith, named 404293_AGBW_129US_188615_Sequence_Listing.txt and created on Jul. 24, 2020, is herein incorporated by reference in its entirety.

### 3. FIELD

The instant disclosure relates to novel antigenic polypeptides and compositions, and uses of such antigenic polypeptides and compositions as immunotherapeutics (e.g., cancer vaccines).

### 4. BACKGROUND

Immunotherapies are becoming important tools in the treatment of cancer. One immunotherapy approach involves the use of therapeutic cancer vaccines comprising cancer-specific antigenic peptides that actively educate a patient's immune system to target and destroy cancer cells. However, the generation of such therapeutic cancer vaccines is limited by the immunogenicity of cancer-specific antigenic peptides.

Accordingly, there is a need in the art for improved immunogenic cancer-specific peptides and for creating effective anti-cancer vaccines comprising these peptides.

### 5. SUMMARY OF INVENTION

The instant disclosure provides novel antigenic polypeptides comprising tumor-associated peptides, and compositions comprising the same. Such antigenic polypeptides and compositions are particularly useful as immunotherapeutics (e.g., cancer vaccines). Also provided are methods of inducing a cellular immune response using such polypeptides and compositions, methods of treating a disease using such polypeptides and compositions, kits comprising such polypeptides and compositions, and methods of making such compositions.

Accordingly, the instant disclosure provides the following, non-limiting, embodiments:

Embodiment 1. An antigenic polypeptide comprising:
an WIC-binding peptide comprising an amino acid sequence selected from the group consisting of SEQ ID NOs: 98-3000 and 8808; and
an HSP-binding peptide comprising the amino acid sequence of X₁X₂X₃X₄X₅X₆X₇ (SEQ ID NO: 1), wherein X₁ is omitted, N, F, or Q; X₂ is W, L, or F; X₃ is L or I; X₄ is R, L, or K; X₅ is L, W, or I; X₆ is T, L, F, K, R, or W; and X₇ is W, G, K, or F.
Embodiment 2. The antigenic polypeptide of embodiment 1, wherein the amino acid sequence of the WIC-binding peptide consists of an amino acid sequence selected from the group consisting of SEQ ID NOs: 98-3000 and 8808.
Embodiment 3. The antigenic polypeptide of embodiment 1 or 2, wherein the HSP-binding peptide comprises the amino acid sequence of:
(a) X₁LX₂LTX₃ (SEQ ID NO: 2), wherein X₁ is W or F; X₂ is R or K; and X₃ is W, F, or G;
(b) NX₁LX₂LTX₃ (SEQ ID NO: 3), wherein X₁ is W or F; X₂ is R or K; and X₃ is W, F, or G;
(c) WLX₁LTX₂ (SEQ ID NO: 4), wherein X₁ is R or K; and X₂ is W or G;
(d) NWLX₁LTX₂ (SEQ ID NO: 5), wherein X₁ is R or K; and X₂ is W or G; or
(e) NWX₁X₂X₃X₄X₅ (SEQ ID NO: 6), wherein X₁ is L or I; X₂ is L, R, or K; X₃ is L or I; X₄ is T, L, F, K, R, or W; and X₅ is W or K.
Embodiment 4. The antigenic polypeptide of embodiment 1 or 2, wherein the HSP-binding peptide comprises an amino acid sequence selected from the group consisting of SEQ ID NOs: 7-42, optionally wherein the amino acid sequence of the HSP-binding peptide consists of an amino acid sequence selected from the group consisting of SEQ ID NOs: 7-42.
Embodiment 5. The antigenic polypeptide of embodiment 1 or 2, wherein the HSP-binding peptide comprises the amino acid sequence of SEQ ID NO: 7, optionally wherein the amino acid sequence of the HSP-binding peptide consists of the amino acid sequence of SEQ ID NO: 7.
Embodiment 6. The antigenic polypeptide of embodiment 1 or 2, wherein the HSP-binding peptide comprises the amino acid sequence of SEQ ID NO: 8, optionally wherein the amino acid sequence of the HSP-binding peptide consists of the amino acid sequence of SEQ ID NO: 8.
Embodiment 7. The antigenic polypeptide of embodiment 1 or 2, wherein the HSP-binding peptide comprises the amino acid sequence of SEQ ID NO: 9, optionally wherein the amino acid sequence of the HSP-binding peptide consists of the amino acid sequence of SEQ ID NO: 9.
Embodiment 8. The antigenic polypeptide of embodiment 1 or 2, wherein the HSP-binding peptide comprises the amino acid sequence of SEQ ID NO: 10, optionally wherein the amino acid sequence of the HSP-binding peptide consists of the amino acid sequence of SEQ ID NO: 10.
Embodiment 9. The antigenic polypeptide of embodiment 1 or 2, wherein the HSP-binding peptide comprises the amino acid sequence of SEQ ID NO: 11, optionally wherein the amino acid sequence of the HSP-binding peptide consists of the amino acid sequence of SEQ ID NO: 11.
Embodiment 10. The antigenic polypeptide of embodiment 1 or 2, wherein the HSP-binding peptide comprises the amino acid sequence of SEQ ID NO: 12, optionally wherein the amino acid sequence of the HSP-binding peptide consists of the amino acid sequence of SEQ ID NO: 12.
Embodiment 11. The antigenic polypeptide of embodiment 1 or 2, wherein the HSP-binding peptide comprises the amino acid sequence of SEQ ID NO: 13, optionally wherein the amino acid sequence of the HSP-binding peptide consists of the amino acid sequence of SEQ ID NO: 13.
Embodiment 12. The antigenic polypeptide of embodiment 1 or 2, wherein the HSP-binding peptide comprises the amino acid sequence of SEQ ID NO: 14, optionally wherein the amino acid sequence of the HSP-binding peptide consists of the amino acid sequence of SEQ ID NO: 14.
Embodiment 13. The antigenic polypeptide of embodiment 1 or 2, wherein the HSP-binding peptide comprises the amino acid sequence of SEQ ID NO: 15, optionally wherein the amino acid sequence of the HSP-binding peptide consists of the amino acid sequence of SEQ ID NO: 15.
Embodiment 14. The antigenic polypeptide of embodiment 1 or 2, wherein the HSP-binding peptide comprises the amino acid sequence of SEQ ID NO: 16, optionally wherein the amino acid sequence of the HSP-binding peptide consists of the amino acid sequence of SEQ ID NO: 16.
Embodiment 15. The antigenic polypeptide of embodiment 1 or 2, wherein the HSP-binding peptide comprises the amino acid sequence of SEQ ID NO: 17, optionally wherein the amino acid sequence of the HSP-binding peptide consists of the amino acid sequence of SEQ ID NO: 17.
Embodiment 16. The antigenic polypeptide of embodiment 1 or 2, wherein the HSP-binding peptide comprises the amino acid sequence of SEQ ID NO: 18, optionally wherein the amino acid sequence of the HSP-binding peptide consists of the amino acid sequence of SEQ ID NO: 18.
Embodiment 17. The antigenic polypeptide of embodiment 1 or 2, wherein the HSP-binding peptide comprises the amino acid sequence of SEQ ID NO: 19, optionally wherein the amino acid sequence of the HSP-binding peptide consists of the amino acid sequence of SEQ ID NO: 19.
Embodiment 18. The antigenic polypeptide of embodiment 1 or 2, wherein the HSP-binding peptide comprises the amino acid sequence of SEQ ID NO: 20, optionally wherein the amino acid sequence of the HSP-binding peptide consists of the amino acid sequence of SEQ ID NO: 20.
Embodiment 19. The antigenic polypeptide of embodiment 1 or 2, wherein the HSP-binding peptide comprises the amino acid sequence of SEQ ID NO: 21, optionally wherein the amino acid sequence of the HSP-binding peptide consists of the amino acid sequence of SEQ ID NO: 21.
Embodiment 20. The antigenic polypeptide of embodiment 1 or 2, wherein the HSP-binding peptide comprises the amino acid sequence of SEQ ID NO: 22, optionally wherein the amino acid sequence of the HSP-binding peptide consists of the amino acid sequence of SEQ ID NO: 22.
Embodiment 21. The antigenic polypeptide of embodiment 1 or 2, wherein the HSP-binding peptide comprises the amino acid sequence of SEQ ID NO: 23, optionally wherein the amino acid sequence of the HSP-binding peptide consists of the amino acid sequence of SEQ ID NO: 23.
Embodiment 22. The antigenic polypeptide of embodiment 1 or 2, wherein the HSP-binding peptide comprises the amino acid sequence of SEQ ID NO: 24, optionally wherein the amino acid sequence of the HSP-binding peptide consists of the amino acid sequence of SEQ ID NO: 24.
Embodiment 23. The antigenic polypeptide of embodiment 1 or 2, wherein the HSP-binding peptide comprises the amino acid sequence of SEQ ID NO: 25, optionally wherein the amino acid sequence of the HSP-binding peptide consists of the amino acid sequence of SEQ ID NO: 25.
Embodiment 24. The antigenic polypeptide of embodiment 1 or 2, wherein the HSP-binding peptide comprises the amino acid sequence of SEQ ID NO: 26, optionally wherein the amino acid sequence of the HSP-binding peptide consists of the amino acid sequence of SEQ ID NO: 26.
Embodiment 25. The antigenic polypeptide of embodiment 1 or 2, wherein the HSP-binding peptide comprises the amino acid sequence of SEQ ID NO: 27, optionally wherein the amino acid sequence of the HSP-binding peptide consists of the amino acid sequence of SEQ ID NO: 27.
Embodiment 26. The antigenic polypeptide of embodiment 1 or 2, wherein the HSP-binding peptide comprises the amino acid sequence of SEQ ID NO: 28, optionally wherein the amino acid sequence of the HSP-binding peptide consists of the amino acid sequence of SEQ ID NO: 28.
Embodiment 27. The antigenic polypeptide of embodiment 1 or 2, wherein the HSP-binding peptide comprises the amino acid sequence of SEQ ID NO: 29, optionally wherein the amino acid sequence of the HSP-binding peptide consists of the amino acid sequence of SEQ ID NO: 29.
Embodiment 28. The antigenic polypeptide of embodiment 1 or 2, wherein the HSP-binding peptide comprises the amino acid sequence of SEQ ID NO: 30, optionally wherein the amino acid sequence of the HSP-binding peptide consists of the amino acid sequence of SEQ ID NO: 30.
Embodiment 29. The antigenic polypeptide of embodiment 1 or 2, wherein the HSP-binding peptide comprises the amino acid sequence of SEQ ID NO: 31, optionally wherein the amino acid sequence of the HSP-binding peptide consists of the amino acid sequence of SEQ ID NO: 31.
Embodiment 30. The antigenic polypeptide of embodiment 1 or 2, wherein the HSP-binding peptide comprises the amino acid sequence of SEQ ID NO: 32, optionally wherein the amino acid sequence of the HSP-binding peptide consists of the amino acid sequence of SEQ ID NO: 32.
Embodiment 31. The antigenic polypeptide of embodiment 1 or 2, wherein the HSP-binding peptide comprises the amino acid sequence of SEQ ID NO: 33, optionally wherein the amino acid sequence of the HSP-binding peptide consists of the amino acid sequence of SEQ ID NO: 33.
Embodiment 32. The antigenic polypeptide of embodiment 1 or 2, wherein the HSP-binding peptide comprises the amino acid sequence of SEQ ID NO: 34, optionally wherein the amino acid sequence of the HSP-binding peptide consists of the amino acid sequence of SEQ ID NO: 34.
Embodiment 33. The antigenic polypeptide of embodiment 1 or 2, wherein the HSP-binding peptide comprises the amino acid sequence of SEQ ID NO: 35, optionally wherein the amino acid sequence of the HSP-binding peptide consists of the amino acid sequence of SEQ ID NO: 35.
Embodiment 34. The antigenic polypeptide of embodiment 1 or 2, wherein the HSP-binding peptide comprises the amino acid sequence of SEQ ID NO: 36, optionally wherein the amino acid sequence of the HSP-binding peptide consists of the amino acid sequence of SEQ ID NO: 36.
Embodiment 35. The antigenic polypeptide of embodiment 1 or 2, wherein the HSP-binding peptide comprises the amino acid sequence of SEQ ID NO: 37, optionally wherein the amino acid sequence of the HSP-binding peptide consists of the amino acid sequence of SEQ ID NO: 37.
Embodiment 36. The antigenic polypeptide of embodiment 1 or 2, wherein the HSP-binding peptide comprises the amino acid sequence of SEQ ID NO: 38, optionally wherein the amino acid sequence of the HSP-binding peptide consists of the amino acid sequence of SEQ ID NO: 38.
Embodiment 37. The antigenic polypeptide of embodiment 1 or 2, wherein the HSP-binding peptide comprises the amino acid sequence of SEQ ID NO: 39, optionally wherein the amino acid sequence of the HSP-binding peptide consists of the amino acid sequence of SEQ ID NO: 39.
Embodiment 38. The antigenic polypeptide of embodiment 1 or 2, wherein the HSP-binding peptide comprises the amino acid sequence of SEQ ID NO: 40, optionally wherein the amino acid sequence of the HSP-binding peptide consists of the amino acid sequence of SEQ ID NO: 40.
Embodiment 39. The antigenic polypeptide of embodiment 1 or 2, wherein the HSP-binding peptide comprises the amino acid sequence of SEQ ID NO: 41, optionally wherein the amino acid sequence of the HSP-binding peptide consists of the amino acid sequence of SEQ ID NO: 41.
Embodiment 40. The antigenic polypeptide of embodiment 1 or 2, wherein the HSP-binding peptide comprises the amino acid sequence of SEQ ID NO: 42, optionally wherein the amino acid sequence of the HSP-binding peptide consists of the amino acid sequence of SEQ ID NO: 42.
Embodiment 41. The antigenic polypeptide of any one of the preceding embodiments, wherein the MHC-binding peptide is 8 to 50 amino acids in length, optionally 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.
Embodiment 42. The antigenic polypeptide of any one of the preceding embodiments, wherein the C-terminus of the WIC-binding peptide is linked to the N-terminus of the HSP-binding peptide.
Embodiment 43. The antigenic polypeptide of any one of embodiments 1-41, wherein the N-terminus of the MHC-binding peptide is linked to the C-terminus of the HSP-binding peptide.
Embodiment 44. The antigenic polypeptide of any one of embodiments 1-43, wherein the HSP-binding peptide is linked to the WIC-binding peptide via a chemical linker.
Embodiment 45. The antigenic polypeptide of any one of embodiments 1-43, wherein the HSP-binding peptide is linked to the WIC-binding peptide via a peptide linker.
Embodiment 46. The antigenic polypeptide of embodiment 45, wherein the peptide linker comprises the amino acid sequence of SEQ ID NO: 43, optionally wherein the amino acid sequence of the peptide linker consists of the amino acid sequence of SEQ ID NO: 43.
Embodiment 47. The antigenic polypeptide of embodiment 45, wherein the peptide linker comprises the amino acid sequence of FR, optionally wherein the amino acid sequence of the peptide linker consists of the amino acid sequence of FR.
Embodiment 48. The antigenic polypeptide of embodiment 46 or 47, wherein the N-terminus of the MHC-binding peptide is linked to the C-terminus of:
(a) the amino acid sequence of X₁X₂X₃X₄X₅X₆X₇FFRK (SEQ ID NO: 68), wherein X₁ is omitted, N, F, or Q; X₂ is W, L, or F; X₃ is L or I; X₄ is R, L, or K; X₅ is L, W, or I; X₆ is T, L, F, K, R, or W; and X₇ is W, G, K, or F;
(b) the amino acid sequence of X₁LX₂LTX₃FFRK (SEQ ID NO: 69), wherein X₁ is W or F; X₂ is R or K; and X₃ is W, F, or G;
(c) the amino acid sequence of NX₁LX₂LTX₃FFRK (SEQ ID NO: 70), wherein X₁ is W or F; X₂ is R or K; and X₃ is W, F, or G;
(d) the amino acid sequence of WLX₁LTX₂FFRK (SEQ ID NO: 71), wherein X₁ is R or K; and X₂ is W or G;
(e) the amino acid sequence of NWLX₁LTX₂FFRK (SEQ ID NO: 72), wherein X₁ is R or K; and X₂ is W or G;
(f) the amino acid sequence of NWX₁X₂X₃X₄X₅FFRK (SEQ ID NO: 73), wherein X₁ is L or I; X₂ is L, R, or K; X₃ is L or I; X₄ is T, L, F, K, R, or W; and X₅ is W or K; or
(g) an amino acid sequence selected from the group consisting of SEQ ID NOs: 74-97.
Embodiment 49. The antigenic polypeptide of embodiment 46 or 47, wherein the N-terminus of the MHC-binding peptide is linked to the C-terminus of the amino acid sequence set forth in SEQ ID NO: 74.
Embodiment 50. The antigenic polypeptide of embodiment 46 or 47, wherein the N-terminus of the MHC-binding peptide is linked to the C-terminus of the amino acid sequence set forth in SEQ ID NO: 75.
Embodiment 51. The antigenic polypeptide of embodiment 46 or 47, wherein the N-terminus of the MHC-binding peptide is linked to the C-terminus of the amino acid sequence set forth in SEQ ID NO: 76.
Embodiment 52. The antigenic polypeptide of embodiment 46 or 47, wherein the N-terminus of the MHC-binding peptide is linked to the C-terminus of the amino acid sequence set forth in SEQ ID NO: 77.
Embodiment 53. The antigenic polypeptide of embodiment 46 or 47, wherein the N-terminus of the MHC-binding peptide is linked to the C-terminus of the amino acid sequence set forth in SEQ ID NO: 78.
Embodiment 54. The antigenic polypeptide of embodiment 46 or 47, wherein the N-terminus of the WIC-binding peptide is linked to the C-terminus of the amino acid sequence set forth in SEQ ID NO: 79.
Embodiment 55. The antigenic polypeptide of embodiment 46 or 47, wherein the N-terminus of the WIC-binding peptide is linked to the C-terminus of the amino acid sequence set forth in SEQ ID NO: 80.
Embodiment 56. The antigenic polypeptide of embodiment 46 or 47, wherein the N-terminus of the WIC-binding peptide is linked to the C-terminus of the amino acid sequence set forth in SEQ ID NO: 81.
Embodiment 57. The antigenic polypeptide of embodiment 46 or 47, wherein the N-terminus of the WIC-binding peptide is linked to the C-terminus of the amino acid sequence set forth in SEQ ID NO: 82.
Embodiment 58. The antigenic polypeptide of embodiment 46 or 47, wherein the N-terminus of the WIC-binding peptide is linked to the C-terminus of the amino acid sequence set forth in SEQ ID NO: 83.
Embodiment 59. The antigenic polypeptide of embodiment 46 or 47, wherein the N-terminus of the WIC-binding peptide is linked to the C-terminus of the amino acid sequence set forth in SEQ ID NO: 84.
Embodiment 60. The antigenic polypeptide of embodiment 46 or 47, wherein the N-terminus of the WIC-binding peptide is linked to the C-terminus of the amino acid sequence set forth in SEQ ID NO: 85.
Embodiment 61. The antigenic polypeptide of embodiment 46 or 47, wherein the N-terminus of the WIC-binding peptide is linked to the C-terminus of the amino acid sequence set forth in SEQ ID NO: 86.
Embodiment 62. The antigenic polypeptide of embodiment 46 or 47, wherein the N-terminus of the WIC-binding peptide is linked to the C-terminus of the amino acid sequence set forth in SEQ ID NO: 87.
Embodiment 63. The antigenic polypeptide of embodiment 46 or 47, wherein the N-terminus of the WIC-binding peptide is linked to the C-terminus of the amino acid sequence set forth in SEQ ID NO: 88.
Embodiment 64. The antigenic polypeptide of embodiment 46 or 47, wherein the N-terminus of the WIC-binding peptide is linked to the C-terminus of the amino acid sequence set forth in SEQ ID NO: 89.
Embodiment 65. The antigenic polypeptide of embodiment 46 or 47, wherein the N-terminus of the WIC-binding peptide is linked to the C-terminus of the amino acid sequence set forth in SEQ ID NO: 90.
Embodiment 66. The antigenic polypeptide of embodiment 46 or 47, wherein the N-terminus of the WIC-binding peptide is linked to the C-terminus of the amino acid sequence set forth in SEQ ID NO: 91.
Embodiment 67. The antigenic polypeptide of embodiment 46 or 47, wherein the N-terminus of the WIC-binding peptide is linked to the C-terminus of the amino acid sequence set forth in SEQ ID NO: 92.
Embodiment 68. The antigenic polypeptide of embodiment 46 or 47, wherein the N-terminus of the WIC-binding peptide is linked to the C-terminus of the amino acid sequence set forth in SEQ ID NO: 93.
Embodiment 69. The antigenic polypeptide of embodiment 46 or 47, wherein the N-terminus of the WIC-binding peptide is linked to the C-terminus of the amino acid sequence set forth in SEQ ID NO: 94.
Embodiment 70. The antigenic polypeptide of embodiment 46 or 47, wherein the N-terminus of the WIC-binding peptide is linked to the C-terminus of the amino acid sequence set forth in SEQ ID NO: 95.
Embodiment 71. The antigenic polypeptide of embodiment 46 or 47, wherein the N-terminus of the WIC-binding peptide is linked to the C-terminus of the amino acid sequence set forth in SEQ ID NO: 96.
Embodiment 72. The antigenic polypeptide of embodiment 46 or 47, wherein the N-terminus of the WIC-binding peptide is linked to the C-terminus of the amino acid sequence set forth in SEQ ID NO: 97.
Embodiment 73. The isolated polypeptide of embodiment 46 or 47, wherein the C-terminus of the WIC-binding peptide is linked to the N-terminus of:
(a) the amino acid sequence of FFRKX₁X₂X₃X₄X₅X₆X₇ (SEQ ID NO: 44), wherein X₁ is omitted, N, F, or Q; X₂ is W, L, or F; X₃ is L or I; X₄ is R, L, or K; X₅ is L, W, or I; X₆ is T, L, F, K, R, or W; and X₇ is W, G, K, or F;
(b) the amino acid sequence of FFRKX₁LX₂LTX₃ (SEQ ID NO: 45), wherein X₁ is W or F; X₂ is R or K; and X₃ is W, F, or G;
(c) the amino acid sequence of FFRKNX₁LX₂LTX₃ (SEQ ID NO: 46), wherein X₁ is W or F; X₂ is R or K; and X₃ is W, F, or G;
(d) the amino acid sequence of FFRKWLX₁LTX₂ (SEQ ID NO: 47), wherein X₁ is R or K; and X₂ is W or G;
(e) the amino acid sequence of FFRKNWLX₁LTX₂ (SEQ ID NO: 48), wherein X₁ is R or K; and X₂ is W or G;
(f) the amino acid sequence of FFRKNWX₁X₂X₃X₄X₅ (SEQ ID NO: 49), wherein X₁ is L or I; X₂ is L, R, or K; X₃ is L or I; X₄ is T, L, F, K, R, or W; and X₅ is W or K; or
(g) an amino acid sequence selected from the group consisting of SEQ ID NOs: 50-67.
Embodiment 74. The antigenic polypeptide of embodiment 46 or 47, wherein the C-terminus of the WIC-binding peptide is linked to the N-terminus of the amino acid sequence set forth in SEQ ID NO: 50.
Embodiment 75. The antigenic polypeptide of embodiment 46 or 47, wherein the C-terminus of the WIC-binding peptide is linked to the N-terminus of the amino acid sequence set forth in SEQ ID NO: 51.
Embodiment 76. The antigenic polypeptide of embodiment 46 or 47, wherein the C-terminus of the WIC-binding peptide is linked to the N-terminus of the amino acid sequence set forth in SEQ ID NO: 52.
Embodiment 77. The antigenic polypeptide of embodiment 46 or 47, wherein the C-terminus of the WIC-binding peptide is linked to the N-terminus of the amino acid sequence set forth in SEQ ID NO: 53.
Embodiment 78. The antigenic polypeptide of embodiment 46 or 47, wherein the C-terminus of the WIC-binding peptide is linked to the N-terminus of the amino acid sequence set forth in SEQ ID NO: 54.
Embodiment 79. The antigenic polypeptide of embodiment 46 or 47, wherein the C-terminus of the WIC-binding peptide is linked to the N-terminus of the amino acid sequence set forth in SEQ ID NO: 55.
Embodiment 80. The antigenic polypeptide of embodiment 46 or 47, wherein the C-terminus of the WIC-binding peptide is linked to the N-terminus of the amino acid sequence set forth in SEQ ID NO: 56.
Embodiment 81. The antigenic polypeptide of embodiment 46 or 47, wherein the C-terminus of the WIC-binding peptide is linked to the N-terminus of the amino acid sequence set forth in SEQ ID NO: 57.
Embodiment 82. The antigenic polypeptide of embodiment 46 or 47, wherein the C-terminus of the WIC-binding peptide is linked to the N-terminus of the amino acid sequence set forth in SEQ ID NO: 58.
Embodiment 83. The antigenic polypeptide of embodiment 46 or 47, wherein the C-terminus of the WIC-binding peptide is linked to the N-terminus of the amino acid sequence set forth in SEQ ID NO: 59.
Embodiment 84. The antigenic polypeptide of embodiment 46 or 47, wherein the C-terminus of the WIC-binding peptide is linked to the N-terminus of the amino acid sequence set forth in SEQ ID NO: 60.
Embodiment 85. The antigenic polypeptide of embodiment 46 or 47, wherein the C-terminus of the WIC-binding peptide is linked to the N-terminus of the amino acid sequence set forth in SEQ ID NO: 61.
Embodiment 86. The antigenic polypeptide of embodiment 46 or 47, wherein the C-terminus of the WIC-binding peptide is linked to the N-terminus of the amino acid sequence set forth in SEQ ID NO: 62.
Embodiment 87. The antigenic polypeptide of embodiment 46 or 47, wherein the C-terminus of the WIC-binding peptide is linked to the N-terminus of the amino acid sequence set forth in SEQ ID NO: 63.
Embodiment 88. The antigenic polypeptide of embodiment 46 or 47, wherein the C-terminus of the WIC-binding peptide is linked to the N-terminus of the amino acid sequence set forth in SEQ ID NO: 64.
Embodiment 89. The antigenic polypeptide of embodiment 46 or 47, wherein the C-terminus of the WIC-binding peptide is linked to the N-terminus of the amino acid sequence set forth in SEQ ID NO: 65.
Embodiment 90. The antigenic polypeptide of embodiment 46 or 47, wherein the C-terminus of the WIC-binding peptide is linked to the N-terminus of the amino acid sequence set forth in SEQ ID NO: 66.
Embodiment 91. The antigenic polypeptide of embodiment 46 or 47, wherein the C-terminus of the WIC-binding peptide is linked to the N-terminus of the amino acid sequence set forth in SEQ ID NO: 67.
Embodiment 92. The antigenic polypeptide of embodiment 1, comprising an amino acid sequence selected from the group consisting of SEQ ID NOs: 3001-8806, 8809, and 8810.
Embodiment 93. The antigenic polypeptide of any one of the preceding embodiments, wherein the antigenic polypeptide is 15 to 100 amino acids in length, optionally 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length.
Embodiment 94. The antigenic polypeptide of embodiment 1, wherein the amino acid sequence of the antigenic polypeptide consists of an amino acid sequence selected from the group consisting of SEQ ID NOs: 3001-8806, 8809, and 8810.
Embodiment 95. The antigenic polypeptide of any one of the preceding embodiments, wherein the antigenic polypeptide is chemically synthesized.
Embodiment 96. The antigenic polypeptide of any one of the preceding embodiments, comprising a phosphopeptide selected from the group consisting of SEQ ID NOs: 98-3000 and 8808, wherein a phosphorylated amino acid residue of the phosphopeptide is replaced by a non-hydrolyzable mimetic of the phosphorylated amino acid residue.
Embodiment 97. A composition comprising: (i) at least one of the antigenic polypeptides of any one of embodiments 1-96; (ii) at least one polypeptide comprising an amino acid sequence selected from the group consisting of SEQ ID NOs: 98-3000 and 8808, optionally, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 different polypeptides comprising an amino acid sequence selected from the group consisting of SEQ ID NOs: 98-3000 and 8808; (iii) at least one polypeptide, wherein the amino acid sequence of the polypeptide consists of an amino acid sequence selected from the group consisting of SEQ ID NOs: 98-3000 and 8808, optionally 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 different polypeptides, wherein the amino acid sequence of each polypeptide consists of an amino acid sequence selected from the group consisting of SEQ ID NOs: 98-3000 and 8808; or (iv) at least one polypeptide, wherein the polypeptide consists of an amino acid sequence selected from the group consisting of SEQ ID NOs: 98-3000 and 8808, optionally 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 different polypeptides, wherein each polypeptide consists of an amino acid sequence selected from the group consisting of SEQ ID NOs: 98-3000 and 8808.
Embodiment 98. A composition comprising a complex of the antigenic polypeptide of any one of embodiments 1-96 and a purified stress protein.
Embodiment 99. The composition of embodiment 98, wherein the stress protein is selected from the group consisting of Hsc70, Hsp70, Hsp90, Hsp110, Grp170, Gp96, Calreticulin, and a mutant or fusion protein thereof.
Embodiment 100. The composition of embodiment 99, wherein the stress protein is an Hsc70, optionally a human Hsc70.
Embodiment 101. The composition of embodiment 100, wherein the Hsc70 comprises the amino acid sequence of SEQ ID NO: 8807.
Embodiment 102. The composition of embodiment 100, wherein the amino acid sequence of the Hsc70 consists of the amino acid sequence of SEQ ID NO: 8807.
Embodiment 103. The composition of any one of embodiments 98-102, wherein the stress protein is a recombinant protein.
Embodiment 104. The composition any one of embodiments 97-103, comprising 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 different antigenic polypeptides.
Embodiment 105. The composition of embodiment 104, wherein each of the different polypeptides comprise the same HSP-binding peptide and a different MHC-binding peptide.
Embodiment 106. The composition of any one of embodiments 97-105, wherein the total amount of the antigenic polypeptide(s) in the composition is about 0.1 to 20 nmol, optionally about 3, 4, 5, or 6 nmol.
Embodiment 107. The composition of any one of embodiments 98-106, wherein the amount of the stress protein in the composition is about 10 μg to 600 μg, optionally about 120 μg, 240 μg, or 480 μg.
Embodiment 108. The composition of any one of embodiments 98-107, wherein the molar ratio of the antigenic polypeptide(s) to the stress protein is about 0.5:1 to about 5:1, optionally about 1:1, 1.25:1, 1.5:1, 2:1, 2.5:1, 3:1, 3.5:1, 4:1, 4.5:1, or 5:1.
Embodiment 109. The composition of any one of embodiments 97-108, wherein the composition further comprises an adjuvant.
Embodiment 110. The composition of embodiment 109, wherein the adjuvant comprises a saponin or an immunostimulatory nucleic acid.
Embodiment 111. The composition of embodiment 110, wherein the adjuvant comprises QS-21.
Embodiment 112. The composition of embodiment 111, wherein the amount of the QS-21 in the composition is about 10 μg to about 200 optionally about 25 μg, 50 μg, 75 μg, 100 μg, 125 μg, 150 μg, 175 μg, or 200 μg.
Embodiment 113. The composition of any one of embodiments 109-112, wherein the adjuvant comprises a TLR agonist, optionally a TLR4 agonist, TLR5 agonist, TLR7 agonist, TLR8 agonist, and/or TLR9 agonist.
Embodiment 114. The composition of any one of embodiments 97-113, further comprising a pharmaceutically acceptable carrier or excipient.
Embodiment 115. The composition of embodiment 114, wherein the composition is in a unit dosage form.
Embodiment 116. A method of inducing a cellular immune response to a polypeptide (e.g., an antigenic polypeptide) in a subject, the method comprising administering to the subject an effective amount of the antigenic polypeptide of any one of embodiments 1-96 or the composition of any one of embodiments 97-115.
Embodiment 117. The method of embodiment 116, wherein the subject has cancer, optionally Acute Myeloid Leukemia (AML) or colorectal cancer.
Embodiment 118. A method of treating a disease in a subject, the method comprising administering to the subject an effective amount of the antigenic polypeptide of any one of embodiments 1-96 or the composition of any one of embodiments 97-115.
Embodiment 119. The method of embodiment 118, wherein the disease is an infection of a pathogenic microbe.
Embodiment 120. The method of any one of embodiments 116-119, wherein the composition is administered to the subject weekly for four weeks.
Embodiment 121. The method of embodiment 120, wherein at least two further doses of the composition are administered biweekly to the subject after the four weekly doses.
Embodiment 122. The method of embodiment 120 or 121, wherein at least one booster dose of the composition is administered three months after the final weekly or biweekly dose.
Embodiment 123. The method of embodiment 122, wherein the composition is further administered every three months for at least 1 year.
Embodiment 124. The method of any one of embodiments 116-123, further comprising administering to the subject lenalidomide, dexamethasone, interleukin-2, recombinant interferon alfa-2b, or PEG-interferon alfa-2b.
Embodiment 125. The method of any one of embodiments 116-124, further comprising administering to the subject an indoleamine dioxygenase-1 (IDO-1) inhibitor.
Embodiment 126. The method of embodiment 125, wherein the IDO-1 inhibitor is 4-amino-N-(3-chloro-4-fluorophenyl)-N′-hydroxy-1,2,5-oxadiazole-3-carboximidamide.
Embodiment 127. The method of any one of embodiments 116-126, further comprising administering to the subject an immune checkpoint antibody.
Embodiment 128. The method of embodiment 127, wherein the immune checkpoint antibody is selected from the group consisting of an agonistic anti-GITR antibody, an agonistic anti-OX40 antibody, an antagonistic anti-PD-1 antibody, an antagonistic anti-CTLA-4 antibody, an antagonistic anti-TIM-3 antibody, an antagonistic anti-LAG-3 antibody, an antagonistic anti-TIGIT antibody, an agonistic anti-CD96 antibody, an antagonistic anti-VISTA antibody, an antagonistic anti-CD73 antibody, an agonistic anti-CD137 antibody, an antagonist anti-CEACAM1 antibody, an agonist anti-ICOS antibody, and/or an antigen-binding fragment thereof.
Embodiment 129. A kit comprising a first container containing the antigenic polypeptide of any one of embodiments 1-96, or the composition of any one of embodiments 97-115 and a second container containing a purified stress protein capable of binding to the antigenic polypeptide.
Embodiment 130. The kit of embodiment 129, wherein the total amount of the polypeptide(s) in the first container is about 0.1 to 20 nmol, optionally about 3, 4, 5, or 6 nmol.
Embodiment 131. The kit of embodiment 129 or 130, wherein the stress protein is selected from the group consisting of Hsc70, Hsp70, Hsp90, Hsp110, Grp170, Gp96, Calreticulin, and a mutant or fusion protein thereof.
Embodiment 132. The kit of embodiment 131, wherein the stress protein is an Hsc70, optionally a human Hsc70.
Embodiment 133. The kit of embodiment 132, wherein the Hsc70 comprises the amino acid sequence of SEQ ID NO: 8807.
Embodiment 134. The kit of embodiment 132, wherein the amino acid sequence of the Hsc70 consists of the amino acid sequence of SEQ ID NO: 8807.
Embodiment 135. The kit of any one of embodiments 129-134, wherein the stress protein is a recombinant protein.
Embodiment 136. The kit of any one of embodiments 129-135, wherein the amount of the stress protein in the second container is about 10 μg to 600 μg, optionally about 120 μg, 240 μg, or 480 μg.
Embodiment 137. The kit of any one of embodiments 129-136, wherein the molar ratio of the polypeptide to the stress protein is about 0.5:1 to 5:1, optionally about 1:1, 1.25:1, 1.5:1, 2:1, 2.5:1, 3:1, 3.5:1, 4:1, 4.5:1, or 5:1.
Embodiment 138. The kit of any one of embodiments 129-137, further comprising a third container containing an adjuvant.
Embodiment 139. The kit of embodiment 138, wherein the adjuvant comprises a saponin or an immunostimulatory nucleic acid.
Embodiment 140. The kit of embodiment 139, wherein the adjuvant comprises QS-21.
Embodiment 141. The kit of embodiment 140, wherein the amount of the QS-21 in the third container is about 10 μg to about 200 μg, optionally about 25 μg, 50 μg, 75 μg, 100 μg, 125 μg, 150 μg, 175 μg, or 200 μg.
Embodiment 142. The kit of any one of embodiments 138-141, wherein the adjuvant comprises a TLR agonist, optionally a TLR4 agonist, TLR5 agonist, TLR7 agonist, TLR8 agonist, and/or TLR9 agonist.
Embodiment 143. A method of making a vaccine, the method comprising mixing one or more of the polypeptides of any one of embodiments 1-96, or the composition of any one of embodiments 97-115, with a purified stress protein under suitable conditions such that the purified stress protein binds to at least one of the polypeptides.
Embodiment 144. The method of embodiment 143, wherein the stress protein is selected from the group consisting of Hsc70, Hsp70, Hsp90, Hsp110, Grp170, Gp96, Calreticulin, and a mutant or fusion protein thereof.
Embodiment 145. The method of embodiment 144, wherein the stress protein is an Hsc70, optionally human a Hsc70.
Embodiment 146. The method of embodiment 145, wherein the Hsc70 comprises the amino acid sequence of SEQ ID NO: 8807.
Embodiment 147. The method of embodiment 145, wherein the amino acid sequence of the Hsc70 consists of the amino acid sequence of SEQ ID NO: 8807.
Embodiment 148. The method of any one of embodiments 143-147, wherein the stress protein is a recombinant protein.
Embodiment 149. The method of any one of embodiments 143-148, wherein the molar ratio of the polypeptide to the stress protein is about 0.5:1 to 5:1, optionally about 1:1, 1.25:1, 1.5:1, 2:1, 2.5:1, 3:1, 3.5:1, 4:1, 4.5:1, or 5:1.
Embodiment 150. The method of any one of embodiments 143-149, wherein the suitable conditions comprise a temperature of about 37° C.

### 6. DETAILED DESCRIPTION

The instant disclosure provides novel antigenic polypeptides comprising tumor-associated peptides, and compositions comprising the same. Such antigenic polypeptides and compositions are particularly useful as immunotherapeutics (e.g., cancer vaccines). Also provided are methods of inducing a cellular immune response using such polypeptides and compositions, methods of treating a disease using such polypeptides and compositions, kits comprising such polypeptides and compositions, and methods of making such compositions.

### 6.1 Definitions

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including”, as well as other forms, such as “includes” and “included”, is not limiting.

As used herein, the terms “about” and “approximately,” when used to modify a numeric value or numeric range, indicate that deviations of 5% to 10% above (e.g., up to 5% to 10% above) and 5% to 10% below (e.g., up to 5% to 10% below) the recited value or range remain within the intended meaning of the recited value or range.

As used herein, the term “antigenic polypeptide” refers to a non-naturally occurring polymer comprising one or more peptides (e.g., an MHC-binding peptide and/or an HSP-binding peptide). An antigenic polypeptide can comprise one or more non-amino-acid-residue structures. In certain embodiments, an antigenic polypeptide comprises a chemical linker, e.g., a chemical linker linking two peptide portions of the polypeptide.

As used herein, the terms “major histocompatibility complex” and “MHC” are used interchangeably and refer to an MHC class I molecule and/or an MHC class II molecule.

As used herein, the terms “human leukocyte antigen” and “HLA” are used interchangeably and refer to major histocompatibility complex (MHC) in humans. An HLA molecule may be a class I MHC molecule (e.g., HLA-A, HLA-B, HLA-C) or a class II MHC molecule (e.g., HLA-DP, HLA-DQ, HLA-DR).

As used herein, the terms “major histocompatibility complex-binding peptide” and “MHC-binding peptide” are used interchangeably and refer to a peptide that binds to or is predicted to bind to an MHC molecule, e.g., such that the peptide is capable of being presented by the MHC molecule to a T-cell.

As used herein, the terms “heat shock protein-binding peptide” and “HSP-binding peptide” are used interchangeably and refer to a peptide that non-covalently binds to a heat shock protein (HSP).

As used herein, the term “peptide linker” refers to a peptide bond or a peptide sequence that links a C-terminal amino acid residue of a first peptide to an N-terminal amino acid residue of a second peptide.

As used herein, the term “chemical linker” refers to any chemical bond or moiety that is capable of linking two molecules (e.g., two peptides), wherein the bond or moiety is not a peptide linker.

As used herein, the term “O-GlcNAcylated” means O-GlcNAc modified, wherein the O-GlcNAc is fused either directly or indirectly to the modified amino acid, as described in Malaker, S. A. et al. “Identification of Glycopeptides as Post-Translationally Modified Neoantigens in Leukemia” Cancer Immunol Res. 5(5):376-384 (2017), which is incorporated by reference in its entirety herein. One of skill in the art would understand the term “hexose-GlcNAcylated” to have the meaning described in Malaker, S. A. et al. “Identification and Characterization of Complex Glycosylated Peptides Presented by the MHC Class II Processing Pathway in Melanoma” J. Proteome Res. 16(1):228-237 (2017), which is incorporated by reference in its entirety herein.

As used herein, the terms “treat,” “treating,” and “treatment” refer to methods that generally involve administration of an agent (e.g., a polypeptide disclosed herein) to a subject having a disease or disorder, or predisposed to having such a disease or disorder, in order to cure, delay, reduce the severity of, or ameliorate one or more symptoms of the disease or disorder, or in order to prolong the survival of the subject beyond that expected in the absence of such treatment.

As used herein, the term “effective amount” in the context of the administration of a therapy to a subject refers to the amount of a therapy that achieves a desired prophylactic or therapeutic effect.

As used herein, the term “subject” includes any human or non-human animal.

### 6.2 Antigenic Polypeptides

In one aspect, the instant disclosure provides an antigenic polypeptide comprising a tumor-associated MHC-binding peptide and an HSP-binding peptide.

Exemplary HSP-binding peptides are set forth in Table 1 herein. Exemplary MHC-binding peptides are set forth in Tables 2 and 3 herein. Exemplary antigenic polypeptides are set forth in Tables 4-7 herein.

TABLE 1
Amino acid sequences of exemplary HSP-binding peptides,
linkers, and HSPs

| | | SEQ ID |
|---|---|---|
| Description | Amino Acid Sequence | NO |
| Consensus | X₁X₂X₃X₄X₅X₆X₇, wherein: | 1 |
| sequence 1 | X₁ is omitted, N, F, or Q; | |
| | X₂ is W, L, or F; | |
| | X₃ is L or I; | |
| | X₄ is R, L, or K; | |
| | X₅ is L, W, or I; | |
| | X₆ is T, L, F, K, R, or W; and | |
| | X₇ is W, G, K, or F | |
| Consensus | X₁LX₂LTX₃, wherein: | 2 |
| sequence 2 | X₁ is W or F; | |
| | X₂ is R or K; and | |
| | X₃ is W, F, or G | |
| Consensus | NX₁LX₂LTX₃, wherein: | 3 |
| sequence 3 | X₁ is W or F; | |
| | X₂ is R or K; and | |
| | X₃ is W, F, or G | |
| Consensus | WLX₁LTX₂, wherein: | 4 |
| sequence 4 | X₁ is R or K; and | |
| | X₂ is W or G | |
| Consensus | NWLX₁LTX₂, wherein: | 5 |
| sequence 5 | X₁ is R or K; and | |
| | X₂ is W or G | |
| Consensus | NWX₁X₂X₃X₄X₅, wherein: | 6 |
| sequence 6 | X₁ is L or I; | |
| | X₂ is L, R, or K; | |
| | X₃ is L or I; | |
| | X₄ is T, L, F, K, R, or W; and | |
| | X₅ is W or K | |
| HSP1 | NLLRLTG | 7 |
| HSP016 | WLRLTW | 8 |
| HSP017 | NWLRLTW | 9 |
| HSP018 | WLKLTW | 10 |
| HSP019 | NWLKLTW | 11 |
| HSP020 | WLRLTG | 12 |
| HSP021 | NWLRLTG | 13 |
| HSP022 | FLRLTF | 14 |
| HSP023 | NFLRLTF | 15 |
| HSP024 | WLRLTF | 16 |
| HSP025 | NWLRLTF | 17 |
| HSP040 | WLKLTF | 18 |
| HSP041 | NWLKLTF | 19 |
| HSP042 | WLKLTG | 20 |
| HSP043 | NWLKLTG | 21 |
| HSP044 | FLRLTW | 22 |
| HSP045 | NFLRLTW | 23 |
| HSP046 | FLRLTG | 24 |
| HSP047 | NFLRLTG | 25 |
| HSP048 | FLKLTW | 26 |
| HSP049 | NFLKLTW | 27 |
| HSP050 | FLKLTF | 28 |
| HSP051 | NFLKLTF | 29 |
| HSP103 | FLKLTG | 30 |
| HSP104 | NFLKLTG | 31 |
| HSP185 | NWLLLTW | 32 |
| HSP186 | NLLRWTG | 33 |
| HSP188 | FWLRLTW | 34 |
| HSP189 | NWLRLLW | 35 |
| HSP190 | NWLRLFW | 36 |
| HSP191 | NWLRLKW | 37 |
| HSP192 | NWIRITW | 38 |
| HSP193 | QWLRLTW | 39 |
| HSP194 | NWLKLKW | 40 |
| HSP195 | NWLKLRW | 41 |
| HSP196 | NWLKLWK | 42 |
| Linker1 | FFRK | 43 |
| Linker2 | FR | N/A |
| Consensus | FFRKX₁X₂X₃X₄X₅X₆X₇, wherein: | 44 |
| sequence 1 | X₁ is omitted, N, F, or Q; | |
| with N- | X₂ is W, L, or F; | |
| terminal | X₃ is L or I; | |
| linker | X₄ is R, L, or K; | |
| | X₅ is L, W, or I; | |
| | X₆ is T, L, F, K, R, or W; and | |
| | X₇ is W, G, K, or F | |
| Consensus | FFRKX₁LX₂LTX₃, wherein: | 45 |
| sequence 2 | X₁ is W or F; | |
| with N- | X₂ is R or K; and | |
| terminal | X₃ is W, F, or G | |
| linker | | |
| Consensus | FFRKNX₁LX₂LTX₃, wherein: | 46 |
| sequence 3 | X₁ is W or F; | |
| with N- | X₂ is R or K; and | |
| terminal | X₃ is W, F, or G | |
| linker | | |
| Consensus | FFRKWLX₁LTX₂, wherein: | 47 |
| sequence 4 | X₁ is R or K; and | |
| with N- | X₂ is W or G | |
| terminal | | |
| linker | | |
| Consensus | FFRKNWLX₁LTX₂, wherein: | 48 |
| sequence 5 | X₁ is R or K; and | |
| with N- | X₂ is W or G | |
| terminal | | |
| linker | | |
| Consensus | FFRKNWX₁X₂X₃X₄X₅, wherein: | 49 |
| sequence 6 | X₁ is L or I; | |
| with N- | X₂ is L, R, or K; | |
| terminal | X₃ is L or I; | |
| linker | X₄ is T, L, F, K, R, or W; and | |
| | X₅ is W or K | |
| Linker1- | FFRKNLLRLTG | 50 |
| HSP1 | | |
| Linker2- | FRNLLRLTG | 51 |
| HSP1 | | |
| HSP001 | FFRKNLLRLTG | 52 |
| HSP003 | FFRKNWLLLTW | 53 |
| HSP004 | FFRKNLLRWTG | 54 |
| HSP006 | FFRKNWLRLTW | 55 |
| HSP012 | FFRKNWLKLTW | 56 |
| HSP013 | FFRKNWIRITW | 57 |
| HSP014 | FFRKQWLRLTW | 58 |
| HSP026 | FFRKNWLRLTG | 59 |
| HSP027 | FFRKNFLRLTF | 60 |
| HSP028 | FRNWLRLTW | 61 |
| HSP029 | FRNWLKLTW | 62 |
| HSP030 | FRNWLRLTG | 63 |
| HSP031 | FRNFLRLTF | 64 |
| HSP055 | FFRKNWLKLKW | 65 |
| HSP057 | FFRKNWLKLRW | 66 |
| HSP058 | FFRKNWLKLWK | 67 |
| Consensus | X₁X₂X₃X₄X₅X₆X₇FFRK, wherein: | 68 |
| sequence 1 | X₁ is omitted, N, F, or Q; | |
| with C- | X₂ is W, L, or F; | |
| terminal | X₃ is L or I; | |
| linker | X₄ is R, L, or K; | |
| | X₅ is L, W, or I; | |
| | X₆ is T, L, F, K, R, or W; and | |
| | X₇ is W, G, K, or F | |
| Consensus | X₁LX₂LTX₃FFRK, wherein: | 69 |
| sequence 2 | X₁ is W or F; | |
| with C- | X₂ is R or K; and | |
| terminal | X₃ is W, F, or G | |
| linker | | |
| Consensus | NX₁LX₂LTX₃FFRK, wherein: | 70 |
| sequence 3 | X₁ is W or F; | |
| with C- | X₂ is R or K; and | |
| terminal | X₃ is W, F, or G | |
| linker | | |
| Consensus | WLX₁LTX₂FFRK, wherein: | 71 |
| sequence 4 | X₁ is R or K; and | |
| with C- | X₂ iS W or G | |
| terminal | | |
| linker | | |
| Consensus | NWLX₁LTX₂FFRK, wherein: | 72 |
| sequence 5 | X₁ is R or K; and | |
| with C- | X₂ iS W or G | |
| terminal | | |
| linker | | |
| Consensus | NWX₁X₂X₃X₄X₅FFRK, wherein: | 73 |
| sequence 6 | X₁ is L or I; | |
| with C- | X₂ is L, R, or K; | |
| terminal | X₃ is L or I; | |
| linker | X₄ is T, L, F, K, R, or W; and | |
| | X₅ is W or K | |
| HSP1- | NLLRLTGFFRK | 74 |
| Linker1 | | |
| HSP1- | NLLRLTGFR | 75 |
| Linker2 | | |
| HSP032 | NWLRLTWFFRK | 76 |
| HSP033 | NWLKLTWFFRK | 77 |
| HSP034 | NWLRLTGFFRK | 78 |
| HSP035 | NFLRLTFFFRK | 79 |
| HSP036 | NWLRLTWFR | 80 |
| HSP037 | NWLKLTWFR | 81 |
| HSP038 | NWLRLTGFR | 82 |
| HSP039 | NFLRLTFFR | 83 |
| HSP197 | NLLRLTWFFRK | 84 |
| HSP198 | NRLLLTGFFRK | 85 |
| HSP199 | NWLLLTWFFRK | 86 |
| HSP200 | NLLRWTGFFRK | 87 |
| HSP201 | NRLWLTGFFRK | 88 |
| HSP202 | FWLRLTWFFRK | 89 |
| HSP203 | NWLRLLWFFRK | 90 |
| HSP204 | NWLRLFWFFRK | 91 |
| HSP205 | NWLRLKWFFRK | 92 |
| HSP206 | NWIRITWFFRK | 93 |
| HSP207 | QWLRLTWFFRK | 94 |
| HSP208 | NWLKLKWFFRK | 95 |
| HSP209 | NWLKLRWFFRK | 96 |
| HSP210 | NWLKLWKFFRK | 97 |
| rh-Hsc70 | SKGPAVGIDLGTTYSCVGVFQHGKVEIIANDQGNRTTPSYVA | 8807 |
| | FTDTERLIGDAAKNQVAMNPTNTVFDAKRLIGRRFDDAVVQS | |
| | DMKHWPFMVVNDAGRPKVQVEYKGETKSFYPEEVSSMVLTKM | |
| | KEIAEAYLGKTVTNAVVTVPAYFNDSQRQATKDAGTIAGLNV | |
| | LRIINEPTAAAIAYGLDKKVGAERNVLIFDLGGGTFDVSILT | |
| | IEDGIFEVKSTAGDTHLGGEDFDNRMVNHFIAEFKRKHKKDI | |
| | SENKRAVRRLRTACERAKRTLSSSTQASIEIDSLYEGIDFYT | |
| | SITRARFEELNADLFRGTLDPVEKALRDAKLDKSQIHDIVLV | |
| | GGSTRIPKIQKLLQDFFNGKELNKSINPDEAVAYGAAVQAAI | |
| | LSGDKSENVQDLLLLDVTPLSLGIETAGGVMTVLIKRNTTIP | |
| | TKQTQTFTTYSDNQPGVLIQVYEGERAMTKDNNLLGKFELTG | |
| | IPPAPRGVPQIEVTFDIDANGILNVSAVDKSTGKENKITITN | |
| | DKGRLSKEDIERMVQEAEKYKAEDEKQRDKVSSKNSLESYAF | |
| | NMKATVEDEKLQGKINDEDKQKILDKCNEIINWLDKNQTAEK | |
| | EEFEHQQKELEKVCNPIITKLYQSAGGMPGGMPGGFPGGGAP | |
| | PSGGASSGPTIEEVD | |

TABLE 2
Amino acid sequences of exemplary
MHC-binding peptides

| SEQ ID NO | Amino Acid Sequence |
|---|---|
| 98 | (AcS)AARESHPHGVKRSAsPDDDLG |
| 99 | AAEsPSFL |
| 100 | AASNFKsPVKTIR |
| 101 | ADLsPEREV |
| 102 | AEDEIGtPRKF |
| 103 | AEDEIGtPRKY |
| 104 | AEEEIGtPRKF |
| 105 | AEEEIGtPRKW |
| 106 | AEEEIGtPRKY |
| 107 | AENARSAsF |
| 108 | AENsPTRQQF |
| 109 | AENsPTRQQW |
| 110 | AENsPTRQQY |
| 111 | AENsSSREL |
| 112 | AEQGsPRVSY |
| 113 | AESsPTAGKKF |
| 114 | AESsPTAGKKL |
| 115 | AESsPTAGKKW |
| 116 | AESsPTAGKKY |
| 117 | AGDsPGSQF |
| 118 | AILsPAFKV |
| 119 | AIMRsPQMV |
| 120 | AIsDLQQL |
| 121 | AKLsETIS |
| 122 | ALAAsPHAV |
| 123 | ALDsGASLLHL |
| 124 | ALDsGASLLHV |
| 125 | ALGNtPPFL |
| 126 | ALGsRESLATI |
| 127 | ALGsRESLATV |
| 128 | ALIHQsLGL |
| 129 | ALIHQsLGV |
| 130 | ALLGSKsPDPYRL |
| 131 | ALLGSKsPDPYRV |
| 132 | ALLsLLKRV |
| 133 | ALMGsPQLV |
| 134 | ALMGsPQLVAA |
| 135 | ALRSsPIMRK |
| 136 | ALRSsPIMRY |
| 137 | ALVsPPALHNA |
| 138 | ALVsPPALHNV |
| 139 | ALYsGVHKK |
| 140 | ALYsGVHKY |
| 141 | ALYsPAQPSL |
| 142 | ALYsPAQPSV |
| 143 | ALYtPQAPK |
| 144 | ALYtPQAPY |
| 145 | AMAAsPHAV |
| 146 | AMDsGASLLHL |
| 147 | AMDsGASLLHV |
| 148 | AMGsRESLATI |
| 149 | AMGsRESLATV |
| 150 | AMLGSKsPDPYRL |
| 151 | AMLGSKsPDPYRV |
| 152 | AMPGsPVEV |
| 153 | AMRSsPIMRK |
| 154 | AMVsPPALHNA |
| 155 | AMVsPPALHNV |
| 156 | AMYsGVHKK |
| 157 | APDsPRAFL |
| 158 | APLARASsL |
| 159 | APPAYEKLs |
| 160 | APPAYEKLsAEQ |
| 161 | APPAYEKLsAEQSPP |
| 162 | APPAYEKLsAEQSPPP |
| 163 | APPAYEKLsAEQSPPPY |
| 164 | APPPLVPAPRPSsPPRGPGPARADR |
| 165 | APRAPsASPLAL |
| 166 | APRDRRAVsF |
| 167 | APRKGsFSAL |
| 168 | APRKGsFSALF |
| 169 | APRKGsFSALL |
| 170 | APRKGsFSALM |
| 171 | APRKGsFSALV |
| 172 | APRNGsGVAL |
| 173 | APRRYsSSF |
| 174 | APRRYsSSL |
| 175 | APRRYsSSM |
| 176 | APRRYsSSV |
| 177 | APRsPPPSRF |
| 178 | APRsPPPSRL |
| 179 | APRsPPPSRM |
| 180 | APRsPPPSRP |
| 181 | APRsPPPSRV |
| 182 | APSLFHLNtL |
| 183 | APSSARAsPLL |
| 184 | APSTYAHLsPAK |
| 185 | APSTYAHLsPAKTPPPP |
| 186 | APSVRsLSL |
| 187 | APSVRSLsL |
| 188 | ARFsPDDKYSF |
| 189 | ARFsPDDKYSK |
| 190 | ARFsPDDKYSL |
| 191 | ARFsPDDKYSM |
| 192 | ARFsPDDKYSR |
| 193 | ARFsPDDKYSY |
| 194 | ASDEIGtPRKF |
| 195 | ASDEIGtPRKY |
| 196 | ASEEIGtPRKF |
| 197 | ASEEIGtPRKY |
| 198 | AsISRLsGEQVDGKG |
| 199 | AsISRLSGEQVDGKG |
| 200 | ASISRLsGEQVDGKG |
| 201 | AsIsRLSGEQVDGKGQ |
| 202 | AsISRLSGEQVDKGKG |
| 203 | ASKAsPTLDFTER |
| 204 | ASKMTQPQSKSAFPLSRKNKGsGsLDG |
| 205 | AsLGFVF |
| 206 | AsPTIEAQGTSPAHDN |
| 207 | AsPTIEAQGTSPAHDNI |
| 208 | AsPTIEAQGTSPAHDNIA |
| 209 | AtAGPRLGF |
| 210 | AtAGPRLGW |
| 211 | AtAGPRLGY |
| 212 | ATDEIGtPRKF |
| 213 | ATDEIGtPRKY |
| 214 | ATEEIGtPRKF |
| 215 | ATEEIGtPRKY |
| 216 | ATWsGSEFEV |
| 217 | ATYtPQAPK |
| 218 | ATYtPQAPKY |
| 219 | AVIHQsLGL |
| 220 | AVIHQsLGV |
| 221 | AVRPTRLsL |
| 222 | AVVsPPALHNA |
| 223 | AVVsPPALHNV |
| 224 | AYEKLsAEQSPP |
| 225 | DAKKsPLAL |
| 226 | DDDWTHLsSKEVDP |
| 227 | DDDWTHLsSKEVDPS |
| 228 | DDDWTHLsSKEVDPST |
| 229 | DDDWTHLsSKEVDPSTG |
| 230 | DDWTHLsSKEVDPS |
| 231 | DEFERIKtF |
| 232 | DEFERIKtW |
| 233 | DEFERIKtY |
| 234 | DEISHRAsF |
| 235 | DEISHRAsW |
| 236 | DEISHRAsY |
| 237 | DERLRINsF |
| 238 | DERLRINsL |
| 239 | DERLRINsW |
| 240 | DERLRINsY |
| 241 | DKLsVIAEDSESGKQ |
| 242 | DKLsVIAEDSESGKQN |
| 243 | DKLsVIAEDSESGKQNP |
| 244 | DKLsVIAEDSESGKQNPG |
| 245 | DKLsVIAEDSESGKQNPGDS |
| 246 | DLKRRsmSI |
| 247 | DLKRRsMSI |
| 248 | DLKSSKAsL |
| 249 | DLRtVEKEL |
| 250 | DLsEEKFL |
| 251 | DLsEEKFV |
| 252 | DLVPLsPLKK |
| 253 | DLWKItKVMD |
| 254 | DMVPLsPLKK |
| 255 | DPTRRFFKVtPPPGSGPQ |
| 256 | DQFERIKtL |
| 257 | DQISHRAsL |
| 258 | DSDPLsPLKY |
| 259 | DSEPLsPLKY |
| 260 | DSsEEKFL |
| 261 | DSsEEKFV |
| 262 | DSVPLsPLKY |
| 263 | DTDPLsPLKY |
| 264 | DTEPLsPLKY |
| 265 | DTVPLSPLKY |
| 266 | DWTHLsSKEVDPS |
| 267 | DWTHLsSKEVDPSTG |
| 268 | EEGsPTMVEKGLEPGVFTL |
| 269 | EELsPTAKF |
| 270 | EELsPTKAF |
| 271 | EEMPENALPsDEDDKDPNDPYRAL |
| 272 | EERRsPPAP |
| 273 | EEsSDDGKKF |
| 274 | EESsDDGKKF |
| 275 | EEsSDDGKKW |
| 276 | EESsDDGKKW |
| 277 | EEsSDDGKKY |
| 278 | EESsDDGKKY |
| 279 | EGEEPTVYsDEEEPKDESARKND |
| 280 | EGsPTMVEKGLEPGVFTL |
| 281 | ELFSsPPAV |
| 282 | ELKKsPTSLK |
| 283 | ELKKsPTSLY |
| 284 | ELLMPHRIsSHF |
| 285 | ELLMPHRIsSHFL |
| 286 | ELRISGsVQL |
| 287 | EMKKsPTSLK |
| 288 | EPAsPAAsISRLsGEQVDGKG |
| 289 | EPAsPAAsISRLSGEQVDGKG |
| 290 | EPKRRsARF |
| 291 | EPKRRsARL |
| 292 | EPKRRsARM |
| 293 | EPKRRsARV |
| 294 | EPRsPSHSF |
| 295 | EPRsPSHSL |
| 296 | EPRsPSHSM |
| 297 | EPRsPSHSV |
| 298 | ERsPLLSQETAGQKP |
| 299 | ERsPLLSQETAGQKPL |
| 300 | ESDsLPRY |
| 301 | ESESLPRY |
| 302 | ESsVRSQEDQLSR |
| 303 | ESsVRSQEDQLSRR |
| 304 | ETDsLPRY |
| 305 | ETEsLPRY |
| 306 | FDKHTLGDsDNES |
| 307 | FEDDDsNEKL |
| 308 | FIEsPSKL |
| 309 | FIEsPSKY |
| 310 | FIGsPTTPAGL |
| 311 | FKMPQEKsPGYS |
| 312 | FKsPVKTIR |
| 313 | FKtQPVTF |
| 314 | FLDNsFEKV |
| 315 | FLDRPPtPLFI |
| 316 | FLDsLRDLI |
| 317 | FLDtPIAKV |
| 318 | FLFDKPVsPLLL |
| 319 | FLGVRPKsA |
| 320 | FLIIRtVLQL |
| 321 | FLITGGGKGsGFSL |
| 322 | FLLsQNFDDE |
| 323 | FLYsGKETK |
| 324 | FLYsGKETY |
| 325 | FPHsLLSVF |
| 326 | FPHsLLSVI |
| 327 | FPHsLLSVL |
| 328 | FPHsLLSVM |
| 329 | FPHsLLSVV |
| 330 | FPIsPVRF |
| 331 | FPIsPVRL |
| 332 | FPIsPVRM |
| 333 | FPIsPVRV |
| 334 | FPLDsPKTLVL |
| 335 | FPRRHsVTL |
| 336 | FPRsPTKSSF |
| 337 | FPRsPTKSSL |
| 338 | FPRsPTKSSLDF |
| 339 | FPRsPTKSSLDL |
| 340 | FPRsPTKSSLDM |
| 341 | FPRsPTKSSLDV |
| 342 | FPRsPTKSSM |
| 343 | FPRsPTKSSV |
| 344 | FRFsGRTEY |
| 345 | FRGRYRsPY |
| 346 | FRKsMVEHY |
| 347 | FRRsPIKSSLDY |
| 348 | FRRsPTKSSF |
| 349 | FRRsPTKSSL |
| 350 | FRRsPTKSSLD |
| 351 | FRRsPTKSSLDF |
| 352 | FRRsPTKSSLDL |
| 353 | FRRsPTKSSLDM |
| 354 | FRRsPTKSSLDV |
| 355 | FRRsPTKSSLDY |
| 356 | FRRsPTKSSM |
| 357 | FRRsPTKSSV |
| 358 | FRsPTKSSLDF |
| 359 | FRsPTKSSLDL |
| 360 | FRsPTKSSLDM |
| 361 | FRsPTKSSLDV |
| 362 | FRYsGKTEF |
| 363 | FRYsGKTEK |
| 364 | FRYsGKTEL |
| 365 | FRYsGKTEM |
| 366 | FRYsGKTER |
| 367 | FRYsGKTEY |
| 368 | FSDsHEGFSY |
| 369 | FSEsHEGFSY |
| 370 | FSEsPSKL |
| 371 | FSEsPSKY |
| 372 | FSIsPVRF |
| 373 | FSIsPVRL |
| 374 | FSIsPVRM |
| 375 | FSIsPVRV |
| 376 | FSsSHEGFSY |
| 377 | FSSsHEGFSY |
| 378 | FTDsHEGFSY |
| 379 | FTEsHEGFSY |
| 380 | FTEsPSKL |
| 381 | FTEsPSKY |
| 382 | FTKsPYQEF |
| 383 | FTsSHEGFSY |
| 384 | FVSKVMIGsPKKV |
| 385 | GALsPSLLHSL |
| 386 | GAQPGRHsF |
| 387 | GAQPGRHsL |
| 388 | GAQPGRHsV |
| 389 | GDDDWTHLsSKEVD |
| 390 | GDDDWTIILsSKEVDP |
| 391 | GDDDWTHLsSKEVDPS |
| 392 | GDDDWTHLsSKEVDPST |
| 393 | GDDDWTHLsSKEVDPSTG |
| 394 | GEAsPSHII |
| 395 | GEEsSDDGKKF |
| 396 | GEEsSDDGKKW |
| 397 | GEEsSDDGKKY |
| 398 | GEEsSDIDGKKF |
| 399 | GEIsPQREV |
| 400 | GERSPLLSQETAGQKP |
| 401 | GERsPLLSQETAGQKPL |
| 402 | GETsPRTKI |
| 403 | GGDDDWTHLsSKEVDPS |
| 404 | GGDDDWTHLsSKEVDPSTG |
| 405 | GGSFGGRSSGsP |
| 406 | GGSFGGRSSGsV |
| 407 | GIDsPSSSV |
| 408 | GIMsPLAKK |
| 409 | GLAPtPPSM |
| 410 | GLDsGFHSV |
| 411 | GLDsLDQVEI |
| 412 | GLGELLRsL |
| 413 | GLIRSRsFIFK |
| 414 | GLIRSRsFIFY |
| 415 | GLIsPELRHL |
| 416 | CLIsPNVQL |
| 417 | GLIsPVWGA |
| 418 | GLItPGGFSSV |
| 419 | GLLDsPTSI |
| 420 | GLLGsPARL |
| 421 | GLLGsPVRA |
| 422 | GLLGsPVRV |
| 423 | GLLsPARLYAI |
| 424 | GLLsPARLYAV |
| 425 | GLLsPRFVDV |
| 426 | GLLsPRHSL |
| 427 | GLSFGGRSSGsP |
| 428 | GLSFGGRSSGsV |
| 429 | GMLGsPVRV |
| 430 | GMLsPARLYAI |
| 431 | GMLsPARLYAV |
| 432 | GMLsPGKSIEV |
| 433 | GPKPLFRRMsS |
| 434 | GPKPLFRRMsSL |
| 435 | GPKPLFRRMsSLV |
| 436 | GPKPLFRRMsSLVG |
| 437 | GPKPLFRRMsSLVGP |
| 438 | GPKPLFRRMsSLVGPT |
| 439 | GPKPLFRRMsSLVGPTQ |
| 440 | GPKPLFRRMsSLVGPTQS |
| 441 | GPPYQRRGsL |
| 442 | GPQPGRHsF |
| 443 | GPQPGRHsL |
| 444 | GPQPGRHsV |
| 445 | GPRPGsPSAF |
| 446 | GPRPGsPSAL |
| 447 | GPRPGsPSAM |
| 448 | GPRPGsPSAV |
| 449 | GPRSAsLL |
| 450 | GPRsASLLSF |
| 451 | GPRSAsLLsF |
| 452 | GPRSASLLsF |
| 453 | GPRsAsLLSL |
| 454 | GPRsASLLSL |
| 455 | GPRSAsLLsL |
| 456 | GPRSAsLLSL |
| 457 | GPRSASLLsL |
| 458 | GPRsASLLSM |
| 459 | GPRSAsLLsM |
| 460 | GPRSASLLsM |
| 461 | GPRsASLLSV |
| 462 | GPKSAsLLSV |
| 463 | GPRSASLLsV |
| 464 | GPRsPKAPP |
| 465 | GPRsPPVTL |
| 466 | GQLsPGVQF |
| 467 | GRKsPPPSF |
| 468 | GRKsPPPSK |
| 469 | GRKsPPPSL |
| 470 | GRKsPPPSM |
| 471 | GRKsPPPSR |
| 472 | GRKsPPPSY |
| 473 | GRLGsPHRF |
| 474 | GRLGsPHRK |
| 475 | GRLGsPHRL |
| 476 | GRLGsPHRM |
| 477 | GRLGsPHRR |
| 478 | GRLGsPHRY |
| 479 | GRLsPAYSL |
| 480 | GRLsPKASQVF |
| 481 | GRLsPKASQVK |
| 482 | GRLsPKASQVL |
| 483 | GRLsPKASQVM |
| 484 | GRLsPKASQVR |
| 485 | GRLsPKASQVY |
| 486 | GRLsPVPVPF |
| 487 | GRLsPVPVPK |
| 488 | GRLsPVPVPL |
| 489 | GRLsPVPVPM |
| 490 | GRLsPVPVPR |
| 491 | GRLsPVPVPY |
| 492 | GRQsPSFKL |
| 493 | GRsSPPPGY |
| 494 | GRSsTASLVKF |
| 495 | GRSsTASLVKK |
| 496 | GRSsTASLVKKK |
| 497 | GRSsTASLVKL |
| 498 | GRSsTASLVKM |
| 499 | GRSsTASLVKR |
| 500 | GRSsTASLVKY |
| 501 | GRtGLPDL |
| 502 | GSALGGGGAGLSGRASGGAQsPLRYLHV |
| 503 | GSDsSDDGKKY |
| 504 | GSEsSDDGKKY |
| 505 | GsPHYFSPF |
| 506 | GsPHYFSPFRPY |
| 507 | GsPTMVEKGLEPGVFTL |
| 508 | GsQLAVMMYL |
| 509 | GTDsSDDGKKY |
| 510 | GTEsSDDGKKY |
| 511 | GTIRSRsFIFK |
| 512 | GTIRSRsFIFY |
| 513 | GtLPKY |
| 514 | GtLRRSDSQQAVK |
| 515 | GtLRRSDSQQAVKS |
| 516 | GtLRRSDSQQAVKSPP |
| 517 | GVAsPTITV |
| 518 | GVVsPTFEL |
| 519 | HEKKAYsF |
| 520 | HKGEIRGASTPFQFRAssP |
| 521 | HLHsPQHKL |
| 522 | HPKRSVsL |
| 523 | HPRsPNVL |
| 524 | HPRsPNVLSF |
| 525 | HPRsPNVLSL |
| 526 | HPRsPNVLSM |
| 527 | HPRsPNVLSV |
| 528 | HPRsPTPTF |
| 529 | HPRSPtPTF |
| 530 | HPRsPTPTL |
| 531 | HPRSPtPTL |
| 532 | HPRsPTPTM |
| 533 | HPRSPtPTM |
| 534 | HPRSPtPTV |
| 535 | HPsSPTPTV |
| 536 | HRLsPVKGEF |
| 537 | HRLsPVKGEK |
| 538 | HRLsPVKGER |
| 539 | HRLsPVKGEY |
| 540 | HRNsMKVFL |
| 541 | HRNsNPVIAEF |
| 542 | HRNsNPVIAEK |
| 543 | HRNsNPVIAEL |
| 544 | HRNsNPVIAER |
| 545 | HRNsNPVIAEY |
| 546 | HRYsTPHAF |
| 547 | HTAsPTGMMK |
| 548 | HVYtPSTTK |
| 549 | IEKIyIMKADTVIVG |
| 550 | IIEtPHKEI |
| 551 | IIEtPHKEY |
| 552 | IISsPLKGY |
| 553 | IISsPLTGK |
| 554 | ILDRtPEKL |
| 555 | ILDRtPEKV |
| 556 | ILDsGIYRI |
| 557 | ILDsGIYRV |
| 558 | ILKPRRsL |
| 559 | ILKsPEIQRA |
| 560 | ILKsPEIQRV |
| 561 | ILQtPQFQM |
| 562 | ILQVsIPSL |
| 563 | IMDRtPEKL |
| 564 | IMDRtPEKV |
| 565 | IMDsGIYRI |
| 566 | IMDsGIYRV |
| 567 | IMKsPEIQRA |
| 568 | IMKsPEIQRV |
| 569 | INKERRSsL |
| 570 | IPVgSSHNSL |
| 571 | IQFsPPFPGA |
| 572 | ISDGtLKY |
| 573 | ISDGtPLKY |
| 574 | ISDSAHtDY |
| 575 | ISDsMHSLY |
| 576 | ISDtPHKEI |
| 577 | ISDtPHKEY |
| 578 | ISEGtLKY |
| 579 | ISEGtPLKY |
| 580 | ISESAHtDY |
| 581 | ISEsMHSLY |
| 582 | ISELPHKEI |
| 583 | ISEtPHKEY |
| 584 | ISFSAHtDY |
| 585 | ISSsMIISLY |
| 586 | IStDRDPL |
| 587 | IStDRDPY |
| 588 | ITDGtLKY |
| 589 | ITDGtPLKY |
| 590 | ITDSAHtDY |
| 591 | ITDsMHSLY |
| 592 | ITDtPHKEI |
| 593 | ITDtPHKEY |
| 594 | ITEGtLKY |
| 595 | ITEGtPLKY |
| 596 | ITESAHtDY |
| 597 | ITEsMHSLY |
| 598 | ITEtPHKEI |
| 599 | ITEtPHKEY |
| 600 | ITQGtLKY |
| 601 | ITQGtPLKK |
| 602 | ITQGtPLKY |
| 603 | ITtDRDPL |
| 604 | ITtDRDPY |
| 605 | IVLsDSEVIQL |
| 606 | IVRyHQL |
| 607 | IVtDRDPL |
| 608 | IVtDRDPY |
| 609 | IYQyIQSRF |
| 610 | KAFsPVR |
| 611 | KAFsPVRSV |
| 612 | KAKsPAPGL |
| 613 | KAKsPAPGV |
| 614 | KARsPGRAF |
| 615 | KARsPGRAL |
| 616 | KARsPGRAM |
| 617 | KARsPGRAV |
| 618 | KASPKRLsL |
| 619 | KAVsLFLcY |
| 620 | KAVsLFLCY |
| 621 | KEGEEPTVYsDEEEPKDESARKND |
| 622 | KEKsPFRET |
| 623 | KELARQIsF |
| 624 | KEMsPTRQF |
| 625 | KEmsPTRQL |
| 626 | KEMsPTRQL |
| 627 | KEMsPTRQW |
| 628 | KEMsPTRQY |
| 629 | KESsPLSSRKI |
| 630 | KFRPPPLsL |
| 631 | KGIsSSSLKEK |
| 632 | KIAsEIAQL |
| 633 | KIDIVsSQKV |
| 634 | KIDsPTKVKK |
| 635 | KIEKIyIMKADTVIVG |
| 636 | KIEsLENLYL |
| 637 | KIFsGVFVK |
| 638 | KIFsGVFVKV |
| 639 | KIFsKQQGK |
| 640 | KIFsKQQGY |
| 641 | KIGsIIFQV |
| 642 | KIKsFEVvF |
| 643 | KIRSsPREAK |
| 644 | KIRSsPREAY |
| 645 | KIRTsPTFR |
| 646 | KIRTsPTFY |
| 647 | KLAsLEREASV |
| 648 | KLAsLLHQV |
| 649 | KLAsPEKLAGL |
| 650 | KLAsPELERL |
| 651 | KLAsPELERV |
| 652 | KLDIVsSQKV |
| 653 | KLDsFLDMQV |
| 654 | KLDsPRVTV |
| 655 | KLDsPTKVKK |
| 656 | KLDsPTKVKY |
| 657 | KLFPDtPLAL |
| 658 | KLFPDtPLAV |
| 659 | KLFsGTVRK |
| 660 | KLFsGVFVKV |
| 661 | KLFsKQQGK |
| 662 | KLFsKQQGY |
| 663 | KLFsPAHKK |
| 664 | KLFsPAIIKY |
| 665 | KLFsPSKEAEL |
| 666 | KLFsPSKEAEV |
| 667 | KLHGsLARAGK |
| 668 | KLHGsLARAGY |
| 669 | KLIDIVsSQKV |
| 670 | KLIDRTEsL |
| 671 | KLIDVsSQKV |
| 672 | KLIsSSSLKEK |
| 673 | KLIsSSSLKEY |
| 674 | KLKDRLPsI |
| 675 | KLKsNPDFLK |
| 676 | KLKsNPDFLKK |
| 677 | KLKsNPDFLKY |
| 678 | KLKsPAPGL |
| 679 | KLKsPAPGV |
| 680 | KLKsQEIFL |
| 681 | KLKSsPLIEKK |
| 682 | KLKSsPLIEKY |
| 683 | KLKtPLVAK |
| 684 | KLKtPLVAR |
| 685 | KLLDFGSLsNLQV |
| 686 | KLLQFYPsL |
| 687 | KLLQFYPsV |
| 688 | KLLsPSDEKL |
| 689 | KLLsPSNEKL |
| 690 | KLLsPSNEKV |
| 691 | KLLSSAQRtL |
| 692 | KLLSSAQRtV |
| 693 | KLLsTEEMEL |
| 694 | KLLsTEEMEV |
| 695 | KLLsVERIK |
| 696 | KLLtPIKEK |
| 697 | KLLtPIKEK |
| 698 | KLMAPDIsL |
| 699 | KLMAPDIsV |
| 700 | KLMIDRTEsV |
| 701 | KLMsDVEDV |
| 702 | KLMsPKADV |
| 703 | KLMsPKADVKL |
| 704 | KLMsPKADVKV |
| 705 | KLPDsPALA |
| 706 | KLPDsPALAK |
| 707 | KLPDsPALAKK |
| 708 | KLPDsPALAKY |
| 709 | KLPDsPALAY |
| 710 | KLPsPAPARK |
| 711 | KLPTsPLKMK |
| 712 | KLPTsPLKMY |
| 713 | KLPTtPVKAK |
| 714 | KLPTtPVKAY |
| 715 | KLQEFLQtL |
| 716 | KLQVtSLSV |
| 717 | KLRsPFLQK |
| 718 | KLRsPFLQY |
| 719 | KLRSsPREAK |
| 720 | KLRTsPTFK |
| 721 | KLsGDQPAAR |
| 722 | KLSGLsF |
| 723 | KLSsLGNLK |
| 724 | KLSsLGNLKK |
| 725 | KLSsLGNLKY |
| 726 | KLSsPRGGMK |
| 727 | KLSsPRGGMKK |
| 728 | KLSsPRGGMKY |
| 729 | KLsVIAEDSESGKQN |
| 730 | KLsVIAEDSESGKQNP |
| 731 | KLsVIAEDSESGKQNPG |
| 732 | KLVSFHDDsDEDL |
| 733 | KLYsEIDIKV |
| 734 | KLYsGNMEK |
| 735 | KMAsLLHQV |
| 736 | KMAsPELERL |
| 737 | KMAsPELERV |
| 738 | KMDIVsSQKV |
| 739 | KMDsFLDMQL |
| 740 | KMDsFLDMQV |
| 741 | KMDsPRVTV |
| 742 | KMDsPTKVKK |
| 743 | KMFPDtPLAL |
| 744 | KMFPDtPLAV |
| 745 | KMFsGTVRK |
| 746 | KMFsGVFVKV |
| 747 | KMFsKQQGK |
| 748 | KMFsPAHKK |
| 749 | KMFsPSKEAEL |
| 750 | KMFsPSKEAEV |
| 751 | KMHGsLARAGK |
| 752 | KMIDIVsSQKV |
| 753 | KMIDRTEsL |
| 754 | KMIsSSSLKEK |
| 755 | KMKsNPDFLK |
| 756 | KMKsNPDFLKK |
| 757 | KMKsNPDFLKY |
| 758 | KMKSsPLIEKK |
| 759 | KMKtPLVAK |
| 760 | KMKtPLVAR |
| 761 | KMLDFGSLsNLOV |
| 762 | KMLDFGSLsNLQV |
| 763 | KMLQFYPsL |
| 764 | KMLsPSNEKL |
| 765 | KMLsPSNEKV |
| 766 | KMLSSAQRtL |
| 767 | KMLSSAQRtV |
| 768 | KMLsVERIK |
| 769 | KMLtPIKEK |
| 770 | KMMAPDIsV |
| 771 | KMMsPKADVKL |
| 772 | KMMsPKADVKV |
| 773 | KMPTsPLKMK |
| 774 | KMPTtPVKAK |
| 775 | KMPTtPVKAY |
| 776 | KMRsPFLQK |
| 777 | KMRSsPREAK |
| 778 | KMRTsPTFK |
| 779 | KMSsLGNLK |
| 780 | KMSsLGNLKK |
| 781 | KMSsLGNLKY |
| 782 | KMSsPRGGMK |
| 783 | KMSsPRGGMKK |
| 784 | KMYsEIDIKV |
| 785 | KMYsGNMEK |
| 786 | KNRsWKYN |
| 787 | KNRsWKYNQ |
| 788 | KNRsWKYNQSISLR |
| 789 | KNRsWKYNQSISLRRP |
| 790 | KPAsPARRF |
| 791 | KPAsPARRL |
| 792 | KPAsPARRM |
| 793 | KPAsPARRV |
| 794 | KPAsPKFIVTF |
| 795 | KPAsPKFIVTL |
| 796 | KPAsPKFIVTM |
| 797 | KPAsPKFIVTV |
| 798 | KPEsRRSSL |
| 799 | KPEsRRsSLL |
| 800 | KPEsRRSsLL |
| 801 | KPEsRRSSLL |
| 802 | KPLIRsQSL |
| 803 | KPLIRSQsL |
| 804 | KPPHsPLVF |
| 805 | KPPHsPLVL |
| 806 | KPPHsPLVM |
| 807 | KPPHsPLVV |
| 808 | KPPsPEHQSF |
| 809 | KPPsPEHQSL |
| 810 | KPPsPEHQSM |
| 811 | KPPsPEHQSV |
| 812 | KPPsPSPIEF |
| 813 | KPPsPSPIEL |
| 814 | KPPsPSPIEM |
| 815 | KPPsPSPIEV |
| 816 | KPPtPGASF |
| 817 | KPPtPGASL |
| 818 | KPPtPGASM |
| 819 | KPPtPGASV |
| 820 | KPPYRSHsF |
| 821 | KPPYRSHsL |
| 822 | KPPYRSHsM |
| 823 | KPPYRSHsV |
| 824 | KPQTRGKtF |
| 825 | KPQTRGKtL |
| 826 | KPQTRGXtM |
| 827 | KPQTRGKtV |
| 828 | KPRPLsMDL |
| 829 | KPRPPPLsF |
| 830 | KPRPPPLsL |
| 831 | KPRPPPLsM |
| 832 | KPRPPPLsP |
| 833 | KPRPPPLsV |
| 834 | KPRRFsRsL |
| 835 | KPRRFsRSL |
| 836 | KPRsPDHVF |
| 837 | KPRsPDHVL |
| 838 | KPRsPDHVM |
| 839 | KPRsPDHVV |
| 840 | KPRsPFSKI |
| 841 | KPRsPPRAF |
| 842 | KPRsPPRAL |
| 843 | KPRsPPRALF |
| 844 | KPRsPPRALL |
| 845 | KPRsPPRALM |
| 846 | KPRsPPRALV |
| 847 | KPRsPPRALVF |
| 848 | KPRsPPRALVL |
| 849 | KPRsPPRALVLF |
| 850 | KPRsPPRALVLL |
| 851 | KPRsPPRALVLM |
| 852 | KPRsPPRALVLP |
| 853 | KPRsPPRALVLV |
| 854 | KPRsPPRALVM |
| 855 | KPRsPPRALVV |
| 856 | KPRsPPRAM |
| 857 | KPRsPPRAV |
| 858 | KPRsPVVEF |
| 859 | KPRsPVVEL |
| 860 | KPRsPVVEM |
| 861 | KPRsPVVEV |
| 862 | KPSsPRGSL |
| 863 | KPSsPRGSLL |
| 864 | KPVsPKSGTL |
| 865 | KPYsPLASF |
| 866 | KPYsPLASL |
| 867 | KPYsPLASM |
| 868 | KPYsPLASV |
| 869 | KQDsLVINL |
| 870 | KRAsFAKSF |
| 871 | KRAsFAKSK |
| 872 | KRAsFAKSL |
| 873 | KRAsFAKSM |
| 874 | KRAsFAKSR |
| 875 | KRAsFAKSV |
| 876 | KRAsFAKSY |
| 877 | KRAsGQAFEF |
| 878 | KRAsGQAFEK |
| 879 | KRAsGQAFEL |
| 880 | KRAsGQAFER |
| 881 | KRAsGQAFEY |
| 882 | KRASSPFRF |
| 883 | KRASsPFRK |
| 884 | KRASsPFRL |
| 885 | KRASsPFRM |
| 886 | KRASsPFRR |
| 887 | KRASsPFRY |
| 888 | KRAsVFVKF |
| 889 | KRAsVFVKK |
| 890 | KRAsVFVKL |
| 891 | KRAsVFVKM |
| 892 | KRAsVFVKR |
| 893 | KRAsVFVKY |
| 894 | KRAsYILRL |
| 895 | KRFsFKF |
| 896 | KRFsFKK |
| 897 | KRFsFKKsF |
| 898 | KRFsFKKSF |
| 899 | KRFsFKKSK |
| 900 | KRFsFKKSL |
| 901 | KRFsFKKSM |
| 902 | KRFsFKKSR |
| 903 | KRFsFKKSY |
| 904 | KRFsFKL |
| 905 | KRFsFKM |
| 906 | KRFsFKR |
| 907 | KRFsFKsSF |
| 908 | KRFsFKY |
| 909 | KRFsGTVRF |
| 910 | KRFsGTVRK |
| 911 | KRFsGTVRL |
| 912 | KRFsGTVRM |
| 913 | KRFsGTVRR |
| 914 | KRFsGTVRY |
| 915 | KRIVIsPKPF |
| 916 | KRKsFTSLY |
| 917 | KRLEKsPSF |
| 918 | KRLEKSPsF |
| 919 | KRLsPAPQF |
| 920 | KRLsPAPQK |
| 921 | KRLsPAPQL |
| 922 | KRLsPAPQM |
| 923 | KRLsPAPQR |
| 924 | KRLsPAPQY |
| 925 | KRLsTSPVRL |
| 926 | KRLsVERIF |
| 927 | KRLsVERIK |
| 928 | KRLsVERIL |
| 929 | KRLsVERIM |
| 930 | KRLsVERIR |
| 931 | KRLsVERIY |
| 932 | KRMsPKEF |
| 933 | KRMsPKEK |
| 934 | KRMsPKEL |
| 935 | KRMsPKER |
| 936 | KRMsPKEY |
| 937 | KRmsPKPEL |
| 938 | KRMsPKPEL |
| 939 | KRMsPKPF |
| 940 | KRMsPKPK |
| 941 | KRMsPKPL |
| 942 | KRMsPKPM |
| 943 | KRMsPKPR |
| 944 | KRMsPKPY |
| 945 | KRPEsPPSI |
| 946 | KRWQsPVTK |
| 947 | KRYsEPVSL |
| 948 | KRYsGNMEF |
| 949 | KRYsGNMEK |
| 950 | KRYsGNMEL |
| 951 | KRYsGNMEM |
| 952 | KRYsGNMER |
| 953 | KRYsGNmEY |
| 954 | KRYsGNMEY |
| 955 | KRYsRALYL |
| 956 | KSDsRQERY |
| 957 | KSEsRQERY |
| 958 | KSGELLAtW |
| 959 | KSKsNPDFLKK |
| 960 | KSKsNPFLKK |
| 961 | KSKtPLVAK |
| 962 | KSKtPLVAR |
| 963 | KSKtPLVAY |
| 964 | KsLVRLLLL |
| 965 | KSSsLGNLKK |
| 966 | KsVKALSSLHGDDQ |
| 967 | KsVKALSSLHGDDQD |
| 968 | KsVKALSSLHGDDQDsEDE |
| 969 | KSVKALSSLHGDDQDsEDE |
| 970 | KTDsRQERY |
| 971 | KTEsRQERY |
| 972 | KtLSPGKNGVVK |
| 973 | KtLSPGKNGVVY |
| 974 | KTMsGTFLL |
| 975 | KTMsPSQMIM |
| 976 | KTPTsPLKMK |
| 977 | KTPTsPLKMY |
| 978 | KTWKGsIGL |
| 979 | KVAsLLHQV |
| 980 | KVDsPVIF |
| 981 | KVHGsLARAGK |
| 982 | KVHGsLARAGY |
| 983 | KVKSsPLIEKK |
| 984 | KVKsSPLIEKL |
| 985 | KVKSsPLIEKL |
| 986 | KVKSsPLIEKY |
| 987 | KVLsKEFHL |
| 988 | KVLSPtAAK |
| 989 | KVLsSLVTL |
| 990 | KVLsTEEMEL |
| 991 | KVLStEEMEL |
| 992 | KVLtPIKeK |
| 993 | KVLtPIKEK |
| 994 | KVLtPIKEY |
| 995 | KVPDsPALAK |
| 996 | KVPDsPALAKK |
| 997 | KVPDsPALAKY |
| 998 | KVPDsPALAY |
| 999 | KVPTsPLKMY |
| 1000 | KVQsLRRAL |
| 1001 | KVQVtSLSV |
| 1002 | KVYsSSEFL |
| 1003 | KYIsGPHEL |
| 1004 | KYsPGKLRGN |
| 1005 | LGGGGAGLSGRASGGAQsPLRYLHV |
| 1006 | LKLsYLTWV |
| 1007 | LLAsPGHISV |
| 1008 | LLDPSRSYsY |
| 1009 | LLDtPVKTQY |
| 1010 | LLFsPVTSL |
| 1011 | LLFsPVTSV |
| 1012 | LLLsEEVEL |
| 1013 | LLNKSsPVK |
| 1014 | LLNKSsPVKK |
| 1015 | LLNKSsPVKY |
| 1016 | LMFsPVTSL |
| 1017 | LMFsPVTSV |
| 1018 | LMFsVTSI |
| 1019 | LMFsVTSL |
| 1020 | LMNKSsPVK |
| 1021 | TiMNKSsPVKK |
| 1022 | LMNKSsPVKY |
| 1023 | LPAsPHQF |
| 1024 | LPAsPHQL |
| 1025 | LPAsPHQM |
| 1026 | LPAsPHQV |
| 1027 | LPAsPRARF |
| 1028 | LPAsPRARL |
| 1029 | LPAsPRARM |
| 1030 | LPAsPRARV |
| 1031 | LPIFSRLsF |
| 1032 | LPIFSRLsI |
| 1033 | LPIFSRLsL |
| 1034 | LPIFSRLsM |
| 1035 | LPIFSRLsV |
| 1036 | LPKGLsASL |
| 1037 | LPKGLSAsL |
| 1038 | LPKsPPYTAF |
| 1039 | LPKsPPYTAL |
| 1040 | LPKsPPYTAM |
| 1041 | LPKsPPYTAV |
| 1042 | LPRGSsPSVF |
| 1043 | LPRGsSPSVL |
| 1044 | LPRGSsPSVL |
| 1045 | LPRGSsPSVM |
| 1046 | LPRGSsPSVV |
| 1047 | LPRmIsHSEL |
| 1048 | LPRMIsHSEL |
| 1049 | LPRPAsPAL |
| 1050 | LPRSSsMAA |
| 1051 | LPRSSsMAAGL |
| 1052 | LPRtPRPEL |
| 1053 | LPVsPRLQL |
| 1054 | LQLsPLKGLSL |
| 1055 | LQNItENQL |
| 1056 | LSDPSRSYsY |
| 1057 | LSDsDTEAKL |
| 1058 | LSDsDTEAKY |
| 1059 | LSDtPVKTQY |
| 1060 | LSEPSRSYsY |
| 1061 | LSEsDTEAKL |
| 1062 | LSEsDTEAKY |
| 1063 | LSEtPVKTQY |
| 1064 | LSKFRMPQPSSGREsPRH |
| 1065 | LSSsVIREL |
| 1066 | LTDPSRSYsY |
| 1067 | LTDPSsPTISSY |
| 1068 | LTDsDTEAKL |
| 1069 | LTDSDTEAKY |
| 1070 | LTDtPVKTQY |
| 1071 | LTEPSRSYsY |
| 1072 | LTEsDTEAKL |
| 1073 | LTEsDTEAKY |
| 1074 | LTEtPVKTOY |
| 1075 | LTEtPVKTQY |
| 1076 | MLAEsPSVPRL |
| 1077 | MLAEsPSVPRV |
| 1078 | MLRsPPRVSK |
| 1079 | MMRsPPRVSK |
| 1080 | MPRPsIKKAQNSQAARQ |
| 1081 | MPRQPsAIRM |
| 1082 | MPRQPsATRF |
| 1083 | MPRQPsATRL |
| 1084 | MPRQPsATRM |
| 1085 | MPRQPsATRV |
| 1086 | MRLsEWLQL |
| 1087 | MRLsRELQF |
| 1088 | MRLsRELQK |
| 1089 | MRLsRELQL |
| 1090 | MRLsRELQM |
| 1091 | MRLsRELQR |
| 1092 | MRLsRELQY |
| 1093 | MSDtYRLKY |
| 1094 | MSEtYRLKY |
| 1095 | MTDtYRLKY |
| 1096 | MTEtYRLKY |
| 1097 | MTRsPPRVSK |
| 1098 | MTRsPPRVSY |
| 1099 | NAPPAYEKLsAE |
| 1100 | NFKsPVKTIR |
| 1101 | NLELSKFRMPQPSSGREsPRH |
| 1102 | NLGsRNHVHQL |
| 1103 | NLLsPDGKMISV |
| 1104 | NLVERKNsK |
| 1105 | NLVERKNsL |
| 1106 | NMDsPGPML |
| 1107 | NMVERKNsK |
| 1108 | NMVERKNsL |
| 1109 | NRAMRRVsSVPSR |
| 1110 | NRAMRRVsSVPSRAQ |
| 1111 | NRsWKYNQSISLR |
| 1112 | NRsWKYNQSISLRRP |
| 1113 | NRYtNRVVTF |
| 1114 | NRYtNRVVTK |
| 1115 | NRYtNRVVTL |
| 1116 | NRYtNRVVTM |
| 1117 | NRYtNRVVTR |
| 1118 | NRYtNRVVTY |
| 1119 | NSDsPLRY |
| 1120 | NSEsPLRY |
| 1121 | NTDsPLRY |
| 1122 | NTEsPLRY |
| 1123 | NYVERKNsK |
| 1124 | NYVERKNsL |
| 1125 | NYVERKNsY |
| 1126 | PARsPVTEI |
| 1127 | PAYEKLsAE |
| 1128 | PAYEKLsAEQSP |
| 1129 | PmVTLsLNL |
| 1130 | PMVTLsLNL |
| 1131 | PNAPPAYEKLsA |
| 1132 | PPAYEKLsA |
| 1133 | PPAYEKLsAEQS |
| 1134 | PPLPEDSIKVIRNMRAAsPPA |
| 1135 | PYDPALGsPSR |
| 1136 | QAASNFKsPVKTIR |
| 1137 | QLDsPQRALY |
| 1138 | QLEsPQRALY |
| 1139 | QLFsPKKGQK |
| 1140 | QMFsPKKGQK |
| 1141 | QPQRRsLRL |
| 1142 | QPRsPGPDYSF |
| 1143 | QPRsPGPDYSL |
| 1144 | QPRsPGPDYSM |
| 1145 | QPRsPGPDYSV |
| 1146 | QPRtPsPLVF |
| 1147 | QPRtPSPLVF |
| 1148 | QPRtPsPLVL |
| 1149 | QPRtPSPLVL |
| 1150 | QPRtPsPLVM |
| 1151 | QPRtPSPLVM |
| 1152 | QPRtPsPLVV |
| 1153 | QPRtPSPLVV |
| 1154 | QPSFPsVLPA |
| 1155 | QRLsPLSAAY |
| 1156 | QSDsPQRALY |
| 1157 | QSEsPQRALY |
| 1158 | QTEsPQRALY |
| 1159 | QTEsPQRALY |
| 1160 | QVAMPVKKSPRRSsSDEQGLSYSSLKNV |
| 1161 | QVFsPKXGQK |
| 1162 | QVFsPKKGQY |
| 1163 | RADsPVHM |
| 1164 | RAFsFSKTPK |
| 1165 | RAFsFSKTPY |
| 1166 | RAFsVKFEV |
| 1167 | RAHsEPLAL |
| 1168 | RAHsSPASL |
| 1169 | RAHSsPASL |
| 1170 | RAKsPISLK |
| 1171 | RAKsPISLY |
| 1172 | RAPsPSSRF |
| 1173 | RAPsPSSRL |
| 1174 | RAPsPSSRM |
| 1175 | RAPsPSSRV |
| 1176 | RARGIsPIVF |
| 1177 | RASsDIVsL |
| 1178 | RASsDIVSL |
| 1179 | RASsLSITV |
| 1180 | REAPsPLmI |
| 1181 | REAPsPLMI |
| 1182 | REAsPAPLA |
| 1183 | REAsPRLRV |
| 1184 | REAsPSRLSV |
| 1185 | REDsTPGKVFL |
| 1186 | REIMGtPEYL |
| 1187 | REKsPGRmL |
| 1188 | REKsPGRML |
| 1189 | REKsPLFQF |
| 1190 | REKsPLFQW |
| 1191 | REKsPLFQY |
| 1192 | RELARKGsL |
| 1193 | RELsPLISL |
| 1194 | REPsPLPEL |
| 1195 | RERsPSPSF |
| 1196 | RESsPTRRL |
| 1197 | REVsPAPAV |
| 1198 | REYGsTSSI |
| 1199 | RFKtQPVTF |
| 1200 | RGDGYGtF |
| 1201 | RGDsPKIDL |
| 1202 | RIDsKDSASEL |
| 1203 | RIGsPLSPK |
| 1204 | RILsGVVTK |
| 1205 | RILsGVVTY |
| 1206 | RILsPSMASK |
| 1207 | RILsPSMASY |
| 1208 | RINsFEEHV |
| 1209 | RIQsKLYRA |
| 1210 | RIQyIQSRF |
| 1211 | RIQyIQSRFY |
| 1212 | RIsHELDS |
| 1213 | RITsLIVHV |
| 1214 | RIVQyIQSR |
| 1215 | RIYQyIQ |
| 1216 | RIYQyIQSK |
| 1217 | RIYQyIQSR |
| 1218 | RIYQyIQSRF |
| 1219 | RIYQyIQSRFK |
| 1220 | RIYQyIQSRFY |
| 1221 | RIYQyIQSRK |
| 1222 | RIYQyIQSRY |
| 1223 | RIYQyIQSY |
| 1224 | RIYQyLQSRF |
| 1225 | RIYQyLQSRFY |
| 1226 | RKLRsLEQL |
| 1227 | RKLsVILIK |
| 1228 | RKLsVILIL |
| 1229 | RKLsVILIY |
| 1230 | RKPsIVTKY |
| 1231 | RKSsIIIRM |
| 1232 | RLAsASRAL |
| 1233 | RLAsFAVRK |
| 1234 | RLAsFAVRY |
| 1235 | RLAsIELPSM |
| 1236 | RLAsIELPSMAV |
| 1237 | RLAsIELPSV |
| 1238 | RLAsLNAEAL |
| 1239 | RLAsLNAEAV |
| 1240 | RLAsLQSEV |
| 1241 | RLAsLSISV |
| 1242 | RLAsPLVHK |
| 1243 | RLAsPLVHY |
| 1244 | RLAsPPPPPK |
| 1245 | RLAsPPPPPY |
| 1246 | RLAsPTSGV |
| 1247 | RLAsPTSGVK |
| 1248 | RLAsPTSGVKK |
| 1249 | RLAsPTSGVKR |
| 1250 | RLAsPTSGVKY |
| 1251 | RLAsRPLLL |
| 1252 | RLAsSATQVHK |
| 1253 | RLAsYLDKV |
| 1254 | RLAsYLDRV |
| 1255 | RLDsTPGKVFL |
| 1256 | RLDsTPGKVFV |
| 1257 | RLDsYLRAP |
| 1258 | RLDsYVR |
| 1259 | RLDsYVRS |
| 1260 | RLDsYVRSL |
| 1261 | RLDsYVRSV |
| 1262 | RLDtGPQSL |
| 1263 | RLEsANRRL |
| 1264 | RLFsFSKTPK |
| 1265 | RLFsKEL |
| 1266 | RLFsKELR |
| 1267 | RLFsKELRC |
| 1268 | RLFsKELRV |
| 1269 | RLFSLsNPSL |
| 1270 | RLFsPTYGL |
| 1271 | RLFsPTYGV |
| 1272 | RLFsQGQDV |
| 1273 | RLFVGsIPK |
| 1274 | RLGsFHELLL |
| 1275 | RLIsFKAEV |
| 1276 | RLIsPYKKK |
| 1277 | RLIsQDVKL |
| 1278 | RLIsQDVKV |
| 1279 | RLKLPsGSK |
| 1280 | RLKLPsGSKK |
| 1281 | RLKLPsGSKY |
| 1282 | RLKsDERPVHI |
| 1283 | RLKsPFRKK |
| 1284 | RLKsPGsGHVK |
| 1285 | RLKsPISLK |
| 1286 | RLKsPISLY |
| 1287 | RLKsPSPKSEK |
| 1288 | RLKsPSPKSER |
| 1289 | RLKtPTSQSYK |
| 1290 | RLKtPTSQSYR |
| 1291 | RLKTtPLRK |
| 1292 | RLKTtPLRR |
| 1293 | RLLDPsSPLAL |
| 1294 | RLLDPSsPLAL |
| 1295 | RLLDRSPsRSAK |
| 1296 | RLLDRSPsRSAY |
| 1297 | RLLsDGQQIIL |
| 1298 | RLLsDLEEL |
| 1299 | RLLsDQTRL |
| 1300 | RLLsFQRYL |
| 1301 | RLLsGVVTK |
| 1302 | RLLsGVVTY |
| 1303 | RLLsHISEA |
| 1304 | RLLsHISEV |
| 1305 | RLLsPLSSA |
| 1306 | RLLsPLSSARL |
| 1307 | RLLsPLSSV |
| 1308 | RLLsPQQPAL |
| 1309 | RLLsPRPSL |
| 1310 | RLLsPRPSLL |
| 1311 | RLLsPSMASK |
| 1312 | RLLsSGVSEI |
| 1313 | RLLsSGVSEV |
| 1314 | RLLsTDAEAV |
| 1315 | RLLsVEIVK |
| 1316 | RLLsVEIVY |
| 1317 | RLLsVHDFDF |
| 1318 | RLLsVILIK |
| 1319 | RLMsMPVAK |
| 1320 | RLMsMPVAY |
| 1321 | RLNtSDFQKL |
| 1322 | RLPNRIPsL |
| 1323 | RLPsFLKKNK |
| 1324 | RLPsLVHGY |
| 1325 | RLPsSTLKK |
| 1326 | RLPsSTLKR |
| 1327 | RLPsSTLKY |
| 1328 | RLQsLIKNI |
| 1329 | RLQsTSERL |
| 1330 | RLQsTSERV |
| 1331 | RLR(sLss)PTVTL |
| 1332 | RLR(sLss)PTVTV |
| 1333 | RLRQsPLATK |
| 1334 | RLRQsPLATR |
| 1335 | RLRQsPLATY |
| 1336 | RLRRsPLLK |
| 1337 | RLRsAGAAQK |
| 1338 | RLRsLSSLREK |
| 1339 | RLRsPPPVSK |
| 1340 | RLRsYEDMI |
| 1341 | RLRTsPITRK |
| 1342 | RLRTsPITRR |
| 1343 | RLSDtPPLL |
| 1344 | RLSsLIRHK |
| 1345 | RLSsLRASTSK |
| 1346 | RLSsPISKK |
| 1347 | RLSsPISKR |
| 1348 | RLSsPISKY |
| 1349 | RLsSPLHFV |
| 1350 | RLSsPLHFV |
| 1351 | RLSsPVLHK |
| 1352 | RLSsPVLHR |
| 1353 | RLSsPVLHY |
| 1354 | RLSsRFSSK |
| 1355 | RLSsRFSSR |
| 1356 | RLSsRFSSY |
| 1357 | RLSsRYSQK |
| 1358 | RLSsRYSQY |
| 1359 | RLSsVKLISK |
| 1360 | RLSsVKLISY |
| 1361 | RLTFsPTYGV |
| 1362 | RLVsLSMRK |
| 1363 | RLVsLSMRY |
| 1364 | RLYKsPLRH |
| 1365 | RLYKsPLRK |
| 1366 | RLYQyIQSK |
| 1367 | RLYQyIQSR |
| 1368 | RLYQyIQSRFK |
| 1369 | RLYQyIQSRFY |
| 1370 | RLYQyIQSY |
| 1371 | RLYQylOSK |
| 1372 | RLYQyLQSRF |
| 1373 | RLYQyLQSRFK |
| 1374 | RLYQyLQSRFY |
| 1375 | RLYQyLQSRK |
| 1376 | RLYsGPMNKV |
| 1377 | RLYsGSRsK |
| 1378 | RLYsGSRsR |
| 1379 | RLYsGSRsY |
| 1380 | RLYsKSRDK |
| 1381 | RLYsPDHRQK |
| 1382 | RLYsPERSK |
| 1383 | RLYsPRNSK |
| 1384 | RLYsPYNHK |
| 1385 | RLYsPYNHR |
| 1386 | RLYsPYNHY |
| 1387 | RLYSRsFSK |
| 1388 | RLYSRsFSY |
| 1389 | RLYsYPRQK |
| 1390 | RLYVTTSTRTYsLG |
| 1391 | RLYVTTSTRTYsLK |
| 1392 | RLYVTTSTRTYsLY |
| 1393 | RMAsPPPPPK |
| 1394 | RMAsPTSGV |
| 1395 | RMAsPTSGVK |
| 1396 | RMAsPTSGVKK |
| 1397 | RMAsPTSGVKR |
| 1398 | RMAsPTSGVKY |
| 1399 | RMAsSATQVHK |
| 1400 | RMDsTPGKVFL |
| 1401 | RMDsTPGKVFV |
| 1402 | RMDsYVRSL |
| 1403 | RMDsYVRSV |
| 1404 | RMFPtPPSL |
| 1405 | RMFsFSKTPK |
| 1406 | RMFsKELRC |
| 1407 | RMFsKELRV |
| 1408 | RMFsPMEEK |
| 1409 | RMFsPMEEKELL |
| 1410 | RMFsPTYGL |
| 1411 | RMFsPTYGV |
| 1412 | RMIsPYKKK |
| 1413 | RMIsQDVKL |
| 1414 | RMIsQDVKV |
| 1415 | RMIsTGSEL |
| 1416 | RMKLPsGSK |
| 1417 | RMKLPsGSKK |
| 1418 | RMKLPsGSKY |
| 1419 | RMKsPFRKK |
| 1420 | RMKsPGsGHVK |
| 1421 | RMKsPSPKSEK |
| 1422 | RMKtPTSQSYK |
| 1423 | RMKtPTSQSYR |
| 1424 | RMKTtPLRK |
| 1425 | RMKTtPLRR |
| 1426 | RMLDRSPsRSAK |
| 1427 | RMLDRSPsRSAY |
| 1428 | RMLsHISEA |
| 1429 | RMLsHISEV |
| 1430 | RMLsLRDQRL |
| 1431 | RMLsPLSSA |
| 1432 | RMLsPLSSV |
| 1433 | RMLsPSMASK |
| 1434 | RMLsSGVSEI |
| 1435 | RMLsSGVSEV |
| 1436 | RMLsVILIK |
| 1437 | RMPsFLKKNK |
| 1438 | RMPsSTLKK |
| 1439 | RMPsSTLKR |
| 1440 | RMQsTSERL |
| 1441 | RMQsTSERV |
| 1442 | RMRQsPLATK |
| 1443 | RMRQsPLATR |
| 1444 | RMRRsPLLK |
| 1445 | RMRsAGAAQK |
| 1446 | RMRsLSSLREK |
| 1447 | RMRsPPPVSK |
| 1448 | RMRTsPITRK |
| 1449 | RMRTsPITRR |
| 1450 | RMSsLIRHK |
| 1451 | RMSsPISKK |
| 1452 | RMSsPISKR |
| 1453 | RMSsPLHFV |
| 1454 | RMSsPVLHK |
| 1455 | RMSsRYSQK |
| 1456 | RMSsVKLISK |
| 1457 | RMGsVKLISY |
| 1458 | RMVsLSMRK |
| 1459 | RMVsLSMRY |
| 1460 | RMYKsPLRH |
| 1461 | RMYKsPLRK |
| 1462 | RMYQyIQSK |
| 1463 | RMYQyIQSR |
| 1464 | RMYQyLQSRF |
| 1465 | RMYQyLQSRFK |
| 1466 | RMYQyLQSRFY |
| 1467 | RMYQyLQSRK |
| 1468 | RMYsFDDVL |
| 1469 | RMYsGSRsK |
| 1470 | RMYsGSRsR |
| 1471 | RMYsKSRDH |
| 1472 | RMYsKSRDK |
| 1473 | RMYsKSRDY |
| 1474 | RMYsPDHRQK |
| 1475 | RMYsPERSK |
| 1476 | RMYsPIIYQA |
| 1477 | RMYsPIPPSL |
| 1478 | RMYsPRNSK |
| 1479 | RMYsPYNHK |
| 1480 | RMYsPYNHR |
| 1481 | RMYsYPRQK |
| 1482 | RMYVTTSTRTYsLG |
| 1483 | RMYVTTSTRTYsLK |
| 1484 | RMYVTTSTRTYsLY |
| 1485 | RNLsSPFIF |
| 1486 | RPAFFsPSL |
| 1487 | RPAKsMDSF |
| 1488 | RPAKsMDSL |
| 1489 | RPAKsMDSM |
| 1490 | RPAKsMDV |
| 1491 | RPAsAGAMF |
| 1492 | RPAsAGAmL |
| 1493 | RPAsAGAML |
| 1494 | RPAsAGAMM |
| 1495 | RPAsAGAMV |
| 1496 | RPAsARAQPGF |
| 1497 | RPAsARAQPGL |
| 1498 | RPAsARAQPGM |
| 1499 | RPAsARAQPGV |
| 1500 | RPAsEARAPGL |
| 1501 | RPAsPAAKF |
| 1502 | RPAsPAAKL |
| 1503 | RPAsPAAKM |
| 1504 | RPAsPAAKV |
| 1505 | RPAsPEPEL |
| 1506 | RPAsPGPSL |
| 1507 | RPAsPKRAKI |
| 1508 | RPAsPKRAKL |
| 1509 | RPAsPKRAKX |
| 1510 | RPAsPKRAQI |
| 1511 | RPAsPKRAQL |
| 1512 | RPAsPKRAQX |
| 1513 | RPAsPQRAKI |
| 1514 | RPAsPQRAKL |
| 1515 | RPAsPQRAKX |
| 1516 | RPAsPQRAQI |
| 1517 | RPAsPQRAQL |
| 1518 | RPAsPQRAQX |
| 1519 | RPAsPSLQL |
| 1520 | RPAsPSLQLL |
| 1521 | RPAsPtAIRRTGSVTSRQT |
| 1522 | RPAsRFEVL |
| 1523 | RPAsYKKKSML |
| 1524 | RPAtGGPGVA |
| 1525 | RPAtGGPGVF |
| 1526 | RPAtGGPGVL |
| 1527 | RPAtGGPGVM |
| 1528 | RPAtGGPGVV |
| 1529 | RPAtPTSQF |
| 1530 | RPAtPTSQL |
| 1531 | KPAtPTSQM |
| 1532 | RPAtPTSQV |
| 1533 | RPDsAHKML |
| 1534 | RPDsPTRPTL |
| 1535 | RPDsRLGKTEF |
| 1536 | RPDsRLGKTEL |
| 1537 | RPDsRLGKTEM |
| 1538 | RPDsRLGKTEV |
| 1539 | RPDVAKRLsL |
| 1540 | RPEsDSGLKF |
| 1541 | RPEsDSGLKL |
| 1542 | RPEsDSGLKM |
| 1543 | RPEsDSGLKV |
| 1544 | RPEsKDRKF |
| 1545 | RPEsKDRKL |
| 1546 | RPEsKDRKM |
| 1547 | RPEsKDRKV |
| 1548 | RPFARsHSF |
| 1549 | RPFARSHsF |
| 1550 | RPFHGISTVsL |
| 1551 | RPFsPREAF |
| 1552 | RPFsPREAL |
| 1553 | RPFsPREAM |
| 1554 | RPFsPREAV |
| 1555 | RPGsLERKF |
| 1556 | RPGsLERKL |
| 1557 | RPGsLERKM |
| 1558 | RPGsLERKV |
| 1559 | RPGsRQAGL |
| 1560 | RPGsRqAGL |
| 1561 | RPHsPEKAF |
| 1562 | RPHsPEKAL |
| 1563 | RPHsPEKAM |
| 1564 | RPHsPEKAV |
| 1565 | RPHtPTGIYM |
| 1566 | RPHtPTPGIYM |
| 1567 | RPIsPGLSF |
| 1568 | RPIsPGLSL |
| 1569 | RPIsPGLSM |
| 1570 | RPIsPGLSV |
| 1571 | RPIsPGLSY |
| 1572 | RPIsPPHTY |
| 1573 | RPIsPRIGAL |
| 1574 | RPItPPRNSA |
| 1575 | RPItPPRNSF |
| 1576 | RPItPPRNSL |
| 1577 | RPItPPRNSM |
| 1578 | RPItPPRNSV |
| 1579 | RPKLSsPAF |
| 1580 | RPKLSsPAL |
| 1581 | RPKLSsPAM |
| 1582 | RPKLSsPAV |
| 1583 | RPKPSSsPF |
| 1584 | RPKPSSsPL |
| 1585 | RPKPSSsPM |
| 1586 | RPKPSSsPV |
| 1587 | RPKsNIVLF |
| 1588 | RPKsNIVLL |
| 1589 | RPKsNIVLM |
| 1590 | RPKsNIVLV |
| 1591 | RPKsPLSKm |
| 1592 | RPKsPLSKM |
| 1593 | RPKsQVAEF |
| 1594 | RPKsQVAEL |
| 1595 | RPKsQVAEM |
| 1596 | RPKsQVAEV |
| 1597 | RPKsVDFDSL |
| 1598 | RPKtPPVVI |
| 1599 | RPLsLLLAL |
| 1600 | RPLsPGGAF |
| 1601 | RPLsPGGAL |
| 1602 | RPLsPGGAM |
| 1603 | RPLsPGGAV |
| 1604 | RPLsPLLF |
| 1605 | RPLsPLLL |
| 1606 | RPLsPLLM |
| 1607 | RPLsPLLV |
| 1608 | RPLsYVL |
| 1609 | RPMsESPHM |
| 1610 | RPNsPSPTAF |
| 1611 | RPNsPSPTAL |
| 1612 | RPNsPSPTAM |
| 1613 | RPNsPSPTAV |
| 1614 | RPPIgTQSSL |
| 1615 | RPPPPPDtPF |
| 1616 | RPPPPPDtPL |
| 1617 | RPPPPPDtPM |
| 1618 | RPPPPPDtPP |
| 1619 | RPPPPPDtPV |
| 1620 | RPPsPGPVF |
| 1621 | RPPsPGPVL |
| 1622 | RPPsPGPVM |
| 1623 | RPPsPGPVV |
| 1624 | RPPsPSSRF |
| 1625 | RPPsPSSRL |
| 1626 | RPPsPSSRM |
| 1627 | RPPsPSSRV |
| 1628 | RPPsSEFLDF |
| 1629 | RPPsSEFLDL |
| 1630 | RPPsSEFLDM |
| 1631 | RPPsSEFLDV |
| 1632 | RPQKTQsII |
| 1633 | RPQRAtSNVF |
| 1634 | RPQRATsNVF |
| 1635 | RPQRAtSNVL |
| 1636 | RPQRATsNVL |
| 1637 | RPQRAtSNVM |
| 1638 | RPQRATsNVM |
| 1639 | RPQRAtSNVV |
| 1640 | RPQRATsNVV |
| 1641 | RPR(sLss)PTVTL |
| 1642 | RPR(sLss)PTVTV |
| 1643 | RPRAAtVV |
| 1644 | RPRAAtVVA |
| 1645 | RPRAAtW |
| 1646 | RPRAAtWA |
| 1647 | RPRANsGGVDF |
| 1648 | RPRANsGGVDL |
| 1649 | RPRANsGGVDM |
| 1650 | RPRANsGGVDV |
| 1651 | RPRARsVDAL |
| 1652 | RPRDtRRISL |
| 1653 | RPRGsESLL |
| 1654 | RPRGsQSLF |
| 1655 | RPRGsQSLL |
| 1656 | RPRGsQSLM |
| 1657 | RPRGsQSLV |
| 1658 | RPRIPsPIGF |
| 1659 | RPRLSsTNSSRF |
| 1660 | RPRPAsSPAL |
| 1661 | RPRPHsAPSF |
| 1662 | RPRPHsAPSL |
| 1663 | RPRPHsAPSM |
| 1664 | RPRPHsAPSV |
| 1665 | RPRPSsAHVGL |
| 1666 | RPRPsSVL |
| 1667 | RPRPsSVLRTL |
| 1668 | RPRPVsPSSF |
| 1669 | RPRPVsPSSL |
| 1670 | RPRPVsPSSLL |
| 1671 | RPRPVsPSSM |
| 1672 | RPRPVsPSSV |
| 1673 | RPRRsSTQF |
| 1674 | RPRRsSTQL |
| 1675 | RPRRsSTQM |
| 1676 | RPRRsSTQV |
| 1677 | RPRsAVEQL |
| 1678 | RPRsAVLF |
| 1679 | RPRsAVLL |
| 1680 | RPRsAVLM |
| 1681 | RPRsAVLV |
| 1682 | RPRSGsTGSSL |
| 1683 | RPRsISVEEF |
| 1684 | RPRsISVEEL |
| 1685 | RPRsISVEEM |
| 1686 | RPRsISVEEV |
| 1687 | RPRsLEVTF |
| 1688 | RPRsLEVTI |
| 1689 | RPRsLEVTL |
| 1690 | RPRsLEVTM |
| 1691 | RPRsLEVTV |
| 1692 | RPRSLsSPTV |
| 1693 | RPRSLsSPTVTF |
| 1694 | RPRSLsSPTVTL |
| 1695 | RPRSLsSPTVTM |
| 1696 | RPRSLsSPTVTV |
| 1697 | RPRsMTVSA |
| 1698 | RPRsMVRSF |
| 1699 | RPRsPAARF |
| 1700 | RPRsPAARL |
| 1701 | RPRsPAARM |
| 1702 | RPRsPAARV |
| 1703 | RPRsPGSNSKV |
| 1704 | RPRsPNMQDL |
| 1705 | RPRsPPGGP |
| 1706 | RPRsPPPRAF |
| 1707 | RPRsPPPRAL |
| 1708 | RPRsPPPRAM |
| 1709 | RPRsPPPRAP |
| 1710 | RPRsPPPRAV |
| 1711 | RPRsPPSSP |
| 1712 | RPRsPRENSF |
| 1713 | RPRsPRENSI |
| 1714 | RPRsPRENSL |
| 1715 | RPRsPRENSM |
| 1716 | RPRsPRENSV |
| 1717 | RPRsPRPPP |
| 1718 | RPRsPRQNLI |
| 1719 | RPRsPRQNSF |
| 1720 | RPRsPRQNSI |
| 1721 | RPRsPRQNSM |
| 1722 | RPRsPRQNSV |
| 1723 | RPRsPSPIF |
| 1724 | RPRsPSPIL |
| 1725 | RPRsPSPIM |
| 1726 | RPRsPSPIS |
| 1727 | RPRSPsPIS |
| 1728 | RPRsPSPIV |
| 1729 | RPRsPTGF |
| 1730 | RPRsPTGL |
| 1731 | RPRsPTGM |
| 1732 | RPRsPTGP |
| 1733 | RPRsPTGPsNSF |
| 1734 | RPRsPTGPSNSF |
| 1735 | RPRsPTGPSNSFL |
| 1736 | RPRsPTGPsNSL |
| 1737 | RPRsPTGPsNSM |
| 1738 | RPRsPTGPsNSV |
| 1739 | RPRsPTGV |
| 1740 | RPRsPTRSF |
| 1741 | RPRsPTRSL |
| 1742 | RPRsPTRSM |
| 1743 | RPRsPTRSV |
| 1744 | RPRsPWGKL |
| 1745 | RPRsQYNTKL |
| 1746 | RPRSTsQSIVSL |
| 1747 | RPRtPLRSL |
| 1748 | RPSGRREsF |
| 1749 | RPSGRREsL |
| 1750 | RPSGRREsM |
| 1751 | RPSGRREsV |
| 1752 | RPsNPQL |
| 1753 | RPSRSsPGF |
| 1754 | RPSRSsPGL |
| 1755 | RPSRSsPGM |
| 1756 | RPSRSsPGV |
| 1757 | RPSsGFYEL |
| 1758 | RPSsLDAEIDSF |
| 1759 | RPSsLDAEIDSL |
| 1760 | RPSsLDAEIDSM |
| 1761 | RPSsLDAEIDSV |
| 1762 | RPSsLPDF |
| 1763 | RPSsLPDL |
| 1764 | RPSsLPDM |
| 1765 | RPSsLPDV |
| 1766 | RPsSPALYF |
| 1767 | RPSsPALYF |
| 1768 | RPsSPALYL |
| 1769 | RPsSPALYM |
| 1770 | RPsSPALYV |
| 1771 | RPStPKSDSEF |
| 1772 | RPStPKSDSEL |
| 1773 | RPSLPKSDSEM |
| 1774 | RPStPKSDSEV |
| 1775 | RPTKIGRRsL |
| 1776 | RPTsFADEL |
| 1777 | RPTsPIQIM |
| 1778 | RPTsRLNRF |
| 1779 | RPTsRLNRL |
| 1780 | RPTsRLNRM |
| 1781 | RPTsRLNRV |
| 1782 | RPVsPFQEF |
| 1783 | RPVsPFQEL |
| 1784 | RPVsPFQEM |
| 1785 | RPVsPFQEV |
| 1786 | RPVsPGKDF |
| 1787 | RPVsPGKDI |
| 1788 | RPVsPGKDL |
| 1789 | RPVsPGKDM |
| 1790 | RPVsPGKDV |
| 1791 | RPVSPsSLL |
| 1792 | RPVsTDFAQY |
| 1793 | RPVtPVSDF |
| 1794 | RPVtPVSDL |
| 1795 | RPVtPVSDM |
| 1796 | RPVtPVSDV |
| 1797 | RPWsNSRGL |
| 1798 | RPWsPAVSA |
| 1799 | RPWsPAVSF |
| 1800 | RPWsPAVSL |
| 1801 | RPWsPAVSM |
| 1802 | RPWsPAVSV |
| 1803 | RPYsPPFFSF |
| 1804 | RPYsPPFFSL |
| 1805 | RPYsPPFFSM |
| 1806 | RPYsPPFFSV |
| 1807 | RPYSPsQAL |
| 1808 | RPYsPSQYAL |
| 1809 | RPYSPsQYAL |
| 1810 | RPYsQVNVL |
| 1811 | RQAsIELPSM |
| 1812 | RQAsIELPSMAV |
| 1813 | RQAsIELPSV |
| 1814 | RQAsLSISV |
| 1815 | RQAsPLVHK |
| 1816 | RQAsPLVIIR |
| 1817 | RQAsPLVHY |
| 1818 | RQDsTPGKVFL |
| 1819 | RQDStPGKVFL |
| 1820 | RQDsTPGKVFV |
| 1821 | RQIsFKAEV |
| 1822 | RQIsQDVKL |
| 1823 | RQIsQDVKV |
| 1824 | RQKsPLFQF |
| 1825 | RQLsALHRA |
| 1826 | RQLsLEGSGLGV |
| 1827 | RQLsSGVSEI |
| 1828 | RQLsSGVSEV |
| 1829 | RQSsSRFNL |
| 1830 | RRAsFAKSF |
| 1831 | RRAsFAKSK |
| 1832 | RRAsFAKSL |
| 1833 | RRAsFAKSM |
| 1834 | RRAsFAKSR |
| 1835 | RRAsIITKY |
| 1836 | RRAsLSEIGF |
| 1837 | RRAsLSEIGK |
| 1838 | RRAsLSEIGY |
| 1839 | RRAsQEANL |
| 1840 | RRASsPFRF |
| 1841 | RRASsPFRK |
| 1842 | RRASsPFRL |
| 1843 | RRASsPFRM |
| 1844 | RRASsPFRR |
| 1845 | RRAsVFVKF |
| 1846 | RRAsVFVKK |
| 1847 | RRAsVFVKL |
| 1848 | RRAsVFVKM |
| 1849 | RRAsVFVKR |
| 1850 | RRDsIVAEF |
| 1851 | RRDsIVAEK |
| 1852 | RRDsIVAEL |
| 1853 | RRDsIVAER |
| 1854 | RRDsIVAEY |
| 1855 | RRDsLQKPGL |
| 1856 | RRFsFEVTL |
| 1857 | RRFsFKF |
| 1858 | RRFsFKK |
| 1859 | RRFsFKKSF |
| 1860 | RRFsFKKSK |
| 1861 | RRFsFKKSL |
| 1862 | RRFsFKKSM |
| 1863 | RRFsFKKSR |
| 1864 | RRFsFKL |
| 1865 | RRFsFKM |
| 1866 | RRFsFKR |
| 1867 | RRFsGTAVY |
| 1868 | RRFsGTVRF |
| 1869 | RRFsGTVRK |
| 1870 | RRFsGTVRL |
| 1871 | RRFsGTVRM |
| 1872 | RRFsGTVRR |
| 1873 | RRFsIATLR |
| 1874 | RRFsLTTLR |
| 1875 | RRFsPDDKYSF |
| 1876 | RRFsPDDKYSK |
| 1877 | RRFsPDDKYSL |
| 1878 | RRFsPDDKYSM |
| 1879 | RRFsPPRRF |
| 1880 | RRFsPPRRK |
| 1881 | RRFsPPRRL |
| 1882 | RRFsPPRRm |
| 1883 | RRFsPPRRM |
| 1884 | RRFsPPRRR |
| 1885 | RRFsPPRRY |
| 1886 | RRFsRLENRY |
| 1887 | RRFsRSDEL |
| 1888 | RRFsRsPIF |
| 1889 | RRFsRSPIF |
| 1890 | RRFsRsPIK |
| 1891 | RRFsRSPIK |
| 1892 | RRFsRsPIL |
| 1893 | RRFsRSPIL |
| 1894 | RRFsRSPIM |
| 1895 | RRFsRsPIR |
| 1896 | RRFsRGPIR |
| 1897 | RRFSRsPIR |
| 1898 | RRFsRsPIRF |
| 1899 | RRFsRSPIRF |
| 1900 | RRFsRsPIRK |
| 1901 | RRFsRSPIRK |
| 1902 | RRFsRsPIRL |
| 1903 | RRFsRSPIRL |
| 1904 | RRFsRsPIRR |
| 1905 | RRFsRSPIRR |
| 1906 | RRFsRsPIRY |
| 1907 | RRFsRSPIRY |
| 1908 | RRFsRsPIY |
| 1909 | RRFsRSPIY |
| 1910 | RRFsRSPK |
| 1911 | RRFSsPPRRM |
| 1912 | RRFsVSTLR |
| 1913 | RRFsVTTMR |
| 1914 | RRFtPPSPAF |
| 1915 | RRFtPPSPAK |
| 1916 | RRFtPPSPAR |
| 1917 | RRFtPPSPAY |
| 1918 | RRGsFEVTL |
| 1919 | RRHsASNLHAL |
| 1920 | RRIDIsPSTF |
| 1921 | RRIDIsPSTK |
| 1922 | RRIDIsPSTLR |
| 1923 | RRIDIsPSTLRK |
| 1924 | RRIDIsPSTR |
| 1925 | RRIDIsPSTY |
| 1926 | RRIsDPEVF |
| 1927 | RRIsDPQVF |
| 1928 | RRIsGVDRF |
| 1929 | RRIsGVDRK |
| 1930 | RRIsGVDRL |
| 1931 | RRIsGVDRM |
| 1932 | RRIsGVDRR |
| 1933 | RRIsGVDRY |
| 1934 | RRIsGVDRYF |
| 1935 | RRIsGVDRYK |
| 1936 | RRIsGVDRYL |
| 1937 | RRIsGVDRYR |
| 1938 | RRIsGVDRYY |
| 1939 | RRIsPAPQR |
| 1940 | RRIsQIQQL |
| 1941 | RRKsOVAEF |
| 1942 | RRKsOVAEK |
| 1943 | RRKsPPPSF |
| 1944 | RRKsPPPSK |
| 1945 | RRKsPPPSL |
| 1946 | RRKsPPPSM |
| 1947 | RRKsPPPSR |
| 1948 | RRKsQLDSF |
| 1949 | RRKsQLDSK |
| 1950 | RRKsQLDSL |
| 1951 | RRKsQLDSM |
| 1952 | RRKsQLDSR |
| 1953 | RRKsQLDSY |
| 1954 | RRKsQVAEF |
| 1955 | RRKsQVAEK |
| 1956 | RRKsQVAEL |
| 1957 | RRKsQVAEM |
| 1958 | RRKsQVAER |
| 1959 | RRKsQVAEV |
| 1960 | RRKsQVAEY |
| 1961 | RRLGsPHRF |
| 1962 | RRLGsPHRK |
| 1963 | RRLGsPHRL |
| 1964 | RRLGsPHRM |
| 1965 | RRLGsPHRR |
| 1966 | RRLsADIRF |
| 1967 | RRLsADIRK |
| 1968 | RRLsADIRL |
| 1969 | RRLsADIRM |
| 1970 | RRLsADIRR |
| 1971 | RRLsADIRY |
| 1972 | RRLsDSPVF |
| 1973 | RRLsELLRY |
| 1974 | RRLsERETR |
| 1975 | RRLsESSAL |
| 1976 | RRLsFLVSF |
| 1977 | RRLsFLVSK |
| 1978 | RRLsFLVSL |
| 1979 | RRLsFLVSM |
| 1980 | RRLsFLVSR |
| 1981 | RRLsFLVSY |
| 1982 | RRLsGGSHSF |
| 1983 | RRLsGGSHSK |
| 1984 | RRLsGGSHSL |
| 1985 | RRLsGGSHSM |
| 1986 | RRLsGGSHSR |
| 1987 | RRLsGGSHSY |
| 1988 | RRLsGPLHTF |
| 1989 | RRLsGPLHTK |
| 1990 | RRLsGPLHTL |
| 1991 | RRLsGPLHTM |
| 1992 | RRLsGPLHTR |
| 1993 | RRLsGPLHTV |
| 1994 | RRLsGPLHTY |
| 1995 | RRLsLFLNV |
| 1996 | RRLsNLPTF |
| 1997 | RRLsNLPTK |
| 1998 | RRLsNLPTR |
| 1999 | RRLsNLPTV |
| 2000 | RRLsNLPTY |
| 2001 | RRLsPAPCF |
| 2002 | RRLsPAPQK |
| 2003 | RRLsPAPQL |
| 2004 | RRLsPAPQM |
| 2005 | RRLsPKASQVF |
| 2006 | RRLs PKASQVK |
| 2007 | RRLsPKASQVL |
| 2008 | RRLsPKASQVM |
| 2009 | RRLsPKASQVR |
| 2010 | RRLsPVPVPF |
| 2011 | RRLsPVPVPK |
| 2012 | RRLsPVPVPL |
| 2013 | RRLsPVPVPM |
| 2014 | RRLsPVPVPR |
| 2015 | RRLsRELCK |
| 2016 | RRLsRELQF |
| 2017 | RRLsRELQL |
| 2018 | RRLsRELQM |
| 2019 | RRLsRELQR |
| 2020 | RRLsRKLSL |
| 2021 | RRLsVERIF |
| 2022 | RRLsVERIK |
| 2023 | RRLsVERIM |
| 2024 | RRLsVERIR |
| 2025 | RRLsYVLFI |
| 2026 | RRLTHLsF |
| 2027 | RRLTHLsK |
| 2028 | RRLTHLsL |
| 2029 | RRLTHLsM |
| 2030 | RRLTHLsR |
| 2031 | RRMsFQKP |
| 2032 | RRMsLLSVF |
| 2033 | RRMsLLSVK |
| 2034 | RRMsLLSVL |
| 2035 | RRMsLLSVM |
| 2036 | RRMsLLSVR |
| 2037 | RRmsLLSVV |
| 2038 | RRMsLLSVV |
| 2039 | RRMsLLSVY |
| 2040 | RRMsLLSW |
| 2041 | RRMsLSVM |
| 2042 | RRMsPIKPL |
| 2043 | RRMsPKAOR |
| 2044 | RRMsPKAQF |
| 2045 | RRMsPKAQK |
| 2046 | RRMsPKAQL |
| 2047 | RRMsPKAQM |
| 2048 | RRMsPKPF |
| 2049 | RRMsPKPK |
| 2050 | RRMsPKPM |
| 2051 | RRMsPKPR |
| 2052 | RRNsAPVSV |
| 2053 | RRNsINRNF |
| 2054 | RRNsNPVIAEF |
| 2055 | RRNsNPVIAEK |
| 2056 | RRNsNPVIAEL |
| 2057 | RRNsNPVIAEM |
| 2058 | RRNsNPVIAER |
| 2059 | RRNsSERTF |
| 2060 | RRNsSERTK |
| 2061 | RRNsSERTL |
| 2062 | RRNsSERTM |
| 2063 | RRNsSERTR |
| 2064 | RRNsSERTY |
| 2065 | RRNsSIVGF |
| 2066 | RRNsSIVGK |
| 2067 | RRNsSIVGL |
| 2068 | RRNsSIVGM |
| 2069 | RRNsSIVGR |
| 2070 | RRNsSIVGY |
| 2071 | RRNsVFQQGF |
| 2072 | RRNsVFQQGK |
| 2073 | RRNsVFQQGL |
| 2074 | RRNsVFQQGM |
| 2075 | RRNsVFQQGR |
| 2076 | RRNsVFQQGY |
| 2077 | RRPsIAPVL |
| 2078 | RRPsLLSEF |
| 2079 | RRPsLVHGF |
| 2080 | RRPsLVHGK |
| 2081 | RRPsLVHGL |
| 2082 | RRPsLVHGM |
| 2083 | RRPsLVHGR |
| 2084 | RRPsLVHGY |
| 2085 | RRPsVFERF |
| 2086 | RRPsVFERK |
| 2087 | RRPsVFERL |
| 2088 | RRPsVFERM |
| 2089 | RRPsVFERR |
| 2090 | RRPsVFERY |
| 2091 | RRPsYRKIF |
| 2092 | RRPsYRKIK |
| 2093 | RRPsYRKIL |
| 2094 | RRPsYRKIM |
| 2095 | RRPsYRKIR |
| 2096 | RRPsYRKIY |
| 2097 | RRPsYTLGF |
| 2098 | RRPsYTLGK |
| 2099 | RRPsYTLGL |
| 2100 | RRPsYTLGM |
| 2101 | RRPsYTLGR |
| 2102 | RRPsYTLGV |
| 2103 | RRPsYTLGY |
| 2104 | RRCsKVEAL |
| 2105 | RRRsLERLL |
| 2106 | RRsFLVSY |
| 2107 | RRSFsLE |
| 2108 | RRSsFLQ |
| 2109 | RRssFLQLF |
| 2110 | RRssFLQVF |
| 2111 | RRSsFLQVF |
| 2112 | RRSsFLQVK |
| 2113 | RRSsFLQVL |
| 2114 | RRssFLQVM |
| 2115 | RRSsFLQVM |
| 2116 | RRSsFLQVR |
| 2117 | RRssFLQW |
| 2118 | RRSsFLQVY |
| 2119 | RRSsIGLRF |
| 2120 | RRSsIGLRK |
| 2121 | RRSsIGLRL |
| 2122 | RRSsIGLRM |
| 2123 | RRSsIGLRR |
| 2124 | RRSsIGLRV |
| 2125 | RRSsIGLRY |
| 2126 | RRsSIQSTF |
| 2127 | RRSsIQSTF |
| 2128 | RRSsIQSTK |
| 2129 | RRSsIQSTL |
| 2130 | RRSsIQSTM |
| 2131 | RRSsIQSTR |
| 2132 | RRSsIQSTY |
| 2133 | RRSsLDAEIDSF |
| 2134 | RRSsLDAEIDSL |
| 2135 | RRSsLDAEIDSM |
| 2136 | RRGsLDAEIDSV |
| 2137 | RRsSQSWSF |
| 2138 | RRSsQSWSF |
| 2139 | RRSsQSWSK |
| 2140 | RRsSQSWSL |
| 2141 | RRSsQSWSL |
| 2142 | RRsSQSWSM |
| 2143 | RRSsQSWSM |
| 2144 | RRSsQSWSR |
| 2145 | RRsSQSWSV |
| 2146 | RRSsQSWSY |
| 2147 | RRSsSVAQV |
| 2148 | RRSsTASLVKF |
| 2149 | RRSsTASLVKK |
| 2150 | RRSsTASLVKL |
| 2151 | RRSsTASLVKM |
| 2152 | RRSsTASLVKR |
| 2153 | RRsSVDLGF |
| 2154 | RRSsVDLGF |
| 2155 | RRsSVDLGK |
| 2156 | RRSsVDLGK |
| 2157 | RRsSVDLGL |
| 2158 | RRSsVDLGL |
| 2159 | RRsSVDLGM |
| 2160 | RRSsVDLGM |
| 2161 | RRsSVDLGR |
| 2162 | RRSsVDLGR |
| 2163 | RRsSVDLGY |
| 2164 | RRSsVDLGY |
| 2165 | RRSsVKVEA |
| 2166 | RRSsVKVEF |
| 2167 | RRSsVKVEK |
| 2168 | RRSsVKVEL |
| 2169 | RRSsVKVEM |
| 2170 | RRSsVKVER |
| 2171 | RRSsVKVEY |
| 2172 | RRTsPITRF |
| 2173 | RRTsPITRK |
| 2174 | RRTsPITRL |
| 2175 | RRTsPITRM |
| 2176 | RRTsPITRR |
| 2177 | RRWQRSsF |
| 2178 | RRWQRSsK |
| 2179 | RRWQRSsL |
| 2180 | RRWQRSsM |
| 2181 | RRWQRSsR |
| 2182 | RRWQRSsY |
| 2183 | RRWQRSsL |
| 2184 | RRYsGKTEF |
| 2185 | RRYsGKTEK |
| 2186 | RRYsGKTEL |
| 2187 | RRYsGKTER |
| 2188 | RRYsGKTEY |
| 2189 | RRYsGNMEF |
| 2190 | RRYsGNMEK |
| 2191 | RRYsGNMEL |
| 2192 | RRYsGNMEM |
| 2193 | RRYsGNMER |
| 2194 | RRYsKFFDL |
| 2195 | RRYsPPIER |
| 2196 | RRYsPPIQ |
| 2197 | RRYsPPIQF |
| 2198 | RRYsPPIQK |
| 2199 | RRYsPPIQL |
| 2200 | RRYsPPIQM |
| 2201 | RRYsPPIQR |
| 2202 | RRYsPPIQY |
| 2203 | RRYsRsPYSF |
| 2204 | RRYsRSPYSF |
| 2205 | RRYSRsPYSF |
| 2206 | RRYsRsPYSK |
| 2207 | RRYsRSPYSK |
| 2208 | RRYSRsPYSK |
| 2209 | RRYsRsPYSL |
| 2210 | RRYsRSPYSL |
| 2211 | RRYSRsPYSL |
| 2212 | RRYsRsPYSM |
| 2213 | RRYsRSPYSM |
| 2214 | RRYSRsPYSM |
| 2215 | RRYsRsPYSR |
| 2216 | RRYsRSPYSR |
| 2217 | RRYSRsPYSR |
| 2218 | RRYtNRVVTK |
| 2219 | RRYtNRVVTL |
| 2220 | RRYtNRVVTM |
| 2221 | RRYtNRVVTR |
| 2222 | RSAsFSRKV |
| 2223 | RSAsPDDDLGSSN |
| 2224 | RSAsSATQVHK |
| 2225 | RSAsSATQVHY |
| 2226 | RSDPSKsPGSLRY |
| 2227 | RSEsPKIDL |
| 2228 | RSEsPKIDY |
| 2229 | RSEsRAQAV |
| 2230 | RSEsRAQAY |
| 2231 | RSEsVGENL |
| 2232 | RSEsVGENY |
| 2233 | RSEsYVEL |
| 2234 | RSEsYVELSQY |
| 2235 | RSEPSKsPGSLRY |
| 2236 | RSEsKDRKF |
| 2237 | RSEsKDRKL |
| 2238 | RSEsKDRKM |
| 2239 | RSEsKDRKV |
| 2240 | RSEsPKIDL |
| 2241 | RSEsPKIDY |
| 2242 | RSEsPPAEL |
| 2243 | RSEsRAQAV |
| 2244 | RSEsRAQAY |
| 2245 | RSEsVGENL |
| 2246 | RSEsVGENY |
| 2247 | RSEsYVELSQY |
| 2248 | RSFsPTMKV |
| 2249 | RSGsLERKF |
| 2250 | RSGsLERKL |
| 2251 | RSGsLERKM |
| 2252 | RSGsLERKV |
| 2253 | RSHSsPASL |
| 2254 | RSIsVGENL |
| 2255 | RSLsESYEL |
| 2256 | RSLsPGGAA |
| 2257 | RSLsPGGAF |
| 2258 | RSLsPGGAL |
| 2259 | RSLsPGGAM |
| 2260 | RSLsPGGAV |
| 2261 | RSLsPLLF |
| 2262 | RSLsPLLL |
| 2263 | RSLsPLLM |
| 2264 | RSLsPLLV |
| 2265 | RSLsQELVGV |
| 2266 | RSLsVEIVK |
| 2267 | RSLsVEIVY |
| 2268 | RSMsMPVAH |
| 2269 | RSMsMPVAK |
| 2270 | RsPEDEYELLMPHRISSH |
| 2271 | RSRRsPLLK |
| 2272 | RSRRsPLLY |
| 2273 | RSRsPLEL |
| 2274 | RSRsPPPVSK |
| 2275 | RSRsPPPVSY |
| 2276 | RSRsPRPAF |
| 2277 | RSRsPRPAI |
| 2278 | RSRsPRPAL |
| 2279 | RSRsPRPAM |
| 2280 | RSRsPRPAV |
| 2281 | RSRsPRPAX |
| 2282 | RSRTsPITRR |
| 2283 | RSRTsPITRY |
| 2284 | RSSsLIRHK |
| 2285 | RSSsLIRHY |
| 2286 | RSVsLSMRK |
| 2287 | RSVsLSMRY |
| 2288 | RsWKYNQSISLRRP |
| 2289 | RSYsGSRsK |
| 2290 | RSYsGSRsR |
| 2291 | RSYsGSRsY |
| 2292 | RSYsPDHRQK |
| 2293 | RSYsPDHRQY |
| 2294 | RSYsPERSK |
| 2295 | RSYsPERSY |
| 2296 | RSYsPRNSR |
| 2297 | RSYsPRNSY |
| 2298 | RSYSRsFSK |
| 2299 | RSYsRSFSR |
| 2300 | RSYSRsFSR |
| 2301 | RSYSRsFSY |
| 2302 | RSYsYPRQK |
| 2303 | RSYsYPRQY |
| 2304 | RSYVTTSTRTYsLG |
| 2305 | RTAsFAVRK |
| 2306 | RTAsFAVRY |
| 2307 | RTAsLIIKV |
| 2308 | RTAsPPPPPK |
| 2309 | RTDPSKsPGSLRY |
| 2310 | RTDsPKIDL |
| 2311 | RTDsPKIDY |
| 2312 | RTDsRAQAV |
| 2313 | RTDsRAQAY |
| 2314 | RTDsYVELSQY |
| 2315 | RTEPSKsPGSLRY |
| 2316 | RTEsDSGLKF |
| 2317 | RTEsDSGLKK |
| 2318 | RTEsDSGLKL |
| 2319 | RTEsDSGLKM |
| 2320 | RTEsDSGLKV |
| 2321 | RTEsPKIDL |
| 2322 | RTEsPKIDY |
| 2323 | RTEsRAQAV |
| 2324 | RTEsRAQAY |
| 2325 | RTEsYVELSQY |
| 2326 | RTFsLDTIL |
| 2327 | RTFsPTYGF |
| 2328 | RTFsPTYGL |
| 2329 | RTFsPTYGM |
| 2330 | RTFsPTYGV |
| 2331 | RTHsLLLLL |
| 2332 | RTLsHISEA |
| 2333 | RTLsHISEV |
| 2334 | RTLsPEIITV |
| 2335 | RTMsEAALVRK |
| 2336 | RTNsPGFQK |
| 2337 | RTPsDVKEL |
| 2338 | RTPsFLKKNK |
| 2339 | RTPsFLKKNY |
| 2340 | RTRsLSSLREK |
| 2341 | RTRsLSSLREY |
| 2342 | RTRsPSPTF |
| 2343 | RTRsPSPTL |
| 2344 | RTRsPSPTM |
| 2345 | RTRsPSPTV |
| 2346 | RTSsFALNL |
| 2347 | RTSsFTEQL |
| 2348 | RTSsFTFQN |
| 2349 | RTSSFtFQN |
| 2350 | RTSsPLFNK |
| 2351 | RTYKsPLRH |
| 2352 | RTYKsPLRK |
| 2353 | RTYKsPLRY |
| 2354 | RTYsGPMNK |
| 2355 | RTYsGPMNKV |
| 2356 | RTYsHGTYR |
| 2357 | RVAsFAVRK |
| 2358 | RVAsFAVRY |
| 2359 | RVAsPLVHK |
| 2360 | RVAsPLVHY |
| 2361 | RVAsPPPPPK |
| 2362 | RVAsPPPPPY |
| 2363 | RVAsPTSGV |
| 2364 | RVAsPTSGVK |
| 2365 | RVAsPTSGVKK |
| 2366 | RVAsPTSGVKR |
| 2367 | RVAsPTSGVY |
| 2368 | RVDsPSHGL |
| 2369 | RVGsLVLNL |
| 2370 | RVIsGVLQL |
| 2371 | RVKLPsGSKK |
| 2372 | RVKsPGsGHVK |
| 2373 | RVKsPGsGHVY |
| 2374 | RVKsPISLK |
| 2375 | RVKsPSPKSER |
| 2376 | RVKsPSPKSEY |
| 2377 | RVKtPTSQSYK |
| 2378 | RVKtPTSQSYR |
| 2379 | RVKtPTSQSYY |
| 2380 | RVKTtPLRR |
| 2381 | RVKTtPLRY |
| 2382 | RVLDRSPsRSAK |
| 2383 | RVLDRSPsRSAY |
| 2384 | RVLHsPPAV |
| 2385 | RVLsGVVTK |
| 2386 | RVLsPLIIK |
| 2387 | RVPsLLVLL |
| 2388 | RVPsSTLKK |
| 2389 | RVPsSTLKY |
| 2390 | RVRKLPsTTL |
| 2391 | RVRQsPLATK |
| 2392 | RVRQsPLATR |
| 2393 | RVRQsPLATY |
| 2394 | RVRRsSFLNAK |
| 2395 | RVRsLSSLREK |
| 2396 | RVRsLSSLREY |
| 2397 | RVRsPTRSF |
| 2398 | RVRsPTRSL |
| 2399 | RVRsPTRSM |
| 2400 | RVRsPTRSP |
| 2401 | RVRsPTRSV |
| 2402 | RVSsPISKK |
| 2403 | RVSsPISKY |
| 2404 | RVSsRFSSK |
| 2405 | RVSsRFSSR |
| 2406 | RVSsRFSSY |
| 2407 | RVSsVKLISK |
| 2408 | RVSsVKLISY |
| 2409 | RVTsAEIKL |
| 2410 | RWsLSMRK |
| 2411 | RWsLSMRY |
| 2412 | RVWEDRPSsA |
| 2413 | RVWsPPRVHKV |
| 2414 | RVYQyIQSR |
| 2415 | RVYQyIQSRFK |
| 2416 | RVYQyIQSRFY |
| 2417 | RVYQyIQSRK |
| 2418 | RVYQyIQSRY |
| 2419 | RVYsPYNHK |
| 2420 | RVYsPYNHR |
| 2421 | RVYsPYNHY |
| 2422 | RVYSRsFSK |
| 2423 | RVYSRsFSY |
| 2424 | RYPsNLQLF |
| 2425 | RYQtQPVTL |
| 2426 | SAARESHPHGVKRSAsPDDDLG |
| 2427 | SARGsPTRPNPPVR |
| 2428 | SARRtPVSY |
| 2429 | SDDEKMPDLE |
| 2430 | sDFHAERAAREK |
| 2431 | SDmPRAHsF |
| 2432 | SDMPRAHsF |
| 2433 | SEFKAMDsI |
| 2434 | SEGsLHRKF |
| 2435 | SEGsLHRKW |
| 2436 | SEGsLHRKY |
| 2437 | SELsPGRSV |
| 2438 | SFDsGSVRL |
| 2439 | SGGAQsPLRYLHVL |
| 2440 | sGGDDDWTHLSSKEVDPST |
| 2441 | sGGDDDWTHLSSKEVDPSTG |
| 2442 | sGGDDDWTHLSSKEVDPSTGE |
| 2443 | sGGDDDWTHLSSKEVDPSTGEL |
| 2444 | sGGDDDWTHLSSKEVDPSTGELQ |
| 2445 | SGPKPLFRRMsSLVGPTQ |
| 2446 | SIDsPQKL |
| 2447 | SIDsPQKY |
| 2448 | SILsFVSGL |
| 2449 | SIMsFHIDL |
| 2450 | SImsPEIQL |
| 2451 | SIMsPEIQL |
| 2452 | SIPtVSGQI |
| 2453 | SISsMEVNV |
| 2454 | SISStPPAV |
| 2455 | SKEDKNGHDGDTHQEDDGEKsD |
| 2456 | SKRGyIGL |
| 2457 | SKtVATFIL |
| 2458 | SLAsLTEKI |
| 2459 | SLDSEDYsL |
| 2460 | SLCsLGDVFL |
| 2461 | SLCsPSYVLY |
| 2462 | SLEsPSYVLY |
| 2463 | SLFGGsVKL |
| 2464 | SLFKRLYsL |
| 2465 | SLFsGDEENA |
| 2466 | SLFsGSYSSL |
| 2467 | SLFsPQNTL |
| 2468 | SLFsPRRNK |
| 2469 | SLFsPRRNY |
| 2470 | SLFsSEESNL |
| 2471 | SLFsSEESNLGA |
| 2472 | SLHDIQLsL |
| 2473 | SLKsPVTVK |
| 2474 | SLLAsPGHISV |
| 2475 | SLLHTSRsL |
| 2476 | SLLNKSsPVK |
| 2477 | SLLNKSsPVKK |
| 2478 | SLLNKSsPVKY |
| 2479 | SLLsLHVDL |
| 2480 | SLLTsPPKA |
| 2481 | SLLTsPPKV |
| 2482 | SLMsGTLESL |
| 2483 | SLMsPGRKK |
| 2484 | SLMsPGRRY |
| 2485 | SLCPRSHsV |
| 2486 | SLQsLETSV |
| 2487 | SLRRsVLMK |
| 2488 | SLRRsVLMY |
| 2489 | SLSsLLVKL |
| 2490 | SLtRSPPRV |
| 2491 | SLTRsPPRV |
| 2492 | SLVDGyFRL |
| 2493 | SLYDRPAsY |
| 2494 | SLYsPVKKK |
| 2495 | SMFsPRRNK |
| 2496 | SMKsPVTVK |
| 2497 | SMLNKSsPVK |
| 2498 | SMLNKSsPVKK |
| 2499 | SMLsQEIQTL |
| 2500 | SMLTsPPKA |
| 2501 | SMLTsPPKV |
| 2502 | SMMsPGRRK |
| 2503 | SMQPRSHsV |
| 2504 | SMRRsVLMK |
| 2505 | SMSsLSREV |
| 2506 | SMtRSPPRV |
| 2507 | SMTRsPPRV |
| 2508 | SMYsPVKKK |
| 2509 | SNFKsPVKTIR |
| 2510 | SPAASISRLsGEQVDGKG |
| 2511 | SPAsPKISF |
| 2512 | SPAsPKISL |
| 2513 | SPAsPKISM |
| 2514 | SPAsPKISV |
| 2515 | SPDsSQSSL |
| 2516 | sPEDEYELLMPHRISSH |
| 2517 | SPEDEYELLMPHRIsSH |
| 2518 | SPEKAGRRsSF |
| 2519 | SPEKAGRRsSL |
| 2520 | SPEKAGRRsSM |
| 2521 | SPEKAGRRsSV |
| 2522 | sPERPFLAILGGAKVADK |
| 2523 | SPERPFLAILGGAKVADKIQ |
| 2524 | SPFKRQLsF |
| 2525 | SPFKRQLsL |
| 2526 | SPFKRQLsM |
| 2527 | SPFKRQLsV |
| 2528 | SPFLsKRSL |
| 2529 | SPGLARKRsF |
| 2530 | SPGLARKRsL |
| 2531 | SPGLARKRsM |
| 2532 | SPGLARKRsV |
| 2533 | SPGsPRPAF |
| 2534 | SPGsPRPAL |
| 2535 | SPGsPRPAM |
| 2536 | SPGsPRPAV |
| 2537 | SPKsPGLKA |
| 2538 | SPKsPGLKF |
| 2539 | SPKsPGLKL |
| 2540 | SPKsPGLKM |
| 2541 | SPKsPGLKV |
| 2542 | SPKsPTAAF |
| 2543 | SPKsPTAAL |
| 2544 | SPKsPTAAM |
| 2545 | SPKsPTAAV |
| 2546 | SPLTKSIsL |
| 2547 | sPPFPVPVYTRQAPKQVIK |
| 2548 | SPRAPVsPLKF |
| 2549 | SPRERsPAL |
| 2550 | SPRGEAsSL |
| 2551 | SPRGEASsL |
| 2552 | SPRPPNsPSI |
| 2553 | SPRRsLGLAL |
| 2554 | SPRRsRSIsF |
| 2555 | SPRRsRSISF |
| 2556 | SPRRsRSIsL |
| 2557 | SPRRsRSISL |
| 2558 | SPRRsRSIsM |
| 2559 | SPKRsRSISM |
| 2560 | SPRRsRSIsV |
| 2561 | SPRRsRSISV |
| 2562 | SPRsITSTF |
| 2563 | SPRsITSTL |
| 2564 | SPRsTTSTM |
| 2565 | SPRsITSTP |
| 2566 | SPRsITSTV |
| 2567 | SPRsPDRTL |
| 2568 | SPRsPGKPF |
| 2569 | SPRsPGKPL |
| 2570 | SPRsPGKPM |
| 2571 | SPRsPGKPV |
| 2572 | SPRsPGRSF |
| 2573 | SPRsPGRSI |
| 2574 | SPRsPGRSL |
| 2575 | SPRsPGRSM |
| 2576 | SPRsPGRSV |
| 2577 | SPRsPGRSX |
| 2578 | SPRsPSGLR |
| 2579 | SPRsPSTTYF |
| 2580 | SPRsPSTTYL |
| 2581 | SPRSPsTTYL |
| 2582 | SPRsPSTTYM |
| 2583 | SPRsPSTTYV |
| 2584 | SPRssQLV |
| 2585 | SPRtPVsPVKF |
| 2586 | SPRTPVsPVKF |
| 2587 | SPRtPVsPVKL |
| 2588 | SPRTPVsPVKL |
| 2589 | SPRtPVsPVKM |
| 2590 | SPRTPVsPVKM |
| 2591 | SPRtPVsPVKV |
| 2592 | SPRTPVsPVKV |
| 2593 | SPSsPSVRRQF |
| 2594 | SPSsPSVRRQL |
| 2595 | SPSsPSVRRQM |
| 2596 | SPSsPSVRRQV |
| 2597 | SPSTSRSGGsSRF |
| 2598 | SPSTSRSGGsSRL |
| 2599 | SPSTSRSGGsSRM |
| 2600 | SPSTSRSGGsSRV |
| 2601 | sPTRPNPPVRNLH |
| 2602 | SPVsPMKEL |
| 2603 | SPVsTRPLEP |
| 2604 | SPVStRPLEP |
| 2605 | SPWHQsF |
| 2606 | SPWHQsL |
| 2607 | SPVVHQsM |
| 2608 | SPVVHQsV |
| 2609 | SQIsPKSWGV |
| 2610 | SRDKHsEY |
| 2611 | SREKHsEI |
| 2612 | SREKHsEl |
| 2613 | SRFNRRVsV |
| 2614 | SRLTHLsF |
| 2615 | SRLTHLsK |
| 2616 | SRLTHLsL |
| 2617 | SRLTHLsM |
| 2618 | SRLTHLsR |
| 2619 | SRLTHLsY |
| 2620 | SRMsPKAQF |
| 2621 | SRMsPKAQK |
| 2622 | SRMsPKAQL |
| 2623 | SRMsPKAQM |
| 2624 | SRMsPKAQR |
| 2625 | SRMsPKAQY |
| 2626 | SRsSRSPYSR |
| 2627 | SRSsSVLsL |
| 2628 | SRSSsVLSL |
| 2629 | SRSSSVLsL |
| 2630 | SRTsPITRF |
| 2631 | SRTsPITRK |
| 2632 | SRTsPITRL |
| 2633 | SRTsPITRM |
| 2634 | SRTsPITRR |
| 2635 | SRTsPITRY |
| 2636 | SRWsGSHQF |
| 2637 | SRWsGSHQK |
| 2638 | SRWsGSHQR |
| 2639 | SRWsGSHQY |
| 2640 | SRYsRsPYSF |
| 2641 | SRYsRSPYSF |
| 2642 | SRYSRsPYSF |
| 2643 | SRYsRsPYSK |
| 2644 | GRYsRSPYSK |
| 2645 | SRYSRsPYSK |
| 2646 | SRYsRsPYSL |
| 2647 | SRYsRSPYSL |
| 2648 | SRYSRsPYSL |
| 2649 | SRYsRsPYSM |
| 2650 | SRYsRSPYSM |
| 2651 | SRYSRsPYSM |
| 2652 | SRYsRsPYSR |
| 2653 | SRYsRSPYSR |
| 2654 | SRYSRsPYSR |
| 2655 | SRYsRsPYSY |
| 2656 | SRYsRSPYSY |
| 2657 | SRYSRsPYSY |
| 2658 | SRYsRtsPYSR |
| 2659 | SSDIsPTRL |
| 2660 | SSDIsPTRY |
| 2661 | SSDKHsEY |
| 2662 | SSDPASQLsY |
| 2663 | SSDsETLRY |
| 2664 | SSDsPQKL |
| 2665 | SSDsPQKY |
| 2666 | SSDsPSYVLY |
| 2667 | SSDsPTNHFF |
| 2668 | SSEIsPTRY |
| 2669 | SSEKHsEY |
| 2670 | SSEPASQLsY |
| 2671 | SSEsETLRY |
| 2672 | SSEsPQKL |
| 2673 | SSEsPQKY |
| 2674 | SSEsPSYVLY |
| 2675 | SSEsPTNHFY |
| 2676 | SSNGKMASRRsEEKEAG |
| 2677 | SSNGKMASRRsEEKEAGEI |
| 2678 | GSPIMRKKVSL |
| 2679 | sSPPFPVPVYTRQAPKQVIK |
| 2680 | SSsPTHAKSAHV |
| 2681 | SSsWRILGSKQSEHRP |
| 2682 | STDIsPTRL |
| 2683 | STDIsPTRY |
| 2684 | STDKHsEY |
| 2685 | STDPASQLsY |
| 2686 | STDsETLRY |
| 2687 | STDsPQKY |
| 2688 | STDsPSYVLY |
| 2689 | STDsPTNHFY |
| 2690 | STEIsPTRL |
| 2691 | STEIsPTRY |
| 2692 | STEKHsEY |
| 2693 | STEPASQLsY |
| 2694 | STEsETLRY |
| 2695 | STEsPQKY |
| 2696 | STEsPSYVLY |
| 2697 | STEsPTNHFY |
| 2698 | STIQNsPTKK |
| 2699 | sTMSLNIITV |
| 2700 | STMsLNIITV |
| 2701 | SVDIsPIRL |
| 2702 | SVDIsPTRL |
| 2703 | SVDIsPTRY |
| 2704 | SVFsPSFGL |
| 2705 | SVGsDYYIQL |
| 2706 | SVKPRRTsL |
| 2707 | SVKsPVTVK |
| 2708 | SVKsPVTVY |
| 2709 | SVLsPS FQL |
| 2710 | SVMDsPKKL |
| 2711 | SVRRsVLMK |
| 2712 | SVRRsVLMY |
| 2713 | SVRsLSLSL |
| 2714 | SVYSGDFGNLEV |
| 2715 | SVYsPVKKK |
| 2716 | SVYsPVKKY |
| 2717 | sYIEHIFEI |
| 2718 | SYPsPVATSY |
| 2719 | sYQKVIELF |
| 2720 | TDKYsKMM |
| 2721 | TEAsPESML |
| 2722 | THKGEIRGASTPFQFRAssP |
| 2723 | TIGEKKEPsDKSVDS |
| 2724 | TKDKYMASRGQKAKsMEG |
| 2725 | TKsVKALSSLHGDD |
| 2726 | TKsVKALSSLHGDDQ |
| 2727 | TKsVKALSSLHGDDQD |
| 2728 | TLAsPSVFKST |
| 2729 | TLAsPSVFKSV |
| 2730 | TLLAsPMLK |
| 2731 | TLMERTVSL |
| 2732 | TLSsPPPGL |
| 2733 | TMAsPGKDNY |
| 2734 | TMAsPSVFKST |
| 2735 | TMAsPSVFKSV |
| 2736 | TMDsPGKDNY |
| 2737 | TMEsPGKDNY |
| 2738 | TMMsPSQFL |
| 2739 | TPAQPQRRsF |
| 2740 | TPAQPQRRsL |
| 2741 | TPAQPQRRsM |
| 2742 | TPAQPQRRsV |
| 2743 | TPDPSKFFSQLsSEHGGDV |
| 2744 | tPDPSKFFSQLSSEHGGDVQ |
| 2745 | TPIsPGRASGF |
| 2746 | TPIsPGRASGL |
| 2747 | TPIsPGRASGM |
| 2748 | TPISPGRASGV |
| 2749 | TPMKKHLsL |
| 2750 | TPRsPPLGF |
| 2751 | TPRsPPLGL |
| 2752 | TPRsPPLGLF |
| 2753 | TPRsPPLGLI |
| 2754 | TPRsPPLGLL |
| 2755 | TPRsPPLGLM |
| 2756 | TPRsPPLGLV |
| 2757 | TPRsPPLGM |
| 2758 | TPRsPPLGV |
| 2759 | TQSSGKsSV |
| 2760 | TRKtPESFL |
| 2761 | TRLsPAKIVLF |
| 2762 | TRLsPAKIVLK |
| 2763 | TRLsPAKIVLR |
| 2764 | TRLsPAKIVLY |
| 2765 | TSAsPGKDNY |
| 2766 | TSDsPGKDNY |
| 2767 | TSDtPDYLLKY |
| 2768 | TSEsPGKDNY |
| 2769 | TSEtPDYLLKY |
| 2770 | TTAsPGKDNY |
| 2771 | TTDsPGKDNY |
| 2772 | TTDtPDYLLKY |
| 2773 | TTEsPGKDNY |
| 2774 | TTEtPDYLLKY |
| 2775 | TTKsVKALSSLHG |
| 2776 | TTKsVKALSSLHGDD |
| 2777 | TTKsVKALSSLHGDDQ |
| 2778 | TTKsVKALSSLHGDDQD |
| 2779 | TTKsVKALSSLHGDDQDS |
| 2780 | TTKsVKALSSLHGDDQDsED |
| 2781 | TTKSVKALSSLHGDDQDsED |
| 2782 | TTKsVKALSSLHGDDQDsEDE |
| 2783 | TTKSVKALSSLHGDDQDsEDE |
| 2784 | TVFsPTLPAA |
| 2785 | TVMsNSSVIHL |
| 2786 | VAKRLsL |
| 2787 | VAMPVKKSPRRSsSDEQGLSYSSLKNV |
| 2788 | VIDsQELSKV |
| 2789 | VLDsPASKK |
| 2790 | VLFPEsPARA |
| 2791 | VLFRtPLASV |
| 2792 | VLFsSPPQM |
| 2793 | VLFSsPPQM |
| 2794 | VLIENVAsL |
| 2795 | VLIGsPKKV |
| 2796 | VLIGsPKKY |
| 2797 | VLKGsRSSEL |
| 2798 | VLKGsRSSEV |
| 2799 | VLKSRKssVTEE |
| 2800 | VLKVMIGsPK |
| 2801 | VLKVMIGsPKK |
| 2802 | VLKVMIGsPKKK |
| 2803 | VLLsPVPEL |
| 2804 | VLLsPVPEV |
| 2805 | VLMK(sPs)PAL |
| 2806 | VLMK(sPs)PAV |
| 2807 | VLQtPPYVK |
| 2808 | VLQtPPYVKK |
| 2809 | VLQtPPYVKY |
| 2810 | VLSDVIPsI |
| 2811 | VLSSLtPAKV |
| 2812 | VLWDTPsI |
| 2813 | VLYsPQMAL |
| 2814 | VMFRtPLASV |
| 2815 | VMIGsKKV |
| 2816 | VMIGsPKKV |
| 2817 | VMIGsPKKY |
| 2818 | VMKVMIGsPK |
| 2819 | VMKVMIGsPKK |
| 2820 | VMKVMIGsPKKK |
| 2821 | VMKVMIGsPKKY |
| 2822 | VMLsPVPEL |
| 2823 | VMLsPVPEV |
| 2824 | VMQtPPYVK |
| 2825 | VMQtPPYVKK |
| 2826 | VPHHGFEDWsQIR |
| 2827 | VPKSGRSSsL |
| 2828 | VPKsPAFAL |
| 2829 | VPLIRKKsL |
| 2830 | VPNAPPAYEKLsAEQSPPPY |
| 2831 | VPREVLRLsF |
| 2832 | VPREVLRLsL |
| 2833 | VPREVLRLsM |
| 2834 | VPREVLRLsV |
| 2835 | VPRPERRsSL |
| 2836 | VPRsPKHAHSSSF |
| 2837 | VPRsPKHAHSSSL |
| 2838 | VPRsPKHAHSSSM |
| 2839 | VPRsPKHAHSSSV |
| 2840 | VPStPKSSL |
| 2841 | VPTsPKSSL |
| 2842 | VPVsPGQQL |
| 2843 | VRAsKDLAQ |
| 2844 | VRQsVTSFPDADAFHHQ |
| 2845 | VSKVMIGsPKKV |
| 2846 | VSKVMIGsPKKY |
| 2847 | VTQtPPYVKK |
| 2848 | VTQtPPYVKY |
| 2849 | WDsPGQEVL |
| 2850 | VYTyIQSRF |
| 2851 | WTHLsSKEVDPS |
| 2852 | WTHLsSKEVDPSTG |
| 2853 | YARsVHEEF |
| 2854 | YAVPRRGsL |
| 2855 | YAYDGKDyI |
| 2856 | YEGsPIKV |
| 2857 | YEKLsAEQSPPP |
| 2858 | YFsPFRPY |
| 2859 | yIQSRF |
| 2860 | YLAsLEKKL |
| 2861 | YLDsGIHSG |
| 2862 | YLDsGIHsGA |
| 2863 | YLDsGIHSGA |
| 2864 | YLDsGIHsGV |
| 2865 | YLDsGIHSGV |
| 2866 | yLGLDVPV |
| 2867 | YLGsISTLVTL |
| 2868 | YLIHsPMSL |
| 2869 | YLLsPLNTL |
| 2870 | YLLsPTKLPSI |
| 2871 | YLLsPTKLPSV |
| 2872 | yLQSRYYRA |
| 2873 | YLQsRYYRA |
| 2874 | YLSDsDTEAKL |
| 2875 | YMDsGIHsGA |
| 2876 | YMDsGIHSGA |
| 2877 | YMDsGIHsGV |
| 2878 | YMDsGIHSGV |
| 2879 | YPDPHsPFAV |
| 2880 | YPGGRRsSL |
| 2881 | YPLsPAKVNQY |
| 2882 | YPLsPTKISEY |
| 2883 | YPLsPTKISQY |
| 2884 | YPRsEDEVEGVM |
| 2885 | YPRsFDEVEGF |
| 2886 | YPRsFDEVEGL |
| 2887 | YPRsFDEVEGM |
| 2888 | YPRsFDEVEGV |
| 2889 | YPRsFDEVEGVF |
| 2890 | YPRsFDEVEGVL |
| 2891 | YPRsFDEVEGVM |
| 2892 | YPRsFDEVEGVV |
| 2893 | YPSFRRsSL |
| 2894 | YPSsPRKAL |
| 2895 | YPSsPRKF |
| 2896 | YPSsPRKL |
| 2897 | YPSsPRKM |
| 2898 | YPSsPRKV |
| 2899 | YPYEFsPVKM |
| 2900 | YQLsPTKLPSI |
| 2901 | YQLsPTKLPSV |
| 2902 | YQRPFsPSAY |
| 2903 | YQRsFDEVEGF |
| 2904 | YQRsFDEVEGL |
| 2905 | YQRsFDEVEGM |
| 2906 | YQRsFDEVEGV |
| 2907 | YQRsFDEVEGVF |
| 2908 | YQRsFDEVEGVL |
| 2909 | YQRsFDEVEGVM |
| 2910 | YQRsFDEVEGVV |
| 2911 | YRYsPQSFL |
| 2912 | YTAGtPYKV |
| 2913 | YYTAGSSsPTHAKSAHV |
| 8808 | RLLsAAENFL |

Lowercase s, t, and y indicate phosphorylated serine, phosphorylated threonine, and phosphorylated tyrosine, respectively.
Lowercase c indicates that the cysteine is present in a cysteine-cysteine disulfide bond.
Lowercase m indicates oxidized methionine.
(AcS) indicates an N-terminally acetylated serine.
(sLss) indicates that at least one serine residue in the amino acid sequence SLSS is phosphorylated.
(sPs) indicates that at least one serine residue in the amino acid sequence SPS is phosphorylated.

TABLE 3
Amino acid sequences of exemplary
MHC-binding peptides

| SEQ ID NO | Amino Acid Sequence |
|---|---|
| 2914 | ALTtsAHSV |
| 2915 | ALTtSAHSV |
| 2916 | ALTTsAHSV |
| 2917 | APP(sts)AAAL |
| 2918 | APPsTSAAAL |
| 2919 | APPsTsAAAL |
| 2920 | APPStSAAAL |
| 2921 | APPSTsAAAL |
| 2922 | APPstSAAAL |
| 2923 | APPStsAAAL |
| 2924 | APP<s>TSAAAL |
| 2925 | APPS<t>SAAAL |
| 2926 | APPST<s>AAAL |
| 2927 | APPS<t>sAAAL |
| 2928 | APP<s><t>SAAAL |
| 2929 | APP<s>T<s>AAAL |
| 2930 | APPS<t><s>AAAL |
| 2931 | APRG<n>VISL |
| 2932 | APRtNGVAM |
| 2933 | APTsAAAL |
| 2934 | APTsASNVM |
| 2935 | APTSAsNVM |
| 2936 | APVsASASV |
| 2937 | APVsSKSSL |
| 2938 | EP(sst)VVSL |
| 2939 | EPsSTVVSL |
| 2940 | EPSsTVVSL |
| 2941 | EPSStVVSL |
| 2942 | GLSsLAEEAA |
| 2943 | HP(sss)AAVL⁽ⁱ⁾ |
| 2944 | HP(sst)ASTAL |
| 2945 | HPMsTASQV |
| 2946 | HPssTAAVL |
| 2947 | HPsStAAVL |
| 2948 | HPSstAAVL |
| 2949 | HPsSTASTAL |
| 2950 | HPSsTASTAL |
| 2951 | HPSStASTAL |
| 2952 | HPTtVASY |
| 2953 | IPIsLHTSL |
| 2954 | IPTsSVLSL |
| 2955 | IPVsKPLSL |
| 2956 | IPV<s>KPLSL |
| 2957 | IPVsSHNSL |
| 2958 | IPVssHNSL |
| 2959 | IPV<s>SHNSL |
| 2960 | IPV[s]SHNSL |
| 2961 | KPPtSQSSVL |
| 2962 | KPP<t>SQSSVL |
| 2963 | KPPTsQSSVL |
| 2964 | KPPT<s>QSSVL |
| 2965 | KPPV<s>FFSL |
| 2966 | KPTLY<n>VSL |
| 2967 | LPRN(st)MM |
| 2968 | LPRNstMM |
| 2969 | LPTsLPSSL |
| 2970 | MPVRPT<t>NTF |
| 2971 | (diMe)MPVRPT<t>NTF |
| 2972 | MPVtSSSFF |
| 2973 | NPVsLPSL |
| 2974 | PPS<t>SAAAL |
| 2975 | PPST<s>AAAL |
| 2976 | RPP(sss)QQL |
| 2977 | RPPItQSSL |
| 2978 | (Me)RPPItQSSL |
| 2979 | (diME)RPPItQSSL |
| 2980 | (diME)RPPI[t]QSSL |
| 2981 | RPPQ<s>SSVSL |
| 2982 | RPPsSSQQL |
| 2983 | RPPSsSQQL |
| 2984 | RPPSSsQQL |
| 2985 | RPPVtKASSF |
| 2986 | RPVtASITTM |
| 2987 | TPASsRAQTL |
| 2988 | TPAsSSSAL |
| 2989 | TPIsQAQKL |
| 2990 | TPVsSANMM |
| 2991 | VLTsNVQTI |
| 2992 | VPAsSTSTL |
| 2993 | VPAtHGQVTY |
| 2994 | VPtTSSSL |
| 2995 | VPTtSSSL |
| 2996 | VPTTsSSL |
| 2997 | VPVsGTQGL |
| 2998 | VPVsNQSSL |
| 2999 | VPVsSASEL |
| 3000 | VPVsVGPSL |

Lowercase s and t indicate O-GlcNAcylated
serine and O-GlnNAcylated threonine,
respectively.
(sts), (sss), (ts), (sst), and (st)
indicates at least one of the serine or
threonine residues is modifed with O-GlnNAc.
(i) indicates that two GlnNAc moeities were
detected, but could not be assigned to
specific amino acids.
(Me) indicates methylation of the following
arginine.
(diMe) indicates asymmetric di-methylation of the
following arginine.
<n> indicates hexose-GlcNAcylated asparagine.
<s> indicates hexose-GlcNAcylated serine.
<t> indicates hexose-GlcNAcylated threonine.
[s] indicates acetyl-GlcNAcylated serine.
[t] indicates acetyl-GlcNAcylated threonine.

TABLE 4
Amino acid sequences of exemplary
antigenic polypeptides

| SEQ ID NO | Amino Acid Sequence |
|---|---|
| 3001 | (AcS)AARESHPHGVKRSAsPDDDLGFFRKNLLRLTG |
| 3002 | AAEsPSFLFFRKNLLRLTG |
| 3003 | AASNFKsPVKTIRFFRKNLLRLTG |
| 3004 | ADLsPEREVFFRKNLLRLTG |
| 3005 | AEDEIGtPRKFFFRKNLLRLTG |
| 3006 | AEDEIGtPRKYFFRKNLLRLTG |
| 3007 | AEEEIGtPRKFFFRKNLLRLTG |
| 3008 | AEEEIGtPRKWFFRKNLLRLTG |
| 3009 | AEEEIGtPRKYFFRKNLLRLTG |
| 3010 | AENARSAsFFFRKNLLRLTG |
| 3011 | AENsPTRQQFFFRKNLLRLTG |
| 3012 | AENsPTRQQWFFRKNLLRLTG |
| 3013 | AENsPTRQQYFFRKNLLRLTG |
| 3014 | AENsSSRELFFRKNLLRLTG |
| 3015 | AEQGsPRVSYFFRKNLLRLTG |
| 3016 | AESsPTAGKKFFFRKNLLRLTG |
| 3017 | AESsPTAGKKLFFRKNLLRLTG |
| 3018 | AESsPTAGKKWFFRKNLLRLTG |
| 3019 | AESsPTAGKKYFFRKNLLRLTG |
| 3020 | AGDsPGSQFFFRKNLLRLTG |
| 3021 | AILsPAFKVFFRKNLLRLTG |
| 3022 | AIMRsPQMVFFRKNLLRLTG |
| 3023 | AIsDLQQLFFRKNLLRLTG |
| 3024 | AKLsETISFFRKNLLRLTG |
| 3025 | ALAAsPHAVFFRKNLLRLTG |
| 3026 | ALDsGASLLHLFFRKNLLRLTG |
| 3027 | ALDsGASLLHVFFRKNLLRLTG |
| 3028 | ALGNtPPFLFFRKNLLRLTG |
| 3029 | ALGsRESLATIFFRKNLLRLTG |
| 3030 | ALGsRESLATVFFRKNLLRLTG |
| 3031 | ALIHQsLGLFFRKNLLRLTG |
| 3032 | ALIHQsLGVFFRKNLLRLTG |
| 3033 | ALLGSKsPDPYRLFFRKNLLRLTG |
| 3034 | ALLGSKsPDPYRVFFRKNLLRLTG |
| 3035 | ALLsLLKRVFFRKNLLRLTG |
| 3036 | ALMGsPQLVFFRKNLLRLTG |
| 3037 | ALMGsPQLVAAFFRKNLLRLTG |
| 3038 | ALRSsPIMRKFFRKNLLRLTG |
| 3039 | ALRSsPIMRYFFRKNLLRLTG |
| 3040 | ALVsPPALHNAFFRKNLLRLTG |
| 3041 | APRRYsSSMFFRKNLLRLTG |
| 3042 | ALVsPPALHNVFFRKNLLRLTG |
| 3043 | ALYsGVHKKFFRKNLLRLTG |
| 3044 | ALYsGVHKYFFRKNLLRLTG |
| 3045 | ALYsPAQPSLFFRKNLLRLTG |
| 3046 | ALYtPQAPYFFRKNLLRLTG |
| 3047 | ALYtPQAPYFFRKNLLRLTG |
| 3048 | AMAAsPHAVFFRKNLLRLTG |
| 3049 | AMDsGASLLHLFFRKNLLRLIG |
| 3050 | AMDsGASLLHVFFRKNLLRLIG |
| 3051 | AMGsRESLATIFFRKNLLRLTG |
| 3052 | AMGsRESLATVFFRKNLLRLIG |
| 3053 | AMLGSKsPDPYRLFFRKNLLRLIG |
| 3054 | AMLGSKsPDPYRVFFRKNLLRLIG |
| 3055 | AMPGsPVEVFFRKNLLRLIG |
| 3056 | AMRSsPIMRKFFRKNLLRLTG |
| 3057 | AMVsPPALHNAFFRKNLLRLIG |
| 3058 | AMVsPPALHNVFFRKNLLRLIG |
| 3059 | AMYsGVHKKFFRKNLLRLIG |
| 3060 | APDsPRAFLFFRKNLLRLTG |
| 3061 | APLARASsLFFRKNLLRLTG |
| 3062 | APPAYEKLsFFRKNLLRLTG |
| 3063 | APPAYEKLsAEQFFRKNLLRLTG |
| 3064 | APPAYEKLsAEQSPPFFRKNLLRLTG |
| 3065 | APPAYEKLsAEQSPPPFFRKNLLRLTG |
| 3066 | APPAYEKLsAEQSPPPYFFRKNLLRLTG |
| 3067 | APPPLVPAPRPSsPPRGPGPARADRFFRKNLLRLTG |
| 3068 | APRAPsASPLALFFRKNLLRLTG |
| 3069 | APRDRRAVsFFFRKNLLRLTG |
| 3070 | APRKGsFSALFFRKNLLRLTG |
| 3071 | APRKGsFSALFFFRKNLLRLTG |
| 3072 | APRKGsFSALLFFRKNLLRLTG |
| 3073 | APRKGsFSALMFFRKNLLRLTG |
| 3074 | APRKGsFSALVFFRKNLLRLTG |
| 3075 | APRNGsGVALFFRKNLLRLTG |
| 3076 | APRRYsSSFFFRKNLLRLTG |
| 3077 | APRRYsSSLFFRKNLLRLTG |
| 3078 | APRRYsSSMFFRKNLLRLTG |
| 3079 | APRRYsSSVFFRKNLLRLTG |
| 3080 | APRsPPPSRFFFRKNLLRLTG |
| 3081 | APRsPPPSRLFFRKNLLRLTG |
| 3082 | APRsPPPSRMFFRKNLLRLTG |
| 3083 | APRsPPPSRPFFRKNLLRLTG |
| 3084 | APRsPPPSRVFFRKNLLRLTG |
| 3085 | APSLFHLNtLFFRKNLLRLTG |
| 3086 | APSSARAsPLLFFRKNLLRLTG |
| 3087 | APSTYAHLsPAKFFRKNLLRLTG |
| 3088 | APSTYAHLsPAKTPPPPFFRKNLLRLTG |
| 3089 | APSVRsLSLFFRKNLLRLTG |
| 3090 | APSVRSLsLFFRKNLLRLTG |
| 3091 | ARFsPDDKYSFFFRKNLLRLTG |
| 3092 | ARFsPDDKYSKFFRKNLLRLTG |
| 3093 | ARFsPDDKYSLFFRKNLLRLTG |
| 3094 | ARFsPDDKYSMFFRKNLLRLTG |
| 3095 | ARFsPDDKYSRFFRKNLLRLTG |
| 3096 | ARFsPDDKYSYFFRKNLLRLTG |
| 3097 | ASDEIGtPRKFFRKNLLRLTG |
| 3098 | ASDEIGtPRKYFFRKNLLRLTG |
| 3099 | ASEEIGtPRKFFFRKNLLRLTG |
| 3100 | ASEEIGtPRKYFFRKNLLRLTG |
| 3101 | AsISRLsGEQVDGKGFFRKNLLRLTG |
| 3102 | AsISRLSGEQVDGKGFFRKNLLRLTG |
| 3103 | ASISRLsGEQVDGKGFFRKNLLRLTG |
| 3104 | AsIsRLSGEQVDGKGQFFRKNLLRLTG |
| 3105 | AsISRLSGEQVDKGKGFFRKNLLRLTG |
| 3106 | ASKAsPTLDFTERFFRKNLLRLTG |
| 3107 | ASKMTQPQSKSAFPLSRKNGsGsLDGFFRKNLLRLTG |
| 3108 | AsLGFVFFFRKNLLRLTG |
| 3109 | AsPTIEAQGTSPAHDNFFRKNLLRLTG |
| 3110 | AsPTIEAQGTSPAHDNIFFRKNLLRLTG |
| 3111 | AsPTIEAQGTSPAHDNIAFFRKNLLRLTG |
| 3112 | AtAGPRLGFFFRKNLLRLTG |
| 3113 | AtAGPRLGWFFRKNLLRLTG |
| 3114 | AtAGPRLGYFFRKNLLRLTG |
| 3115 | ATDEIGtPRKFFFRKNLLRLTG |
| 3116 | ATDEIGtPRKYFFRKNLLRLTG |
| 3117 | ATEEIGtPRKFFFRKNLLRLTG |
| 3118 | ATEEIGtPRKYFFRKNLLRLTG |
| 3119 | ATWsGSEFEVFFRKNLLRLTG |
| 3120 | ATYtPQAPKFFRKNLLRLTG |
| 3121 | ATYtPQAPKYFFRKNLLRLTG |
| 3122 | AVIHQsLGLFFRKNLLRLTG |
| 3123 | AVIHQsLGVFFRKNLLRLTG |
| 3124 | AVRPTRLsLFFRKNLLRLTG |
| 3125 | AVVsPPALHNAFFRKNLLRLTG |
| 3126 | AVVsPPALHNVFFRKNLLRLTG |
| 3127 | AYEKLsAEQSPPFFRKNLLRLTG |
| 3128 | DAKKsPLALFFRKNLLRLTG |
| 3129 | DDDWTHLsSKEVDPFFRKNLLRLTG |
| 3130 | DDDWTHLsSKEVDPSFFRKNLLRLTG |
| 3131 | DDDWTHLsSKEVDPSTFFRKNLLRLTG |
| 3132 | DDDWTHLsSKEVDPSTGFFRKNLLRLT |
| 3133 | DDWTHLsSKEVDPSFFRKNLLRLTG |
| 3134 | DEFERIKtFFFRKNLLRLTG |
| 3135 | DEFERIKtWFFRKNLLRLTG |
| 3136 | DEFERIKtYFFRKNLLRLTG |
| 3137 | DEISHRAsFFFRKNLLRLTG |
| 3138 | DEISHRAsWFFRKNLLRLTG |
| 3139 | DEISHRAsYFFRKNLLRLTG |
| 3140 | DERLRINsFFFRKNLLRLTG |
| 3141 | DERLRINsLFFRKNLLRLTG |
| 3142 | DERLRINsWFFRKNLLRLTG |
| 3143 | DERLRINsYFFRKNLLRLTG |
| 3144 | DKLsVIAEDSESGKQFFRKNLLRLTG |
| 3145 | DKLsVIAEDSESGKQNFFRKNLLRLTG |
| 3146 | DKLsVIAEDSESGKQNPFFRKNLLRLTG |
| 3147 | DKLsVIAEDSESGKQNPGFFRKNLLRLTG |
| 3148 | DKLsVIAEDSESGKQNPGDSFFRKNLLRLTG |
| 3149 | DLKRRsmSIFFRKNLLRLTG |
| 3150 | DLKRRsMSIFFRKNLLRLTG |
| 3151 | DLKSSKAsLFFRKNLLRLTG |
| 3152 | DLRtVEKELFFRKNLLRLTG |
| 3153 | DLsEEKFLFFRKNLLRLTG |
| 3154 | DLsEEKFVFFRKNLLRLTG |
| 3155 | DLVPLsPLKKFFRKNLLRLTG |
| 3156 | DLWKItKVMDFFRKNLLRLTG |
| 3157 | DMVPLsPLKKFFRKNLLRLTG |
| 3158 | DPTRRFFKVtPPPGSGPQFFRKNLLRLTG |
| 3159 | DQFERIKtLFFRKNLLRLTG |
| 3160 | DQISHRAsLFFRKNLLRLTG |
| 3161 | DSDPLsPLKYFFRKNLLRLTG |
| 3162 | DSEPLsPLKYFFRKNLLRLTG |
| 3163 | DSsEEKFLFFRKNLLRLTG |
| 3164 | DSsEEKFVFFRKNLLRLTG |
| 3165 | DSVPLsPLKYFFRKNLLRLTG |
| 3166 | DTDPLsPLKYFFRKNLLRLTG |
| 3167 | DTEPLsPLKYFFRKNLLRLTG |
| 3168 | DTVPLsPLKYFFRKNLLRLTG |
| 3169 | DWTHLsSKEVDPSFFRKNLLRLTG |
| 3170 | DWTHLsSKEVDPSTGFFRKNLLRLTG |
| 3171 | EEGsPTMVEKGLEPGVFTLFFRKNLLRLTG |
| 3172 | EELsPTAKFFFRKNLLRLTG |
| 3173 | EELsPTAKFFFRKNLLRLTG |
| 3174 | EEMPENALPsDEDDKDPNDPYRALFFRKNLLRLTG |
| 3175 | EERRsPPAPFFRKNLLRLTG |
| 3176 | EEsSDDGKKFFFRKNLLRLTG |
| 3177 | EEsSDDGKKFFFRKNLLRLTG |
| 3178 | EEsSDDGKKWFFRKNLLRLTG |
| 3179 | EESsDDGKKWFFRKNLLRLTG |
| 3180 | EEsSDDGKKYFFRKNLLRLTG |
| 3181 | EESsDDGKKYFFRKNLLRLTG |
| 3182 | EGEEPTVYsDEEEPKDESARKNDFFRK |
| 3183 | EGsPTMVEKGLEPGVFTLFFRKNLLRLTG |
| 3184 | ELFSsPPAVFFRKNLLRLTG |
| 3185 | ELKKsPTSLKFFRKNLLRLTG |
| 3186 | ELKKsPTSLYFFRKNLLRLTG |
| 3187 | ELLMPHRIsSHFFFRKNLLRLTG |
| 3188 | ELLMPHRIsSHFLFFRKNLLRLTG |
| 3189 | ELRISGsVQLFFRKNLLRLTG |
| 3190 | EMKKsPTSLKFFRKNLLRLTG |
| 3191 | EPAsPAAsISRLsGEQVDGKGFFRKNLLRLTG |
| 3192 | EPAsPAAsISRLSGEQVDGKGFFRKNLLRLTG |
| 3193 | EPKRRsARFFFRKNLLRLTG |
| 3194 | EPKRRsARLFFRKNLLRLTG |
| 3195 | EPKRRsARMFFRKNLLRLTG |
| 3196 | EPKRRsARVFFRKNLLRLTG |
| 3197 | EPRsPSHSFFFRKNLLRLTG |
| 3198 | EPRsPSHSLFFRKNLLRLTG |
| 3199 | EPRsPSHSMFFRKNLLRLTG |
| 3200 | EPRsPSHSVFFRKNLLRLTG |
| 3201 | ERsPLLSQETAGQKPFFRKNLLRLTG |
| 3202 | ERsPLLSQETAGQKPLFFRKNLLRLTG |
| 3203 | ESDsLPRYFFRKNLLRLTG |
| 3204 | ESEsLPRYFFRKNLLRLTG |
| 3205 | ESsVRSQEDQLSRFFRKNLLRLTG |
| 3206 | ESsVRSQEDQLSRRFFRKNLLRLTG |
| 3207 | ETDsLPRYFFRKNLLRLTG |
| 3208 | ETEsLPRYFFRKNLLRLTG |
| 3209 | FDKHTLGDsDNESFFRKNLLRLTG |
| 3210 | FEDDDsNEKLFFRKNLLRLTG |
| 3211 | FIEsPSKLFFRKNLLRLTG |
| 3212 | FIEsPSKYFFRKNLLRLTG |
| 3213 | FIGsPTTPAGLFFRKNLLRLTG |
| 3214 | FKMPQEKsPGYSFFRKNLLRLTG |
| 3215 | FKsPVKTIRFFRKNLLRLTG |
| 3216 | FKtQPVTFFFRKNLLRLTG |
| 3217 | FLDNsFEKVFFRKNLLRLTG |
| 3218 | FLDRPPtPLFIFFRKNLLRLTG |
| 3219 | FLDsLRDLIFFRKNLLRLTG |
| 3220 | FLDtPIAKVFFRKNLLRLTG |
| 3221 | FLFDKPVsPLLLFFRKNLLRLTG |
| 3222 | FLGVRPKsAFFRKNLLRLTG |
| 3223 | FLIIRtVLQLFFRKNLLRLTG |
| 3224 | FLITGGGKGsGFSLFFRKNLLRLTG |
| 3225 | FLLsQNFDDEFFRKNLLRLTG |
| 3226 | FLYsGKETYFFRKNLLRLTG |
| 3227 | FPHsLLSVFFFRKNLLRLTG |
| 3228 | FPHsLLSVIFFRKNLLRLTG |
| 3229 | FPHsLLSVIFFRKNLLRLTG |
| 3230 | FPHsLLSVLFFRKNLLRLTG |
| 3231 | FPHsLLSVMFFRKNLLRLTG |
| 3232 | FPHsLLSVVFFRKNLLRLTG |
| 3233 | FPIsPVRFFFRKNLLRLTG |
| 3234 | FPIsPVRLFFRKNLLRLTG |
| 3235 | FPIsPVRMFFRKNLLRLTG |
| 3236 | FPIsPVRVFFRKNLLRLTG |
| 3237 | FPLDsPKTLVLFFRKNLLRLTG |
| 3238 | FPRRHsVTLFFRKNLLRLTG |
| 3239 | FPRsPTKSSFFFRKNLLRLTG |
| 3240 | FPRsPTKSSLFFRKNLLRLTG |
| 3241 | FPRsPTKSSLDFFFRKNLLRLTG |
| 3242 | FPRsPTKSSLDLFFRKNLLRLTG |
| 3243 | FPRsPTKSSLDMFFRKNLLRLTG |
| 3244 | FPRsPTKSSLDVFFRKNLLRLTG |
| 3245 | FPRsPTKSSMFFRKNLLRLTG |
| 3246 | FPRsPTKSSVFFRKNLLRLTG |
| 3247 | FRFsGRTEYFFRKNLLRLTG |
| 3248 | FRGRYRsPYFFRKNLLRLTG |
| 3249 | FRKsMVEHYFFRKNLLRLTG |
| 3250 | FRRsPIKSSLDYFFRKNLLRLTG |
| 3251 | FRRsPTKSSFFFRKNLLRLTG |
| 3252 | FRRsPTKSSLFFRKNLLRLTG |
| 3253 | FRRsPTKSSLDFFRKNLLRLTG |
| 3254 | FRRsPTKSSLDFFFRKNLLRLTG |
| 3255 | FRRsPTKSSLDLFFRKNLLRLTG |
| 3256 | FRRsPTKSSLDMFFRKNLLRLTG |
| 3257 | FRRsPTKSSLDVFFRKNLLRLTG |
| 3258 | FRRsPTKSSLDYFFRKNLLRLTG |
| 3259 | FRRsPTKSSMFFRKNLLRLTG |
| 3260 | FRRsPTKSSVFFRKNLLRLTG |
| 3261 | FRsPTKSSLDFFFRKNLLRLTG |
| 3262 | FRsPTKSSLDLFFRKNLLRLTG |
| 3263 | FRsPTKSSLDMFFRKNLLRLTG |
| 3264 | FRsPTKSSLDVFFRKNLLRLTG |
| 3265 | FRYsGKTEFFFRKNLLRLTG |
| 3266 | FRYsGKTEKFFRKNLLRLTG |
| 3267 | FRYsGKTELFFRKNLLRLTG |
| 3268 | FRYsGKTEMFFRKNLLRLTG |
| 3269 | FRYsGKTERFFRKNLLRLTG |
| 3270 | FRYsGKTEYFFRKNLLRLTG |
| 3271 | FSDsHEGFSYFFRKNLLRLTG |
| 3272 | FSEsHEGFSYFFRKNLLRLTG |
| 3273 | FSEsPSKLFFRKNLLRLTG |
| 3274 | FSEsPSKYFFRKNLLRLTG |
| 3275 | FSIsPVRFFFRKNLLRLTG |
| 3276 | FSIsPVRLFFRKNLLRLTG |
| 3277 | FSIsPVRMFFRKNLLRLTG |
| 3278 | FSIsPVRVFFRKNLLRLTG |
| 3279 | FSsSHEGFSYFFRKNLLRLTG |
| 3280 | FSSsHEGFSYFFRKNLLRLTG |
| 3281 | FTDsHEGFSYFFRKNLLRLTG |
| 3282 | FTEsHEGFSYFFRKNLLRLTG |
| 3283 | FTEsPSKLFFRKNLLRLTG |
| 3284 | FTEsPSKYFFRKNLLRLTG |
| 3285 | FTKsPYQEFFFRKNLLRLTG |
| 3286 | FTsSHEGFSYFFRKNLLRLTG |
| 3287 | FVSKVMIGsPKKVFFRKNLLRLTG |
| 3288 | GALsPSLLHSLFFRKNLLRLTG |
| 3289 | GAQPGRHsFFFRKNLLRLTG |
| 3290 | GAQPGRHsLFFRKNLLRLTG |
| 3291 | GAQPGRHsVFFRKNLLRLTG |
| 3292 | GDDDWTHLsSKEVDFFRKNLLRLTG |
| 3293 | GDDDWTHLsSKEVDPFFRKNLLRLTG |
| 3294 | GDDDWTHLsSKEVDPSFFRKNLLRLTG |
| 3295 | GDDDWTHLsSKEVDPSTFFRKNLLRLTG |
| 3296 | GDDDWTHLsSKEVDPSTGFFRKNLLRLTG |
| 3297 | GEAsPSHIIFFRKNLLRLTG |
| 3298 | GEEsSDDGKKFFFRKNLLRLTG |
| 3299 | GEEsSDDGKKWFFRKNLLRLTG |
| 3300 | GEEsSDDGKMKYFFRKNLLRLTG |
| 3301 | GEEsSDIDGKKFFFRKNLLRLTG |
| 3302 | GEIsPQREVFFRKNLLRLTG |
| 3303 | GERsPLLSQETAGQKPFFRKNLLRLTG |
| 3304 | GERsPLLSQETAGQKPLFFRKNLLRLTG |
| 3305 | GETsPRTKIFFRKNLLRLTG |
| 3306 | GGDDDWTHLsSKEVDPSFFRKNLLRLTG |
| 3307 | GGDDDWTHLsSKEVDPSTGFFRKNLLRLTG |
| 3308 | GGSFGGRSSGsPFFRKNLLRLTG |
| 3309 | GGSFGGRSSGsVFFRKNLLRLTG |
| 3310 | GIDsPSSSVFFRKNLLRLTG |
| 3311 | GIMsPLAKKFFRKNLLRLTG |
| 3312 | GLAPtPPSMFFRKNLLRLTG |
| 3313 | GLDsGFHSVFFRKNLLRLTG |
| 3314 | GLDsLDQVEIFFRKNLLRLTG |
| 3315 | GLGELLRsLFFRKNLLRLTG |
| 3316 | GLIRSRsFIFKFFRKNLLRLTG |
| 3317 | GLIRSRsFIFYFFRKNLLRLTG |
| 3318 | GLIsPELRHLFFRKNLLRLTG |
| 3319 | GLIsPNVQLFFRKNLLRLTG |
| 3320 | GLIsPVWGAFFRKNLLRLTG |
| 3321 | GLItPGGFSSVFFRKNLLRLTG |
| 3322 | GLLDsPTSIFFRKNLLRLTG |
| 3323 | GLLGSpARLFFRKNLLRLTG |
| 3324 | GLLGsPVRAFFRKNLLRLTG |
| 3325 | GLLGsPVRVFFRKNLLRLTG |
| 3326 | GLLsPARLYAIFFRKNLLRLTG |
| 3327 | GLLsPARLYAVFFRKNLLRLTG |
| 3328 | GLLsPRFVDVFFRKNLLRLTG |
| 3329 | GLLsPRHSLFFRKNLLRLTG |
| 3330 | GLSFGGRSSGsPFFRKNLLRLTG |
| 3331 | GLSFGGRSSGsVFFRKNLLRLTG |
| 3332 | GMLGsPVRVFFRKNLLRLTG |
| 3333 | GMLsPARLYAIFFRKNLLRLTG |
| 3334 | GMLsPARLYAVFFRKNLLRLTG |
| 3335 | GMLsPGKSIEVFFRKNLLRLTG |
| 3336 | GPKPLFRRMsSFFRKNLLRLTG |
| 3337 | GPKPLFRRMsSLFFRKNLLRLTG |
| 3338 | GPKPLFRRMsSLVFFRKNLLRLTG |
| 3339 | GPKPLFRRMsSLVGFFRKNLLRLTG |
| 3340 | GPKPLFRRMsSLVGPFFRKNLLRLTG |
| 3341 | GPKPLFRRMsSLVGPTFFRKNLLRLTG |
| 3342 | GPKPLFRRMsSLVGPTQFFRKNLLRLTG |
| 3343 | GPKPLFRRMsSLVGPTQSFFRKNLLRLTG |
| 3344 | GPPYQRRGsLFFRKNLLRLTG |
| 3345 | GPQPGRHsFFFRKNLLRLTG |
| 3346 | GPQPGRHsLFFRKNLLRLTG |
| 3347 | GPQPGRHsVFFRKNLLRLTG |
| 3348 | GPRPGsPSAFFFRKNLLRLTG |
| 3349 | GPRPGsPSALFFRKNLLRLTG |
| 3350 | GPRPGsPSAMFFRKNLLRLTG |
| 3351 | GPRPGsPSAVFFRKNLLRLTG |
| 3352 | GPRSAsLLFFRKNLLRLTG |
| 3353 | GPRsASLLSFFFRKNLLRLTG |
| 3354 | GPRSAsLLsFFFRKNLLRLTG |
| 3355 | GPRSASLLsFFFRKNLLRLTG |
| 3356 | GPRsAsLLSLFFRKNLLRLTG |
| 3357 | GPRsASLLSLFFRKNLLRLTG |
| 3358 | GPRSAsLLsLFFRKNLLRLTG |
| 3359 | GPRSAsLLSLFFRKNLLRLTG |
| 3360 | GPRSASLLsLFFRKNLLRLTG |
| 3361 | GPRsASLLSMFFRKNLLRLTG |
| 3362 | GPRSAsLLsMFFRKNLLRLTG |
| 3363 | GPRSASLLsMFFRKNLLRLTG |
| 3364 | GPRsASLLSVFFRKNLLRLTG |
| 3365 | GPRSAsLLsVFFRKNLLRLTG |
| 3366 | GPRSASLLsVFFRKNLLRLTG |
| 3367 | GPRsPKAPPFFRKNLLRLTG |
| 3368 | GPRsPPVTLFFRKNLLRLTG |
| 3369 | GQLsPGVQFFFRKNLLRLTG |
| 3370 | GRKsPPPSKFFRKNLLRLTG |
| 3371 | GRKsPPPSKFFRKNLLRLTG |
| 3372 | GRKsPPPSLFFRKNLLRLTG |
| 3373 | GRKsPPPSMFFRKNLLRLTG |
| 3374 | GRKsPPPSRFFRKNLLRLTG |
| 3375 | GRKsPPPSYFFRKNLLRLTG |
| 3376 | GRLGsPHRFFFRKNLLRLTG |
| 3377 | GRLGsPHRKFFRKNLLRLTG |
| 3378 | GRLGsPHRLFFRKNLLRLTG |
| 3379 | GRLGsPHRMFFRKNLLRLTG |
| 3380 | GRLGsPHRRFFRKNLLRLTG |
| 3381 | GRLGsPHRYFFRKNLLRLTG |
| 3382 | GRLsPAYSLFFRKNLLRLTG |
| 3383 | GRLsPKASQVFFFRKNLLRLTG |
| 3384 | GRLsPKASQVKFFRKNLLRLTG |
| 3385 | GRLsPKASQVLFFRKNLLRLTG |
| 3386 | GRLsPKASQVMFFRKNLLRLTG |
| 3387 | GRLsPKASQVRFFRKNLLRLTG |
| 3388 | GRLsPKASQVYFFRKNLLRLTG |
| 3389 | GRLsPVPVPFFFRKNLLRLTG |
| 3390 | GRLsPVPVPKFFRKNLLRLTG |
| 3391 | GRLsPVPVPLFFRKNLLRLTG |
| 3392 | GRLsPVPVPMFFRKNLLRLTG |
| 3393 | GRLsPVPVPRFFRKNLLRLTG |
| 3394 | GRLsPVPVPYFFRKNLLRLTG |
| 3395 | GRQsPSFKLFFRKNLLRLTG |
| 3396 | GRsSPPPGYFFRKNLLRLTG |
| 3397 | GRSsTASLVKFFFRKNLLRLTG |
| 3398 | GRSsTASLVKKFFRKNLLRLTG |
| 3399 | GRSsTASLVKKKFFRKNLLRLTG |
| 3400 | GRSsTASLVKLFFRKNLLRLTG |
| 3401 | GRSsTASLVKMFFRKNLLRLTG |
| 3402 | GRSsTASLVKRFFRKNLLRLTG |
| 3403 | GRSsTASLVKYFFRKNLLRLTG |
| 3404 | GRtGLPDLFFRKNLLRLTG |
| 3405 | GSALGGGGAGLSGRASGGAQsPLRYLHVFFRKNLLRLTG |
| 3406 | GSDsSDDGKKYFFRKNLLRLTG |
| 3407 | GSEsSDDGKKYFFRKNLLRLTG |
| 3408 | GsPHYFSPFFFRKNLLRLTG |
| 3409 | GsPHYFSPFRPYFFRKNLLRLTG |
| 3410 | GsPTMVEKGLEPGVFTLFFRKNLLRLTG |
| 3411 | GsQLAVMMYLFFRKNLLRLTG |
| 3412 | GTDsSDDGKKYFFRKNLLRLTG |
| 3413 | GTEsSDDGKKYFFRKNLLRLTG |
| 3414 | GTIRSRsFIFKFFRKNLLRLTG |
| 3415 | GTIRSRsFIFYFFRKNLLRLTG |
| 3416 | GtLPKYFFRKNLLRLTG |
| 3417 | GtLRRSDSQQAVKFFRKNLLRLTG |
| 3418 | GtLRRSDSQQAVKSFFRKNLLRLTG |
| 3419 | GtLRRSDSQQAVKSPPFFRKNLLRLTG |
| 3420 | GVAsPTITVFFRKNLLRLTG |
| 3421 | GVVsPTFELFFRKNLLRLTG |
| 3422 | HEKKAYsFFFRKNLLRLTG |
| 3423 | HKGEIRGASTPFQFRAssPFFRKNLLRLTG |
| 3424 | HLHsPQHKLFFRKNLLRLTG |
| 3425 | HPKRSVsLFFRKNLLRLTG |
| 3426 | HPRsPNVLFFRKNLLRLTG |
| 3427 | HPRsPNVLSFFFRKNLLRLTG |
| 3428 | HPRsPNVLSLFFRKNLLRLTG |
| 3429 | HPRsPNVLSMFFRKNLLRLTG |
| 3430 | HPRsPNVLSVFFRKNLLRLTG |
| 3431 | HPRsPTPTFFFRKNLLRLTG |
| 3432 | HPRSPtPTFFFRKNLLRLTG |
| 3433 | HPRsPTPTLFFRKNLLRLTG |
| 3434 | HPRSPtPTLFFRKNLLRLTG |
| 3435 | HPRsPTPTMFFRKNLLRLTG |
| 3436 | HPRSPtPTMFFRKNLLRLTG |
| 3437 | HPRSPtPTVFFRKNLLRLTG |
| 3438 | HPsSPTPTVFFRKNLLRLTG |
| 3439 | HRLsPVKGEFFFRKNLLRLTG |
| 3440 | HRLsPVKGEKFFRKNLLRLTG |
| 3441 | HRLsPVKGERFFRKNLLRLTG |
| 3442 | HRLsPVKGEYFFRKNLLRLTG |
| 3443 | HRNsMKVFLFFRKNLLRLTG |
| 3444 | HRNsNPVIAEFFFRKNLLRLTG |
| 3445 | HRNsNPVIAEKFFRKNLLRLTG |
| 3446 | HRNsNPVIAELFFRKNLLRLTG |
| 3447 | HRNsNPVIAERFFRKNLLRLTG |
| 3448 | HRNsNPVIAEYFFRKNLLRLTG |
| 3449 | HRYsTPHAFFFRKNLLRLTG |
| 3450 | HTAsPTGMMKFFRKNLLRLTG |
| 3451 | HVYtPSTTKFFRKNLLRLTG |
| 3452 | IEKIyIMKADTVIVGFFRKNLLRLTG |
| 3453 | IIEtPHKEIFFRKNLLRLTG |
| 3454 | IIEtPHKEYFFRKNLLRLTG |
| 3455 | IISsPLKGYFFRKNLLRLTG |
| 3456 | IISsPLTGKFFRKNLLRLTG |
| 3457 | ILDRtPEKLFFRKNLLRLTG |
| 3458 | ILDRtPEKVFFRKNLLRLTG |
| 3459 | ILDsGIYRIFFRKNLLRLTG |
| 3460 | ILDsGIYRVFFRKNLLRLTG |
| 3461 | ILKPRRsLFFRKNLLRLTG |
| 3462 | ILKsPEIQRAFFRKNLLRLTG |
| 3463 | ILKsPEIQRVFFRKNLLRLTG |
| 3464 | ILQtPQFQMFFRKNLLRLTG |
| 3465 | ILQVsIPSLFFRKNLLRLTG |
| 3466 | IMDRtPEKLFFRKNLLRLTG |
| 3467 | IMDRtPEKVFFRKNLLRLTG |
| 3468 | IMDsGIYRIFFRKNLLRLTG |
| 3469 | IMDsGIYRVFFRKNLLRLTG |
| 3470 | IMKsPEIQRAFFRKNLLRLTG |
| 3471 | IMKsPEIQVRFFRKNLLRLTG |
| 3472 | INKERRSsLFFRKNLLRLTG |
| 3473 | IPVgSSHNSLFFRKNLLRLTG |
| 3474 | IQFsPPFPGAFFRKNLLRLTG |
| 3475 | ISDGtLKYFFRKNLLRLTG |
| 3476 | ISDGtPLKYFFRKNLLRLTG |
| 3477 | ISDSAHtDYFFRKNLLRLTG |
| 3478 | ISDsMHSLYFFRKNLLRLTG |
| 3479 | ISDtPHKEIFFRKNLLRLTG |
| 3480 | ISDtPHKEYFFRKNLLRLTG |
| 3481 | ISEGtLKYFFRKNLLRLTG |
| 3482 | ISEGtPLKYFFRKNLLRLTG |
| 3483 | ISESAHtDYFFRKNLLRLTG |
| 3484 | ISEsMHSLYFFRKNLLRLTG |
| 3485 | ISEtPHKEIFFRKNLLRLTG |
| 3486 | ISEtPHKEYFFRKNLLRLTG |
| 3487 | ISFSAHtDYFFRKNLLRLIG |
| 3488 | ISSsMHSLYFFRKNLLRLTG |
| 3489 | IStDRDPLFFRKNLLRLTG |
| 3490 | IStDRDPYFFRKNLLRLIG |
| 3491 | ITDGtLKYFFRKNLLRLTG |
| 3492 | ITDGtPLKYFFRKNLLRLTG |
| 3493 | ITDSAHtDYFFRKNLLRLTG |
| 3494 | ITDsMHSLYFFRKNLLRLTG |
| 3495 | ITDtPHKEIFFRKNLLRLTG |
| 3496 | ITDtPHKEYFFRKNLLRLTG |
| 3497 | ITEGtLKYFFRKNLLRLTG |
| 3498 | ITEGtPLKYFFRKNLLRLTG |
| 3499 | ITESAHtDYFFRKNLLRLTG |
| 3500 | ITEsMHSLYFFRKNLLRLTG |
| 3501 | ITEtPHKEIFFRKNLLRLTG |
| 3502 | ITEtPHKEYFFRKNLLRLTG |
| 3503 | ITQGtLKYFFRKNLLRLTG |
| 3504 | ITQGtPLKKFFRKNLLRLTG |
| 3505 | ITQGtPLKYFFRKNLLRLTG |
| 3506 | ITtDRDPLFFRKNLLRLTG |
| 3507 | ITtDRDPYFFRKNLLRLTG |
| 3508 | IVLsDSEVIQLFFRKNLLRLTG |
| 3509 | IVRyHQLFFRKNLLRLTG |
| 3510 | IVtDRDPLFFRKNLLRLTG |
| 3511 | IVtDRDPYFFRKNLLRLTG |
| 3512 | IYQyIQSRFFFRKNLLRLTG |
| 3513 | KAFsPVRFFRKNLLRLTG |
| 3514 | KAFsPVRSVFFRKNLLRLTG |
| 3515 | KAKsPAPGLFFRKNLLRLTG |
| 3516 | KAKsPAPGVFFRKNLLRLTG |
| 3517 | KARsPGRAFFFRKNLLRLTG |
| 3518 | KARsPGRALFFRKNLLRLTG |
| 3519 | KARsPGRAMFFRKNLLRLTG |
| 3520 | KARsPGRAVFFRKNLLRLTG |
| 3521 | KASPKRLsLFFRKNLLRLTG |
| 3522 | KAVsLFLcYFFRKNLLRLTG |
| 3523 | KAVsLFLCYFFRKNLLRLTG |
| 3524 | KEGEEPTVYsDEEEPKDESARKNDFFRKNLLRLTG |
| 3525 | KEKsPFRETFFRKNLLRLTG |
| 3526 | KELARQIsFFFRKNLLRLTG |
| 3527 | KEMsPTRQFFFRKNLLRLTG |
| 3528 | KEmsPTRQLFFRKNLLRLTG |
| 3529 | KEMsPTRQLFFRKNLLRLTG |
| 3530 | KEMsPTRQWFFRKNLLRLTG |
| 3531 | KEMsPTRQYFFRKNLLRLTG |
| 3532 | KESsPLSSRKIFFRKNLLRLTG |
| 3533 | KFRPPPLsLFFRKNLLRLTG |
| 3534 | KGIsSSSLKEKFFRKNLLRLTG |
| 3535 | KIAsEIAQLFFRKNLLRLTG |
| 3536 | KIDIVsSQKVFFRKNLLRLTG |
| 3537 | KIDsPTKVKKFFRKNLLRLTG |
| 3538 | KIEKIyIMKADTVIVGFFRKNLLRLTG |
| 3539 | KIEsLENLYLFFRKNLLRLTG |
| 3540 | KIFsGVFVKFFRKNLLRLTG |
| 3541 | KIFsGVFVKVFFRKNLLRLTG |
| 3542 | KIFsKQQGKFFRKNLLRLTG |
| 3543 | KIFsKQQGYFFRKNLLRLTG |
| 3544 | KIGsIIFQVFFRKNLLRLTG |
| 3545 | KIKsFEVVFFFRKNLLRLTG |
| 3546 | KIRSsPREAKFFRKNLLRLTG |
| 3547 | KIRSsPREAYFFRKNLLRLTG |
| 3548 | KIRTsPTFRFFRKNLLRLTG |
| 3549 | KIRTsPTFYFFRKNLLRLTG |
| 3550 | KLAsLEREASVFFRKNLLRLTG |
| 3551 | KLAsLLHQVFFRKNLLRLTG |
| 3552 | KLAsPEKLAGLFFRKNLLRLTG |
| 3553 | KLAsPELERLFFRKNLLRLTG |
| 3554 | KLAsPELERVFFRKNLLRLTG |
| 3555 | KLDIVsSQKVFFRKNLLRLTG |
| 3556 | KLDsFLDMQVFFRKNLLRLTG |
| 3557 | KLDsPRVTVFFRKNLLRLTG |
| 3558 | KLDsPTKVKKFFRKNLLRLTG |
| 3559 | KLDsPTKVKYFFRKNLLRLTG |
| 3560 | KLFPDtPLALFFRKNLLRLTG |
| 3561 | KLFPDtPLAVFFRKNLLRLTG |
| 3562 | KLFsGTVRKFFRKNLLRLTG |
| 3563 | KLFsGVFVKVFFRKNLLRLTG |
| 3564 | KLFsKQQGKFFRKNLLRLTG |
| 3565 | KLFsKQQGYFFRKNLLRLTG |
| 3566 | KLFsPAHKKFFRKNLLRLTG |
| 3567 | KLFsPAHKYFFRKNLLRLTG |
| 3568 | KLFsPSKEAELFFRKNLLRLTG |
| 3569 | KLFsPSKEAEVFFRKNLLRLTG |
| 3570 | KLHGsLARAGKFFRKNLLRLTG |
| 3571 | KLHGsLARAGYFFRKNLLRLTG |
| 3572 | KLIDIVsSQKVFFRKNLLRLTG |
| 3573 | KLIDRTEsLFFRKNLLRLTG |
| 3574 | KLIDVsSQKVFFRKNLLRLTG |
| 3575 | KLIsSSSLKEKFFRKNLLRLTG |
| 3576 | KLIsSSSLKEYFFRKNLLRLTG |
| 3577 | KLKDRLPsIFFRKNLLRLTG |
| 3578 | KLKsNPDFLKFFRKNLLRLTG |
| 3579 | KLKsNPDFLKKFFRKNLLRLTG |
| 3580 | KLKsNPDFLKYFFRKNLLRLTG |
| 3581 | KLKsPAPGLFFRKNLLRLTG |
| 3582 | KLKsPAPGVFFRKNLLRLTG |
| 3583 | KLKsQEIFLFFRKNLLRLTG |
| 3584 | KLKSsPLIEKKFFRKNLLRLTG |
| 3585 | KLKSsPLIEKYFFRKNLLRLTG |
| 3586 | KLKtPLVAKFFRKNLLRLTG |
| 3587 | KLKtPLVARFFRKNLLRLTG |
| 3588 | KLLDFGSLsNLQVFFRKNLLRLTG |
| 3589 | KLLQFYPsLFFRKNLLRLTG |
| 3590 | KLLQFYPsVFFRKNLLRLTG |
| 3591 | KLLsPSDEKLFFRKNLLRLTG |
| 3592 | KLLsPSNEKLFFRKNLLRLTG |
| 3593 | KLLsPSNEKVFFRKNLLRLTG |
| 3594 | KLLSSAQRtLFFRKNLLRLTG |
| 3595 | KLLSSAQRtVFFRKNLLRLTG |
| 3596 | KLLsTEEMELFFRKNLLRLTG |
| 3597 | KLLsTEEMEVFFRKNLLRLTG |
| 3598 | KLLsVERIKFFRKNLLRLTG |
| 3599 | KLLtPIKEKFFRKNLLRLTG |
| 3600 | KLLtPIKEYFFRKNLLRLTG |
| 3601 | KLMAPDIsLFFRKNLLRLTG |
| 3602 | KLMAPDIsVFFRKNLLRLTG |
| 3603 | KLMIDRTEsVFFRKNLLRLTG |
| 3604 | KLMsDVEDVFFRKNLLRLTG |
| 3605 | KLMsPKADVFFRKNLLRLTG |
| 3606 | KLMsPKADVKLFFRKNLLRLTG |
| 3607 | KLMsPKADVKVFFRKNLLRLTG |
| 3608 | KLPDsPALAFFRKNLLRLTG |
| 3609 | KLPDsPALAKFFRKNLLRLTG |
| 3610 | KLPDsPALAKKFFRKNLLRLTG |
| 3611 | KLPDsPALAKYFFRKNLLRLTG |
| 3612 | KLPDsPALAYFFRKNLLRLTG |
| 3613 | KLPsPAPARKFFRKNLLRLTG |
| 3614 | KLPTsPLKMKFFRKNLLRLTG |
| 3615 | KLPTsPLKMYFFRKNLLRLTG |
| 3616 | KLPTtPVKAKFFRKNLLRLTG |
| 3617 | KLPTtPVKAYFFRKNLLRLTG |
| 3618 | KLQEFLQtLFFRKNLLRLTG |
| 3619 | KLQVtSLSVFFRKNLLRLTG |
| 3620 | KLRsPFLQKFFRKNLLRLTG |
| 3621 | KLRsPFLQYFFRKNLLRLTG |
| 3622 | KLRSsPREAKFFRKNLLRLTG |
| 3623 | KLRTsPTFKFFRKNLLRLTG |
| 3624 | KLsGDQPAARFFRKNLLRLTG |
| 3625 | KLSGLsFFFRKNLLRLTG |
| 3626 | KLSsLGNLKFFRKNLLRLTG |
| 3627 | KLSsLGNLKKFFRKNLLRLTG |
| 3628 | KLSsLGNLKYFFRKNLLRLTG |
| 3629 | KLSsPRGGMKFFRKNLLRLTG |
| 3630 | KLSsPRGGMKKFFRKNLLRLTG |
| 3631 | KLSsPRGGMKYFFRKNLLRLTG |
| 3632 | KLsVIAEDSESGKQNFFRKNLLRLTG |
| 3633 | KLsVIAEDSESGKQNPFFRKNLLRLTG |
| 3634 | KLsVIAEDSESGKQNPGFFRKNLLRLTG |
| 3635 | KLVSFHDDsDEDLFFRKNLLRLTG |
| 3636 | KLYsEIDIKVFFRKNLLRLTG |
| 3637 | KLYsGNMEKFFRKNLLRLTG |
| 3638 | KMAsLLHQVFFRKNLLRLTG |
| 3639 | KMAsPELERLFFRKNLLRLTG |
| 3640 | KMAsPELERVFFRKNLLRLTG |
| 3641 | KMDIVsSQKVFFRKNLLRLTG |
| 3642 | KMDsFLDMQLFFRKNLLRLTG |
| 3643 | KMDsFLDMQVFFRKNLLRLTG |
| 3644 | KMDsPRVTVFFRKNLLRLTG |
| 3645 | KMDsPTKVKKFFRKNLLRLTG |
| 3646 | KMFPDtPLALFFRKNLLRLTG |
| 3647 | KMFPDtPLAVFFRKNLLRLTG |
| 3648 | KMFsGTVRKFFRKNLLRLTG |
| 3649 | KMFsGVFVKVFFRKNLLRLTG |
| 3650 | KMFsKQQGKFFRKNLLRLTG |
| 3651 | KMFsPAHKKFFRKNLLRLTG |
| 3652 | KMFsPSKEAELFFRKNLLRLTG |
| 3653 | KMFsPSKEAEVFFRKNLLRLTG |
| 3654 | KMHGsLARAGKFFRKNLLRLTG |
| 3655 | KMIDIVsSQKVFFRKNLLRLTG |
| 3656 | KMIDRTEsLFFRKNLLRLTG |
| 3657 | KMIsSSSLKEKFFRKNLLRLTG |
| 3658 | KMKsNPDFLKFFRKNLLRLTG |
| 3659 | KMKsNPDFLKKFFRKNLLRLTG |
| 3660 | KMKsNPDFLKYFFRKNLLRLTG |
| 3661 | KMKSsPLIEKKFFRKNLLRLTG |
| 3662 | KMKtPLVAKFFRKNLLRLTG |
| 3663 | KMKtPLVARFFRKNLLRLTG |
| 3664 | KMLDFGSLsNLOVFFRKNLLRLTG |
| 3665 | KMLDFGSLsNLQVFFRKNLLRLTG |
| 3666 | KMLQFYPsLFFRKNLLRLTG |
| 3667 | KMLsPSNEKLFFRKNLLRLTG |
| 3668 | KMLsPSNEKVFFRKNLLRLTG |
| 3669 | KMLSSAQRtLFFRKNLLRLTG |
| 3670 | KMLSSAQRtVFFRKNLLRLTG |
| 3671 | KMLsVERIKFFRKNLLRLTG |
| 3672 | KMLtPIKEKFFRKNLLRLTG |
| 3673 | KKMAPDIsVFFRKNLLRLIG |
| 3674 | KM:MsPKADVKLFFRKNLLRLTG |
| 3675 | KM:MsPKADVKVFFRKNLLRLTG |
| 3676 | KMPTsPLKMKFFRKNLLRLTG |
| 3677 | KMPTtPVKAKFFRKNLLRLTG |
| 3678 | KMPTtPVKAYFFRKNLLRLTG |
| 3679 | KMRsPFLQKFFRKNLLRLTG |
| 3680 | KMRSsPREAKFFRKNLLRLTG |
| 3681 | KMRTsPTFKFFRKNLLRLTG |
| 3682 | KMSsLGNLKFFRKNLLRLTG |
| 3683 | KMSsLGNLKKFFRKNLLRLTG |
| 3684 | KMSsLGNLKYFFRKNLLRLTG |
| 3685 | KMSsPRGGMKFFRKNLLRLTG |
| 3686 | KMSsPRGGMKKFFRKNLLRLTG |
| 3687 | KMYsEIDIKVFFRKNLLRLTG |
| 3688 | KMYsGNMEKFFRKNLLRLTG |
| 3689 | KNRsWKYNFFRKNLLRLTG |
| 3690 | KNRsWKYNQFFRKNLLRLTG |
| 3691 | KNRsWKYNQSISLRFFRKNLLRLTG |
| 3692 | KNRsWKYNQSISLRRPFFRKNLLRLTG |
| 3693 | KPAsPARRFFFRKNLLRLTG |
| 3694 | KPAsPARRLFFRKNLLRLTG |
| 3695 | KPAsPARRMFFRKNLLRLTG |
| 3696 | KPAsPARRVFFRKNLLRLTG |
| 3697 | KPAsPKFIVTFFFRKNLLRLTG |
| 3698 | KPAsPKFIVTLFFRKNLLRLTG |
| 3699 | KPAsPKFIVTMFFRKNLLRLTG |
| 3700 | KPAsPKFIVTVFFRKNLLRLTG |
| 3701 | KPEsRRSSLFFRKNLLRLTG |
| 3702 | KPEsRRsSLLFFRKNLLRLTG |
| 3703 | KPEsRRSsLLFFRKNLLRLTG |
| 3704 | KPEsRRSSLLFFRKNLLRLTG |
| 3705 | KPLIRsQSLFFRKNLLRLTG |
| 3706 | KPLIRSQsLFFRKNLLRLTG |
| 3707 | KPPHsPLVFFFRKNLLRLTG |
| 3708 | KPPHsPLVLFFRKNLLRLTG |
| 3709 | KPPHsPLVMFFRKNLLRLTG |
| 3710 | KPPHsPLVVFFRKNLLRLTG |
| 3711 | KPPsPEHQSFFFRKNLLRLTG |
| 3712 | KPPsPEHQSLFFRKNLLRLTG |
| 3713 | KPPsPEHQSMFFRKNLLRLTG |
| 3714 | KPPsPEHQSVFFRKNLLRLTG |
| 3715 | KPPsPSPIEFFFRKNLLRLTG |
| 3716 | KPPsPSPIELFFRKNLLRLTG |
| 3717 | KPPsPSPIEMFFRKNLLRLTG |
| 3718 | KPPsPSPIEVFFRKNLLRLTG |
| 3719 | KPPtPGASFFFRKNLLRLTG |
| 3720 | KPPtPGASLFFRKNLLRLTG |
| 3721 | KPPtPGASMFFRKNLLRLTG |
| 3722 | KPPtPGASVFFRKNLLRLTG |
| 3723 | KPPYRSHsFFFRKNLLRLTG |
| 3724 | KPPYRSHsLFFRKNLLRLTG |
| 3725 | KPPYRSHsMFFRKNLLRLTG |
| 3726 | KPPYRSHsVFFRKNLLRLTG |
| 3727 | KPQTRGKtFFFRKNLLRLTG |
| 3728 | KPQTRGKtLFFRKNLLRLTG |
| 3729 | KPQTRGKtMFFRKNLLRLTG |
| 3730 | KPQTRGKtVFFRKNLLRLTG |
| 3731 | KPRPLsMDLFFRKNLLRLTG |
| 3732 | KPRPPPLsFFFRKNLLRLTG |
| 3733 | KPRPPPLsLFFRKNLLRLTG |
| 3734 | KPRPPPLsMFFRKNLLRLTG |
| 3735 | KPRPPPLsPFFRKNLLRLTG |
| 3736 | KPRPPPLsVFFRKNLLRLTG |
| 3737 | KPRRFsRsLFFRKNLLRLTG |
| 3738 | KPRRFsRSLFFRKNLLRLTG |
| 3739 | KPRsPDHVFFFRKNLLRLTG |
| 3740 | KPRsPDHVLFFRKNLLRLTG |
| 3741 | KPRsPDHVMFFRKNLLRLTG |
| 3742 | KPRsPDHVVFFRKNLLRLTG |
| 3743 | KPRsPFSKIFFRKNLLRLTG |
| 3744 | KPRsPPRAFFFRKNLLRLTG |
| 3745 | KPRsPPRALFFRKNLLRLTG |
| 3746 | KPRsPPRALFFFRKNLLRLTG |
| 3747 | KPRsPPRALLFFRKNLLRLTG |
| 3748 | KPRsPPRALMFFRKNLLRLTG |
| 3749 | KPRsPPRALVFFRKNLLRLTG |
| 3750 | KPRsPPRALVFFFRKNLLRLTG |
| 3751 | KPRsPPRALVLFFRKNLLRLTG |
| 3752 | KPRsPPRALVLFFFRKNLLRLTG |
| 3753 | KPRsPPRALVLLFFRKNLLRLTG |
| 3754 | KPRsPPRALVLMFFRKNLLRLTG |
| 3755 | KPRsPPRALVLPFFRKNLLRLTG |
| 3756 | KPRsPPRALVLVFFRKNLLRLTG |
| 3757 | KPRsPPRALVMFFRKNLLRLTG |
| 3758 | KPRsPPRALVVFFRKNLLRLTG |
| 3759 | KPRsPPRAMFFRKNLLRLTG |
| 3760 | KPRsPPRAVFFRKNLLRLTG |
| 3761 | KPRsPVVEFFFRKNLLRLTG |
| 3762 | KPRsPVVELFFRKNLLRLTG |
| 3763 | KPRsPVVEMFFRKNLLRLTG |
| 3764 | KPRsPVVEVFFRKNLLRLTG |
| 3765 | KPSsPRGSLFFRKNLLRLTG |
| 3766 | KPSsPRGSLLFFRKNLLRLTG |
| 3767 | KPVsPKSGTLFFRKNLLRLTG |
| 3768 | KPYsPLASFFFRKNLLRLTG |
| 3769 | KPYsPLASLFFRKNLLRLTG |
| 3770 | KPYsPLASMFFRKNLLRLTG |
| 3771 | KPYsPLASVFFRKNLLRLTG |
| 3772 | KQDsLVINLFFRKNLLRLTG |
| 3773 | KRAsFAKSFFFRKNLLRLTG |
| 3774 | KRAsFAKSKFFRKNLLRLTG |
| 3775 | KRAsFAKSLFFRKNLLRLTG |
| 3776 | KRAsFAKSMFFRKNLLRLTG |
| 3777 | KRAsFAKSRFFRKNLLRLTG |
| 3778 | KRAsFAKSVFFRKNLLRLTG |
| 3779 | KRAsFAKSYFFRKNLLRLTG |
| 3780 | KRAsGQAFEFFFRKNLLRLTG |
| 3781 | KRAsGQAFEKFFRKNLLRLTG |
| 3782 | KRAsGQAFELFFRKNLLRLTG |
| 3783 | KRAsGQAFERFFRKNLLRLTG |
| 3784 | KRAsGQAFEYFFRKNLLRLTG |
| 3785 | KRASsPFRFFFRKNLLRLTG |
| 3786 | KRASsPFRKFFRKNLLRLTG |
| 3787 | KRASsPFRLFFRKNLLRLTG |
| 3788 | KRASsPFRMFFRKNLLRLTG |
| 3789 | KRASsPFRRFFRKNLLRLTG |
| 3790 | KRASsPFRYFFRKNLLRLTG |
| 3791 | KRAsVFVKFFFRKNLLRLTG |
| 3792 | KRAsVFVKKFFRKNLLRLTG |
| 3793 | KRAsVFVKLFFRKNLLRLTG |
| 3794 | KRAsVFVKMFFRKNLLRLTG |
| 3795 | KRAsVFVKRFFRKNLLRLTG |
| 3796 | KRAsVFVKYFFRKNLLRLTG |
| 3797 | KRAsYILRLFFRKNLLRLTG |
| 3798 | KRFsFKFFFRKNLLRLTG |
| 3799 | KRFsFKKFFRKNLLRLTG |
| 3800 | KRFsFKKsFFFRKNLLRLTG |
| 3801 | KRFsFKKSFFFRKNLLRLTG |
| 3802 | KRFsFKKSKFFRKNLLRLTG |
| 3803 | KRFsFKKSLFFRKNLLRLTG |
| 3804 | KRFsFKKSMFFRKNLLRLTG |
| 3805 | KRFsFKKSRFFRKNLLRLTG |
| 3806 | KRFsFKKSYFFRKNLLRLTG |
| 3807 | KRFsFKLFFRKNLLRLTG |
| 3808 | KRFsFKMFFRKNLLRLTG |
| 3809 | KRFsFKRFFRKNLLRLTG |
| 3810 | KRFsFKsSFFFRKNLLRLTG |
| 3811 | KRFsFKYFFRKNLLRLTG |
| 3812 | KRFsGTVRFFFRKNLLRLTG |
| 3813 | KRFsGTVRKFFRKNLLRLTG |
| 3814 | KRFsGTVRLFFRKNLLRLTG |
| 3815 | KRFsGTVRMFFRKNLLRLTG |
| 3816 | KRFsGTVRRFFRKNLLRLTG |
| 3817 | KRFsGTVRYFFRKNLLRLTG |
| 3818 | KRIVIsPKPFFFRKNLLRLTG |
| 3819 | KRKsFTSLYFFRKNLLRLTG |
| 3820 | KRLEKsPSFFFRKNLLRLTG |
| 3821 | KRLEKSPsFFFRKNLLRLTG |
| 3822 | KRLsPAPQFFFRKNLLRLTG |
| 3823 | KRLsPAPQKFFRKNLLRLTG |
| 3824 | KRLsPAPQLFFRKNLLRLTG |
| 3825 | KRLsPAPQMFFRKNLLRLTG |
| 3826 | KRLsPAPQRFFRKNLLRLTG |
| 3827 | KRLsPAPQYFFRKNLLRLTG |
| 3828 | KRLsTSPVRLFFRKNLLRLTG |
| 3829 | KRLsVERIFFFRKNLLRLTG |
| 3830 | KRLsVERIKFFRKNLLRLTG |
| 3831 | KRLsVERILFFRKNLLRLTG |
| 3832 | KRLsVERIMFFRKNLLRLTG |
| 3833 | KRLsVERIRFFRKNLLRLTG |
| 3834 | KRLsVERIYFFRKNLLRLTG |
| 3835 | KRMsPKEFFFRKNLLRLTG |
| 3836 | KRMsPKEKFFRKNLLRLTG |
| 3837 | KRMsPKELFFRKNLLRLTG |
| 3838 | KRMsPKERFFRKNLLRLTG |
| 3839 | KRMsPKEYFFRKNLLRLTG |
| 3840 | KRmsPKPELFFRKNLLRLTG |
| 3841 | KRMsPKPELFFRKNLLRLTG |
| 3842 | KRMsPKPFFFRKNLLRLTG |
| 3843 | KRMsPKPKFFRKNLLRLTG |
| 3844 | KRMsPKPLFFRKNLLRLTG |
| 3845 | KRMsPKPMFFRKNLLRLTG |
| 3846 | KRMsPKPRFFRKNLLRLTG |
| 3847 | KRMsPKPYFFRKNLLRLTG |
| 3848 | KRPEsPPSIFFRKNLLRLTG |
| 3849 | KRWQsPVTKFFRKNLLRLTG |
| 3850 | KRYsEPVSLFFRKNLLRLTG |
| 3851 | KRYsGNMEFFFRKNLLRLTG |
| 3852 | KRYsGNMEKFFRKNLLRLTG |
| 3853 | KRYsGNMELFFRKNLLRLTG |
| 3854 | KRYsGNMEMFFRKNLLRLTG |
| 3855 | KRYsGNMERFFRKNLLRLTG |
| 3856 | KRYsGNmEYFFRKNLLRLTG |
| 3857 | KRYsGNMEYFFRKNLLRLTG |
| 3858 | KRYsRALYLFFRKNLLRLTG |
| 3859 | KSDsRQERYFFRKNLLRLTG |
| 3860 | KSEsRQERYFFRKNLLRLTG |
| 3861 | KSGELLAtWFFRKNLLRLTG |
| 3862 | KSKsNPDFLKKFFRKNLLRLTG |
| 3863 | KSKsNPFLKKFFRKNLLRLTG |
| 3864 | KSKtPLVAKFFRKNLLRLTG |
| 3865 | KSKtPLVARFFRKNLLRLTG |
| 3866 | KSKtPLVAYFFRKNLLRLTG |
| 3867 | KsLVRLLLLFFRKNLLRLTG |
| 3868 | KSSsLGNLKKFFRKNLLRLTG |
| 3869 | KsVKALSSLHGDDQFFRKNLLRLTG |
| 3870 | KsVKALSSLHGDDQDFFRKNLLRLTG |
| 3871 | KsVKALSSLHGDDQDsEDEFFRKNLLRLTG |
| 3872 | KSVKALSSLHGDDQDsEDEFFRKNLLRLTG |
| 3873 | KTDsRQEYRFFRKNLLRLTG |
| 3874 | KTEsRQERYFFRKNLLRLTG |
| 3875 | KtLSPGKNGVVKFFRKNLLRLTG |
| 3876 | KtLSPGKNGVVYFFRKNLLRLTG |
| 3877 | KTMsGTFLLFFRKNLLRLTG |
| 3878 | KTMsPSQMIMFFRKNLLRLTG |
| 3879 | KTPTsPLKMKFFRKNLLRLTG |
| 3880 | KTPTsPLKMYFFRKNLLRLTG |
| 3881 | KTWKGsIGLFFRKNLLRLTG |
| 3882 | KVAsLLHQVFFRKNLLRLTG |
| 3883 | KVDsPVIFFFRKNLLRLTG |
| 3884 | KVHGsLARAGKFFRKNLLRLTG |
| 3885 | KVHGsLARAGYFFRKNLLRLTG |
| 3886 | KVKSsPLIEKKFFRKNLLRLTG |
| 3887 | KVKsSPLIEKLFFRKNLLRLTG |
| 3888 | KVKSsPLIEKLFFRKNLLRLTG |
| 3889 | KVKSsPLIEKYFFRKNLLRLTG |
| 3890 | KVLsKEFHLFFRKNLLRLTG |
| 3891 | KVLSPtAAKFFRKNLLRLTG |
| 3892 | KVLsSLVTLFFRKNLLRLTG |
| 3893 | KVLsTEEMELFFRKNLLRLTG |
| 3894 | KVLStEEMELFFRKNLLRLTG |
| 3895 | KVLtPIKeKFFRKNLLRLTG |
| 3896 | KVLtPIKEKFFRKNLLRLTG |
| 3897 | KVLtPIKEYFFRKNLLRLTG |
| 3898 | KVPDsPALAKFFRKNLLRLTG |
| 3899 | KVPDsPALAKKFFRKNLLRLTG |
| 3900 | KVPDsPALAKYFFRKNLLRLTG |
| 3901 | KVPDsPALAYFFRKNLLRLTG |
| 3902 | KVPTsPLKMYFFRKNLLRLTG |
| 3903 | KVQsLRRALFFRKNLLRLTG |
| 3904 | KVQVtSLSVFFRKNLLRLTG |
| 3905 | KVYsSSEFLFFRKNLLRLTG |
| 3906 | KYIsGPHELFFRKNLLRLTG |
| 3907 | KYsPGKLRGNFFRKNLLRLTG |
| 3908 | LGGGGAGLSGRASGGAQsPLRYLHVFFRKNLLRLTG |
| 3909 | LKLsYLTWVFFRKNLLRLTG |
| 3910 | LLAsPGHISVFFRKNLLRLTG |
| 3911 | LLDPSRSYsYFFRKNLLRLTG |
| 3912 | LLDtPVKTQYFFRKNLLRLTG |
| 3913 | LLFsPVTSLFFRKNLLRLTG |
| 3914 | LLFsPVTSVFFRKNLLRLTG |
| 3915 | LLLsEEVELFFRKNLLRLTG |
| 3916 | LLNKSsPVKFFRKNLLRLTG |
| 3917 | LLNKSsPVKKFFRKNLLRLTG |
| 3918 | LLNKSsPVKYFFRKNLLRLTG |
| 3919 | LMFsPVTSLFFRKNLLRLTG |
| 3920 | LMFsPVTSVFFRKNLLRLTG |
| 3921 | LMFsVTSIFFRKNLLRLTG |
| 3922 | LMFsVTSLFFRKNLLRLTG |
| 3923 | LMNKSsPVKFFRKNLLRLTG |
| 3924 | LMNKSsPVKKFFRKNLLRLTG |
| 3925 | LMNKSsPVKYFFRKNLLRLTG |
| 3926 | LPAsPHQFFFRKNLLRLTG |
| 3927 | LPAsPHQLFFRKNLLRLTG |
| 3928 | LPAsPHQMFFRKNLLRLTG |
| 3929 | LPAsPHQVFFRKNLLRLTG |
| 3930 | LPAsPRARFFFRKNLLRLTG |
| 3931 | LPAsPRARLFFRKNLLRLTG |
| 3932 | LPAsPRARMFFRKNLLRLTG |
| 3933 | LPAsPRARVFFRKNLLRLTG |
| 3934 | LPIFSRLsFFFRKNLLRLTG |
| 3935 | LPIFSRLsIFFRKNLLRLTG |
| 3936 | LPIFSRLsLFFRKNLLRLTG |
| 3937 | LPIFSRLsMFFRKNLLRLTG |
| 3938 | LPIFSRLsVFFRKNLLRLTG |
| 3939 | LPKGLsASLFFRKNLLRLTG |
| 3940 | LPKGLSAsLFFRKNLLRLTG |
| 3941 | LPKsPPYTAFFFRKNLLRLTG |
| 3942 | LPKsPPYTALFFRKNLLRLTG |
| 3943 | LPKsPPYTAMFFRKNLLRLTG |
| 3944 | LPKsPPYTAVFFRKNLLRLTG |
| 3945 | LPRGSsPSVFFFRKNLLRLTG |
| 3946 | LPRGsSPSVLFFRKNLLRLTG |
| 3947 | LPRGSsPSVLFFRKNLLRLTG |
| 3948 | LPRGSsPSVMFFRKNLLRLTG |
| 3949 | LPRGSsPSVVFFRKNLLRLTG |
| 3950 | LPRmIsHSELFFRKNLLRLTG |
| 3951 | LPRMIsHSELFFRKNLLRLTG |
| 3952 | LPRPAsPALFFRKNLLRLTG |
| 3953 | LPRSSsMAAFFRKNLLRLTG |
| 3954 | LPRSSsMAAGLFFRKNLLRLTG |
| 3955 | LPRtPRPELFFRKNLLRLTG |
| 3956 | LPVsPRLQLFFRKNLLRLTG |
| 3957 | LQLsPLKGLSLFFRKNLLRLTG |
| 3958 | LQNItENQLFFRKNLLRLTG |
| 3959 | LSDPSRSYsYFFRKNLLRLTG |
| 3960 | LSDsDTEAKLFFRKNLLRLTG |
| 3961 | LSDsDTEAKYFFRKNLLRLTG |
| 3962 | LSDtPVKTQYFFRKNLLRLTG |
| 3963 | LSEPSRSYsYFFRKNLLRLTG |
| 3964 | LSEsDTEAKLFFRKNLLRLTG |
| 3965 | LSEsDTEAKYFFRKNLLRLTG |
| 3966 | LSEtPVKTQYFFRKNLLRLTG |
| 3967 | LSKFRMPQPSSGREsPRHFFRKNLLRLTG |
| 3968 | LSSsVIRELFFRKNLLRLTG |
| 3969 | LTDPSRSYsYFFRKNLLRLTG |
| 3970 | LTDPSsPTISSYFFRKNLLRLTG |
| 3971 | LTDsDTEAKLFFRKNLLRLTG |
| 3972 | LTDsDTEAKYFFRKNLLRLTG |
| 3973 | LTDtPVKTQYFFRKNLLRLTG |
| 3974 | LTEPSRSYsYFFRKNLLRLTG |
| 3975 | LTEsDTEAKLFFRKNLLRLTG |
| 3976 | LTEsDTEAKYFFRKNLLRLTG |
| 3977 | LTEtPVKTOYFFRKNLLRLTG |
| 3978 | LTEtPVKTQYFFRKNLLRLTG |
| 3979 | MLAEsPSVPRLFFRKNLLRLTG |
| 3980 | MLAEsPSVPRVFFRKNLLRLTG |
| 3981 | MLRsPPRVSKFFRKNLLRLTG |
| 3982 | MMRsPPRVSKFFRKNLLRLTG |
| 3983 | MPRPsIKKAQNSQAARQFFRKNLLRLTG |
| 3984 | MPRQPsAIRMFFRKNLLRLTG |
| 3985 | MPRQPsATRFFFRKNLLRLTG |
| 3986 | MPRQPsATRLFFRKNLLRLTG |
| 3987 | MPRQPsATRMFFRKNLLRLTG |
| 3988 | MPRQPsATRVFFRKNLLRLTG |
| 3989 | MRLsEWLQLFFRKNLLRLTG |
| 3990 | MRLsRELQFFFRKNLLRLTG |
| 3991 | MRLsRELQKFFRKNLLRLTG |
| 3992 | MRLsRELQLFFRKNLLRLTG |
| 3993 | MRLsRELQMFFRKNLLRLTG |
| 3994 | MRLsRELQRFFRKNLLRLTG |
| 3995 | MRLsRELQYFFRKNLLRLTG |
| 3996 | MSDtYRLKYFFRKNLLRLTG |
| 3997 | MSEtYRLKYFFRKNLLRLTG |
| 3998 | MTDtYRLKYFFRKNLLRLTG |
| 3999 | MTEtYRLKYFFRKNLLRLTG |
| 4000 | MTRsPPRVSKFFRKNLLRLTG |
| 4001 | MTRsPPRVSYFFRKNLLRLTG |
| 4002 | NAPPAYEKLsAEFFRKNLLRLTG |
| 4003 | NFKsPVKTIRFFRKNLLRLTG |
| 4004 | NLELSKFRMPQPSSGREsPRHFFRKNLLRLTG |
| 4005 | NLGsRNHVQLFFRKNLLRLTG |
| 4006 | NLLsPDGKMISVFFRKNLLRLTG |
| 4007 | NLVERKNsKFFRKNLLRLTG |
| 4008 | NLVERKNsLFFRKNLLRLTG |
| 4009 | NMDsPGPMLFFRKNLLRLTG |
| 4010 | NMVERKNsKFFRKNLLRLTG |
| 4011 | NMVERKNsLFFRKNLLRLTG |
| 4012 | NRAMRRVsSVPSRFFRKNLLRLTG |
| 4013 | NRAMRRVsSVPSRAQFFRKNLLRLTG |
| 4014 | NRsWKYNQSISLRFFRKNLLRLTG |
| 4015 | NRsWKYNQSISLRRPFFRKNLLRLTG |
| 4016 | NRYtNRVVTFFFRKNLLRLTG |
| 4017 | NRYtNRVVTKFFRKNLLRLTG |
| 4018 | NRYtNRVVTLFFRKNLLRLTG |
| 4019 | NRYtNRVVTMFFRKNLLRLTG |
| 4020 | NRYtNRVVTRFFRKNLLRLTG |
| 4021 | NRYtNRVVTYFFRKNLLRLTG |
| 4022 | NSDsPLRYFFRKNLLRLTG |
| 4023 | NSEsPLRYFFRKNLLRLTG |
| 4024 | NTDsPLRYFFRKNLLRLTG |
| 4025 | NTEsPLRYFFRKNLLRLTG |
| 4026 | NYVERKNsKFFRKNLLRLTG |
| 4027 | NYVERKNsLFFRKNLLRLTG |
| 4028 | NYVERKNsYFFRKNLLRLTG |
| 4029 | PARsPVTEIFFRKNLLRLTG |
| 4030 | PAYEKLsAEFFRKNLLRLTG |
| 4031 | PAYEKLsAEQSPFFRKNLLRLTG |
| 4032 | PmVTLsLNLFFRKNLLRLTG |
| 4033 | PMVTLsLNLFFRKNLLRLTG |
| 4034 | PNAPPAYEKLsAFFRKNLLRLTG |
| 4035 | PPAYEKLsAFFRKNLLRLTG |
| 4036 | PPAYEKLsAEQSFFRKNLLRLTG |
| 4037 | PPLPEDSIKVIRNMRAAsPPAFFRKNLLRLTG |
| 4038 | PYDPALGsPSRFFRKNLLRLTG |
| 4039 | QAASNFKsPVKTIRFFRKNLLRLTG |
| 4040 | QLDsPQRALYFFRKNLLRLTG |
| 4041 | QLEsPQRALYFFRKNLLRLTG |
| 4042 | QLFsPKKGQKFFRKNLLRLTG |
| 4043 | QMFsPKKGQKFFRKNLLRLTG |
| 4044 | QPQRRsLRLFFRKNLLRLTG |
| 4045 | QPRsPGPDYSFFFRKNLLRLTG |
| 4046 | QPRsPGPDYSLFFRKNLLRLTG |
| 4047 | QPRsPGPDYSMFFRKNLLRLTG |
| 4048 | QPRsPGPDYSVFFRKNLLRLTG |
| 4049 | QPRtPsPLVFFFRKNLLRLTG |
| 4050 | QPRtPSPLVFFFRKNLLRLTG |
| 4051 | QPRtPsPLVLFFRKNLLRLTG |
| 4052 | QPRtPSPLVLFFRKNLLRLTG |
| 4053 | QPRtPsPLVMFFRKNLLRLTG |
| 4054 | QPRtPSPLVMFFRKNLLRLTG |
| 4055 | QPRtPsPLVVFFRKNLLRLTG |
| 4056 | QPRtPSPLVVFFRKNLLRLTG |
| 4057 | QPSFPsVLPAFFRKNLLRLTG |
| 4058 | QRLsPLSAAYFFRKNLLRLTG |
| 4059 | QSDsPQRALYFFRKNLLRLTG |
| 4060 | QSEsPQRALYFFRKNLLRLTG |
| 4061 | QTDsPQRALYFFRKNLLRLTG |
| 4062 | QTEsPQRALYFFRKNLLRLTG |
| 4063 | QVAMPVKKSPRRSsSDEQGLSYSSLKNVFFRKNLLRLTG |
| 4064 | QVFsPKKGQKFFRKNLLRLTG |
| 4065 | QVFsPKKGQYFFRKNLLRLTG |
| 4066 | RADsPVHMFFRKNLLRLTG |
| 4067 | RAFsFSKTPKFFRKNLLRLTG |
| 4068 | RAFsFSKTPYFFRKNLLRLTG |
| 4069 | RAFsVKFEVFFRKNLLRLTG |
| 4070 | RAHsEPLALFFRKNLLRLTG |
| 4071 | RAHsSPASLFFRKNLLRLTG |
| 4072 | RAHSsPASLFFRKNLLRLTG |
| 4073 | RAKsPISLKFFRKNLLRLTG |
| 4074 | RAKsPISLYFFRKNLLRLTG |
| 4075 | RAPsPSSRFFFRKNLLRLTG |
| 4076 | RAPsPSSRLFFRKNLLRLTG |
| 4077 | RAPsPSSRMFFRKNLLRLTG |
| 4078 | RAPsPSSRVFFRKNLLRLTG |
| 4079 | RARGIsPIVFFFRKNLLRLTG |
| 4080 | RASsDIVsLFFRKNLLRLTG |
| 4081 | RASsDIVSLFFRKNLLRLTG |
| 4082 | RASsLSITVFFRKNLLRLTG |
| 4083 | REAPsPLmIFFRKNLLRLTG |
| 4084 | REAPsPLMIFFRKNLLRLTG |
| 4085 | REAsPAPLAFFRKNLLRLTG |
| 4086 | REAsPRLRVFFRKNLLRLTG |
| 4087 | REAsPSRLSVFFRKNLLRLTG |
| 4088 | REDsTPGKVFLFFRKNLLRLTG |
| 4089 | REIMGtPEYLFFRKNLLRLTG |
| 4090 | REKsPGRmLFFRKNLLRLTG |
| 4091 | REKsPGRMLFFRKNLLRLTG |
| 4092 | REKsPLFQFFFRKNLLRLTG |
| 4093 | REKsPLFQWFFRKNLLRLTG |
| 4094 | REKsPLFQYFFRKNLLRLTG |
| 4095 | RELARKGsLFFRKNLLRLTG |
| 4096 | RELsPLISLFFRKNLLRLTG |
| 4097 | REPsPLPELFFRKNLLRLTG |
| 4098 | RERsPSPSFFFRKNLLRLTG |
| 4099 | RESsPTRRLFFRKNLLRLTG |
| 4100 | REVsPAPAVFFRKNLLRLTG |
| 4101 | REYGsTSSIFFRKNLLRLTG |
| 4102 | RFKtQPVTFFFRKNLLRLTG |
| 4103 | RGDGYGtFFFRKNLLRLTG |
| 4104 | TGDsPKIDLFFRKNLLRLTG |
| 4105 | RIDsKDSASELFFRKNLLRLTG |
| 4106 | RIGsPLSPKFFRKNLLRLTG |
| 4107 | RISsGVVTKFFRKNLLRLTG |
| 4108 | RILsGVVTYFFRKNLLRLTG |
| 4109 | RILsPSMAKSFFRKNLLRLTG |
| 4110 | RILsPSMASYFFRKNLLRLTG |
| 4111 | RINsFEEHVFFRKNLLRLTG |
| 4112 | RIQsKLYRAFFRKNLLRLTG |
| 4113 | RIQyIQSRFFFRKNLLRLTG |
| 4114 | RIQyIQSRFYFFRKNLLRLTG |
| 4115 | RIsHELDSFFRKNLLRLTG |
| 4116 | RITsLIVHVFFRKNLLRLTG |
| 4117 | RIVQyIQSRFFRKNLLRLTG |
| 4118 | RIYQyIQFFRKNLLRLTG |
| 4119 | RIYQyIQSKFFRKNLLRLTG |
| 4120 | RIYQyIQSRFFRKNLLRLTG |
| 4121 | RIYQyIQSRFFFRKNLLRLTG |
| 4122 | RIYQyIQSRFKFFRKNLLRLTG |
| 4123 | RIYQyIQSRFYFFRKNLLRLTG |
| 4124 | RIYQyIQSRKFFRKNLLRLTG |
| 4125 | RIYQyIQSRYFFRKNLLRLTG |
| 4126 | RIYQyIQSYFFRKNLLRLTG |
| 4127 | RIYQyLQSRFFFRKNLLRLTG |
| 4128 | RIYQyLQSRFYFFRKNLLRLTG |
| 4129 | RKLRsLEQLFFRKNLLRLTG |
| 4130 | RKLsVILIKFFRKNLLRLTG |
| 4131 | RKLsVILILFFRKNLLRLTG |
| 4132 | RKLsVILIYFFRKNLLRLTG |
| 4133 | RKPsIVTKYFFRKNLLRLTG |
| 4134 | RKSsIIIRMFFRKNLLRLTG |
| 4135 | RLAsASRALFFRKNLLRLTG |
| 4136 | RLAsFAVRKFFRKNLLRLTG |
| 4137 | RLAsFAVRYFFRKNLLRLTG |
| 4138 | RLAsIELPSMFFRKNLLRLTG |
| 4139 | RLAsIELPSMAVFFRKNLLRLTG |
| 4140 | RLAsIELPSVFFRKNLLRLTG |
| 4141 | RLAsLNAEALFFRKNLLRLTG |
| 4142 | RLAsLNAEAVFFRKNLLRLTG |
| 4143 | RLAsLQSEVFFRKNLLRLTG |
| 4144 | RLAsLSISVFFRKNLLRLTG |
| 4145 | RLAsPLVHKFFRKNLLRLTG |
| 4146 | RLAsPLVHYFFRKNLLRLTG |
| 4147 | RLAsPPPPPKFFRKNLLRLTG |
| 4148 | RLAsPPPPPYFFRKNLLRLTG |
| 4149 | RLAsPTSGVFFRKNLLRLTG |
| 4150 | RLAsPTSGVKFFRKNLLRLTG |
| 4151 | RLAsPTSGVKKFFRKNLLRLTG |
| 4152 | RLAsPTSGVKRFFRKNLLRLTG |
| 4153 | RLAsPTSGVKYFFRKNLLRLTG |
| 4154 | RLAsRPLLLFFRKNLLRLTG |
| 4155 | RLAsSATQVHKFFRKNLLRLTG |
| 4156 | RLAsYLDKVFFRKNLLRLTG |
| 4157 | RLAsYLDRVFFRKNLLRLTG |
| 4158 | RLDsTPGKVFLFFRKNLLRLTG |
| 4159 | RLDsTPGKVFVFFRKNLLRLTG |
| 4160 | RLDsYLRAPFFRKNLLRLTG |
| 4161 | RLDsYVRFFRKNLLRLTG |
| 4162 | RLDsYVRSFFRKNLLRLTG |
| 4163 | RLDsYVRSLFFRKNLLRLTG |
| 4164 | RLDsYVRSVFFRKNLLRLTG |
| 4165 | RLDtGPQSLFFRKNLLRLTG |
| 4166 | RLEsANRRLFFRKNLLRLTG |
| 4167 | RLFsFSKTPKFFRKNLLRLTG |
| 4168 | RLFsKELFFRKNLLRLTG |
| 4169 | RLFsKELRFFRKNLLRLTG |
| 4170 | RLFsKELRCFFRKNLLRLTG |
| 4171 | RLFsKELRVFFRKNLLRLTG |
| 4172 | RLFSLsNPSLFFRKNLLRLTG |
| 4173 | RLFsPTYGLFFRKNLLRLTG |
| 4174 | RLFsPTYGVFFRKNLLRLTG |
| 4175 | RLFsQGQDVFFRKNLLRLTG |
| 4176 | RLFVGsIPKFFRKNLLRLTG |
| 4177 | RLGsFHELLLFFRKNLLRLTG |
| 4178 | RLIsFKAEVFFRKNLLRLTG |
| 4179 | RLIsPYKKKFFRKNLLRLTG |
| 4180 | RLIsQDVKLFFRKNLLRLTG |
| 4181 | RLIsQDVKVFFRKNLLRLTG |
| 4182 | RLKLPsGSKFFRKNLLRLTG |
| 4183 | RLKLPsGSKKFFRKNLLRLTG |
| 4184 | RLKLPsGSKYFFRKNLLRLTG |
| 4185 | RLKsDERPVHIFFRKNLLRLTG |
| 4186 | RLKsPFRKKFFRKNLLRLTG |
| 4187 | RLKsPGsGHVKFFRKNLLRLTG |
| 4188 | RLKsPISLKFFRKNLLRLTG |
| 4189 | RLKsPISLYFFRKNLLRLTG |
| 4190 | RLKsPSPKSEKFFRKNLLRLTG |
| 4191 | RLKsPSPKSERFFRKNLLRLTG |
| 4192 | RLKtPTSQSYKFFRKNLLRLTG |
| 4193 | RLKtPTSQSYRFFRKNLLRLTG |
| 4194 | RLKTtPLRKFFRKNLLRLTG |
| 4195 | RLKTtPLRRFFRKNLLRLTG |
| 4196 | RLLDPsSPLALFFRKNLLRLTG |
| 4197 | RLLDPSsPLALFFRKNLLRLTG |
| 4198 | RLLDRSPsRSAKFFRKNLLRLTG |
| 4199 | RLLDRSPsRSAYFFRKNLLRLTG |
| 4200 | RLLsDGQQHLFFRKNLLRLTG |
| 4201 | RLLsDLEELFFRKNLLRLTG |
| 4202 | RLLsDQTRLFFRKNLLRLTG |
| 4203 | RLLsFQRYLFFRKNLLRLTG |
| 4204 | RLLsGVVTKFFRKNLLRLTG |
| 4205 | RLLsGVVTYFFRKNLLRLTG |
| 4206 | RLLsHISEAFFRKNLLRLTG |
| 4207 | RLLsHISEVFFRKNLLRLTG |
| 4208 | RLLsPLSSAFFRKNLLRLTG |
| 4209 | RLLsPLSSARLFFRKNLLRLTG |
| 4210 | RLLsPLSSVFFRKNLLRLTG |
| 4211 | RLLsPQQPALFFRKNLLRLTG |
| 4212 | RLLsPRPSLFFRKNLLRLTG |
| 4213 | RLLsPRPSLLFFRKNLLRLTG |
| 4214 | RLLsPSMASKFFRKNLLRLTG |
| 4215 | RLLsSGVSEIFFRKNLLRLTG |
| 4216 | RLLsSGVSEVFFRKNLLRLTG |
| 4217 | RLLsTDAEAVFFRKNLLRLTG |
| 4218 | RLLsVEIVKFFRKNLLRLTG |
| 4219 | RLLsVEIVYFFRKNLLRLTG |
| 4220 | RLLsVHDFDFFFRKNLLRLTG |
| 4221 | RLLsVILIKFFRKNLLRLTG |
| 4222 | RLMsMPVAKFFRKNLLRLTG |
| 4223 | RLMsMPVAYFFRKNLLRLTG |
| 4224 | RLNtSDFQKLFFRKNLLRLTG |
| 4225 | RLPNRIPsLFFRKNLLRLTG |
| 4226 | RLPsFLKKNKFFRKNLLRLTG |
| 4227 | RLPsLVHGYFFRKNLLRLTG |
| 4228 | RLPsSTLKKFFRKNLLRLTG |
| 4229 | RLPsSTLKRFFRKNLLRLTG |
| 4230 | RLPsSTLKYFFRKNLLRLTG |
| 4231 | RLQsLIKNIFFRKNLLRLTG |
| 4232 | RLQsTSERLFFRKNLLRLTG |
| 4233 | RLQsTSERVFFRKNLLRLTG |
| 4234 | RLR(sLss)PTVTLFFRKNLLRLTG |
| 4235 | RLR(sLss)PTVTVFFRKNLLRLTG |
| 4236 | RLRQsPLATKFFRKNLLRLTG |
| 4237 | RLRQsPLATRFFRKNLLRLTG |
| 4238 | RLRQsPLATYFFRKNLLRLTG |
| 4239 | RLRRsPLLKFFRKNLLRLTG |
| 4240 | RLRsAGAAQKFFRKNLLRLTG |
| 4241 | RLRsLSSLREKFFRKNLLRLTG |
| 4242 | RLRsPPPVSKFFRKNLLRLTG |
| 4243 | RLRsYEDMIFFRKNLLRLTG |
| 4244 | RLRTsPITRKFFRKNLLRLTG |
| 4245 | RLRTsPITRRFFRKNLLRLTG |
| 4246 | RLSDtPPLLFFRKNLLRLTG |
| 4247 | RLSsLIRHKFFRKNLLRLTG |
| 4248 | RLSsLRASTSKFFRKNLLRLTG |
| 4249 | RLSsPISKKFFRKNLLRLTG |
| 4250 | RLSsPISKRFFRKNLLRLTG |
| 4251 | RLSsPISKYFFRKNLLRLTG |
| 4252 | RLsSPLHFVFFRKNLLRLTG |
| 4253 | RLSsPLHFVFFRKNLLRLTG |
| 4254 | RLSsPVLHKFFRKNLLRLTG |
| 4255 | RLSsPVLHRFFRKNLLRLTG |
| 4256 | RLSsPVLHYFFRKNLLRLTG |
| 4257 | RLSsRFSSKFFRKNLLRLTG |
| 4258 | RLSsRFSSRFFRKNLLRLTG |
| 4259 | RLSsRFSSYFFRKNLLRLTG |
| 4260 | RLSsRYSQKFFRKNLLRLTG |
| 4261 | RLSsRYSQYFFRKNLLRLTG |
| 4262 | RLSsVKLISKFFRKNLLRLTG |
| 4263 | RLSsVKLISYFFRKNLLRLTG |
| 4264 | RLTFsPTYGVFFRKNLLRLTG |
| 4265 | RLVsLSMRKFFRKNLLRLTG |
| 4266 | RLVsLSMRYFFRKNLLRLTG |
| 4267 | RLYKsPLRHFFRKNLLRLTG |
| 4268 | RLYKsPLRKFFRKNLLRLTG |
| 4269 | RLYQyIQSKFFRKNLLRLTG |
| 4270 | RLYQyIQSRFFRKNLLRLTG |
| 4271 | RLYQyIQSRFKFFRKNLLRLTG |
| 4272 | RLYQyIQSRFYFFRKNLLRLTG |
| 4273 | RLYQyIQSYFFRKNLLRLTG |
| 4274 | RLYQylOSKFFRKNLLRLTG |
| 4275 | RLYQyLQSRFFFRKNLLRLIG |
| 4276 | RLYQyLQSRFKFFRKNLLRLIG |
| 4277 | RLYQyLQSRFYFFRKNLLRLTG |
| 4278 | RLYQyLQSRKFFRKNLLRLIG |
| 4279 | RLYsGPMNKVFFRKNLLRLTG |
| 4280 | RLYsGSRsKFFRKNLLRLTG |
| 4281 | RLYsGSRsRFFRKNLLRLTG |
| 4282 | RLYsGSRsYFFRKNLLRLTG |
| 4283 | RLYsKSRDKFFRKNLLRLTG |
| 4284 | RLYsPDHRQKFFRKNLLRLTG |
| 4285 | RLYsPERSKFFRKNLLRLTG |
| 4286 | RLYsPRNSKFFRKNLLRLTG |
| 4287 | RLYsPYNHKFFRKNLLRLTG |
| 4288 | RLYsPYNHRFFRKNLLRLTG |
| 4289 | RLYsPYNHYFFRKNLLRLTG |
| 4290 | RLYSRsFSKFFRKNLLRLTG |
| 4291 | RLYSRsFSYFFRKNLLRLTG |
| 4292 | RLYsYPRQKFFRKNLLRLTG |
| 4293 | RLYVTTSTRTYsLGFFRKNLLRLTG |
| 4294 | RLYVTTSTRTYsLKFFRKNLLRLTG |
| 4295 | RLYVTTSTRTYsLYFFRKNLLRLTG |
| 4296 | RMAsPPPPPKFFRKNLLRLTG |
| 4297 | RMAsPTSGVFFRKNLLRLTG |
| 4298 | RMAsPTSGVKFFRKNLLRLTG |
| 4299 | RMAsPTSGVKKFFRKNLLRLTG |
| 4300 | RMAsPTSGVKRFFRKNLLRLTG |
| 4301 | RMAsPTSGVKYFFRKNLLRLTG |
| 4302 | RMAsSATQVHKFFRKNLLRLTG |
| 4303 | RMDsTPGKVFLFFRKNLLRLTG |
| 4304 | RMDsTPGKVFVFFRKNLLRLTG |
| 4305 | RMDsYVRSLFFRKNLLRLTG |
| 4306 | RMDsYVRSVFFRKNLLRLTG |
| 4307 | RMFPtPPSLFFRKNLLRLTG |
| 4308 | RMFsFSKTPKFFRKNLLRLTG |
| 4309 | RMFsKELRCFFRKNLLRLTG |
| 4310 | RMFsKELRVFFRKNLLRLTG |
| 4311 | RMFsPMEEKFFRKNLLRLTG |
| 4312 | RMFsPMEEKELLFFRKNLLRLTG |
| 4313 | RMFsPTYGLFFRKNLLRLTG |
| 4314 | RMFsPTYGVFFRKNLLRLTG |
| 4315 | RMIsPYKKKFFRKNLLRLTG |
| 4316 | RMIsQDVKLFFRKNLLRLTG |
| 4317 | RMIsQDVKVFFRKNLLRLTG |
| 4318 | RMIsTGSELFFRKNLLRLTG |
| 4319 | RMKLPsGSKFFRKNLLRLTG |
| 4320 | RMKLPsGSKKFFRKNLLRLTG |
| 4321 | RMKLPsGSKYFFRKNLLRLTG |
| 4322 | RMKsPFRKKFFRKNLLRLTG |
| 4323 | RMKsPGsGHVKFFRKNLLRLTG |
| 4324 | RMKsPSPKSEKFFRKNLLRLTG |
| 4325 | RMKtPTSQSYKFFRKNLLRLTG |
| 4326 | RMKtPTSQSYRFFRKNLLRLTG |
| 4327 | RMKTtPLRKFFRKNLLRLTG |
| 4328 | RMKTtPLRRFFRKNLLRLTG |
| 4329 | RMLDRSPsRSAKFFRKNLLRLTG |
| 4330 | RMLDRSPsRSAYFFRKNLLRLTG |
| 4331 | RMLsHISEAFFRKNLLRLTG |
| 4332 | RMLsHISEVFFRKNLLRLTG |
| 4333 | RMLsLRDQRLFFRKNLLRLTG |
| 4334 | RMLsPLSSAFFRKNLLRLTG |
| 4335 | RMLsPLSSVFFRKNLLRLTG |
| 4336 | RMLsPSMASKFFRKNLLRLTG |
| 4337 | RMLsSGVSEIFFRKNLLRLTG |
| 4338 | RMLsSGVSEVFFRKNLLRLTG |
| 4339 | RMLsVILIKFFRKNLLRLTG |
| 4340 | RMPsFLKKNKFFRKNLLRLTG |
| 4341 | RMPsSTLKKFFRKNLLRLTG |
| 4342 | RMPsSTLKRFFRKNLLRLTG |
| 4343 | RMQsTSERLFFRKNLLRLTG |
| 4344 | RMQsTSERVFFRKNLLRLTG |
| 4345 | RMRQsPLATKFFRKNLLRLTG |
| 4346 | RMRQsPLATRFFRKNLLRLTG |
| 4347 | RMRRsPLLKFFRKNLLRLTG |
| 4348 | RMRsAGAAQKFFRKNLLRLTG |
| 4349 | RMRsLSSLREKFFRKNLLRLTG |
| 4350 | RMRsPPPVSKFFRKNLLRLTG |
| 4351 | RMRTsPITRKFFRKNLLRLTG |
| 4352 | RMRTsPITRRFFRKNLLRLTG |
| 4353 | RMSsLIRHKFFRKNLLRLTG |
| 4354 | RMSsPISKKFFRKNLLRLTG |
| 4355 | RMSsPISKRFFRKNLLRLTG |
| 4356 | RMSsPLHFVFFRKNLLRLTG |
| 4357 | RMSsPVLHKFFRKNLLRLTG |
| 4358 | RMSsRYSQKFFRKNLLRLTG |
| 4359 | RMSsVKLISKFFRKNLLRLTG |
| 4360 | RMSsVKLISYFFRKNLLRLTG |
| 4361 | RMVsLSMRKFFRKNLLRLTG |
| 4362 | RMVsLSMRYFFRKNLLRLTG |
| 4363 | RMYKsPLRHFFRKNLLRLTG |
| 4364 | RMYKsPLRKFFRKNLLRLTG |
| 4365 | RMYQyIQSKFFRKNLLRLTG |
| 4366 | RMYQyIQSRFFRKNLLRLTG |
| 4367 | RMYQyLQSRFFFRKNLLRLIG |
| 4368 | RMYQyLQSRFKFFRKNLLRLIG |
| 4369 | RMYQyLQSRFYFFRKNLLRLIG |
| 4370 | RMYQyLQSRKFFRKNLLRLIG |
| 4371 | RMYsFDDVLFFRKNLLRLTG |
| 4372 | RMYsGSRsKFFRKNLLRLTG |
| 4373 | RMYsGSRsRFFRKNLLRLTG |
| 4374 | RMYsKSRDHFFRKNLLRLTG |
| 4375 | RMYsKSRDKFFRKNLLRLTG |
| 4376 | RMYsKSRDYFFRKNLLRLTG |
| 4377 | RMYsPDHRQKFFRKNLLRLTG |
| 4378 | RMYsPERSKFFRKNLLRLTG |
| 4379 | RMYsPIIYQAFFRKNLLRLTG |
| 4380 | RMYsPIPPSLFFRKNLLRLTG |
| 4381 | RMYsPRNSKFFRKNLLRLTG |
| 4382 | RMYsPYNHKFFRKNLLRLTG |
| 4383 | RMYsPYNHRFFRKNLLRLTG |
| 4384 | RMYsYPRQKFFRKNLLRLTG |
| 4385 | RMYVTTSTRTYsLGFFRKNLLRLTG |
| 4386 | RMYVTTSTRTYsLKFFRKNLLRLTG |
| 4387 | RMYVTTSTRTYsLYFFRKNLLRLTG |
| 4388 | RNLsSPFIFFFRKNLLRLTG |
| 4389 | RPAFFsPSLFFRKNLLRLTG |
| 4390 | RPAKsMDSFFFRKNLLRLTG |
| 4391 | RPAKsMDSLFFRKNLLRLTG |
| 4392 | RPAKsMDSMFFRKNLLRLTG |
| 4393 | RPAKsMDVFFRKNLLRLTG |
| 4394 | RPAsAGAMFFFRKNLLRLTG |
| 4395 | RPAsAGAmLFFRKNLLRLTG |
| 4396 | RPAsAGAMLFFRKNLLRLTG |
| 4397 | RPAsAGAMMFFRKNLLRLTG |
| 4398 | RPAsAGAMVFFRKNLLRLTG |
| 4399 | RPAsARAQPGFFFRKNLLRLTG |
| 4400 | RPAsARAQPGLFFRKNLLRLTG |
| 4401 | RPAsARAQPGMFFRKNLLRLTG |
| 4402 | RPAsARAQPGVFFRKNLLRLTG |
| 4403 | RPAsEARAPGLFFRKNLLRLTG |
| 4404 | RPAsPAAKFFFRKNLLRLTG |
| 4405 | RPAsPAAKLFFRKNLLRLTG |
| 4406 | RPAsPAAKMFFRKNLLRLTG |
| 4407 | RPAsPAAKVFFRKNLLRLTG |
| 4408 | RPAsPEPELFFRKNLLRLTG |
| 4409 | RPAsPGPSLFFRKNLLRLTG |
| 4410 | RPAsPKRAKIFFRKNLLRLTG |
| 4411 | RPAsPKRAKLFFRKNLLRLTG |
| 4412 | RPAsPKRAKXFFRKNLLRLTG |
| 4413 | RPAsPKRAQIFFRKNLLRLTG |
| 4414 | RPAsPKRAQLFFRKNLLRLTG |
| 4415 | RPAsPKRAQXFFRKNLLRLTG |
| 4416 | RPAsPQRAKIFFRKNLLRLTG |
| 4417 | RPAsPQRAKLFFRKNLLRLTG |
| 4418 | RPAsPQRAKXFFRKNLLRLTG |
| 4419 | RPAsPQRAQIFFRKNLLRLTG |
| 4420 | RPAsPQRAQLFFRKNLLRLTG |
| 4421 | RPAsPQRAQXFFRKNLLRLTG |
| 4422 | RPAsPSLQLFFRKNLLRLTG |
| 4423 | RPAsPSLQLLFFRKNLLRLTG |
| 4424 | RPAsPtAIRRIGSVTSRQTFFRKNLLRLTG |
| 4425 | RPAsRFEVLFFRKNLLRLTG |
| 4426 | RPAsYKKKSMLFFRKNLLRLTG |
| 4427 | RPAtGGPGVAFFRKNLLRLTG |
| 4428 | RPAtGGPGVFFFRKNLLRLTG |
| 4429 | RPAtGGPGVLFFRKNLLRLTG |
| 4430 | RPAtGGPGVMFFRKNLLRLTG |
| 4431 | RPAtGGPGVVFFRKNLLRLTG |
| 4432 | RPAtPTSQFFFRKNLLRLTG |
| 4433 | RPAtPTSQLFFRKNLLRLTG |
| 4434 | RPAtPTSQMFFRKNLLRLTG |
| 4435 | RPAtPTSQVFFRKNLLRLTG |
| 4436 | RPDsAHKMLFFRKNLLRLTG |
| 4437 | RPDsPTRPTLFFRKNLLRLTG |
| 4438 | RPDsRLGKTEFFFRKNLLRLTG |
| 4439 | RPDsRLGKTELFFRKNLLRLTG |
| 4440 | RPDsRLGKTEMFFRKNLLRLTG |
| 4441 | RPDsRLGKTEVFFRKNLLRLTG |
| 4442 | RPDVAKRLsLFFRKNLLRLTG |
| 4443 | RPEsDSGLKFFFRKNLLRLTG |
| 4444 | RPEsDSGLKLFFRKNLLRLTG |
| 4445 | RPEsDSGLKMFFRKNLLRLTG |
| 4446 | RPEsDSGLKVFFRKNLLRLTG |
| 4447 | RPEsKDRKFFFRKNLLRLTG |
| 4448 | RPEsKDRKLFFRKNLLRLTG |
| 4449 | RPEsKDRKMFFRKNLLRLTG |
| 4450 | RPEsKDRKVFFRKNLLRLTG |
| 4451 | RPFARsHSFFFRKNLLRLTG |
| 4452 | RPFARSHsFFFRKNLLRLTG |
| 4453 | RPFHGISTVsLFFRKNLLRLTG |
| 4454 | RPFsPREAFFFRKNLLRLTG |
| 4455 | RPFsPREALFFRKNLLRLTG |
| 4456 | RPFsPREAMFFRKNLLRLTG |
| 4457 | RPFsPREAVFFRKNLLRLTG |
| 4458 | RPGsLERKFFFRKNLLRLTG |
| 4459 | RPGsLERKLFFRKNLLRLTG |
| 4460 | RPGsLERKMFFRKNLLRLTG |
| 4461 | RPGsLERKVFFRKNLLRLTG |
| 4462 | RPGsRQAGLFFRKNLLRLTG |
| 4463 | RPGsRqAGLFFRKNLLRLTG |
| 4464 | RPHsPEKAFFFRKNLLRLTG |
| 4465 | RPHsPEKALFFRKNLLRLTG |
| 4466 | RPHsPEKAMFFRKNLLRLTG |
| 4467 | RPHsPEKAVFFRKNLLRLTG |
| 4468 | RPHtPTGIYMFFRKNLLRLTG |
| 4469 | RPHtPTPGIYMFFRKNLLRLTG |
| 4470 | RPIsPGLSFFFRKNLLRLTG |
| 4471 | RPIsPGLSLFFRKNLLRLTG |
| 4472 | RPIsPGLSMFFRKNLLRLTG |
| 4473 | RPIsPGLSVFFRKNLLRLTG |
| 4474 | RPIsPGLSYFFRKNLLRLTG |
| 4475 | RPIsPPHTYFFRKNLLRLTG |
| 4476 | RPIsPRIGALFFRKNLLRLTG |
| 4477 | RPItPPRNSAFFRKNLLRLTG |
| 4478 | RPItPPRNSFFFRKNLLRLTG |
| 4479 | RPItPPRNSLFFRKNLLRLTG |
| 4480 | RPItPPRNSMFFRKNLLRLTG |
| 4481 | RPItPPRNSVFFRKNLLRLTG |
| 4482 | RPKLSsPAFFFRKNLLRLTG |
| 4483 | RPKLSsPALFFRKNLLRLTG |
| 4484 | RPKLSsPAMFFRKNLLRLTG |
| 4485 | RPKLSsPAVFFRKNLLRLTG |
| 4486 | RPKPSSsPFFFRKNLLRLTG |
| 4487 | RPKPSSsPLFFRKNLLRLTG |
| 4488 | RPKPSSsPMFFRKNLLRLTG |
| 4489 | RPKPSSsPVFFRKNLLRLTG |
| 4490 | RPKsNIVLFFFRKNLLRLTG |
| 4491 | RPKsNIVLLFFRKNLLRLTG |
| 4492 | RPKsNIVLMFFRKNLLRLTG |
| 4493 | RPKsNIVLVFFRKNLLRLTG |
| 4494 | RPKsPLSKmFFRKNLLRLTG |
| 4495 | RPKsPLSKMFFRKNLLRLTG |
| 4496 | RPKsQVAEFFFRKNLLRLTG |
| 4497 | RPKsQVAELFFRKNLLRLTG |
| 4498 | RPKsQVAEMFFRKNLLRLTG |
| 4499 | RPKsQVAEVFFRKNLLRLTG |
| 4500 | RPKsVDFDSLFFRKNLLRLTG |
| 4501 | RPKtPPVVIFFRKNLLRLTG |
| 4502 | RPLsLLLALFFRKNLLRLTG |
| 4503 | RPLsPGGAFFFRKNLLRLTG |
| 4504 | RPLsPGGALFFRKNLLRLTG |
| 4505 | RPLsPGGAMFFRKNLLRLTG |
| 4506 | RPLsPGGAVFFRKNLLRLTG |
| 4507 | RPLsPLLFFFRKNLLRLTG |
| 4508 | RPLsPLLLFFRKNLLRLTG |
| 4509 | RPLsPLLMFFRKNLLRLTG |
| 4510 | RPLsPLLVFFRKNLLRLTG |
| 4511 | RPLsVVYVLFFRKNLLRLTG |
| 4512 | RPMsESPHMFFRKNLLRLTG |
| 4513 | RPNsPSPTAFFFRKNLLRLTG |
| 4514 | RPNsPSPTALFFRKNLLRLTG |
| 4515 | RPNsPSPTAMFFRKNLLRLTG |
| 4516 | RPNsPSPTAVFFRKNLLRLTG |
| 4517 | RPPIgTQSSLFFRKNLLRLTG |
| 4518 | RPPPPPDtPFFFRKNLLRLTG |
| 4519 | RPPPPPDtPLFFRKNLLRLTG |
| 4520 | RPPPPPDtPMFFRKNLLRLTG |
| 4521 | RPPPPPDtPPFFRKNLLRLTG |
| 4522 | RPPPPPDtPVFFRKNLLRLTG |
| 4523 | RPPsPGPVFFFRKNLLRLTG |
| 4524 | RPPsPGPVLFFRKNLLRLTG |
| 4525 | RPPsPGPVMFFRKNLLRLTG |
| 4526 | RPPsPGPVVFFRKNLLRLTG |
| 4527 | RPPsPSSRFFFRKNLLRLTG |
| 4528 | RPPsPSSRLFFRKNLLRLTG |
| 4529 | RPPsPSSRMFFRKNLLRLTG |
| 4530 | RPPsPSSRVFFRKNLLRLTG |
| 4531 | RPPsSEFLDFFFRKNLLRLTG |
| 4532 | RPPsSEFLDLFFRKNLLRLTG |
| 4533 | RPPsSEFLDMFFRKNLLRLTG |
| 4534 | RPPsSEFLDVFFRKNLLRLTG |
| 4535 | RPQKTQsIIFFRKNLLRLTG |
| 4536 | RPQRAtSNVFFFRKNLLRLTG |
| 4537 | RPQRATsNVFFFRKNLLRLTG |
| 4538 | RPQRAtSNVLFFRKNLLRLTG |
| 4539 | RPQRATsNVLFFRKNLLRLTG |
| 4540 | RPQRAtSNVMFFRKNLLRLTG |
| 4541 | RPQRATsNVMFFRKNLLRLIG |
| 4542 | RPQRAtSNVVFFRKNLLRLTG |
| 4543 | RPQRATsNVVFFRKNLLRLTG |
| 4544 | RPR(sLss)PTVTLFFRKNLLRLTG |
| 4545 | RPR(sLss)PTVTVFFRKNLLRLTG |
| 4546 | RPRAAtVVFFRKNLLRLTG |
| 4547 | RPRAAtVVAFFRKNLLRLTG |
| 4548 | RPRAAtWFFRKNLLRLTG |
| 4549 | RPRAAtWAFFRKNLLRLTG |
| 4550 | RPRANsGGVDFFFRKNLLRLTG |
| 4551 | RPRANsGGVDLFFRKNLLRLTG |
| 4552 | RPRANsGGVDMFFRKNLLRLTG |
| 4553 | RPRANsGGVDVFFRKNLLRLTG |
| 4554 | RPRARsVDALFFRKNLLRLTG |
| 4555 | RPRDtRRISLFFRKNLLRLTG |
| 4556 | RPRGsESLLFFRKNLLRLTG |
| 4557 | RPRGsQSLFFFRKNLLRLTG |
| 4558 | RPRGsQSLLFFRKNLLRLTG |
| 4559 | RPRGsQSLMFFRKNLLRLTG |
| 4560 | RPRGsQSLVFFRKNLLRLTG |
| 4561 | RPRIPsPIGFFFRKNLLRLTG |
| 4562 | RPRLSsTNSSRFFFRKNLLRLTG |
| 4563 | RPRPAsSPALFFRKNLLRLTG |
| 4564 | RPRPHsAPSFFFRKNLLRLTG |
| 4565 | RPRPHsAPSLFFRKNLLRLTG |
| 4566 | RPRPHsAPSMFFRKNLLRLTG |
| 4567 | RPRPHsAPSVFFRKNLLRLTG |
| 4568 | RPRPSsAHVGLFFRKNLLRLTG |
| 4569 | RPRPsSVLFFRKNLLRLTG |
| 4570 | RPRPsSVLRTLFFRKNLLRLTG |
| 4571 | RPRPVsPSSFFFRKNLLRLTG |
| 4572 | RPRPVsPSSLFFRKNLLRLTG |
| 4573 | RPRPVsPSSLLFFRKNLLRLTG |
| 4574 | RPRPVsPSSMFFRKNLLRLTG |
| 4575 | RPRPVsPSSVFFRKNLLRLTG |
| 4576 | RPRRsSTQFFFRKNLLRLTG |
| 4577 | RPRRsSTQLFFRKNLLRLTG |
| 4578 | RPRRsSTQMFFRKNLLRLTG |
| 4579 | RPRRsSTQVFFRKNLLRLTG |
| 4580 | RPRsAVEQLFFRKNLLRLTG |
| 4581 | RPRsAVLFFFRKNLLRLTG |
| 4582 | RPRsAVLLFFRKNLLRLTG |
| 4583 | RPRsAVLMFFRKNLLRLTG |
| 4584 | RPRsAVLVFFRKNLLRLTG |
| 4585 | RPRSGsTGSSLFFRKNLLRLTG |
| 4586 | RPRsISVEEFFFRKNLLRLTG |
| 4587 | RPRsISVEELFFRKNLLRLTG |
| 4588 | RPRsISVEEMFFRKNLLRLTG |
| 4589 | RPRsISVEEVFFRKNLLRLTG |
| 4590 | RPRsLEVTFFFRKNLLRLTG |
| 4591 | RPRsLEVTIFFRKNLLRLTG |
| 4592 | RPRsLEVTLFFRKNLLRLTG |
| 4593 | RPRsLEVTMFFRKNLLRLTG |
| 4594 | RPRsLEVTVFFRKNLLRLTG |
| 4595 | RPRSLsSPTVFFRKNLLRLTG |
| 4596 | RPRSLsSPTVTFFFRKNLLRLTG |
| 4597 | RPRSLsSPTVTLFFRKNLLRLTG |
| 4598 | RPRSLsSPTVTMFFRKNLLRLTG |
| 4599 | RPRSLsSPTVTVFFRKNLLRLTG |
| 4600 | RPRsMTVSAFFRKNLLRLTG |
| 4601 | RPRsMVRSFFFRKNLLRLTG |
| 4602 | RPRsPAARFFFRKNLLRLTG |
| 4603 | RPRsPAARLFFRKNLLRLTG |
| 4604 | RPRsPAARMFFRKNLLRLTG |
| 4605 | RPRsPAARVFFRKNLLRLTG |
| 4606 | RPRsPGSNSKVFFRKNLLRLTG |
| 4607 | RPRsPNMQDLFFRKNLLRLTG |
| 4608 | RPRsPPGGPFFRKNLLRLTG |
| 4609 | RPRsPPPRAFFFRKNLLRLTG |
| 4610 | RPRsPPPRALFFRKNLLRLTG |
| 4611 | RPRsPPPRAMFFRKNLLRLTG |
| 4612 | RPRsPPPRAPFFRKNLLRLTG |
| 4613 | RPRsPPPRAVFFRKNLLRLTG |
| 4614 | RPRsPPSSPFFRKNLLRLTG |
| 4615 | RPRsPRENSFFFRKNLLRLTG |
| 4616 | RPRsPRENSIFFRKNLLRLTG |
| 4617 | RPRsPRENSLFFRKNLLRLTG |
| 4618 | RPRsPRENSMFFRKNLLRLTG |
| 4619 | RPRsPRENSVFFRKNLLRLTG |
| 4620 | RPRsPRPPPFFRKNLLRLTG |
| 4621 | RPRsPRQNLIFFRKNLLRLTG |
| 4622 | RPRsPRQNSFFFRKNLLRLTG |
| 4623 | RPRsPRQNSIFFRKNLLRLTG |
| 4624 | RPRsPRQNSMFFRKNLLRLTG |
| 4625 | RPRsPRQNSVFFRKNLLRLTG |
| 4626 | RPRsPSPIFFFRKNLLRLTG |
| 4627 | RPRsPSPILFFRKNLLRLTG |
| 4628 | RPRsPSPIMFFRKNLLRLTG |
| 4629 | RPRsPSPISFFRKNLLRLTG |
| 4630 | RPRSPsPISFFRKNLLRLTG |
| 4631 | RPRsPSPIVFFRKNLLRLTG |
| 4632 | RPRsPTGFFFRKNLLRLTG |
| 4633 | RPRsPTGLFFRKNLLRLTG |
| 4634 | RPRsPTGMFFRKNLLRLTG |
| 4635 | RPRsPTGPFFRKNLLRLTG |
| 4636 | RPRsPTGPsNSFFFRKNLLRLTG |
| 4637 | RPRsPTGPSNSFFFRKNLLRLTG |
| 4638 | RPRsPTGPSNSFLFFRKNLLRLTG |
| 4639 | RPRsPTGPsNSLFFRKNLLRLTG |
| 4640 | RPRsPTGPsNSMFFRKNLLRLTG |
| 4641 | RPRsPTGPsNSVFFRKNLLRLTG |
| 4642 | RPRsPTGVFFRKNLLRLTG |
| 4643 | RPRsPTRSFFFRKNLLRLTG |
| 4644 | RPRsPTRSLFFRKNLLRLTG |
| 4645 | RPRsPTRSMFFRKNLLRLTG |
| 4646 | RPRsPTRSVFFRKNLLRLTG |
| 4647 | RPRsPWGKLFFRKNLLRLTG |
| 4648 | RPRsQYNTKLFFRKNLLRLTG |
| 4649 | RPRSTsQSIVSLFFRKNLLRLTG |
| 4650 | RPRtPLRSLFFRKNLLRLTG |
| 4651 | RPSGRREsFFFRKNLLRLTG |
| 4652 | RPSGRREsLFFRKNLLRLTG |
| 4653 | RPSGRREsMFFRKNLLRLTG |
| 4654 | RPSGRREsVFFRKNLLRLTG |
| 4655 | RPsNPQLFFRKNLLRLTG |
| 4656 | RPSRSsPGFFFRKNLLRLTG |
| 4657 | RPSRSsPGLFFRKNLLRLTG |
| 4658 | RPSRSsPGMFFRKNLLRLTG |
| 4659 | RPSRSsPGVFFRKNLLRLTG |
| 4660 | RPSsGFYELFFRKNLLRLTG |
| 4661 | RPSsLDAEIDSFFFRKNLLRLTG |
| 4662 | RPSsLDAEIDSLFFRKNLLRLTG |
| 4663 | RPSsLDAEIDSMFFRKNLLRLTG |
| 4664 | RPSsLDAEIDSVFFRKNLLRLTG |
| 4665 | RPSsLPDFFFRKNLLRLTG |
| 4666 | RPSsLPDLFFRKNLLRLTG |
| 4667 | RPSsLPDMFFRKNLLRLTG |
| 4668 | RPSsLPDVFFRKNLLRLTG |
| 4669 | RPsSPALYFFFRKNLLRLTG |
| 4670 | RPSsPALYFFFRKNLLRLTG |
| 4671 | RPsSPALYLFFRKNLLRLTG |
| 4672 | RPsSPALYMFFRKNLLRLTG |
| 4673 | RPsSPALYVFFRKNLLRLTG |
| 4674 | RPStPKSDSEFFFRKNLLRLTG |
| 4675 | RPStPKSDSELFFRKNLLRLTG |
| 4676 | RPStPKSDSEMFFRKNLLRLTG |
| 4677 | RPStPKSDSEVFFRKNLLRLTG |
| 4678 | RPTKIGRRsLFFRKNLLRLTG |
| 4679 | RPTsFADELFFRKNLLRLTG |
| 4680 | RPTsPIQIMFFRKNLLRLTG |
| 4681 | RPTsRLNRFFFRKNLLRLTG |
| 4682 | RPTsRLNRLFFRKNLLRLTG |
| 4683 | RPTsRLNRMFFRKNLLRLTG |
| 4684 | RPTsRLNRVFFRKNLLRLTG |
| 4685 | RPVsPFQEFFFRKNLLRLTG |
| 4686 | RPVsPFQELFFRKNLLRLTG |
| 4687 | RPVsPFQEMFFRKNLLRLTG |
| 4688 | RPVsPFQEVFFRKNLLRLTG |
| 4689 | RPVsPGKDFFFRKNLLRLTG |
| 4690 | RPVsPGKDIFFRKNLLRLTG |
| 4691 | RPVsPGKDLFFRKNLLRLTG |
| 4692 | RPVsPGKDMFFRKNLLRLTG |
| 4693 | RPVsPGKDVFFRKNLLRLTG |
| 4694 | RPVSPsSLLFFRKNLLRLTG |
| 4695 | RPVsTDFAQYFFRKNLLRLTG |
| 4696 | RPVtPVSDFFFRKNLLRLTG |
| 4697 | RPVtPVSDLFFRKNLLRLTG |
| 4698 | RPVtPVSDMFFRKNLLRLTG |
| 4699 | RPVtPVSDVFFRKNLLRLTG |
| 4700 | RPWsNSRGLFFRKNLLRLTG |
| 4701 | RPWsPAVSAFFRKNLLRLTG |
| 4702 | RPWsPAVSFFFRKNLLRLTG |
| 4703 | RPWsPAVSLFFRKNLLRLTG |
| 4704 | RPWsPAVSMFFRKNLLRLTG |
| 4705 | RPWsPAVSVFFRKNLLRLTG |
| 4706 | RPYsPPFFSFFFRKNLLRLTG |
| 4707 | RPYsPPFFSLFFRKNLLRLTG |
| 4708 | RPYsPPFFSMFFRKNLLRLTG |
| 4709 | RPYsPPFFSVFFRKNLLRLTG |
| 4710 | RPYSPsQALFFRKNLLRLTG |
| 4711 | RPYsPSQYALFFRKNLLRLTG |
| 4712 | RPYSPsQYALFFRKNLLRLTG |
| 4713 | RPYsQVNVLFFRKNLLRLTG |
| 4714 | RQAsIELPSMFFRKNLLRLTG |
| 4715 | RQAsIELPSMAVFFRKNLLRLTG |
| 4716 | RQAsIELPSVFFRKNLLRLTG |
| 4717 | RQAsLSISVFFRKNLLRLTG |
| 4718 | RQAsPLVHKFFRKNLLRLTG |
| 4719 | RQAsPLVHRFFRKNLLRLTG |
| 4720 | RQAsPLVHYFFRKNLLRLTG |
| 4721 | RQDsTPGKVFLFFRKNLLRLTG |
| 4722 | RQDStPGKVFLFFRKNLLRLTG |
| 4723 | RQDsTPGKVFVFFRKNLLRLTG |
| 4724 | RQIsFKAEVFFRKNLLRLTG |
| 4725 | RQIsQDVKLFFRKNLLRLTG |
| 4726 | RQIsQDVKVFFRKNLLRLTG |
| 4727 | RQKsPLFQFFFRKNLLRLTG |
| 4728 | RQLsALHRAFFRKNLLRLTG |
| 4729 | RQLsLEGSGLGVFFRKNLLRLTG |
| 4730 | RQLsSGVSEIFFRKNLLRLTG |
| 4731 | RQLsSGVSEVFFRKNLLRLTG |
| 4732 | RQSsSRFNLFFRKNLLRLTG |
| 4733 | RRAsFAKSFFFRKNLLRLTG |
| 4734 | RRAsFAKSKFFRKNLLRLTG |
| 4735 | RRAsFAKSLFFRKNLLRLTG |
| 4736 | RRAsFAKSMFFRKNLLRLTG |
| 4737 | RRAsFAKSRFFRKNLLRLTG |
| 4738 | RRAsIITKYFFRKNLLRLTG |
| 4739 | RRAsLSEIGFFFRKNLLRLTG |
| 4740 | RRAsLSEIGKFFRKNLLRLTG |
| 4741 | RRAsLSEIGYFFRKNLLRLTG |
| 4742 | RRAsQEANLFFRKNLLRLTG |
| 4743 | RRASsPFRFFFRKNLLRLTG |
| 4744 | RRASsPFRKFFRKNLLRLTG |
| 4745 | RRASsPFRLFFRKNLLRLTG |
| 4746 | RRASsPFRMFFRKNLLRLTG |
| 4747 | RRASsPFRRFFRKNLLRLTG |
| 4748 | RRAsVFVKFFFRKNLLRLTG |
| 4749 | RRAsVFVKKFFRKNLLRLTG |
| 4750 | RRAsVFVKLFFRKNLLRLTG |
| 4751 | RRAsVFVKMFFRKNLLRLTG |
| 4752 | RRAsVFVKRFFRKNLLRLTG |
| 4753 | RRDsIVAEFFFRKNLLRLTG |
| 4754 | RRDsIVAEKFFRKNLLRLTG |
| 4755 | RRDsIVAELFFRKNLLRLTG |
| 4756 | RRDsIVAERFFRKNLLRLTG |
| 4757 | RRDsIVAEYFFRKNLLRLTG |
| 4758 | RRDsLQKPGLFFRKNLLRLTG |
| 4759 | RRFsFEVTLFFRKNLLRLTG |
| 4760 | RRFsFKFFFRKNLLRLTG |
| 4761 | RRFsFKKFFRKNLLRLTG |
| 4762 | RRFsFKKSFFFRKNLLRLTG |
| 4763 | RRFsFKKSKFFRKNLLRLTG |
| 4764 | RRFsFKKSLFFRKNLLRLTG |
| 4765 | RRFsFKKSMFFRKNLLRLTG |
| 4766 | RRFsFKKSRFFRKNLLRLTG |
| 4767 | RRFsFKLFFRKNLLRLTG |
| 4768 | RRFsFKMFFRKNLLRLTG |
| 4769 | RRFsFKRFFRKNLLRLTG |
| 4770 | RRFsGTAVYFFRKNLLRLTG |
| 4771 | RRFsGTVRFFFRKNLLRLTG |
| 4772 | RRFsGTVRKFFRKNLLRLTG |
| 4773 | RRFsGTVRLFFRKNLLRLTG |
| 4774 | RRFsGTVRMFFRKNLLRLTG |
| 4775 | RRFsGTVRRFFRKNLLRLTG |
| 4776 | RRFsIATLRFFRKNLLRLTG |
| 4777 | RRFsLTTLRFFRKNLLRLTG |
| 4778 | RRFsPDDKYSFFFRKNLLRLTG |
| 4779 | RRFsPDDKYSKFFRKNLLRLTG |
| 4780 | RRFsPDDKYSLFFRKNLLRLTG |
| 4781 | RRFsPDDKYSMFFRKNLLRLTG |
| 4782 | RRFsPPRRFFFRKNLLRLTG |
| 4783 | RRFsPPRRKFFRKNLLRLTG |
| 4784 | RRFsPPRRLFFRKNLLRLTG |
| 4785 | RRFsPPRRmFFRKNLLRLTG |
| 4786 | RRFsPPRRMFFRKNLLRLTG |
| 4787 | RRFsPPRRRFFRKNLLRLTG |
| 4788 | RRFsPPRRYFFRKNLLRLTG |
| 4789 | RRFsRLENRYFFRKNLLRLTG |
| 4790 | RRFsRSDELFFRKNLLRLTG |
| 4791 | RRFsRsPIFFFRKNLLRLTG |
| 4792 | RRFsRSPIFFFRKNLLRLTG |
| 4793 | RRFsRsPIKFFRKNLLRLTG |
| 4794 | RRFsRSPIKFFRKNLLRLTG |
| 4795 | RRFsRsPILFFRKNLLRLTG |
| 4796 | RRFsRSPILFFRKNLLRLTG |
| 4797 | RRFsRSPIMFFRKNLLRLTG |
| 4798 | RRFsRsPIRFFRKNLLRLTG |
| 4799 | RRFsRSPIRFFRKNLLRLTG |
| 4800 | RRFSRsPIRFFRKNLLRLTG |
| 4801 | RRFsRsPIRFFFRKNLLRLTG |
| 4802 | RRFsRSPIRFFFRKNLLRLTG |
| 4803 | RRFsRsPIRKFFRKNLLRLTG |
| 4804 | RRFsRSPIRKFFRKNLLRLTG |
| 4805 | RRFsRsPIRLFFRKNLLRLTG |
| 4806 | RRFsRSPIRLFFRKNLLRLTG |
| 4807 | RRFsRsPIRRFFRKNLLRLTG |
| 4808 | RRFsRSPIRRFFRKNLLRLTG |
| 4809 | RRFsRsPIRYFFRKNLLRLTG |
| 4810 | RRFsRSPIRYFFRKNLLRLTG |
| 4811 | RRFsRsPIYFFRKNLLRLTG |
| 4812 | RRFsRSPIYFFRKNLLRLTG |
| 4813 | RRFsRSPKFFRKNLLRLTG |
| 4814 | RRFSsPPRRMFFRKNLLRLTG |
| 4815 | RRFsVSTLRFFRKNLLRLTG |
| 4816 | RRFsVTTMRFFRKNLLRLTG |
| 4817 | RRFtPPSPAFFFRKNLLRLTG |
| 4818 | RRFtPPSPAKFFRKNLLRLTG |
| 4819 | RRFtPPSPARFFRKNLLRLTG |
| 4820 | RRFtPPSPAYFFRKNLLRLTG |
| 4821 | RRGsFEVTLFFRKNLLRLTG |
| 4822 | RRHsASNLHALFFRKNLLRLTG |
| 4823 | RRIDIsPSTFFFRKNLLRLTG |
| 4824 | RRIDIsPSTKFFRKNLLRLTG |
| 4825 | RRIDIsPSTLRFFRKNLLRLTG |
| 4826 | RRIDIsPSTLRKFFRKNLLRLTG |
| 4827 | RRIDIsPSTRFFRKNLLRLTG |
| 4828 | RRIDIsPSTYFFRKNLLRLTG |
| 4829 | RRIsDPEVFFFRKNLLRLTG |
| 4830 | RRIsDPQVFFFRKNLLRLTG |
| 4831 | RRIsGVDRFFFRKNLLRLTG |
| 4832 | RRIsGVDRKFFRKNLLRLTG |
| 4833 | RRIsGVDRLFFRKNLLRLTG |
| 4834 | RRIsGVDRMFFRKNLLRLTG |
| 4835 | RRIsGVDRRFFRKNLLRLTG |
| 4836 | RRIsGVDRYFFRKNLLRLTG |
| 4837 | RRIsGVDRYFFFRKNLLRLTG |
| 4838 | RRIsGVDRYKFFRKNLLRLTG |
| 4839 | RRIsGVDRYLFFRKNLLRLTG |
| 4840 | RRIsGVDRYRFFRKNLLRLTG |
| 4841 | RRIsGVDRYYFFRKNLLRLTG |
| 4842 | RRIsPAPQRFFRKNLLRLTG |
| 4843 | RRIsQIQQLFFRKNLLRLTG |
| 4844 | RRKsOVAEFFFRKNLLRLTG |
| 4845 | RRKsOVAEKFFRKNLLRLTG |
| 4846 | RRKsPPPSFFFRKNLLRLTG |
| 4847 | RRKsPPPSKFFRKNLLRLTG |
| 4848 | RRKsPPPSLFFRKNLLRLTG |
| 4849 | RRKsPPPSMFFRKNLLRLTG |
| 4850 | RRKsPPPSRFFRKNLLRLTG |
| 4851 | RRKsQLDSFFFRKNLLRLTG |
| 4852 | RRKsQLDSKFFRKNLLRLTG |
| 4853 | RRKsQLDSLFFRKNLLRLTG |
| 4854 | RRKsQLDSMFFRKNLLRLTG |
| 4855 | RRKsQLDSRFFRKNLLRLTG |
| 4856 | RRKsQLDSYFFRKNLLRLTG |
| 4857 | RRKsQVAEFFFRKNLLRLTG |
| 4858 | RRKsQVAEKFFRKNLLRLTG |
| 4859 | RRKsQVAELFFRKNLLRLTG |
| 4860 | RRKsQVAEMFFRKNLLRLTG |
| 4861 | RRKsQVAERFFRKNLLRLTG |
| 4862 | RRKsQVAEVFFRKNLLRLTG |
| 4863 | RRKsQVAEYFFRKNLLRLTG |
| 4864 | RRLGsPHRFFFRKNLLRLTG |
| 4865 | RRLGsPHRKFFRKNLLRLTG |
| 4866 | RRLGsPHRLFFRKNLLRLTG |
| 4867 | RRLGsPHRMFFRKNLLRLTG |
| 4868 | RRLGsPHRRFFRKNLLRLTG |
| 4869 | RRLsADIRFFFRKNLLRLTG |
| 4870 | RRLsADIRKFFRKNLLRLTG |
| 4871 | RRLsADIRLFFRKNLLRLTG |
| 4872 | RRLsADIRMFFRKNLLRLTG |
| 4873 | RRLsADIRRFFRKNLLRLTG |
| 4874 | RRLsADIRYFFRKNLLRLTG |
| 4875 | RRLsDSPVFFFRKNLLRLTG |
| 4876 | RRLsELLRYFFRKNLLRLTG |
| 4877 | RRLsERETRFFRKNLLRLTG |
| 4878 | RRLsESSALFFRKNLLRLTG |
| 4879 | RRLsFLVSFFFRKNLLRLTG |
| 4880 | RRLsFLVSKFFRKNLLRLTG |
| 4881 | RRLsFLVSLFFRKNLLRLTG |
| 4882 | RRLsFLVSMFFRKNLLRLTG |
| 4883 | RRLsFLVSRFFRKNLLRLTG |
| 4884 | RRLsFLVSYFFRKNLLRLTG |
| 4885 | RRLsGGSHSFFFRKNLLRLTG |
| 4886 | RRLsGGSHSKFFRKNLLRLTG |
| 4887 | RRLsGGSHSLFFRKNLLRLTG |
| 4888 | RRLsGGSHSMFFRKNLLRLTG |
| 4889 | RRLsGGSHSRFFRKNLLRLTG |
| 4890 | RRLsGGSHSYFFRKNLLRLTG |
| 4891 | RRLsGPLHTFFFRKNLLRLTG |
| 4892 | RRLsGPLHTKFFRKNLLRLTG |
| 4893 | RRLsGPLHTLFFRKNLLRLTG |
| 4894 | RRLsGPLHTMFFRKNLLRLTG |
| 4895 | RRLsGPLHTRFFRKNLLRLTG |
| 4896 | RRLsGPLHTVFFRKNLLRLTG |
| 4897 | RRLsGPLHTYFFRKNLLRLTG |
| 4898 | RRLsLFLNVFFRKNLLRLTG |
| 4899 | RRLsNLPTFFFRKNLLRLTG |
| 4900 | RRLsNLPTKFFRKNLLRLTG |
| 4901 | RRLsNLPTRFFRKNLLRLTG |
| 4902 | RRLsNLPTVFFRKNLLRLTG |
| 4903 | RRLsNLPTYFFRKNLLRLTG |
| 4904 | RRLsPAPOFFFRKNLLRLTG |
| 4905 | RRLsPAPQKFFRKNLLRLTG |
| 4906 | RRLsPAPQLFFRKNLLRLTG |
| 4907 | RRLsPAPQMFFRKNLLRLTG |
| 4908 | RRLsPKASQVFFFRKNLLRLTG |
| 4909 | RRLsPKASQVKFFRKNLLRLTG |
| 4910 | RRLsPKASQVLFFRKNLLRLTG |
| 4911 | RRLsPKASQVMFFRKNLLRLTG |
| 4912 | RRLsPKASQVRFFRKNLLRLTG |
| 4913 | RRLsPVPVPFFFRKNLLRLTG |
| 4914 | RRLsPVPVPKFFRKNLLRLTG |
| 4915 | RRLsPVPVPLFFRKNLLRLTG |
| 4916 | RRLsPVPVPMFFRKNLLRLTG |
| 4917 | RRLsPVPVPRFFRKNLLRLTG |
| 4918 | RRLsRELOKFFRKNLLRLTG |
| 4919 | RRLsRELQFFFRKNLLRLTG |
| 4920 | RRLsRELQLFFRKNLLRLTG |
| 4921 | RRLsRELQMFFRKNLLRLTG |
| 4922 | RRLsRELQRFFRKNLLRLTG |
| 4923 | RRLsRKLSLFFRKNLLRLTG |
| 4924 | RRLsVERIFFFRKNLLRLTG |
| 4925 | RRLsVERIKFFRKNLLRLTG |
| 4926 | RRLsVERIMFFRKNLLRLTG |
| 4927 | RRLsVERIRFFRKNLLRLTG |
| 4928 | RRLsYVLFIFFRKNLLRLTG |
| 4929 | RRLTHLsFFFRKNLLRLTG |
| 4930 | RRLTHLsKFFRKNLLRLTG |
| 4931 | RRLTHLsLFFRKNLLRLTG |
| 4932 | RRLTHLsMFFRKNLLRLTG |
| 4933 | RRLTHLsRFFRKNLLRLTG |
| 4934 | RRMsFQKPFFRKNLLRLTG |
| 4935 | RRMsLLSVFFFRKNLLRLTG |
| 4936 | RRMsLLSVKFFRKNLLRLTG |
| 4937 | RRMsLLSVLFFRKNLLRLTG |
| 4938 | RRMsLLSVMFFRKNLLRLTG |
| 4939 | RRMsLLSVRFFRKNLLRLTG |
| 4940 | RRmsLLSVVFFRKNLLRLTG |
| 4941 | RRMsLLSVVFFRKNLLRLTG |
| 4942 | RRMsLLSVYFFRKNLLRLTG |
| 4943 | RRMsLLSWFFRKNLLRLTG |
| 4944 | RRMsLSVMFFRKNLLRLTG |
| 4945 | RRMsPIKPLFFRKNLLRLTG |
| 4946 | RRMsPKAORFFRKNLLRLTG |
| 4947 | RRMsPKAQFFFRKNLLRLTG |
| 4948 | RRMsPKAQKFFRKNLLRLTG |
| 4949 | RRMsPKAQLFFRKNLLRLTG |
| 4950 | RRMsPKAQMFFRKNLLRLTG |
| 4951 | RRMsPKPFFFRKNLLRLTG |
| 4952 | RRMsPKPKFFRKNLLRLTG |
| 4953 | RRMsPKPMFFRKNLLRLTG |
| 4954 | RRMsPKPRFFRKNLLRLTG |
| 4955 | RRNsAPVSVFFRKNLLRLTG |
| 4956 | RRNsINRNFFFRKNLLRLTG |
| 4957 | RRNsNPVIAEFFFRKNLLRLTG |
| 4958 | RRNsNPVIAEKFFRKNLLRLTG |
| 4959 | RRNsNPVIAELFFRKNLLRLTG |
| 4960 | RRNsNPVIAEMFFRKNLLRLTG |
| 4961 | RRNsNPVIAERFFRKNLLRLTG |
| 4962 | RRNsSERTFFFRKNLLRLTG |
| 4963 | RRNsSERTKFFRKNLLRLTG |
| 4964 | RRNsSERTLFFRKNLLRLTG |
| 4965 | RRNsSERTMFFRKNLLRLTG |
| 4966 | RRNsSERTRFFRKNLLRLTG |
| 4967 | RRNsSERTYFFRKNLLRLTG |
| 4968 | RRNsSIVGFFFRKNLLRLTG |
| 4969 | RRNsSIVGKFFRKNLLRLTG |
| 4970 | RRNsSIVGLFFRKNLLRLTG |
| 4971 | RRNsSIVGMFFRKNLLRLTG |
| 4972 | RRNsSIVGRFFRKNLLRLTG |
| 4973 | RRNsSIVGYFFRKNLLRLTG |
| 4974 | RRNsVFQQGFFFRKNLLRLTG |
| 4975 | RRNsVFQQGKFFRKNLLRLTG |
| 4976 | RRNsVFQQGLFFRKNLLRLTG |
| 4977 | RRNsVFQQGMFFRKNLLRLTG |
| 4978 | RRNsVFQQGRFFRKNLLRLTG |
| 4979 | RRNsVFQQGYFFRKNLLRLTG |
| 4980 | RRPsIAPVLFFRKNLLRLTG |
| 4981 | RRPsLLSEFFFRKNLLRLTG |
| 4982 | RRPsLVHGFFFRKNLLRLTG |
| 4983 | RRPsLVHGKFFRKNLLRLTG |
| 4984 | RRPsLVHGLFFRKNLLRLTG |
| 4985 | RRPsLVHGMFFRKNLLRLTG |
| 4986 | RRPsLVHGRFFRKNLLRLTG |
| 4987 | RRPsLVHGYFFRKNLLRLTG |
| 4988 | RRPsVFERFFFRKNLLRLTG |
| 4989 | RRPsVFERKFFRKNLLRLTG |
| 4990 | RRPsVFERLFFRKNLLRLTG |
| 4991 | RRPsVFERMFFRKNLLRLTG |
| 4992 | RRPsVFERRFFRKNLLRLTG |
| 4993 | RRPsVFERYFFRKNLLRLTG |
| 4994 | RRPsYRKIFFFRKNLLRLTG |
| 4995 | RRPsYRKIKFFRKNLLRLTG |
| 4996 | RRPsYRKILFFRKNLLRLTG |
| 4997 | RRPsYRKIMFFRKNLLRLTG |
| 4998 | RRPsYRKIRFFRKNLLRLTG |
| 4999 | RRPsYRKIYFFRKNLLRLTG |
| 5000 | RRPsYTLGFFFRKNLLRLTG |
| 5001 | RRPsYTLGKFFRKNLLRLTG |
| 5002 | RRPsYTLGLFFRKNLLRLTG |
| 5003 | RRPsYTLGMFFRKNLLRLTG |
| 5004 | RRPsYTLGRFFRKNLLRLTG |
| 5005 | RRPsYTLGVFFRKNLLRLTG |
| 5006 | RRPsYTLGYFFRKNLLRLTG |
| 5007 | RRQsKVEALFFRKNLLRLTG |
| 5008 | RRRsLERLLFFRKNLLRLTG |
| 5009 | RRsFLVSYFFRKNLLRLTG |
| 5010 | RRSFsLEFFRKNLLRLTG |
| 5011 | RRSsFLQFFRKNLLRLTG |
| 5012 | RRssFLQLFFFRKNLLRLTG |
| 5013 | RRssFLQVFFFRKNLLRLTG |
| 5014 | RRSsFLQVFFFRKNLLRLTG |
| 5015 | RRSsFLQVKFFRKNLLRLTG |
| 5016 | RRSsFLQVLFFRKNLLRLTG |
| 5017 | RRssFLQVMFFRKNLLRLTG |
| 5018 | RRSsFLQVMFFRKNLLRLTG |
| 5019 | RRSsFLQVRFFRKNLLRLTG |
| 5020 | RRssFLQVVFFRKNLLRLTG |
| 5021 | RRSsFLQVYFFRKNLLRLTG |
| 5022 | RRSsIGLRFFFRKNLLRLTG |
| 5023 | RRSsIGLRKFFRKNLLRLTG |
| 5024 | RRSsIGLRLFFRKNLLRLTG |
| 5025 | RRSsIGLRMFFRKNLLRLTG |
| 5026 | RRSsIGLRRFFRKNLLRLTG |
| 5027 | RRSsIGLRVFFRKNLLRLTG |
| 5028 | RRSsIGLRYFFRKNLLRLTG |
| 5029 | RRsSIQSTFFFRKNLLRLTG |
| 5030 | RRSsIQSTFFFRKNLLRLTG |
| 5031 | RRSsIQSTKFFRKNLLRLTG |
| 5032 | RRSsIQSTLFFRKNLLRLTG |
| 5033 | RRSsIQSTMFFRKNLLRLTG |
| 5034 | RRSsIQSTRFFRKNLLRLTG |
| 5035 | RRSsIQSTYFFRKNLLRLTG |
| 5036 | RRSsLDAEIDSFFFRKNLLRLTG |
| 5037 | RRSsLDAEIDSLFFRKNLLRLTG |
| 5038 | RRSsLDAEIDSMFFRKNLLRLTG |
| 5039 | RRSsLDAEIDSVFFRKNLLRLTG |
| 5040 | RRsSQSWSFFFRKNLLRLTG |
| 5041 | RRSsQSWSFFFRKNLLRLTG |
| 5042 | RRSsQSWSKFFRKNLLRLTG |
| 5043 | RRsSQSWSLFFRKNLLRLTG |
| 5044 | RRSsQSWSLFFRKNLLRLTG |
| 5045 | RRsSQSWSMFFRKNLLRLTG |
| 5046 | RRSsQSWSMFFRKNLLRLTG |
| 5047 | RRSsQSWSRFFRKNLLRLTG |
| 5048 | RRsSQSWSVFFRKNLLRLTG |
| 5049 | RRSsQSWSYFFRKNLLRLTG |
| 5050 | RRSsSVAQVFFRKNLLRLTG |
| 5051 | RRSsTASLVKFFFRKNLLRLTG |
| 5052 | RRSsTASLVKKFFRKNLLRLTG |
| 5053 | RRSsTASLVKLFFRKNLLRLTG |
| 5054 | RRSsTASLVKMFFRKNLLRLTG |
| 5055 | RRSsTASLVKRFFRKNLLRLTG |
| 5056 | RRsSVDLGFFFRKNLLRLTG |
| 5057 | RRSsVDLGFFFRKNLLRLTG |
| 5058 | RRsSVDLGKFFRKNLLRLTG |
| 5059 | RRSsVDLGKFFRKNLLRLTG |
| 5060 | RRsSVDLGLFFRKNLLRLTG |
| 5061 | RRSsVDLGLFFRKNLLRLTG |
| 5062 | RRsSVDLGMFFRKNLLRLTG |
| 5063 | RRSsVDLGMFFRKNLLRLTG |
| 5064 | RRsSVDLGRFFRKNLLRLTG |
| 5065 | RRSsVDLGRFFRKNLLRLTG |
| 5066 | RRsSVDLGYFFRKNLLRLTG |
| 5067 | RRSsVDLGYFFRKNLLRLTG |
| 5068 | RRSsVKVEAFFRKNLLRLTG |
| 5069 | RRSsVKVEFFFRKNLLRLTG |
| 5070 | RRSsVKVEKFFRKNLLRLTG |
| 5071 | RRSsVKVELFFRKNLLRLTG |
| 5072 | RRSsVKVEMFFRKNLLRLTG |
| 5073 | RRSsVKVERFFRKNLLRLTG |
| 5074 | RRSsVKVEYFFRKNLLRLTG |
| 5075 | RRTsPITRFFFRKNLLRLTG |
| 5076 | RRTsPITRKFFRKNLLRLTG |
| 5077 | RRTsPITRLFFRKNLLRLTG |
| 5078 | RRTsPITRMFFRKNLLRLTG |
| 5079 | RRTsPITRRFFRKNLLRLTG |
| 5080 | RRVVQRSsFFFRKNLLRLTG |
| 5081 | RRVVQRSsKFFRKNLLRLTG |
| 5082 | RRVVQRSsLFFRKNLLRLTG |
| 5083 | RRVVQRSsMFFRKNLLRLTG |
| 5084 | RRVVQRSsRFFRKNLLRLTG |
| 5085 | RRVVQRSsYFFRKNLLRLTG |
| 5086 | RRWQRSsLFFRKNLLRLTG |
| 5087 | RRYsGKTEFFFRKNLLRLTG |
| 5088 | RRYsGKTEKFFRKNLLRLTG |
| 5089 | RRYsGKTELFFRKNLLRLTG |
| 5090 | RRYsGKTERFFRKNLLRLTG |
| 5091 | RRYsGKTEYFFRKNLLRLTG |
| 5092 | RRYsGNMEFFFRKNLLRLTG |
| 5093 | RRYsGNMEKFFRKNLLRLTG |
| 5094 | RRYsGNMELFFRKNLLRLTG |
| 5095 | RRYsGNMEMFFRKNLLRLTG |
| 5096 | RRYsGNMERFFRKNLLRLTG |
| 5097 | RRYsKFFDLFFRKNLLRLTG |
| 5098 | RRYsPPIERFFRKNLLRLTG |
| 5099 | RRYsPPIQFFRKNLLRLTG |
| 5100 | RRYsPPIQFFFRKNLLRLTG |
| 5101 | RRYsPPIQKFFRKNLLRLTG |
| 5102 | RRYsPPIQLFFRKNLLRLTG |
| 5103 | RRYsPPIQMFFRKNLLRLTG |
| 5104 | RRYsPPIQRFFRKNLLRLTG |
| 5105 | RRYsPPIQYFFRKNLLRLTG |
| 5106 | RRYsRsPYSFFFRKNLLRLTG |
| 5107 | RRYsRSPYSFFFRKNLLRLTG |
| 5108 | RRYSRsPYSFFFRKNLLRLTG |
| 5109 | RRYsRsPYSKFFRKNLLRLTG |
| 5110 | RRYsRSPYSKFFRKNLLRLTG |
| 5111 | RRYSRsPYSKFFRKNLLRLTG |
| 5112 | RRYsRsPYSLFFRKNLLRLTG |
| 5113 | RRYsRSPYSLFFRKNLLRLTG |
| 5114 | RRYSRsPYSLFFRKNLLRLTG |
| 5115 | RRYsRsPYSMFFRKNLLRLTG |
| 5116 | RRYsRSPYSMFFRKNLLRLTG |
| 5117 | RRYSRsPYSMFFRKNLLRLTG |
| 5118 | RRYsRsPYSRFFRKNLLRLTG |
| 5119 | RRYsRSPYSRFFRKNLLRLTG |
| 5120 | RRYSRsPYSRFFRKNLLRLTG |
| 5121 | RRYtNRVVTKFFRKNLLRLTG |
| 5122 | RRYtNRVVTLFFRKNLLRLTG |
| 5123 | RRYtNRVVTMFFRKNLLRLTG |
| 5124 | RRYtNRVVTRFFRKNLLRLTG |
| 5125 | RSAsFSRKVFFRKNLLRLTG |
| 5126 | RSAsPDDDLGSSNFFRKNLLRLTG |
| 5127 | RSAsSATQVHKFFRKNLLRLTG |
| 5128 | RSAsSATQVHYFFRKNLLRLTG |
| 5129 | RSDPSKsPGSLRYFFRKNLLRLTG |
| 5130 | RSDsPKIDLFFRKNLLRLTG |
| 5131 | RSDsPKIDYFFRKNLLRLTG |
| 5132 | RSDsRAQAVFFRKNLLRLTG |
| 5133 | RSDsRAQAYFFRKNLLRLTG |
| 5134 | RSDsVGENLFFRKNLLRLTG |
| 5135 | RSDsVGENYFFRKNLLRLTG |
| 5136 | RSDsYVELFFRKNLLRLTG |
| 5137 | RSDsYVELSQYFFRKNLLRLTG |
| 5138 | RSEPSKsPGSLRYFFRKNLLRLTG |
| 5139 | RSEsKDRKFFFRKNLLRLTG |
| 5140 | RSEsKDRKLFFRKNLLRLTG |
| 5141 | RSEsKDRKMFFRKNLLRLTG |
| 5142 | RSEsKDRKVFFRKNLLRLTG |
| 5143 | RSEsPKIDLFFRKNLLRLTG |
| 5144 | RSEsPKIDYFFRKNLLRLTG |
| 5145 | RSEsPPAELFFRKNLLRLTG |
| 5146 | RSEsRAQAVFFRKNLLRLTG |
| 5147 | RSEsRAQAYFFRKNLLRLTG |
| 5148 | RSEsVGENLFFRKNLLRLTG |
| 5149 | RSEsVGENYFFRKNLLRLTG |
| 5150 | RSEsYVELSQYFFRKNLLRLTG |
| 5151 | RSFsPTMKVFFRKNLLRLTG |
| 5152 | RSGsLERKFFFRKNLLRLTG |
| 5153 | RSGsLERKLFFRKNLLRLTG |
| 5154 | RSGsLERKMFFRKNLLRLTG |
| 5155 | RSGsLERKVFFRKNLLRLTG |
| 5156 | RSHSsPASLFFRKNLLRLTG |
| 5157 | RSIsVGENLFFRKNLLRLTG |
| 5158 | RSLsESYELFFRKNLLRLTG |
| 5159 | RSLsPGGAAFFRKNLLRLTG |
| 5160 | RSLsPGGAFFFRKNLLRLTG |
| 5161 | RSLsPGGALFFRKNLLRLTG |
| 5162 | RSLsPGGAMFFRKNLLRLTG |
| 5163 | RSLsPGGAVFFRKNLLRLTG |
| 5164 | RSLsPLLFFFRKNLLRLTG |
| 5165 | RSLsPLLLFFRKNLLRLTG |
| 5166 | RSLsPLLMFFRKNLLRLTG |
| 5167 | RSLsPLLVFFRKNLLRLTG |
| 5168 | RSLsQELVGVFFRKNLLRLTG |
| 5169 | RSLsVEIVKFFRKNLLRLTG |
| 5170 | RSLsVEIVYFFRKNLLRLTG |
| 5171 | RSMsMPVAHFFRKNLLRLTG |
| 5172 | RSMsMPVAKFFRKNLLRLTG |
| 5173 | RsPEDEYELLMPHRISSHFFRKNLLRLTG |
| 5174 | RSRRsPLLKFFRKNLLRLTG |
| 5175 | RSRRsPLLYFFRKNLLRLTG |
| 5176 | RSRsPLELFFRKNLLRLTG |
| 5177 | RSRsPPPVSKFFRKNLLRLTG |
| 5178 | RSRsPPPVSYFFRKNLLRLTG |
| 5179 | RSRsPRPAFFFRKNLLRLTG |
| 5180 | RSRsPRPAIFFRKNLLRLTG |
| 5181 | RSRsPRPALFFRKNLLRLTG |
| 5182 | RSRsPRPAMFFRKNLLRLTG |
| 5183 | RSRsPRPAVFFRKNLLRLTG |
| 5184 | RSRsPRPAXFFRKNLLRLTG |
| 5185 | RSRTsPITRRFFRKNLLRLTG |
| 5186 | RSRTsPITRYFFRKNLLRLTG |
| 5187 | RSSsLIRHKFFRKNLLRLTG |
| 5188 | RSSsLIRHYFFRKNLLRLTG |
| 5189 | RSVsLSMRKFFRKNLLRLTG |
| 5190 | RSVsLSMRYFFRKNLLRLTG |
| 5191 | RsWKYNQSISLRRPFFRKNLLRLTG |
| 5192 | RSYsGSRsKFFRKNLLRLTG |
| 5193 | RSYsGSRsRFFRKNLLRLTG |
| 5194 | RSYsGSRsYFFRKNLLRLTG |
| 5195 | RSYsPDHRQKFFRKNLLRLTG |
| 5196 | RSYsPDHRQYFFRKNLLRLTG |
| 5197 | RSYsPERSKFFRKNLLRLTG |
| 5198 | RSYsPERSYFFRKNLLRLTG |
| 5199 | RSYsPRNSRFFRKNLLRLTG |
| 5200 | RSYsPRNSYFFRKNLLRLTG |
| 5201 | RSYSRsFSKFFRKNLLRLTG |
| 5202 | RSYsRSFSRFFRKNLLRLTG |
| 5203 | RSYSRsFSRFFRKNLLRLTG |
| 5204 | RSYSRsFSYFFRKNLLRLTG |
| 5205 | RSYsYPRQKFFRKNLLRLTG |
| 5206 | RSYsYPRQYFFRKNLLRLTG |
| 5207 | RSYVTTSTRTYsLGFFRKNLLRLTG |
| 5208 | RTAsFAVRKFFRKNLLRLTG |
| 5209 | RTAsFAVRYFFRKNLLRLTG |
| 5210 | RTAsLIIKVFFRKNLLRLTG |
| 5211 | RTAsPPPPPKFFRKNLLRLTG |
| 5212 | RTDPSKsPGSLRYFFRKNLLRLTG |
| 5213 | RTDsPKIDLFFRKNLLRLTG |
| 5214 | RTDsPKIDYFFRKNLLRLTG |
| 5215 | RTDsRAQAVFFRKNLLRLTG |
| 5216 | RTDsRAQAYFFRKNLLRLTG |
| 5217 | RTDsYVELSQYFFRKNLLRLTG |
| 5218 | RTEPSKsPGSLRYFFRKNLLRLTG |
| 5219 | RTEsDSGLKFFFRKNLLRLTG |
| 5220 | RTEsDSGLKKFFRKNLLRLTG |
| 5221 | RTEsDSGLKLFFRKNLLRLTG |
| 5222 | RTEsDSGLKMFFRKNLLRLTG |
| 5223 | RTEsDSGLKVFFRKNLLRLTG |
| 5224 | RTEsPKIDLFFRKNLLRLTG |
| 5225 | RTEsPKIDYFFRKNLLRLTG |
| 5226 | RTEsRAQAVFFRKNLLRLTG |
| 5227 | RTEsRAQAYFFRKNLLRLTG |
| 5228 | RTEsYVELSQYFFRKNLLRLTG |
| 5229 | RTFsLDTILFFRKNLLRLTG |
| 5230 | RTFsPTYGFFFRKNLLRLTG |
| 5231 | RTFsPTYGLFFRKNLLRLTG |
| 5232 | RTFsPTYGMFFRKNLLRLTG |
| 5233 | RTFsPTYGVFFRKNLLRLTG |
| 5234 | RTHsLLLLLFFRKNLLRLTG |
| 5235 | RTLsHISEAFFRKNLLRLTG |
| 5236 | RTLsHISEVFFRKNLLRLTG |
| 5237 | RTLsPEIITVFFRKNLLRLTG |
| 5238 | RTMsEAALVRKFFRKNLLRLTG |
| 5239 | RTNsPGFQKFFRKNLLRLTG |
| 5240 | RTPsDVKELFFRKNLLRLTG |
| 5241 | RTPsFLKKNKFFRKNLLRLTG |
| 5242 | RTPsFLKKNYFFRKNLLRLTG |
| 5243 | RTRsLSSLREKFFRKNLLRLTG |
| 5244 | RTRsLSSLREYFFRKNLLRLTG |
| 5245 | RTRsPSPTFFFRKNLLRLTG |
| 5246 | RTRsPSPTLFFRKNLLRLTG |
| 5247 | RTRsPSPTMFFRKNLLRLTG |
| 5248 | RTRsPSPTVFFRKNLLRLTG |
| 5249 | RTSsFALNLFFRKNLLRLTG |
| 5250 | RTSsFTEQLFFRKNLLRLTG |
| 5251 | RTSsFTFQNFFRKNLLRLTG |
| 5252 | RTSSFtFQNFFRKNLLRLTG |
| 5253 | RTSsPLFNKFFRKNLLRLTG |
| 5254 | RTYKsPLRHFFRKNLLRLTG |
| 5255 | RTYKsPLRKFFRKNLLRLTG |
| 5256 | RTYKsPLRYFFRKNLLRLTG |
| 5257 | RTYsGPMNKFFRKNLLRLTG |
| 5258 | RTYsGPMNKVFFRKNLLRLTG |
| 5259 | RTYsHGTYRFFRKNLLRLTG |
| 5260 | RVAsFAVRKFFRKNLLRLTG |
| 5261 | RVAsFAVRYFFRKNLLRLTG |
| 5262 | RVAsPLVHKFFRKNLLRLTG |
| 5263 | RVAsPLVHYFFRKNLLRLTG |
| 5264 | RVAsPPPPPKFFRKNLLRLTG |
| 5265 | RVAsPPPPPYFFRKNLLRLTG |
| 5266 | RVAsPTSGVFFRKNLLRLTG |
| 5267 | RVAsPTSGVKFFRKNLLRLTG |
| 5268 | RVAsPTSGVKKFFRKNLLRLTG |
| 5269 | RVAsPTSGVKRFFRKNLLRLTG |
| 5270 | RVAsPTSGVYFFRKNLLRLTG |
| 5271 | RVDsPSHGLFFRKNLLRLTG |
| 5272 | RVGsLVLNLFFRKNLLRLTG |
| 5273 | RVIsGVLQLFFRKNLLRLTG |
| 5274 | RVKLPsGSKKFFRKNLLRLTG |
| 5275 | RVKsPGsGHVKFFRKNLLRLTG |
| 5276 | RVKsPGsGHVYFFRKNLLRLTG |
| 5277 | RVKsPISLKFFRKNLLRLTG |
| 5278 | RVKsPSPKSERFFRKNLLRLTG |
| 5279 | RVKsPSPKSEYFFRKNLLRLTG |
| 5280 | RVKtPTSQSYKFFRKNLLRLTG |
| 5281 | RVKtPTSQSYRFFRKNLLRLTG |
| 5282 | RVKtPTSQSYYFFRKNLLRLTG |
| 5283 | RVKTtPLRRFFRKNLLRLTG |
| 5284 | RVKTtPLRYFFRKNLLRLTG |
| 5285 | RVLDRSPsRSAKFFRKNLLRLTG |
| 5286 | RVLDRSPsRSAYFFRKNLLRLTG |
| 5287 | RVLHsPPAVFFRKNLLRLTG |
| 5288 | RVLsGVVTKFFRKNLLRLTG |
| 5289 | RVLsPLIIKFFRKNLLRLTG |
| 5290 | RVPsLLVLLFFRKNLLRLTG |
| 5291 | RVPsSTLKKFFRKNLLRLTG |
| 5292 | RVPsSTLKYFFRKNLLRLTG |
| 5293 | RVRKLPsTTLFFRKNLLRLTG |
| 5294 | RVRQsPLATKFFRKNLLRLTG |
| 5295 | RVRQsPLATRFFRKNLLRLTG |
| 5296 | RVRQsPLATYFFRKNLLRLTG |
| 5297 | RVRRsSFLNAKFFRKNLLRLTG |
| 5298 | RVRsLSSLREKFFRKNLLRLTG |
| 5299 | RVRsLSSLREYFFRKNLLRLTG |
| 5300 | RVRsPTRSFFFRKNLLRLTG |
| 5301 | RVRsPTRSLFFRKNLLRLTG |
| 5302 | RVRsPTRSMFFRKNLLRLTG |
| 5303 | RVRsPTRSPFFRKNLLRLTG |
| 5304 | RVRsPTRSVFFRKNLLRLTG |
| 5305 | RVSsPISKKFFRKNLLRLTG |
| 5306 | RVSsPISKYFFRKNLLRLTG |
| 5307 | RVSsRFSSKFFRKNLLRLTG |
| 5308 | RVSsRFSSRFFRKNLLRLTG |
| 5309 | RVSsRFSSYFFRKNLLRLTG |
| 5310 | RVSsVKLISKFFRKNLLRLTG |
| 5311 | RVSsVKLISYFFRKNLLRLTG |
| 5312 | RVTsAEIKLFFRKNLLRLTG |
| 5313 | RVVsLSMRKFFRKNLLRLTG |
| 5314 | RVVsLSMRYFFRKNLLRLTG |
| 5315 | RVWEDRPSsAFFRKNLLRLTG |
| 5316 | RVWsPPRVHKVFFRKNLLRLTG |
| 5317 | RVYQyIQSRFFRKNLLRLTG |
| 5318 | RVYQyIQSRFKFFRKNLLRLTG |
| 5319 | RVYQyIQSRFYFFRKNLLRLTG |
| 5320 | RVYQyIQSRKFFRKNLLRLTG |
| 5321 | RVYQyIQSRYFFRKNLLRLTG |
| 5322 | RVYsPYNHKFFRKNLLRLTG |
| 5323 | RVYsPYNHRFFRKNLLRLTG |
| 5324 | RVYsPYNHYFFRKNLLRLTG |
| 5325 | RVYSRsFSKFFRKNLLRLTG |
| 5326 | RVYSRsFSYFFRKNLLRLTG |
| 5327 | RYPsNLQLFFFRKNLLRLTG |
| 5328 | RYQtQPVTLFFRKNLLRLTG |
| 5329 | SAARESHPHGVKRSAsPDDDLGFFRKNLLRLTG |
| 5330 | SARGsPTRPNPPVRFFRKNLLRLTG |
| 5331 | SARRtPVSYFFRKNLLRLTG |
| 5332 | sDDEKMPDLEFFRKNLLRLTG |
| 5333 | sDFHAERAAREKFFRKNLLRLTG |
| 5334 | SDmPRAHsFFFRKNLLRLTG |
| 5335 | SDMPRAHsFFFRKNLLRLTG |
| 5336 | SEFKAMDsIFFRKNLLRLTG |
| 5337 | SEGsLHRKFFFRKNLLRLTG |
| 5338 | SEGsLHRKWFFRKNLLRLTG |
| 5339 | SEGsLHRKYFFRKNLLRLTG |
| 5340 | SELsPGRSVFFRKNLLRLTG |
| 5341 | SFDsGSVRLFFRKNLLRLTG |
| 5342 | SGGAQsPLRYLHVLFFRKNLLRLTG |
| 5343 | sGGDDDWTHLSSKEVDPSTFFRKNLLRLTG |
| 5344 | sGGDDDWTHLSSKEVDPSTGFFRKNLLRLTG |
| 5345 | sGGDDDWTHLSSKEVDPSTGEFFRKNLLRLTG |
| 5346 | sGGDDDWTHLSSKEVDPSTGELFFRKNLLRLTG |
| 5347 | sGGDDDWTHLSSKEVDPSTGELQFFRKNLLRLTG |
| 5348 | SGPKPLFRRMsSLVGPTQFFRKNLLRLTG |
| 5349 | SIDsPQKLFFRKNLLRLTG |
| 5350 | SIDsPQKYFFRKNLLRLTG |
| 5351 | SILsFVSGLFFRKNLLRLTG |
| 5352 | SIMsFHIDLFFRKNLLRLTG |
| 5353 | SImsPEIQLFFRKNLLRLTG |
| 5354 | SIMsPEIQLFFRKNLLRLTG |
| 5355 | SIPtVSGQIFFRKNLLRLTG |
| 5356 | SISsMEVNVFFRKNLLRLIG |
| 5357 | SISStPPAVFFRKNLLRLTG |
| 5358 | SKEDKNGHDGDTHQEDDGEKsDFFRKNLLRLTG |
| 5359 | SKRGyIGLFFRKNLLRLTG |
| 5360 | SKtVATFILFFRKNLLRLTG |
| 5361 | SLAsLTE,KIFFRKNLLRLTG |
| 5362 | SLDSEDYsLFFRKNLLRLTG |
| 5363 | SLDsLGDVFLFFRKNLLRLTG |
| 5364 | SLDsPSYVLYFFRKNLLRLTG |
| 5365 | SLEsPSYVLYFFRKNLLRLTG |
| 5366 | SLFGGsVKLFFRKNLLRLTG |
| 5367 | SLFKRLYsLFFRKNLLRLTG |
| 5368 | SLFsGDEENAFFRKNLLRLTG |
| 5369 | SLFsGSYSSLFFRKNLLRLTG |
| 5370 | SLFsPQNTLFFRKNLLRLTG |
| 5371 | SLFsPRRNKFFRKNLLRLTG |
| 5372 | SLFsPRRNYFFRKNLLRLTG |
| 5373 | SLFsSEESNLFFRKNLLRLTG |
| 5374 | SLFsSEESNLGAFFRKNLLRLTG |
| 5375 | SLHDIQLsLFFRKNLLRLTG |
| 5376 | SLKsPVTVKFFRKNLLRLTG |
| 5377 | SLLAsPGHISVFFRKNLLRLTG |
| 5379 | SLLNKSsPVKFFRKNLLRLTG |
| 5380 | SLLNKSsPVKKFFRKNLLRLTG |
| 5381 | SLLNKSsPVKYFFRKNLLRLTG |
| 5382 | SLLsLHVDLFFRKNLLRLTG |
| 5383 | SLLTsPPKAFFRKNLLRLTG |
| 5384 | SLLTsPPKVFFRKNLLRLTG |
| 5385 | SLMsGTLESLFFRKNLLRLTG |
| 5386 | SLMsPGRRKFFRKNLLRLTG |
| 5387 | SLMsPGRRYFFRKNLLRLTG |
| 5388 | SLQPRSHsVFFRKNLLRLTG |
| 5389 | SLQsLETSVFFRKNLLRLTG |
| 5390 | SLRRsVLMKFFRKNLLRLTG |
| 5391 | SLRRsVLMYFFRKNLLRLTG |
| 5392 | SLSsLLVKLFFRKNLLRLTG |
| 5393 | SLtRSPPRVFFRKNLLRLTG |
| 5394 | SLTRsPPRVFFRKNLLRLTG |
| 5395 | SLVDGyFRLFFRKNLLRLTG |
| 5396 | SLYDRPAsYFFRKNLLRLTG |
| 5397 | SLYsPVKKKFFRKNLLRLTG |
| 5398 | SMFsPRRNKFFRKNLLRLTG |
| 5399 | SMKsPVTVKFFRKNLLRLTG |
| 5400 | SMLNKSsPVKFFRKNLLRLTG |
| 5401 | SMLNKSsPVKKFFRKNLLRLTG |
| 5402 | SMLsQEIQTLFFRKNLLRLTG |
| 5403 | SMLTsPPKAFFRKNLLRLTG |
| 5404 | SMLTsPPKVFFRKNLLRLTG |
| 5405 | SMMsPGRRKFFRKNLLRLTG |
| 5406 | SMQPRSHsVFFRKNLLRLTG |
| 5407 | SMRRsVLMKFFRKNLLRLTG |
| 5408 | SMSsLSREVFFRKNLLRLTG |
| 5409 | SMtRSPPRVFFRKNLLRLTG |
| 5410 | SMTRsPPRVFFRKNLLRLTG |
| 5411 | SMYsPVKKKFFRKNLLRLTG |
| 5412 | SNFKsPVKTIRFFRKNLLRLTG |
| 5413 | SPAASISRLsGEQVDGKGFFRKNLLRLTG |
| 5414 | SPAsPKISFFFRKNLLRLTG |
| 5415 | SPAsPKISLFFRKNLLRLTG |
| 5416 | SPAsPKISMFFRKNLLRLTG |
| 5417 | SPAsPKISVFFRKNLLRLTG |
| 5418 | SPDsSQSSLFFRKNLLRLTG |
| 5419 | sPEDEYELLMPHRISSHFFRKNLLRLTG |
| 5420 | SPEDEYELLMPHRIsSHFFRKNLLRLTG |
| 5421 | SPEKAGRRsSFFFRKNLLRLTG |
| 5422 | SPEKAGRRsSLFFRKNLLRLTG |
| 5423 | SPEKAGRRsSMFFRKNLLRLTG |
| 5424 | SPEKAGRRsSVFFRKNLLRLTG |
| 5425 | sPERPFLATLGGAKVADKFFRKNLLRLTG |
| 5426 | sPERPFLATLGGAKVADKIQFFRKNLLRLTG |
| 5427 | SPFKRQLsFFFRKNLLRLTG |
| 5428 | SPFKRQLsLFFRKNLLRLTG |
| 5429 | SPFKRQLsMFFRKNLLRLTG |
| 5430 | SPFKRQLsVFFRKNLLRLTG |
| 5431 | SPFLsKRSLFFRKNLLRLTG |
| 5432 | SPGLARKRsFFFRKNLLRLTG |
| 5433 | SPGLARKRsLFFRKNLLRLTG |
| 5434 | SPGLARKRsMFFRKNLLRLTG |
| 5435 | SPGLARKRsVFFRKNLLRLTG |
| 5436 | SPGsPRPAFFFRKNLLRLTG |
| 5437 | SPGsPRPALFFRKNLLRLTG |
| 5438 | SPGsPRPAMFFRKNLLRLTG |
| 5439 | SPGsPRPAVFFRKNLLRLTG |
| 5440 | SPKsPGLKAFFRKNLLRLTG |
| 5441 | SPKsPGLKFFFRKNLLRLTG |
| 5442 | SPKsPGLKLFFRKNLLRLTG |
| 5443 | SPKsPGLKMFFRKNLLRLTG |
| 5444 | SPKsPGLKVFFRKNLLRLTG |
| 5445 | SPKsPTAAFFFRKNLLRLTG |
| 5446 | SPKsPTAALFFRKNLLRLTG |
| 5447 | SPKsPTAAMFFRKNLLRLTG |
| 5448 | SPKsPTAAVFFRKNLLRLTG |
| 5449 | SPLTKSIsLFFRKNLLRLTG |
| 5450 | sPPFPVPVYTRQAPKQVIKFFRKNLLRLTG |
| 5451 | SPRAPVsPLKFFFRKNLLRLTG |
| 5452 | SPRERsPALFFRKNLLRLTG |
| 5453 | SPRGEAsSLFFRKNLLRLTG |
| 5454 | SPRGEASsLFFRKNLLRLTG |
| 5455 | SPRPPNsPSIFFRKNLLRLTG |
| 5456 | SPRRsLGLALFFRKNLLRLTG |
| 5457 | SPRRsRSIsFFFRKNLLRLTG |
| 5458 | SPRRsRSISFFFRKNLLRLTG |
| 5459 | SPRRsRSIsLFFRKNLLRLTG |
| 5460 | SPRRsRSISLFFRKNLLRLTG |
| 5461 | SPRRsRSIsMFFRKNLLRLTG |
| 5462 | SPRRsRSISMFFRKNLLRLTG |
| 5463 | SPRRsRSIsVFFRKNLLRLTG |
| 5464 | SPRRsRSISVFFRKNLLRLTG |
| 5465 | SPRsITSTFFFRKNLLRLTG |
| 5466 | SPRsITSTLFFRKNLLRLTG |
| 5467 | SPRsITSTMFFRKNLLRLTG |
| 5468 | SPRsITSTPFFRKNLLRLTG |
| 5469 | SPRsITSTVFFRKNLLRLTG |
| 5470 | SPRsPDRTLFFRKNLLRLTG |
| 5471 | SPRsPGKPFFFRKNLLRLTG |
| 5472 | SPRsPGKPLFFRKNLLRLTG |
| 5473 | SPRsPGKPMFFRKNLLRLTG |
| 5474 | SPRsPGKPVFFRKNLLRLTG |
| 5475 | SPRsPGRSFFFRKNLLRLTG |
| 5476 | SPRsPGRSIFFRKNLLRLTG |
| 5477 | SPRsPGRSLFFRKNLLRLTG |
| 5478 | SPRsPGRSMFFRKNLLRLTG |
| 5479 | SPRsPGRSVFFRKNLLRLTG |
| 5480 | SPRsPGRSXFFRKNLLRLTG |
| 5481 | SPRsPSGLRFFRKNLLRLTG |
| 5482 | SPRsPSTTYFFFRKNLLRLTG |
| 5483 | SPRsPSTTYLFFRKNLLRLTG |
| 5484 | SPRSPsTTYLFFRKNLLRLTG |
| 5485 | SPRsPSTTYMFFRKNLLRLTG |
| 5486 | SPRsPSTTYVFFRKNLLRLTG |
| 5487 | SPRssQLVFFRKNLLRLTG |
| 5488 | SPRtPVsPVKFFFRKNLLRLTG |
| 5489 | SPRTPVsPVKFFFRKNLLRLTG |
| 5490 | SPRtPVsPVKLFFRKNLLRLTG |
| 5491 | SPRTPVsPVKLFFRKNLLRLTG |
| 5492 | SPRtPVsPVKMFFRKNLLRLTG |
| 5493 | SPRTPVsPVKMFFRKNLLRLTG |
| 5494 | SPRtPVsPVKVFFRKNLLRLTG |
| 5495 | SPRTPVsPVKVFFRKNLLRLTG |
| 5496 | SPSsPSVRRQFFFRKNLLRLTG |
| 5497 | SPSsPSVRRQLFFRKNLLRLTG |
| 5498 | SPSsPSVRRQMFFRKNLLRLTG |
| 5499 | SPSsPSVRRQVFFRKNLLRLTG |
| 5500 | SPSTSRSGGsSRFFFRKNLLRLTG |
| 5501 | SPSTSRSGGsSRLFFRKNLLRLTG |
| 5502 | SPSTSRSGGsSRMFFRKNLLRLTG |
| 5503 | SPSTSRSGGsSRVFFRKNLLRLTG |
| 5504 | sPTRPNPPVRNLHFFRKNLLRLTG |
| 5505 | SPVsPMKELFFRKNLLRLTG |
| 5506 | SPVsTRPLEPFFRKNLLRLTG |
| 5507 | SPVStRPLEPFFRKNLLRLTG |
| 5508 | SPVVHQsFFFRKNLLRLTG |
| 5509 | SPVVHQsLFFRKNLLRLTG |
| 5510 | SPVVHQsMFFRKNLLRLTG |
| 5511 | SPVVHQsVFFRKNLLRLTG |
| 5512 | SQIsPKSWGVFFRKNLLRLTG |
| 5513 | SRDKHsEYFFRKNLLRLTG |
| 5514 | SREKHsEIFFRKNLLRLTG |
| 5515 | SREKHsElFFRKNLLRLTG |
| 5516 | SRFNRRVsVFFRKNLLRLTG |
| 5517 | SRLTHLsFFFRKNLLRLTG |
| 5518 | SRLTHLsKFFRKNLLRLTG |
| 5519 | SRLTHLsLFFRKNLLRLTG |
| 5520 | SRLTHLsMFFRKNLLRLTG |
| 5521 | SRLTHLsRFFRKNLLRLTG |
| 5522 | SRLTHLsYFFRKNLLRLTG |
| 5523 | SRMsPKAQFFFRKNLLRLTG |
| 5524 | SRMsPKAQKFFRKNLLRLTG |
| 5525 | SRMsPKAQLFFRKNLLRLTG |
| 5526 | SRMsPKAQMFFRKNLLRLTG |
| 5527 | SRMsPKAQRFFRKNLLRLTG |
| 5528 | SRMsPKAQYFFRKNLLRLTG |
| 5529 | SRsSRSPYSRFFRKNLLRLTG |
| 5530 | SRSsSVLsLFFRKNLLRLTG |
| 5531 | SRSSsVLSLFFRKNLLRLTG |
| 5532 | SRSSSVLsLFFRKNLLRLTG |
| 5533 | SRTsPITRFFFRKNLLRLTG |
| 5534 | SRTsPITRKFFRKNLLRLTG |
| 5535 | SRTsPITRLFFRKNLLRLTG |
| 5536 | SRTsPITRMFFRKNLLRLTG |
| 5537 | SRTsPITRRFFRKNLLRLTG |
| 5538 | SRTsPITRYFFRKNLLRLTG |
| 5539 | SRWsGSHQFFFRKNLLRLTG |
| 5540 | SRWsGSHQKFFRKNLLRLTG |
| 5541 | SRWsGSHQRFFRKNLLRLTG |
| 5542 | SRWsGSHQYFFRKNLLRLTG |
| 5543 | SRYsRsPYSFFFRKNLLRLTG |
| 5544 | SRYsRSPYSFFFRKNLLRLTG |
| 5545 | SRYSRsPYSFFFRKNLLRLTG |
| 5546 | SRYsRsPYSKFFRKNLLRLTG |
| 5547 | SRYsRSPYSKFFRKNLLRLTG |
| 5548 | SRYSRsPYSKFFRKNLLRLTG |
| 5549 | SRYsRsPYSLFFRKNLLRLTG |
| 5550 | SRYsRSPYSLFFRKNLLRLTG |
| 5551 | SRYSRsPYSLFFRKNLLRLTG |
| 5552 | SRYsRsPYSMFFRKNLLRLTG |
| 5553 | SRYsRSPYSMFFRKNLLRLTG |
| 5554 | SRYSRsPYSMFFRKNLLRLTG |
| 5555 | SRYsRsPYSRFFRKNLLRLTG |
| 5556 | SRYsRSPYSRFFRKNLLRLTG |
| 5557 | SRYSRsPYSRFFRKNLLRLTG |
| 5558 | SRYsRsPYSYFFRKNLLRLTG |
| 5559 | SRYsRSPYSYFFRKNLLRLTG |
| 5560 | SRYSRsPYSYFFRKNLLRLTG |
| 5561 | SRYsRtsPYSRFFRKNLLRLTG |
| 5562 | SSDIsPTRLFFRKNLLRLTG |
| 5563 | SSDIsPTRYFFRKNLLRLTG |
| 5564 | SSDKHsEYFFRKNLLRLTG |
| 5565 | SSDPASQLsYFFRKNLLRLTG |
| 5566 | SSDsETLRYFFRKNLLRLTG |
| 5567 | SSDsPQKLFFRKNLLRLTG |
| 5568 | SSDsPQKYFFRKNLLRLTG |
| 5569 | SSDsPSYVLYFFRKNLLRLTG |
| 5570 | SSDsPTNHFFFFRKNLLRLTG |
| 5571 | SSEIsPTRYFFRKNLLRLTG |
| 5572 | SSEKHsEYFFRKNLLRLTG |
| 5573 | SSEPASQLsYFFRKNLLRLTG |
| 5574 | SSEsETLRYFFRKNLLRLTG |
| 5575 | SSEsPQKLFFRKNLLRLTG |
| 5576 | SSEsPQKYFFRKNLLRLTG |
| 5577 | SSEsPSYVLYFFRKNLLRLTG |
| 5578 | SSEsPTNHFYFFRKNLLRLTG |
| 5579 | SSNGMKASRRsEEKEAGFFRKNLLRLTG |
| 5580 | SSNGKMASRRsEEKEAGEIFFRKNLLRLTG |
| 5581 | SsPIMRKKVSLFFRKNLLRLTG |
| 5582 | sSPPFPVPVYTRQAPKQVIKFFRKNLLRLTG |
| 5583 | SSsPTHAKSAHVFFRKNLLRLTG |
| 5584 | SSsWRILGSKQSEHRPFFRKNLLRLTG |
| 5585 | STDIsPTRLFFRKNLLRLTG |
| 5586 | STDIsPTRYFFRKNLLRLTG |
| 5587 | STDKHsEYFFRKNLLRLTG |
| 5588 | STDPASQLsYFFRKNLLRLTG |
| 5589 | STDsETLRYFFRKNLLRLTG |
| 5590 | STDsPQKYFFRKNLLRLTG |
| 5591 | STDsPSYVLYFFRKNLLRLTG |
| 5592 | STDsPTNHFYFFRKNLLRLTG |
| 5593 | STEIsPTRLFFRKNLLRLTG |
| 5594 | STEIsPTRYFFRKNLLRLTG |
| 5595 | STEKHsEYFFRKNLLRLTG |
| 5596 | STEPASQLsYFFRKNLLRLTG |
| 5597 | STEsETLRYFFRKNLLRLTG |
| 5598 | STEsPQKYFFRKNLLRLTG |
| 5599 | STEsPSYVLYFFRKNLLRLTG |
| 5600 | STEsPTNHFYFFRKNLLRLTG |
| 5601 | STIQNsPTKKFFRKNLLRLTG |
| 5602 | sTMSLNIITVFFRKNLLRLTG |
| 5603 | STMsLNIITVFFRKNLLRLTG |
| 5604 | SVDIsPIRLFFRKNLLRLTG |
| 5605 | SVDIsPTRLFFRKNLLRLTG |
| 5606 | SVDIsPTRYFFRKNLLRLTG |
| 5607 | SVFsPSFGLFFRKNLLRLTG |
| 5608 | SVGsDYYIQLFFRKNLLRLTG |
| 5609 | SVKPRRTsLFFRKNLLRLTG |
| 5610 | SVKsPVTVKFFRKNLLRLTG |
| 5611 | SVKsPVTVYFFRKNLLRLTG |
| 5612 | SVLsPSFQLFFRKNLLRLTG |
| 5613 | SVMDsPKKLFFRKNLLRLTG |
| 5614 | SVRRsVLMKFFRKNLLRLTG |
| 5615 | SVRRsVLMYFFRKNLLRLTG |
| 5616 | SVRsLSLSLFFRKNLLRLTG |
| 5617 | SVYsGDFGNLEVFFRKNLLRLTG |
| 5618 | SVYsPVKKKFFRKNLLRLTG |
| 5619 | SVYsPVKKYFFRKNLLRLTG |
| 5620 | sYIEHIFEIFFRKNLLRLTG |
| 5621 | SYPsPVATSYFFRKNLLRLTG |
| 5622 | sYQKVIELFFFRKNLLRLTG |
| 5623 | TDKYsKKMFFRKNLLRLIG |
| 5624 | TEAsPESMLFFRKNLLRLTG |
| 5625 | THKGEIRGASTPFQFRAssPFFRKNLLRLTG |
| 5626 | TIGEKKEPsDKSVDSFFRKNLLRLTG |
| 5627 | TKDKYMASRGQKAKsMEGFFRKNLLRLTG |
| 5628 | TKsVKALSSLHGDDFFRKNLLRLTG |
| 5629 | TKsVKALSSLHGDDQFFRKNLLRLTG |
| 5630 | TKsVKALSSLHGDDQDFFRKNLLRLTG |
| 5631 | TLAsPSVFKSTFFRKNLLRLTG |
| 5632 | TLAsPSVFKSVFFRKNLLRLTG |
| 5633 | TLLAsPMLKFFRKNLLRLTG |
| 5634 | TLMERTVsLFFRKNLLRLTG |
| 5635 | TLSsPPPGLFFRKNLLRLTG |
| 5636 | TMAsPGKDNYFFRKNLLRLTG |
| 5637 | TMAsPSVFKSTFFRKNLLRLTG |
| 5638 | TMAsPSVFKSVFFRKNLLRLTG |
| 5639 | TMDsPGKDNYFFRKNLLRLTG |
| 5640 | TMEsPGKDNYFFRKNLLRLTG |
| 5641 | TMMsPSQFLFFRKNLLRLTG |
| 5642 | TPAQPQRRsFFFRKNLLRLTG |
| 5643 | TPAQPQRRsLFFRKNLLRLTG |
| 5644 | TPAQPQRRsMFFRKNLLRLTG |
| 5645 | TPAQPQRRsVFFRKNLLRLTG |
| 5646 | TPDPSKFFSQLsSEHGGDVFFRKNLLRLTG |
| 5647 | tPDPSKFFSQLSSEHGGDVQFFRKNLLRLTG |
| 5648 | TPIsPGRASGFFFRKNLLRLTG |
| 5649 | TPIsPGRASGLFFRKNLLRLTG |
| 5650 | TPIsPGRASGMFFRKNLLRLTG |
| 5651 | TPIsPGRASGVFFRKNLLRLTG |
| 5652 | TPMKKHLsLFFRKNLLRLTG |
| 5653 | TPRsPPLGFFFRKNLLRLTG |
| 5654 | TPRsPPLGLFFRKNLLRLTG |
| 5655 | TPRsPPLGLFFFRKNLLRLTG |
| 5656 | TPRsPPLGLIFFRKNLLRLTG |
| 5657 | TPRsPPLGLLFFRKNLLRLTG |
| 5658 | TPRsPPLGLMFFRKNLLRLTG |
| 5659 | TPRsPPLGLVFFRKNLLRLTG |
| 5660 | TPRsPPLGMFFRKNLLRLTG |
| 5661 | TPRsPPLGVFFRKNLLRLTG |
| 5662 | TQSSGKsSVFFRKNLLRLTG |
| 5663 | TRKtPESFLFFRKNLLRLTG |
| 5664 | TRLsPAKIVLFFFRKNLLRLTG |
| 5665 | TRLsPAKIVLKFFRKNLLRLTG |
| 5666 | TRLsPAKIVLRFFRKNLLRLTG |
| 5667 | TRLsPAKIVLYFFRKNLLRLTG |
| 5668 | TSAsPGKDNYFFRKNLLRLTG |
| 5669 | TSDsPGKDNYFFRKNLLRLTG |
| 5670 | TSDtPDYLLKYFFRKNLLRLTG |
| 5671 | TSEsPGKDNYFFRKNLLRLTG |
| 5672 | TSEtPDYLLKYFFRKNLLRLTG |
| 5673 | TTAsPGKDNYFFRKNLLRLTG |
| 5674 | TTDsPGKDNYFFRKNLLRLTG |
| 5675 | TTDtPDYLLKYFFRKNLLRLTG |
| 5676 | TTEsPGKDNYFFRKNLLRLTG |
| 5677 | TTEtPDYLLKYFFRKNLLRLTG |
| 5678 | TTKsVKALSSLHGFFRKNLLRLTG |
| 5679 | TTKsVKALSSLHGDDFFRKNLLRLTG |
| 5680 | TTKsVKALSSLHGDDQFFRKNLLRLTG |
| 5681 | TKKsVKALSSHLGDDQDFFRKNLLRLTG |
| 5682 | TTKsVKALSSLHGDDQDSFFRKNLLRLTG |
| 5683 | TTKsVKALSSLHGDDQDsEDFFRKNLLRLTG |
| 5684 | TTKSVKALSSHGDDQDsEDFFRKNLLRLTG |
| 5685 | TTKsVKALSSLHGDDQDsEDEFFRKNLLRLTG |
| 5686 | TTKSVKALSSHGDDQSsEDEFFRKNLLRLTG |
| 5687 | TVFsPTLPAAFFRKNLLRLTG |
| 5688 | TVMsNSSVIHLFFRKNLLRLTG |
| 5689 | VAKRLsLFFRKNLLRLTG |
| 5690 | VAMPVKKSPRRSsSDEQGLSYSSLKNVFFRKNLLRLTG |
| 5691 | VIDsQELSKVFFRKNLLRLTG |
| 5692 | VLDsPASKKFFRKNLLRLTG |
| 5693 | VLFPEsPARAFFRKNLLRLTG |
| 5694 | VLFRtPLASVFFRKNLLRLTG |
| 5695 | VLFsSPPQMFFRKNLLRLTG |
| 5696 | VLFSsPPQMFFRKNLLRLTG |
| 5697 | VLIENVAsLFFRKNLLRLTG |
| 5698 | VLIGsPKKVFFRKNLLRLTG |
| 5699 | VLIGsPKKYFFRKNLLRLTG |
| 5700 | VLKGsRSSELFFRKNLLRLTG |
| 5701 | VLKGsRSSEVFFRKNLLRLTG |
| 5702 | VLKSRKssVTEEFFRKNLLRLTG |
| 5703 | VLKVMIGsPKFFRKNLLRLTG |
| 5704 | VLKVMIGsPKKFFRKNLLRLTG |
| 5705 | VLKVMIGsPKKKFFRKNLLRLTG |
| 5706 | VLLsPVPELFFRKNLLRLTG |
| 5707 | VLLsPVPEVFFRKNLLRLTG |
| 5708 | VLMK(sPs)PALFFRKNLLRLTG |
| 5709 | VLMK(sPs)PAVFFRKNLLRLTG |
| 5710 | VLQtPPYVKFFRKNLLRLTG |
| 5711 | VLQtPPYVKKFFRKNLLRLTG |
| 5712 | VLQtPPYVKYFFRKNLLRLTG |
| 5713 | VLSDVIPsIFFRKNLLRLTG |
| 5714 | VLSSLtPAKVFFRKNLLRLTG |
| 5715 | VLVVDTPsIFFRKNLLRLTG |
| 5716 | VLYsPQMALFFRKNLLRLTG |
| 5717 | VMFRtPLASVFFRKNLLRLTG |
| 5718 | VMIGsKKVFFRKNLLRLTG |
| 5719 | VMIGsPKKVFFRKNLLRLTG |
| 5720 | VMIGsPKKYFFRKNLLRLTG |
| 5721 | VMKVMIGsPKFFRKNLLRLTG |
| 5722 | VMKVMIGsPKKFFRKNLLRLTG |
| 5723 | VMKVMIGsPKKKFFRKNLLRLTG |
| 5724 | VMKVMIGsPKKYFFRKNLLRLTG |
| 5725 | VMLsPVPELFFRKNLLRLTG |
| 5726 | VMLsPVPEVFFRKNLLRLTG |
| 5727 | VMQtPPYVKFFRKNLLRLTG |
| 5728 | VMQtPPYVKKFFRKNLLRLTG |
| 5729 | VPHHGFEDWsQIRFFRKNLLRLTG |
| 5730 | VPKSGRSSsLFFRKNLLRLTG |
| 5731 | VPKsPAFALFFRKNLLRLTG |
| 5732 | VPLIRKKsLFFRKNLLRLTG |
| 5733 | VPNAPPAYEKLsAEQSPPPYFFRKNLLRLTG |
| 5734 | VPREVLRLsFFFRKNLLRLTG |
| 5735 | VPREVLRLsLFFRKNLLRLTG |
| 5736 | VPREVLRLsMFFRKNLLRLTG |
| 5737 | VPREVLRLsVFFRKNLLRLTG |
| 5738 | VPRPERRsSLFFRKNLLRLTG |
| 5739 | VPRsPKHAHSSSFFFRKNLLRLTG |
| 5740 | VPRsPKHAHSSSLFFRKNLLRLTG |
| 5741 | VPRsPKHAHSSSMFFRKNLLRLTG |
| 5742 | VPRsPKHAHSSSVFFRKNLLRLTG |
| 5743 | VPStPKSSLFFRKNLLRLTG |
| 5744 | VPTsPKSSLFFRKNLLRLTG |
| 5745 | VPVsPGQQLFFRKNLLRLTG |
| 5746 | VRAsKDLAQFFRKNLLRLTG |
| 5747 | VRQsVTSFPDADAFHHQFFRKNLLRLTG |
| 5748 | VSKVMIGsPKKVFFRKNLLRLTG |
| 5749 | VSKVMIGsPKKYFFRKNLLRLTG |
| 5750 | VTQtPPYVKKFFRKNLLRLTG |
| 5751 | VTQtPPYVKYFFRKNLLRLTG |
| 5752 | VVDsPGQEVLFFRKNLLRLTG |
| 5753 | VYTyIQSRFFFRKNLLRLTG |
| 5754 | WTHLsSKEVDPSFFRKNLLRLTG |
| 5755 | WTHLsSKEVDPSTGFFRKNLLRLTG |
| 5756 | YARsVHEEFFFRKNLLRLTG |
| 5757 | YAVPRRGsLFFRKNLLRLTG |
| 5758 | YAYDGKDyIFFRKNLLRLTG |
| 5759 | YEGsPIKVFFRKNLLRLTG |
| 5760 | YEKLsAEQSPPPFFRKNLLRLTG |
| 5761 | YFsPFRPYFFRKNLLRLTG |
| 5762 | yIQSRFFFRKNLLRLTG |
| 5763 | YLAsLEKKLFFRKNLLRLTG |
| 5764 | YLDsGIHSGFFRKNLLRLTG |
| 5765 | YLDsGIHsGAFFRKNLLRLTG |
| 5766 | YLDsGIHSGAFFRKNLLRLTG |
| 5767 | YLDsGIHsGVFFRKNLLRLTG |
| 5768 | YLDsGIHSGVFFRKNLLRLTG |
| 5769 | yLGLDVPVFFRKNLLRLTG |
| 5770 | YLGsISTLVTLFFRKNLLRLTG |
| 5771 | YLIHsPMSLFFRKNLLRLTG |
| 5772 | YLLsPLNTLFFRKNLLRLTG |
| 5773 | YLLsPTKLPSIFFRKNLLRLTG |
| 5774 | YLLsPTKLPSVFFRKNLLRLTG |
| 5775 | yLQSRYYRAFFRKNLLRLTG |
| 5776 | YLQsRYYRAFFRKNLLRLTG |
| 5777 | YLSDsDTEAKLFFRKNLLRLTG |
| 5778 | YMDsGIHsGAFFRKNLLRLTG |
| 5779 | YMDsGIHSGAFFRKNLLRLTG |
| 5780 | YMDsGIHsGVFFRKNLLRLTG |
| 5781 | YMDsGIHSGVFFRKNLLRLTG |
| 5782 | YPDPHsPFAVFFRKNLLRLTG |
| 5783 | YPGGRRsSLFFRKNLLRLTG |
| 5784 | YPLsPAKVNQYFFRKNLLRLTG |
| 5785 | YPLsPTKISEYFFRKNLLRLTG |
| 5786 | YPLsPTKISQYFFRKNLLRLTG |
| 5787 | YPRsEDEVEGVMFFRKNLLRLIG |
| 5788 | YPRsFDEVEGFFFRKNLLRLTG |
| 5789 | YPRsFDEVEGLFFRKNLLRLTG |
| 5790 | YPRsFDEVEGMFFRKNLLRLTG |
| 5791 | YPRsFDEVEGVFFRKNLLRLTG |
| 5792 | YPRsFDEVEGVFFFRKNLLRLTG |
| 5793 | YPRsFDEVEGVLFFRKNLLRLTG |
| 5794 | YPRsFDEVEGVMFFRKNLLRLTG |
| 5795 | YPRsFDEVEGVVFFRKNLLRLTG |
| 5796 | YPSFRRsSLFFRKNLLRLTG |
| 5797 | YPSsPRKALFFRKNLLRLTG |
| 5798 | YPSsPRKFFFRKNLLRLTG |
| 5799 | YPSsPRKLFFRKNLLRLTG |
| 5800 | YPSsPRKMFFRKNLLRLTG |
| 5801 | YPSsPRKVFFRKNLLRLTG |
| 5802 | YPYEFsPVKMFFRKNLLRLTG |
| 5803 | YQLsPTKLPSIFFRKNLLRLTG |
| 5804 | YQLsPTKLPSVFFRKNLLRLTG |
| 5805 | YQRPFsPSAYFFRKNLLRLTG |
| 5806 | YQRsFDEVEGFFFRKNLLRLTG |
| 5807 | YQRsFDEVEGLFFRKNLLRLTG |
| 5808 | YQRsFDEVEGMFFRKNLLRLTG |
| 5809 | YQRsFDEVEGVFFRKNLLRLTG |
| 5810 | YQRsFDEVEGVFFFRKNLLRLTG |
| 5811 | YQRsFDEVEGVLFFRKNLLRLTG |
| 5812 | YQRsFDEVEGVMFFRKNLLRLTG |
| 5813 | YQRsFDEVEGVVFFRKNLLRLTG |
| 5814 | YRYsPQSFLFFRKNLLRLTG |
| 5815 | YTAGtPYKVFFRKNLLRLTG |
| 5816 | YYTAGSSsPTHAKSAHVFFRKNLLRLTG |
| 8809 | RLLsAAENFLFFRKNLLRLTG |

Lowercase s, t, and y indicate phosphorylated serine,
phosphorylated threonine, and phosphorylated tyrosine,
respectively.
Lowercase c indicates that the cysteine is present in a
cysteine—cysteine disulfide bond.
Lowercase m indicates oxidized methionine.
(AcS) indicates an N-terminally acetylated serine.
(sLss) indicates that at least one serine residue in the
amino acid sequence. SLSS is phosphorylated.
(sPs) indicates that at least one serine residue in
the amino acid sequence SPS is phosphorylated.

TABLE 5
Amino acid sequences of
exemplary antigenic polypeptides

| SEQ | |
|---|---|
| ID | |
| NO | Amino Acid Sequence |
| 5817 | (AcS)AARESHPHGVKRSAsPDDDLGFFRKNWLRLTW |
| 5818 | AAEsPSFLFFRKNWLRLTW |
| 5819 | AASNFKsPVKTIRFFRKNWLRLTW |
| 5820 | ADLsPEREVFFRKNWLRLTW |
| 5821 | AEDEIGtPRKFFFRKNWLRLTW |
| 5822 | AEDEIGtPRKYFFRKNWLRLTW |
| 5823 | AEEEIGtPRKFFFRKNWLRLTW |
| 5824 | AEEEIGtPRKWFFRKNWLRLTW |
| 5825 | AEEEIGtPRKYFFRKNWLRLTW |
| 5826 | AENARSAsFFFRKNWLRLTW |
| 5827 | AENsPTRQQFFFRKNWLRLTW |
| 5828 | AENsPTRQQWFFRKNWLRLTW |
| 5829 | AENsPTRQQYFFRKNWLRLTW |
| 5830 | AENsSSRELFFRKNWLRLTW |
| 5831 | AEQGsPRVSYFFRKNWLRLTW |
| 5832 | AESsPTAGKKFFFRKNWLRLTW |
| 5833 | AESsPTAGKKLFFRKNWLRLTW |
| 5834 | AESsPTAGKKWFFRKNWLRLTW |
| 5835 | AESsPTAGKKYFFRKNWLRLTW |
| 5836 | AGDsPGSQFFFRKNWLRLTW |
| 5837 | AILsPAFKVFFRKNWLRLTW |
| 5838 | AIMRsPQMVFFRKNWLRLTW |
| 5839 | AIsDLQQLFFRKNWLRLTW |
| 5840 | AKLsETISFFRKNWLRLTW |
| 5841 | ALAAsPHAVFFRKNWLRLTW |
| 5842 | ALDsGASLLHLFFRKNWLRLTW |
| 5843 | ALDsGASLLHVFFRKNWLRLTW |
| 5844 | ALGNtPPFLFFRKNWLRLTW |
| 5845 | ALGsRESLATIFFRKNWLRLTW |
| 5846 | ALGsRESLATVFFRKNWLRLTW |
| 5847 | ALIHQsLGLFFRKNWLRLTW |
| 5848 | ALIHQsLGVFFRKNWLRLTW |
| 5849 | ALLGSKsPDPYRLFFRKNWLRLTW |
| 5850 | ALLGSKsPDPYRVFFRKNWLRLTW |
| 5851 | ALLsLLKRVFFRKNWLRLTW |
| 5852 | ALMGsPQLVFFRKNWLRLTW |
| 5853 | ALMGsPQLVAAFFRKNWLRLTW |
| 5854 | ALRSsPIMRKFFRKNWLRLTW |
| 5855 | ALRSsPIMRYFFRKNWLRLTW |
| 5856 | ALVsPPALHNAFFRKNWLRLTW |
| 5857 | ALVsPPALHNVFFRKNWLRLTW |
| 5858 | ALYsGVHKKFFRKNWLRLTW |
| 5859 | ALYsGVHKYFFRKNWLRLTW |
| 5860 | ALYsPAQPSLFFRKNWLRLTW |
| 5861 | ALYsPAQPSVFFRKNWLRLTW |
| 5862 | ALYtPQAPKFFRKNWLRLTW |
| 5863 | ALYtPQAPYFFRKNWLRLTW |
| 5864 | AMAAsPHAVFFRKNWLRLTW |
| 5865 | AMDsGASLLHLFFRKNWLRLTW |
| 5866 | AMDsGASLLHVFFRKNWLRLTW |
| 5867 | AMGsRESLATIFFRKNWLRLTW |
| 5868 | AMGsRESLATVFFRKNWLRLTW |
| 5869 | AMLGSKsPDPYRLFFRKNWLRLTW |
| 5870 | AMLGSKsPDPYRVFFRKNWLRLTW |
| 5871 | AMPGsPVEVFFRKNWLRLTW |
| 5872 | AMRSsPIMRKFFRKNWLRLTW |
| 5873 | AMVsPPALHNAFFRKNWLRLTW |
| 5874 | AMVsPPALHNVFFRKNWLRLTW |
| 5875 | AMYsGVHKKFFRKNWLRLTW |
| 5876 | APDsPRAFLFFRKNWLRLTW |
| 5877 | APLARASsLFFRKNWLRLTW |
| 5878 | APPAYEKLsFFRKNWLRLTW |
| 5879 | APPAYEKLsAEQFFRKNWLRLTW |
| 5880 | APPAYEKLsAEQSPPFFRKNWLRLTW |
| 5881 | APPAYEKLsAEQSPPPFFRKNWLRLTW |
| 5882 | APPAYEKLsAEQSPPPYFFRKNWLRLTW |
| 5883 | APPPLVPAPRPSsPPRGPGPARADRFFRKNWLRLTW |
| 5884 | APRAPsASPLALFFRKNWLRLTW |
| 5885 | APRDRRAVsFFFRKNWLRLTW |
| 5886 | APRKGsFSALFFRKNWLRLTW |
| 5887 | APRKGsFSALFFFRKNWLRLTW |
| 5888 | APRKGsFSALLFFRKNWLRLTW |
| 5889 | APRKGsFSALMFFRKNWLRLTW |
| 5890 | APRKGsFSALVFFRKNWLRLTW |
| 5891 | APRNGsGVALFFRKNWLRLTW |
| 5892 | APRRYsSSFFFRKNWLRLTW |
| 5893 | APRRYsSSLFFRKNWLRLTW |
| 5894 | APRRYsSSMFFRKNWLRLTW |
| 5895 | APRRYsSSVFFRKNWLRLTW |
| 5896 | APRsPPPSRFFFRKNWLRLTW |
| 5897 | APRsPPPSRLFFRKNWLRLTW |
| 5898 | APRsPPPSRMFFRKNWLRLTW |
| 5899 | APRsPPPSRPFFRKNWLRLTW |
| 5900 | APRsPPPSRVFFRKNWLRLTW |
| 5901 | APSLFHLNtLFFRKNWLRLTW |
| 5902 | APSSARAsPLLFFRKNWLRLTW |
| 5903 | APSTYAHLsPAKFFRKNWLRLTW |
| 5904 | APSTYAHLsPAKTPPPPFFRKNWLRLTW |
| 5905 | APSVRsLSLFFRKNWLRLTW |
| 5906 | APSVRSLsLFFRKNWLRLTW |
| 5907 | ARFsPDDKYSFFFRKNWLRLTW |
| 5908 | ARFsPDDKYSKFFRKNWLRLTW |
| 5909 | ARFsPDDKYSLFFRKNWLRLTW |
| 5910 | ARFsPDDKYSMFFRKNWLRLTW |
| 5911 | ARFsPDDKYSRFFRKNWLRLTW |
| 5912 | ARFsPDDKYSYFFRKNWLRLTW |
| 5913 | ASDEIGtPRKFFFRKNWLRLTW |
| 5914 | ASDEIGtPRKYFFRKNWLRLTW |
| 5915 | ASEEIGtPRKFFFRKNWLRLTW |
| 5916 | ASEEIGtPRKYFFRKNWLRLTW |
| 5917 | AsISRLsGEQVDGKGFFRKNWLRLTW |
| 5918 | AsISRLSGEQVDGKGFFRKNWLRLTW |
| 5919 | ASISRLsGEQVDGKGFFRKNWLRLTW |
| 5920 | AsIsRLSGEQVDGKGQFFRKNWLRLTW |
| 5921 | AsISRLSGEQVDKGKGFFRKNWLRLTW |
| 5922 | ASKAsPTLDFTERFFRKNWLRLTW |
| 5923 | ASKMTQPQSKSAFPLSRKNKGsGsLDGFFRKNWLRLTW |
| 5924 | AsLGFVFFFRKNWLRLTW |
| 5925 | AsPTIEAQGTSPAHDNFFRKNWLRLTW |
| 5926 | AsPTIEAQGTSPAHDNIFFRKNWLRLTW |
| 5927 | AsPTIEAQGTSPAHDNIAFFRKNWLRLTW |
| 5928 | AtAGPRLGFFFRKNWLRLTW |
| 5929 | AtAGPRLGWFFRKNWLRLTW |
| 5930 | AtAGPRLGYFFRKNWLRLTW |
| 5931 | ATDEIGtPRKFFFRKNWLRLTW |
| 5932 | ATDEIGtPRKYFFRKNWLRLTW |
| 5933 | ATEEIGtPRKFFFRKNWLRLTW |
| 5934 | ATEEIGtPRKYFFRKNWLRLTW |
| 5935 | ATWsGSEFEVFFRKNWLRLTW |
| 5936 | ATYtPQAPKFFRKNWLRLTW |
| 5937 | ATYtPQAPKYFFRKNWLRLTW |
| 5938 | AVIHQsLGLFFRKNWLRLTW |
| 5939 | AVIHQsLGVFFRKNWLRLTW |
| 5940 | AVRPTRLsLFFRKNWLRLTW |
| 5941 | AVVsPPALHNAFFRKNWLRLTW |
| 5942 | AVVsPPALHNVFFRKNWLRLTW |
| 5943 | AYEKLsAEQSPPFFRKNWLRLTW |
| 5944 | DAKKsPLALFFRKNWLRLTW |
| 5945 | DDDWTHLsSKEVDPFFRKNWLRLTW |
| 5946 | DDDWTHLsSKEVDPSFFRKNWLRLTW |
| 5947 | DDDWTHLsSKEVDPSTFFRKNWLRLTW |
| 5948 | DDDWTHLsSKEVDPSTGFFRKNWLRLTW |
| 5949 | DDWTHLsSKEVDPSFFRKNWLRLTW |
| 5950 | DEFERIKtFFFRKNWLRLTW |
| 5951 | DEFERIKtWFFRKNWLRLTW |
| 5952 | DEFERIKtYFFRKNWLRLTW |
| 5953 | DEISHRAsFFFRKNWLRLTW |
| 5954 | DEISHRAsWFFRKNWLRLTW |
| 5955 | DEISHRAsYFFRKNWLRLTW |
| 5956 | DERLRINsFFFRKNWLRLTW |
| 5957 | DERLRINsLFFRKNWLRLTW |
| 5958 | DERLRINsWFFRKNWLRLTW |
| 5959 | DERLRINsYFFRKNWLRLTW |
| 5960 | DKLsVIAEDSESGKQFFRKNWLRLTW |
| 5961 | DKLsVIAEDSESGKQNFFRKNWLRLTW |
| 5962 | DKLsVIAEDSESGKQNPFFRKNWLRLTW |
| 5963 | DKLsVIAEDSESGKQNPGFFRKNWLRLTW |
| 5964 | DKLsVIAEDSESGKQNPGDSFFRKNWLRLTW |
| 5965 | DLKRRsmSIFFRKNWLRLTW |
| 5966 | DLKRRsMSIFFRKNWLRLTW |
| 5967 | DLKSSKAsLFFRKNWLRLTW |
| 5968 | DLRtVEKELFFRKNWLRLTW |
| 5969 | DLsEEKFLFFRKNWLRLTW |
| 5970 | DLsEEKFVFFRKNWLRLTW |
| 5971 | DLVPLsPLKKFFRKNWLRLTW |
| 5972 | DLWKItKVMDFFRKNWLRLTW |
| 5973 | DMVPLsPLKKFFRKNWLRLTW |
| 5974 | DPTRRFFKVtPPPGSGPQFFRKNWLRLTW |
| 5975 | DQFERIKtLFFRKNWLRLTW |
| 5976 | DQISHRAsLFFRKNWLRLTW |
| 5977 | DSDPLsPLKYFFRKNWLRLTW |
| 5978 | DSEPLsPLKYFFRKNWLRLTW |
| 5979 | DSsEEKFLFFRKNWLRLTW |
| 5980 | DSsEEKFVFFRKNWLRLTW |
| 5981 | DSVPLsPLKYFFRKNWLRLTW |
| 5982 | DTDPLsPLKYFFRKNWLRLTW |
| 5983 | DTEPLsPLKYFFRKNWLRLTW |
| 5984 | DTVPLsPLKYFFRKNWLRLTW |
| 5985 | DWTHLsSKEVDPSFFRKNWLRLTW |
| 5986 | DWTHLsSKEVDPSTGFFRKNWLRLTW |
| 5987 | EEGsPTMVEKGLEPGVFTLFFRKNWLRLTW |
| 5988 | EELsPTAKFFFRKNWLRLTW |
| 5989 | EELsPTKAFFFRKNWLRLTW |
| 5990 | EEMPENALPsDEDDKDPNDPYRALFFRKNWLRLTW |
| 5991 | EERRsPPAPFFRKNWLRLTW |
| 5992 | EEsSDDGKKFFFRKNWLRLTW |
| 5993 | EESsDDGKKFFFRKNWLRLTW |
| 5994 | EEsSDDGKKWFFRKNWLRLTW |
| 5995 | EESsDDGKKWFFRKNWLRLTW |
| 5996 | EEsSDDGKKYFFRKNWLRLTW |
| 5997 | EESsDDGKKYFFRKNWLRLTW |
| 5998 | EGEEPTVYsDEEEPKDESARKNDFFRKNWLRLTW |
| 5999 | EGsPTMVEKGLEPGVFTLFFRKNWLRLTW |
| 6000 | ELFSsPPAVFFRKNWLRLTW |
| 6001 | ELKKsPTSLKFFRKNWLRLTW |
| 6002 | ELKKsPTSLYFFRKNWLRLTW |
| 6003 | ELLMPHRIsSHFFFRKNWLRLTW |
| 6004 | ELLMPHRIsSHFLFFRKNWLRLTW |
| 6005 | ELRISGsVQLFFRKNWLRLTW |
| 6006 | EMKKsPTSLKFFRKNWLRLTW |
| 6007 | EPAsPAAsISRLsGEQVDGKGFFRKNWLRLTW |
| 6008 | EPAsPAAsISRLSGEQVDGKGFFRKNWLRLTW |
| 6009 | EPKRRsARFFFRKNWLRLTW |
| 6010 | EPKRRsARLFFRKNWLRLTW |
| 6011 | EPKRRsARMFFRKNWLRLTW |
| 6012 | EPKRRsARVFFRKNWLRLTW |
| 6013 | EPRsPSHSFFFRKNWLRLTW |
| 6014 | EPRsPSHSLFFRKNWLRLTW |
| 6015 | EPRsPSHSMFFRKNWLRLTW |
| 6016 | EPRsPSHSVFFRKNWLRLTW |
| 6017 | ERsPLLSQETAGQKPFFRKNWLRLTW |
| 6018 | ERsPLLSQETAGQKPLFFRKNWLRLTW |
| 6019 | ESDsLPRYFFRKNWLRLTW |
| 6020 | ESEsLPRYFFRKNWLRLTW |
| 6021 | ESsVRSQEDQLSRFFRKNWLRLTW |
| 6022 | ESsVRSQEDQLSRRFFRKNWLRLTW |
| 6023 | ETDsLPRYFFRKNWLRLTW |
| 6024 | ETEsLPRYFFRKNWLRLTW |
| 6025 | FDKHTLGDsDNESFFRKNWLRLTW |
| 6026 | FEDDDsNEKLFFRKNWLRLTW |
| 6027 | FIEsPSKLFFRKNWLRLTW |
| 6028 | FIEsPSKYFFRKNWLRLTW |
| 6029 | FIGsPTTPAGLFFRKNWLRLTW |
| 6030 | FKMPQEKsPGYSFFRKNWLRLTW |
| 6031 | FKsPVKTIRFFRKNWLRLTW |
| 6032 | FKtQPVTFFFRKNWLRLTW |
| 6033 | FLDNsFEKVFFRKNWLRLTW |
| 6034 | FLDRPPtPLFIFFRKNWLRLTW |
| 6035 | FLDsLRDLIFFRKNWLRLTW |
| 6036 | FLDtPIAKVFFRKNWLRLTW |
| 6037 | FLFDKPVsPLLLFFRKNWLRLTW |
| 6038 | FLGVRPKsAFFRKNWLRLTW |
| 6039 | FLIIRtVLQLFFRKNWLRLTW |
| 6040 | FLITGGGKGsGFSLFFRKNWLRLTW |
| 6041 | FLLsQNFDDEFFRKNWLRLTW |
| 6042 | FLYsGKETKFFRKNWLRLTW |
| 6043 | FLYsGKETYFFRKNWLRLTW |
| 6044 | FPHsLLSVFFFRKNWLRLTW |
| 6045 | FPHsLLSVIFFRKNWLRLTW |
| 6046 | FPHsLLSVLFFRKNWLRLTW |
| 6047 | FPHsLLSVMFFRKNWLRLTW |
| 6048 | FPHsLLSVVFFRKNWLRLTW |
| 6049 | FPIsPVRFFFRKNWLRLTW |
| 6050 | FPIsPVRLFFRKNWLRLTW |
| 6051 | FPIsPVRMFFRKNWLRLTW |
| 6052 | FPIsPVRVFFRKNWLRLTW |
| 6053 | FPLDsPKTLVLFFRKNWLRLTW |
| 6054 | FPRRHsVTLFFRKNWLRLTW |
| 6055 | FPRsPTKSSFFFRKNWLRLTW |
| 6056 | FPRsPTKSSLFFRKNWLRLTW |
| 6057 | FPRsPTKSSLDFFFRKNWLRLTW |
| 6058 | FPRsPTKSSLDLFFRKNWLRLTW |
| 6059 | FPRsPTKSSLDMFFRKNWLRLTW |
| 6060 | FPRsPTKSSLDVFFRKNWLRLTW |
| 6061 | FPRsPTKSSMFFRKNWLRLTW |
| 6062 | FPRsPTKSSVFFRKNWLRLTW |
| 6063 | FRFsGRTEYFFRKNWLRLTW |
| 6064 | FRGRYRsPYFFRKNWLRLTW |
| 6065 | FRKsMVEHYFFRKNWLRLTW |
| 6066 | FRRsPIKSSLDYFFRKNWLRLTW |
| 6067 | FRRsPTKSSFFFRKNWLRLTW |
| 6068 | FRRsPTKSSLFFRKNWLRLTW |
| 6069 | FRRsPTKSSLDFFRKNWLRLTW |
| 6070 | FRRsPTKSSLDFFFRKNWLRLTW |
| 6071 | FRRsPTKSSLDLFFRKNWLRLTW |
| 6072 | FRRsPTKSSLDMFFRKNWLRLTW |
| 6073 | FRRsPTKSSLDVFFRKNWLRLTW |
| 6074 | FRRsPTKSSLDYFFRKNWLRLTW |
| 6075 | FRRsPTKSSMFFRKNWLRLTW |
| 6076 | FRRsPTKSSVFFRKNWLRLTW |
| 6077 | FRsPTKSSLDFFFRKNWLRLTW |
| 6078 | FRsPTKSSLDLFFRKNWLRLTW |
| 6079 | FRsPTKSSLDMFFRKNWLRLTW |
| 6080 | FRsPTKSSLDVFFRKNWLRLTW |
| 6081 | FRYsGKTEFFFRKNWLRLTW |
| 6082 | FRYsGKTEKFFRKNWLRLTW |
| 6083 | FRYsGKTELFFRKNWLRLTW |
| 6084 | FRYsGKTEMFFRKNWLRLTW |
| 6085 | FRYsGKTERFFRKNWLRLTW |
| 6086 | FRYsGKTEYFFRKNWLRLTW |
| 6087 | FSDsHEGFSYFFRKNWLRLTW |
| 6088 | FSEsHEGFSYFFRKNWLRLTW |
| 6089 | FSEsPSKLFFRKNWLRLTW |
| 6090 | FSEsPSKYFFRKNWLRLTW |
| 6091 | FSIsPVRFFFRKNWLRLTW |
| 6092 | FSIsPVRLFFRKNWLRLTW |
| 6093 | FSIsPVRMFFRKNWLRLTW |
| 6094 | FSIsPVRVFFRKNWLRLTW |
| 6095 | FSsSHEGFSYFFRKNWLRLTW |
| 6096 | FSSsHEGFSYFFRKNWLRLTW |
| 6097 | FTDsHEGFSYFFRKNWLRLTW |
| 6098 | FTEsHEGFSYFFRKNWLRLTW |
| 6099 | FTEsPSKLFFRKNWLRLTW |
| 6100 | FTEsPSKYFFRKNWLRLTW |
| 6101 | FTKsPYQEFFFRKNWLRLTW |
| 6102 | FTsSHEGFSYFFRKNWLRLTW |
| 6103 | FVSKVMIGsPKKVFFRKNWLRLTW |
| 6104 | GALsPSLLHSLFFRKNWLRLTW |
| 6105 | GAQPGRHsFFFRKNWLRLTW |
| 6106 | GAQPGRHsLFFRKNWLRLTW |
| 6107 | GAQPGRHsVFFRKNWLRLTW |
| 6108 | GDDDWTHLsSKEVDFFRKNWLRLTW |
| 6109 | GDDDWTHLsSKEVDPFFRKNWLRLTW |
| 6110 | GDDDWTHLsSKEVDPSFFRKNWLRLTW |
| 6111 | GDDDWTHLsSKEVDPSTFFRKNWLRLTW |
| 6112 | GDDDWTHLsSKEVDPSTGFFRKNWLRLTW |
| 6113 | GEAsPSHIIFFRKNWLRLTW |
| 6114 | GEEsSDDGKKFFFRKNWLRLTW |
| 6115 | GEEsSDDGKKWFFRKNWLRLTW |
| 6116 | GEEsSDDGKKYFFRKNWLRLTW |
| 6117 | GEEsSDIDGKKFFFRKNWLRLTW |
| 6118 | GEIsPQREVFFRKNWLRLTW |
| 6119 | GERsPLLSQETAGQKPFFRKNWLRLTW |
| 6120 | GERsPLLSQETAGQKPLFFRKNWLRLTW |
| 6121 | GETsPRTKIFFRKNWLRLTW |
| 6122 | GGDDDWTHLsSKEVDPSFFRKNWLRLTW |
| 6123 | GGDDDWTHLsSKEVDPSTGFFRKNWLRLTW |
| 6124 | GGSFGGRSSGsPFFRKNWLRLTW |
| 6125 | GGSFGGRSSGsVFFRKNWLRLTW |
| 6126 | GIDsPSSSVFFRKNWLRLTW |
| 6127 | GIMsPLAKKFFRKNWLRLTW |
| 6128 | GLAPtPPSMFFRKNWLRLTW |
| 6129 | GLDsGFHSVFFRKNWLRLTW |
| 6130 | GLDsLDQVEIFFRKNWLRLTW |
| 6131 | GLGELLRsLFFRKNWLRLTW |
| 6132 | GLIRSRsFIFKFFRKNWLRLTW |
| 6133 | GLIRSRsFIFYFFRKNWLRLTW |
| 6134 | GLIsPELRHLFFRKNWLRLTW |
| 6135 | GLIsPNVQLFFRKNWLRLTW |
| 6136 | GLIsPVWGAFFRKNWLRLTW |
| 6137 | GLItPGGFSSVFFRKNWLRLTW |
| 6138 | GLLDsPTSIFFRKNWLRLTW |
| 6139 | GLLGsPARLFFRKNWLRLTW |
| 6140 | GLLGsPVRAFFRKNWLRLTW |
| 6141 | GLLGsPVRVFFRKNWLRLTW |
| 6142 | GLLsPARLYAIFFRKNWLRLTW |
| 6143 | GLLsPARLYAVFFRKNWLRLTW |
| 6144 | GLLsPRFVDVFFRKNWLRLTW |
| 6145 | GLLsPRHSLFFRKNWLRLTW |
| 6146 | GLSFGGRSSGsPFFRKNWLRLTW |
| 6147 | GLSFGGRSSGsVFFRKNWLRLTW |
| 6148 | GMLGsPVRVFFRKNWLRLTW |
| 6149 | GMLsPARLYAIFFRKNWLRLTW |
| 6150 | GMLsPARLYAVFFRKNWLRLTW |
| 6151 | GMLsPGKSIEVFFRKNWLRLTW |
| 6152 | GPKPLFRRMsSFFRKNWLRLTW |
| 6153 | GPKPLFRRMsSLFFRKNWLRLTW |
| 6154 | GPKPLFRRMsSLVFFRKNWLRLTW |
| 6155 | GPKPLFRRMsSLVGFFRKNWLRLTW |
| 6156 | GPKPLFRRMsSLVGPFFRKNWLRLTW |
| 6157 | GPKPLFRRMsSLVGPTFFRKNWLRLTW |
| 6158 | GPKPLFRRMsSLVGPTQFFRKNWLRLTW |
| 6159 | GPKPLFRRMsSLVGPTQSFFRKNWLRLTW |
| 6160 | GPPYQRRGsLFFRKNWLRLTW |
| 6161 | GPQPGRHsFFFRKNWLRLTW |
| 6162 | GPQPGRHsLFFRKNWLRLTW |
| 6163 | GPQPGRHsVFFRKNWLRLTW |
| 6164 | GPRPGsPSAFFFRKNWLRLTW |
| 6165 | GPRPGsPSALFFRKNWLRLTW |
| 6166 | GPRPGsPSAMFFRKNWLRLTW |
| 6167 | GPRPGsPSAVFFRKNWLRLTW |
| 6168 | GPRSAsLLFFRKNWLRLTW |
| 6169 | GPRsASLLSFFFRKNWLRLTW |
| 6170 | GPRSAsLLsFFFRKNWLRLTW |
| 6171 | GPRSASLLsFFFRKNWLRLTW |
| 6172 | GPRsAsLLSLFFRKNWLRLTW |
| 6173 | GPRsASLLSLFFRKNWLRLTW |
| 6174 | GPRSAsLLsLFFRKNWLRLTW |
| 6175 | GPRSAsLLSLFFRKNWLRLTW |
| 6176 | GPRSASLLsLFFRKNWLRLTW |
| 6177 | GPRsASLLSMFFRKNWLRLTW |
| 6178 | GPRSAsLLsMFFRKNWLRLTW |
| 6179 | GPRSASLLSMFFRKNWLRLTW |
| 6180 | GPRsASLLSVFFRKNWLRLTW |
| 6181 | GPRSAsLLsVFFRKNWLRLTW |
| 6182 | GPRSASLLSVFFRKNWLRLTW |
| 6183 | GPRsPKAPPFFRKNWLRLTW |
| 6184 | GPRsPPVTLFFRKNWLRLTW |
| 6185 | GQLsPGVQFFFRKNWLRLTW |
| 6186 | GRKsPPPSFFFRKNWLRLTW |
| 6187 | GRKsPPPSKFFRKNWLRLTW |
| 6188 | GRKsPPPSLFFRKNWLRLTW |
| 6189 | GRKsPPPSMFFRKNWLRLTW |
| 6190 | GRKsPPPSRFFRKNWLRLTW |
| 6191 | GRKsPPPSYFFRKNWLRLTW |
| 6192 | GRLGsPHRFFFRKNWLRLTW |
| 6193 | GRLGsPHRKFFRKNWLRLTW |
| 6194 | GRLGsPHRLFFRKNWLRLTW |
| 6195 | GRLGsPHRMFFRKNWLRLTW |
| 6196 | GRLGsPHRRFFRKNWLRLTW |
| 6197 | GRLGsPHRYFFRKNWLRLTW |
| 6198 | GRLsPAYSLFFRKNWLRLTW |
| 6199 | GRLsPKASQVFFFRKNWLRLTW |
| 6200 | GRLsPKASQVKFFRKNWLRLTW |
| 6201 | GRLsPKASQVLFFRKNWLRLTW |
| 6202 | GRLsPKASQVMFFRKNWLRLTW |
| 6203 | GRLsPKASQVRFFRKNWLRLTW |
| 6204 | GRLsPKASQVYFFRKNWLRLTW |
| 6205 | GRLsPVPVPFFFRKNWLRLTW |
| 6206 | GRLsPVPVPKFFRKNWLRLTW |
| 6207 | GRLsPVPVPLFFRKNWLRLTW |
| 6208 | GRLsPVPVPMFFRKNWLRLTW |
| 6209 | GRLsPVPVPRFFRKNWLRLTW |
| 6210 | GRLsPVPVPYFFRKNWLRLTW |
| 6211 | GRQsPSFKLFFRKNWLRLTW |
| 6212 | GRsSPPPGYFFRKNWLRLTW |
| 6213 | GRSsTASLVKFFFRKNWLRLTW |
| 6214 | GRSsTASLVKKFFRKNWLRLTW |
| 6215 | GRSsTASLVKKKFFRKNWLRLTW |
| 6216 | GRSsTASLVKLFFRKNWLRLTW |
| 6217 | GRSSTASLVKMFFRKNWLRLTW |
| 6218 | GRSSTASLVKRFFRKNWLRLTW |
| 6219 | GRSSTASLVKYFFRKNWLRLTW |
| 6220 | GRtGLPDLFFRKNWLRLTW |
| 6221 | GSALGGGGAGLSGRASGGAQsPLRYLHVFFRKNWLRLTW |
| 6222 | GSDsSDDGKKYFFRKNWLRLTW |
| 6223 | GSEsSDDGKKYFFRKNWLRLTW |
| 6224 | GsPHYFSPFFFRKNWLRLTW |
| 6225 | GsPHYFSPFRPYFFRKNWLRLTW |
| 6226 | GsPTMVEKGLEPGVFTLFFRKNWLRLTW |
| 6227 | GsQLAVMMYLFFRKNWLRLTW |
| 6228 | GTDsSDDGKKYFFRKNWLRLTW |
| 6229 | GTEsSDDGKKYFFRKNWLRLTW |
| 6230 | GTIRSRsFIFKFFRKNWLRLTW |
| 6231 | GTIRSRsFIFYFFRKNWLRLTW |
| 6232 | GtLPKYFFRKNWLRLTW |
| 6233 | GtLRRSDSQQAVKFFRKNWLRLTW |
| 6234 | GtLRRSDSQQAVKSFFRKNWLRLTW |
| 6235 | GtLRRSDSQQAVKSPPFFRKNWLRLTW |
| 6236 | GVAsPTITVFFRKNWLRLTW |
| 6237 | GVVsPTFELFFRKNWLRLTW |
| 6238 | HEKKAYsFFFRKNWLRLTW |
| 6239 | HKGEIRGASTPFQFRAsSPFFRKNWLRLTW |
| 6240 | HLHsPQHKLFFRKNWLRLTW |
| 6241 | HPKRSVsLFFRKNWLRLTW |
| 6242 | HPRsPNVLFFRKNWLRLTW |
| 6243 | HPRsPNVLSFFFRKNWLRLTW |
| 6244 | HPRsPNVLSLFFRKNWLRLTW |
| 6245 | HPRsPNVLSMFFRKNWLRLTW |
| 6246 | HPRsPNVLSVFFRKNWLRLTW |
| 6247 | HPRsPTPTFFFRKNWLRLTW |
| 6248 | HPRSPtPTFFFRKNWLRLTW |
| 6249 | HPRsPTPTLFFRKNWLRLTW |
| 6250 | HPRSPtPTLFFRKNWLRLTW |
| 6251 | HPRsPTPTMFFRKNWLRLTW |
| 6252 | HPRSPtPTMFFRKNWLRLTW |
| 6253 | HPRSPtPTVFFRKNWLRLTW |
| 6254 | HPsSPTPTVFFRKNWLRLTW |
| 6255 | HRLsPVKGEFFFRKNWLRLTW |
| 6256 | HRLsPVKGEKFFRKNWLRLTW |
| 6257 | HRLsPVKGERFFRKNWLRLTW |
| 6258 | HRLsPVKGEYFFRKNWLRLTW |
| 6259 | HRNsMKVFLFFRKNWLRLTW |
| 6260 | HRNsNPVIAEFFFRKNWLRLTW |
| 6261 | HRNsNPVIAEKFFRKNWLRLTW |
| 6262 | HRNsNPVIAELFFRKNWLRLTW |
| 6263 | HRNsNPVIAERFFRKNWLRLTW |
| 6264 | HRNsNPVIAEYFFRKNWLRLTW |
| 6265 | HRYsTPHAFFFRKNWLRLTW |
| 6266 | HTAsPTGMMKFFRKNWLRLTW |
| 6267 | HVYtPSTTKFFRKNWLRLTW |
| 6268 | IEKIyIMKADTVIVGFFRKNWLRLTW |
| 6269 | IIEtPHKEIFFRKNWLRLTW |
| 6270 | IIEtPHKEYFFRKNWLRLTW |
| 6271 | IISsPLKGYFFRKNWLRLTW |
| 6272 | IISsPLTGKFFRKNWLRLTW |
| 6273 | ILDRtPEKLFFRKNWLRLTW |
| 6274 | ILDRtPEKVFFRKNWLRLTW |
| 6275 | ILDsGIYRIFFRKNWLRLTW |
| 6276 | ILDsGIYRVFFRKNWLRLTW |
| 6277 | ILKPRRsLFFRKNWLRLTW |
| 6278 | ILKsPEIQRAFFRKNWLRLTW |
| 6279 | ILKsPEIQRVFFRKNWLRLTW |
| 6280 | ILQtPQFQMFFRKNWLRLTW |
| 6281 | ILQVsIPSLFFRKNWLRLTW |
| 6282 | IMDRtPEKLFFRKNWLRLTW |
| 6283 | IMDRtPEKVFFRKNWLRLTW |
| 6284 | IMDsGIYRIFFRKNWLRLTW |
| 6285 | IMDsGIYRVFFRKNWLRLTW |
| 6286 | IMKsPEIQRAFFRKNWLRLTW |
| 6287 | IMKsPEIQRVFFRKNWLRLTW |
| 6288 | INKERRSsLFFRKNWLRLTW |
| 6289 | IPVgSSHNSLFFRKNWLRLTW |
| 6290 | IQFsPPFPGAFFRKNWLRLTW |
| 6291 | ISDGtLKYFFRKNWLRLTW |
| 6292 | ISDGtPLKYFFRKNWLRLTW |
| 6293 | ISDSAHtDYFFRKNWLRLTW |
| 6294 | ISDsMHSLYFFRKNWLRLTW |
| 6295 | ISDtPHKEIFFRKNWLRLTW |
| 6296 | ISDtPHKEYFFRKNWLRLTW |
| 6297 | ISEGtLKYFFRKNWLRLTW |
| 6298 | ISEGtPLKYFFRKNWLRLTW |
| 6299 | ISESAHtDYFFRKNWLRLTW |
| 6300 | ISEsMHSLYFFRKNWLRLTW |
| 6301 | ISEtPHKEIFFRKNWLRLTW |
| 6302 | ISEtPHKEYFFRKNWLRLTW |
| 6303 | ISFSAHtDYFFRKNWLRLTW |
| 6304 | ISSsMHSLYFFRKNWLRLTW |
| 6305 | IStDRDPLFFRKNWLRLTW |
| 6306 | IStDRDPYFFRKNWLRLTW |
| 6307 | ITDGtLKYFFRKNWLRLTW |
| 6308 | ITDGtPLKYFFRKNWLRLTW |
| 6309 | ITDSAHtDYFFRKNWLRLTW |
| 6310 | ITDsMHSLYFFRKNWLRLTW |
| 6311 | ITDtPHKEIFFRKNWLRLTW |
| 6312 | ITDtPHKEYFFRKNWLRLTW |
| 6313 | ITEGtLKYFFRKNWLRLTW |
| 6314 | ITEGtPLKYFFRKNWLRLTW |
| 6315 | ITESAHtDYFFRKNWLRLTW |
| 6316 | ITEsMHSLYFFRKNWLRLTW |
| 6317 | ITEtPHKEIFFRKNWLRLTW |
| 6318 | ITEtPHKEYFFRKNWLRLTW |
| 6319 | ITQGtLKYFFRKNWLRLTW |
| 6320 | ITQGtPLKKFFRKNWLRLTW |
| 6321 | ITQGtPLKYFFRKNWLRLTW |
| 6322 | ITtDRDPLFFRKNWLRLTW |
| 6323 | ITtDRDPYFFRKNWLRLTW |
| 6324 | IVLsDSEVIQLFFRKNWLRLTW |
| 6325 | IVRyHQLFFRKNWLRLTW |
| 6326 | IVtDRDPLFFRKNWLRLTW |
| 6327 | IVtDRDPYFFRKNWLRLTW |
| 6328 | IYQyIQSRFFFRKNWLRLTW |
| 6329 | KAFsPVRFFRKNWLRLTW |
| 6330 | KAFsPVRSVFFRKNWLRLTW |
| 6331 | KAKsPAPGLFFRKNWLRLTW |
| 6332 | KAKsPAPGVFFRKNWLRLTW |
| 6333 | KARsPGRAFFFRKNWLRLTW |
| 6334 | KARsPGRALFFRKNWLRLTW |
| 6335 | KARsPGRAMFFRKNWLRLTW |
| 6336 | KARsPGRAVFFRKNWLRLTW |
| 6337 | KASPKRLsLFFRKNWLRLTW |
| 6338 | KAVsLFLcYFFRKNWLRLTW |
| 6339 | KAVsLFLCYFFRKNWLRLTW |
| 6340 | KEGEEPTVYsDEEEPKDESARKNDFFRKNWLRLTW |
| 6341 | KEKsPFRETFFRKNWLRLTW |
| 6342 | KELARQIsFFFRKNWLRLTW |
| 6343 | KEMsPTRQFFFRKNWLRLTW |
| 6344 | KEmsPTRQLFFRKNWLRLTW |
| 6345 | KEMSPTRQLFFRKNWLRLTW |
| 6346 | KEMsPTRQWFFRKNWLRLTW |
| 6347 | KEMsPTRQYFFRKNWLRLTW |
| 6348 | KESsPLSSRKIFFRKNWLRLTW |
| 6349 | KFRPPPLsLFFRKNWLRLTW |
| 6350 | KGIsSSSLKEKFFRKNWLRLTW |
| 6351 | KIAsEIAQLFFRKNWLRLTW |
| 6352 | KIDIVsSQKVFFRKNWLRLTW |
| 6353 | KIDsPTKVKKFFRKNWLRLTW |
| 6354 | KIEKIyIMKADTVIVGFFRKNWLRLTW |
| 6355 | KIEsLENLYLFFRKNWLRLTW |
| 6356 | KIFsGVFVKFFRKNWLRLTW |
| 6357 | KIFsGVFVKVFFRKNWLRLTW |
| 6358 | KIFsKQQGKFFRKNWLRLTW |
| 6359 | KIFsKQQGYFFRKNWLRLTW |
| 6360 | KIGsIIFQVFFRKNWLRLTW |
| 6361 | KIKsFEWFFFRKNWLRLTW |
| 6362 | KIRSsPREAKFFRKNWLRLTW |
| 6363 | KIRSsPREAYFFRKNWLRLTW |
| 6364 | KIRTsPTFRFFRKNWLRLTW |
| 6365 | KIRTsPTFYFFRKNWLRLTW |
| 6366 | KLAsLEREASVFFRKNWLRLTW |
| 6367 | KLAsLLHQVFFRKNWLRLTW |
| 6368 | KLAsPEKLAGLFFRKNWLRLTW |
| 6369 | KLAsPELERLFFRKNWLRLTW |
| 6370 | KLAsPELERVFFRKNWLRLTW |
| 6371 | KLDIVsSQKVFFRKNWLRLTW |
| 6372 | KLDsFLDMQVFFRKNWLRLTW |
| 6373 | KLDsPRVTVFFRKNWLRLTW |
| 6374 | KLDsPTKVKKFFRKNWLRLTW |
| 6375 | KLDsPTKVKYFFRKNWLRLTW |
| 6376 | KLFPDtPLALFFRKNWLRLTW |
| 6377 | KLFPDtPLAVFFRKNWLRLTW |
| 6378 | KLFsGTVRKFFRKNWLRLTW |
| 6379 | KLFsGVFVKVFFRKNWLRLTW |
| 6380 | KLFsKQQGKFFRKNWLRLTW |
| 6381 | KLFsKQQGYFFRKNWLRLTW |
| 6382 | KLFsPAHKKFFRKNWLRLTW |
| 6383 | KLFsPAHKYFFRKNWLRLTW |
| 6384 | KLFsPSKEAELFFRKNWLRLTW |
| 6385 | KLFsPSKEAEVFFRKNWLRLTW |
| 6386 | KLHGsLARAGKFFRKNWLRLTW |
| 6387 | KLHGsLARAGYFFRKNWLRLTW |
| 6388 | KLIDIVsSQKVFFRKNWLRLTW |
| 6389 | KLIDRTEsLFFRKNWLRLTW |
| 6390 | KLIDVsSQKVFFRKNWLRLTW |
| 6391 | KLIsSSSLKEKFFRKNWLRLTW |
| 6392 | KLIsSSSLKEYFFRKNWLRLTW |
| 6393 | KLKDRLPsIFFRKNWLRLTW |
| 6394 | KLKsNPDFLKFFRKNWLRLTW |
| 6395 | KLKsNPDFLKKFFRKNWLRLTW |
| 6396 | KLKsNPDFLKYFFRKNWLRLTW |
| 6397 | KLKsPAPGLFFRKNWLRLTW |
| 6398 | KLKsPAPGVFFRKNWLRLTW |
| 6399 | KLKsQEIFLFFRKNWLRLTW |
| 6400 | KLKSsPLIEKKFFRKNWLRLTW |
| 6401 | KLKSsPLIEKYFFRKNWLRLTW |
| 6402 | KLKtPLVAKFFRKNWLRLTW |
| 6403 | KLKtPLVARFFRKNWLRLTW |
| 6404 | KLLDFGSLsNLQVFFRKNWLRLTW |
| 6405 | KLLQFYPsLFFRKNWLRLTW |
| 6406 | KLLQFYPsVFFRKNWLRLTW |
| 6407 | KLLsPSDEKLFFRKNWLRLTW |
| 6408 | KLLsPSNEKLFFRKNWLRLTW |
| 6409 | KLLsPSNEKVFFRKNWLRLTW |
| 6410 | KLLSSAQRtLFFRKNWLRLTW |
| 6411 | KLLSSAQRtVFFRKNWLRLTW |
| 6412 | KLLsTEEMELFFRKNWLRLTW |
| 6413 | KLLsTEEMEVFFRKNWLRLTW |
| 6414 | KLLsVERIKFFRKNWLRLTW |
| 6415 | KLLtPIKEKFFRKNWLRLTW |
| 6416 | KLLtPIKEYFFRKNWLRLTW |
| 6417 | KLMAPDIsLFFRKNWLRLTW |
| 6418 | KLMAPDIsVFFRKNWLRLTW |
| 6419 | KLMIDRTEsVFFRKNWLRLTW |
| 6420 | KLMsDVEDVFFRKNWLRLTW |
| 6421 | KLMsPKADVFFRKNWLRLTW |
| 6422 | KLMsPKADVKLFFRKNWLRLTW |
| 6423 | KLMsPKADVKVFFRKNWLRLTW |
| 6424 | KLPDsPALAFFRKNWLRLTW |
| 6425 | KLPDsPALAKFFRKNWLRLTW |
| 6426 | KLPDsPALAKKFFRKNWLRLTW |
| 6427 | KLPDsPALAKYFFRKNWLRLTW |
| 6428 | KLPDsPALAYFFRKNWLRLTW |
| 6429 | KLPsPAPARKFFRKNWLRLTW |
| 6430 | KLPTsPLKMKFFRKNWLRLTW |
| 6431 | KLPTsPLKMYFFRKNWLRLTW |
| 6432 | KLPTtPVKAKFFRKNWLRLTW |
| 6433 | KLPTtPVKAYFFRKNWLRLTW |
| 6434 | KLQEFLQtLFFRKNWLRLTW |
| 6435 | KLQVtSLSVFFRKNWLRLTW |
| 6436 | KLRsPFLQKFFRKNWLRLTW |
| 6437 | KLRsPFLQYFFRKNWLRLTW |
| 6438 | KLRSsPREAKFFRKNWLRLTW |
| 6439 | KLRTsPTFKFFRKNWLRLTW |
| 6440 | KLsGDQPAARFFRKNWLRLTW |
| 6441 | KLSGLsFFFRKNWLRLTW |
| 6442 | KLSsLGNLKFFRKNWLRLTW |
| 6443 | KLSsLGNLKKFFRKNWLRLTW |
| 6444 | KLSsLGNLKYFFRKNWLRLTW |
| 6445 | KLSsPRGGMKFFRKNWLRLTW |
| 6446 | KLSsPRGGMKKFFRKNWLRLTW |
| 6447 | KLSsPRGGMKYFFRKNWLRLTW |
| 6448 | KLsVIAEDSESGKQNFFRKNWLRLTW |
| 6449 | KLsVIAEDSESGKQNPFFRKNWLRLTW |
| 6450 | KLsVIAEDSESGKQNPGFFRKNWLRLTW |
| 6451 | KLVSFHDDsDEDLFFRKNWLRLTW |
| 6452 | KLYsEIDIKVFFRKNWLRLTW |
| 6453 | KLYsGNMEKFFRKNWLRLTW |
| 6454 | KMAsLLHQVFFRKNWLRLTW |
| 6455 | KMAsPELERLFFRKNWLRLTW |
| 6456 | KMAsPELERVFFRKNWLRLTW |
| 6457 | KMDIVsSQKVFFRKNWLRLTW |
| 6458 | KMDsFLDMQLFFRKNWLRLTW |
| 6459 | KMDsFLDMQVFFRKNWLRLTW |
| 6460 | KMDsPRVTVFFRKNWLRLTW |
| 6461 | KMDsPTKVKKFFRKNWLRLTW |
| 6462 | KMFPDtPLALFFRKNWLRLTW |
| 6463 | KMFPDtPLAVFFRKNWLRLTW |
| 6464 | KMFsGTVRKFFRKNWLRLTW |
| 6465 | KMFsGVFVKVFFRKNWLRLTW |
| 6466 | KMFsKQQGKFFRKNWLRLTW |
| 6467 | KMFsPAHKKFFRKNWLRLTW |
| 6468 | KMFsPSKEAELFFRKNWLRLTW |
| 6469 | KMFsPSKEAEVFFRKNWLRLTW |
| 6470 | KMHGsLARAGKFFRKNWLRLTW |
| 6471 | KMIDIVsSQKVFFRKNWLRLTW |
| 6472 | KMIDRTEsLFFRKNWLRLTW |
| 6473 | KMIsSSSLKEKFFRKNWLRLTW |
| 6474 | KMKsNPDFLKFFRKNWLRLTW |
| 6475 | KMKsNPDFLKKFFRKNWLRLTW |
| 6476 | KMKsNPDFLKYFFRKNWLRLTW |
| 6477 | KMKSsPLIEKKFFRKNWLRLTW |
| 6478 | KMKtPLVAKFFRKNWLRLTW |
| 6479 | KMKtPLVARFFRKNWLRLTW |
| 6480 | KMLDFGSLsNLOVFFRKNWLRLTW |
| 6481 | KMLDFGSLsNLQVFFRKNWLRLTW |
| 6482 | KMLQFYPsLFFRKNWLRLTW |
| 6483 | KMLsPSNEKLFFRKNWLRLTW |
| 6484 | KMLsPSNEKVFFRKNWLRLTW |
| 6485 | KMLSSAQRtLFFRKNWLRLTW |
| 6486 | KMLSSAQRtVFFRKNWLRLTW |
| 6487 | KMLsVERIKFFRKNWLRLTW |
| 6488 | KMLtPIKEKFFRKNWLRLTW |
| 6489 | KMMAPDIsVFFRKNWLRLTW |
| 6490 | KMMsPKADVKLFFRKNWLRLTW |
| 6491 | KMMsPKADVKVFFRKNWLRLTW |
| 6492 | KMPTsPLKMKFFRKNWLRLTW |
| 6493 | KMPTtPVKAKFFRKNWLRLTW |
| 6494 | KMPTtPVKAYFFRKNWLRLTW |
| 6495 | KMRsPFLQKFFRKNWLRLTW |
| 6496 | KMRSsPREAKFFRKNWLRLTW |
| 6497 | KMRTsPTFKFFRKNWLRLTW |
| 6498 | KMSsLGNLKFFRKNWLRLTW |
| 6499 | KMSsLGNLKKFFRKNWLRLTW |
| 6500 | KMSsLGNLKYFFRKNWLRLTW |
| 6501 | KMSsPRGGMKFFRKNWLRLTW |
| 6502 | KMSsPRGGMKKFFRKNWLRLTW |
| 6503 | KMYsEIDIKVFFRKNWLRLTW |
| 6504 | KMYsGNMEKFFRKNWLRLTW |
| 6505 | KNRsWKYNFFRKNWLRLTW |
| 6506 | KNRsWKYNQFFRKNWLRLTW |
| 6507 | KNRsWKYNQSISLRFFRKNWLRLTW |
| 6508 | KNRsWKYNQSISLRRPFFRKNWLRLTW |
| 6509 | KPAsPARRFFFRKNWLRLTW |
| 6510 | KPAsPARRLFFRKNWLRLTW |
| 6511 | KPAsPARRMFFRKNWLRLTW |
| 6512 | KPAsPARRVFFRKNWLRLTW |
| 6513 | KPAsPKFIVTFFFRKNWLRLTW |
| 6514 | KPAsPKFIVTLFFRKNWLRLTW |
| 6515 | KPAsPKFIVTMFFRKNWLRLTW |
| 6516 | KPAsPKFIVTVFFRKNWLRLTW |
| 6517 | KPEsRRSSLFFRKNWLRLTW |
| 6518 | KPEsRRsSLLFFRKNWLRLTW |
| 6519 | KPEsRRSsLLFFRKNWLRLTW |
| 6520 | KPESRRSSLLFFRKNWLRLTW |
| 6521 | KPLIRsQSLFFRKNWLRLTW |
| 6522 | KPLIRSQsLFFRKNWLRLTW |
| 6523 | KPPHsPLVFFFRKNWLRLTW |
| 6524 | KPPHsPLVLFFRKNWLRLTW |
| 6525 | KPPHsPLVMFFRKNWLRLTW |
| 6526 | KPPHsPLWFFRKNWLRLTW |
| 6527 | KPPsPEHQSFFFRKNWLRLTW |
| 6528 | KPPsPEHQSLFFRKNWLRLTW |
| 6529 | KPPsPEHQSMFFRKNWLRLTW |
| 6530 | KPPsPEHQSVFFRKNWLRLTW |
| 6531 | KPPsPSPIEFFFRKNWLRLTW |
| 6532 | KPPsPSPIELFFRKNWLRLTW |
| 6533 | KPPsPSPIEMFFRKNWLRLTW |
| 6534 | KPPsPSPIEVFFRKNWLRLTW |
| 6535 | KPPtPGASFFFRKNWLRLTW |
| 6536 | KPPtPGASLFFRKNWLRLTW |
| 6537 | KPPtPGASMFFRKNWLRLTW |
| 6538 | KPPtPGASVFFRKNWLRLTW |
| 6539 | KPPYRSHsFFFRKNWLRLTW |
| 6540 | KPPYRSHsLFFRKNWLRLTW |
| 6541 | KPPYRSHsMFFRKNWLRLTW |
| 6542 | KPPYRSHsVFFRKNWLRLTW |
| 6543 | KPQTRGKtFFFRKNWLRLTW |
| 6544 | KPQTRGKtLFFRKNWLRLTW |
| 6545 | KPQTRGKtMFFRKNWLRLTW |
| 6546 | KPQTRGKtVFFRKNWLRLTW |
| 6547 | KPRPLsMDLFFRKNWLRLTW |
| 6548 | KPRPPPLsFFFRKNWLRLTW |
| 6549 | KPRPPPLsLFFRKNWLRLTW |
| 6550 | KPRPPPLsMFFRKNWLRLTW |
| 6551 | KPRPPPLsPFFRKNWLRLTW |
| 6552 | KPRPPPLsVFFRKNWLRLTW |
| 6553 | KPRRFsRsLFFRKNWLRLTW |
| 6554 | KPRRFsRSLFFRKNWLRLTW |
| 6555 | KPRsPDHVFFFRKNWLRLTW |
| 6556 | KPRsPDHVLFFRKNWLRLTW |
| 6557 | KPRsPDHVMFFRKNWLRLTW |
| 6558 | KPRsPDHWFFRKNWLRLTW |
| 6559 | KPRsPFSKIFFRKNWLRLTW |
| 6560 | KPRsPPRAFFFRKNWLRLTW |
| 6561 | KPRsPPRALFFRKNWLRLTW |
| 6562 | KPRsPPRALFFFRKNWLRLTW |
| 6563 | KPRsPPRALLFFRKNWLRLTW |
| 6564 | KPRsPPRALMFFRKNWLRLTW |
| 6565 | KPRsPPRALVFFRKNWLRLTW |
| 6566 | KPRsPPRALVFFFRKNWLRLTW |
| 6567 | KPRsPPRALVLFFRKNWLRLTW |
| 6568 | KPRsPPRALVLFFFRKNWLRLTW |
| 6569 | KPRsPPRALVLLFFRKNWLRLTW |
| 6570 | KPRsPPRALVLMFFRKNWLRLTW |
| 6571 | KPRsPPRALVLPFFRKNWLRLTW |
| 6572 | KPRsPPRALVLVFFRKNWLRLTW |
| 6573 | KPRsPPRALVMFFRKNWLRLTW |
| 6574 | KPRsPPRALWFFRKNWLRLTW |
| 6575 | KPRsPPRAMFFRKNWLRLTW |
| 6576 | KPRsPPRAVFFRKNWLRLTW |
| 6577 | KPRsPWEFFFRKNWLRLTW |
| 6578 | KPRsPWELFFRKNWLRLTW |
| 6579 | KPRsPWEMFFRKNWLRLTW |
| 6580 | KPRsPWEVFFRKNWLRLTW |
| 6581 | KPSsPRGSLFFRKNWLRLTW |
| 6582 | KPSsPRGSLLFFRKNWLRLTW |
| 6583 | KPVsPKSGTLFFRKNWLRLTW |
| 6584 | KPYsPLASFFFRKNWLRLTW |
| 6585 | KPYsPLASLFFRKNWLRLTW |
| 6586 | KPYsPLASMFFRKNWLRLTW |
| 6587 | KPYsPLASVFFRKNWLRLTW |
| 6588 | KQDsLVINLFFRKNWLRLTW |
| 6589 | KRAsFAKSFFFRKNWLRLTW |
| 6590 | KRAsFAKSKFFRKNWLRLTW |
| 6591 | KRAsFAKSLFFRKNWLRLTW |
| 6592 | KRAsFAKSMFFRKNWLRLTW |
| 6593 | KRAsFAKSRFFRKNWLRLTW |
| 6594 | KRAsFAKSVFFRKNWLRLTW |
| 6595 | KRAsFAKSYFFRKNWLRLTW |
| 6596 | KRAsGQAFEFFFRKNWLRLTW |
| 6597 | KRAsGQAFEKFFRKNWLRLTW |
| 6598 | KRAsGQAFELFFRKNWLRLTW |
| 6599 | KRAsGQAFERFFRKNWLRLTW |
| 6600 | KRAsGQAFEYFFRKNWLRLTW |
| 6601 | KRASsPFRFFFRKNWLRLTW |
| 6602 | KRASsPFRKFFRKNWLRLTW |
| 6603 | KRASsPFRLFFRKNWLRLTW |
| 6604 | KRASsPFRMFFRKNWLRLTW |
| 6605 | KRASsPFRRFFRKNWLRLTW |
| 6606 | KRASsPFRYFFRKNWLRLTW |
| 6607 | KRAsVFVKFFFRKNWLRLTW |
| 6608 | KRAsVFVKKFFRKNWLRLTW |
| 6609 | KRAsVFVKLFFRKNWLRLTW |
| 6610 | KRAsVFVKMFFRKNWLRLTW |
| 6611 | KRAsVFVKRFFRKNWLRLTW |
| 6612 | KRAsVFVKYFFRKNWLRLTW |
| 6613 | KRAsYILRLFFRKNWLRLTW |
| 6614 | KRFsFKFFFRKNWLRLTW |
| 6615 | KRFsFKKFFRKNWLRLTW |
| 6616 | KRFsFKKsFFFRKNWLRLTW |
| 6617 | KRFsFKKSFFFRKNWLRLTW |
| 6618 | KRFsFKKSKFFRKNWLRLTW |
| 6619 | KRFsFKKSLFFRKNWLRLTW |
| 6620 | KRFsFKKSMFFRKNWLRLTW |
| 6621 | KRFsFKKSRFFRKNWLRLTW |
| 6622 | KRFsFKKSYFFRKNWLRLTW |
| 6623 | KRFsFKLFFRKNWLRLTW |
| 6624 | KRFsFKMFFRKNWLRLTW |
| 6625 | KRFsFKRFFRKNWLRLTW |
| 6626 | KRFsFKsSFFFRKNWLRLTW |
| 6627 | KRFsFKYFFRKNWLRLTW |
| 6628 | KRFsGTVRFFFRKNWLRLTW |
| 6629 | KRFsGTVRKFFRKNWLRLTW |
| 6630 | KRFsGTVRLFFRKNWLRLTW |
| 6631 | KRFsGTVRMFFRKNWLRLTW |
| 6632 | KRFsGTVRRFFRKNWLRLTW |
| 6633 | KRFsGTVRYFFRKNWLRLTW |
| 6634 | KRIVIsPKPFFFRKNWLRLTW |
| 6635 | KRKsFTSLYFFRKNWLRLTW |
| 6636 | KRLEKsPSFFFRKNWLRLTW |
| 6637 | KRLEKSPsFFFRKNWLRLTW |
| 6638 | KRLsPAPQFFFRKNWLRLTW |
| 6639 | KRLsPAPQKFFRKNWLRLTW |
| 6640 | KRLsPAPQLFFRKNWLRLTW |
| 6641 | KRLsPAPQMFFRKNWLRLTW |
| 6642 | KRLsPAPQRFFRKNWLRLTW |
| 6643 | KRLsPAPQYFFRKNWLRLTW |
| 6644 | KRLsTSPVRLFFRKNWLRLTW |
| 6645 | KRLsVERIFFFRKNWLRLTW |
| 6646 | KRLsVERIKFFRKNWLRLTW |
| 6647 | KRLsVERILFFRKNWLRLTW |
| 6648 | KRLsVERIMFFRKNWLRLTW |
| 6649 | KRLsVERIRFFRKNWLRLTW |
| 6650 | KRLsVERIYFFRKNWLRLTW |
| 6651 | KRMsPKEFFFRKNWLRLTW |
| 6652 | KRMsPKEKFFRKNWLRLTW |
| 6653 | KRMsPKELFFRKNWLRLTW |
| 6654 | KRMsPKERFFRKNWLRLTW |
| 6655 | KRMsPKEYFFRKNWLRLTW |
| 6656 | KRmsPKPELFFRKNWLRLTW |
| 6657 | KRMsPKPELFFRKNWLRLTW |
| 6658 | KRMsPKPFFFRKNWLRLTW |
| 6659 | KRMsPKPKFFRKNWLRLTW |
| 6660 | KRMsPKPLFFRKNWLRLTW |
| 6661 | KRMsPKPMFFRKNWLRLTW |
| 6662 | KRMsPKPRFFRKNWLRLTW |
| 6663 | KRMsPKPYFFRKNWLRLTW |
| 6664 | KRPEsPPSIFFRKNWLRLTW |
| 6665 | KRWQsPVTKFFRKNWLRLTW |
| 6666 | KRYsEPVSLFFRKNWLRLTW |
| 6667 | KRYsGNMEFFFRKNWLRLTW |
| 6668 | KRYsGNMEKFFRKNWLRLTW |
| 6669 | KRYsGNMELFFRKNWLRLTW |
| 6670 | KRYsGNMEMFFRKNWLRLTW |
| 6671 | KRYsGNMERFFRKNWLRLTW |
| 6672 | KRYsGNmEYFFRKNWLRLTW |
| 6673 | KRYsGNMEYFFRKNWLRLTW |
| 6674 | KRYsRALYLFFRKNWLRLTW |
| 6675 | KSDsRQERYFFRKNWLRLTW |
| 6676 | KSEsRQERYFFRKNWLRLTW |
| 6677 | KSGELLAtWFFRKNWLRLTW |
| 6678 | KSKsNPDFLKKFFRKNWLRLTW |
| 6679 | KSKsNPFLKKFFRKNWLRLTW |
| 6680 | KSKtPLVAKFFRKNWLRLTW |
| 6681 | KSKtPLVARFFRKNWLRLTW |
| 6682 | KSKtPLVAYFFRKNWLRLTW |
| 6683 | KsLVRLLLLFFRKNWLRLTW |
| 6684 | KSSsLGNLKKFFRKNWLRLTW |
| 6685 | KsVKALSSLHGDDQFFRKNWLRLTW |
| 6686 | KsVKALSSLHGDDQDFFRKNWLRLTW |
| 6687 | KsVKALSSLHGDDQDsEDEFFRKNWLRLTW |
| 6688 | KSVKALSSLHGDDQDsEDEFFRKNWLRLTW |
| 6689 | KTDsRQERYFFRKNWLRLTW |
| 6690 | KTEsRQERYFFRKNWLRLTW |
| 6691 | KtLSPGKNGWKFFRKNWLRLTW |
| 6692 | KtLSPGKNGWYFFRKNWLRLTW |
| 6693 | KTMsGTFLLFFRKNWLRLTW |
| 6694 | KTMsPSQMIMFFRKNWLRLTW |
| 6695 | KTPTsPLKMKFFRKNWLRLTW |
| 6696 | KTPTsPLKMYFFRKNWLRLTW |
| 6697 | KTWKGsIGLFFRKNWLRLTW |
| 6698 | KVAsLLHQVFFRKNWLRLTW |
| 6699 | KVDsPVIFFFRKNWLRLTW |
| 6700 | KVHGsLARAGKFFRKNWLRLTW |
| 6701 | KVHGsLARAGYFFRKNWLRLTW |
| 6702 | KVKSsPLIEKKFFRKNWLRLTW |
| 6703 | KVKsSPLIEKLFFRKNWLRLTW |
| 6704 | KVKSsPLIEKLFFRKNWLRLTW |
| 6705 | KVKSsPLIEKYFFRKNWLRLTW |
| 6706 | KVLsKEFHLFFRKNWLRLTW |
| 6707 | KVLSPtAAKFFRKNWLRLTW |
| 6708 | KVLsSLVTLFFRKNWLRLTW |
| 6709 | KVLsTEEMELFFRKNWLRLTW |
| 6710 | KVLStEEMELFFRKNWLRLTW |
| 6711 | KVLtPIKeKFFRKNWLRLTW |
| 6712 | KVLtPIKEKFFRKNWLRLTW |
| 6713 | KVLtPIKEYFFRKNWLRLTW |
| 6714 | KVPDsPALAKFFRKNWLRLTW |
| 6715 | KVPDsPALAKKFFRKNWLRLTW |
| 6716 | KVPDsPALAKYFFRKNWLRLTW |
| 6717 | KVPDsPALAYFFRKNWLRLTW |
| 6718 | KVPTsPLKMYFFRKNWLRLTW |
| 6719 | KVQsLRRALFFRKNWLRLTW |
| 6720 | KVQVtSLSVFFRKNWLRLTW |
| 6721 | KVYsSSEFLFFRKNWLRLTW |
| 6722 | KYIsGPHELFFRKNWLRLTW |
| 6723 | KYsPGKLRGNFFRKNWLRLTW |
| 6724 | LGGGGAGLSGRASGGAQsPLRYLHVFFRKNWLRLTW |
| 6725 | LKLsYLTWVFFRKNWLRLTW |
| 6726 | LLAsPGHISVFFRKNWLRLTW |
| 6727 | LLDPSRSYsYFFRKNWLRLTW |
| 6728 | LLDtPVKTQYFFRKNWLRLTW |
| 6729 | LLFsPVTSLFFRKNWLRLTW |
| 6730 | LLFsPVTSVFFRKNWLRLTW |
| 6731 | LLLsEEVELFFRKNWLRLTW |
| 6732 | LLNKSsPVKFFRKNWLRLTW |
| 6733 | LLNKSsPVKKFFRKNWLRLTW |
| 6734 | LLNKSsPVKYFFRKNWLRLTW |
| 6735 | LMFsPVTSLFFRKNWLRLTW |
| 6736 | LMFsPVTSVFFRKNWLRLTW |
| 6737 | LMFsVTSIFFRKNWLRLTW |
| 6738 | LMFsVTSLFFRKNWLRLTW |
| 6739 | LMNKSsPVKFFRKNWLRLTW |
| 6740 | LMNKSsPVKKFFRKNWLRLTW |
| 6741 | LMNKSsPVKYFFRKNWLRLTW |
| 6742 | LPAsPHQFFFRKNWLRLTW |
| 6743 | LPAsPHQLFFRKNWLRLTW |
| 6744 | LPAsPHQMFFRKNWLRLTW |
| 6745 | LPAsPHQVFFRKNWLRLTW |
| 6746 | LPAsPRARFFFRKNWLRLTW |
| 6747 | LPAsPRARLFFRKNWLRLTW |
| 6748 | LPAsPRARMFFRKNWLRLTW |
| 6749 | LPAsPRARVFFRKNWLRLTW |
| 6750 | LPIFSRLsFFFRKNWLRLTW |
| 6751 | LPIFSRLsIFFRKNWLRLTW |
| 6752 | LPIFSRLsLFFRKNWLRLTW |
| 6753 | LPIFSRLsMFFRKNWLRLTW |
| 6754 | LPIFSRLsVFFRKNWLRLTW |
| 6755 | LPKGLsASLFFRKNWLRLTW |
| 6756 | LPKGLSAsLFFRKNWLRLTW |
| 6757 | LPKsPPYTAFFFRKNWLRLTW |
| 6758 | LPKsPPYTALFFRKNWLRLTW |
| 6759 | LPKsPPYTAMFFRKNWLRLTW |
| 6760 | LPKsPPYTAVFFRKNWLRLTW |
| 6761 | LPRGSsPSVFFFRKNWLRLTW |
| 6762 | LPRGsSPSVLFFRKNWLRLTW |
| 6763 | LPRGSsPSVLFFRKNWLRLTW |
| 6764 | LPRGSsPSVMFFRKNWLRLTW |
| 6765 | LPRGSsPSWFFRKNWLRLTW |
| 6766 | LPRmIsHSELFFRKNWLRLTW |
| 6767 | LPRMIsHSELFFRKNWLRLTW |
| 6768 | LPRPAsPALFFRKNWLRLTW |
| 6769 | LPRSSsMAAFFRKNWLRLTW |
| 6770 | LPRSSsMAAGLFFRKNWLRLTW |
| 6771 | LPRtPRPELFFRKNWLRLTW |
| 6772 | LPVsPRLQLFFRKNWLRLTW |
| 6773 | LQLsPLKGLSLFFRKNWLRLTW |
| 6774 | LQNItENQLFFRKNWLRLTW |
| 6775 | LSDPSRSYsYFFRKNWLRLTW |
| 6776 | LSDsDTEAKLFFRKNWLRLTW |
| 6777 | LSDsDTEAKYFFRKNWLRLTW |
| 6778 | LSDtPVKTQYFFRKNWLRLTW |
| 6779 | LSEPSRSYsYFFRKNWLRLTW |
| 6780 | LSEsDTEAKLFFRKNWLRLTW |
| 6781 | LSEsDTEAKYFFRKNWLRLTW |
| 6782 | LSEtPVKTQYFFRKNWLRLTW |
| 6783 | LSKFRMPQPSSGREsPRHFFRKNWLRLTW |
| 6784 | LSSsVIRELFFRKNWLRLTW |
| 6785 | LTDPSRSYsYFFRKNWLRLTW |
| 6786 | LTDPSsPTISSYFFRKNWLRLTW |
| 6787 | LTDsDTEAKLFFRKNWLRLTW |
| 6788 | LTDsDTEAKYFFRKNWLRLTW |
| 6789 | LTDtPVKTQYFFRKNWLRLTW |
| 6790 | LTEPSRSYsYFFRKNWLRLTW |
| 6791 | LTEsDTEAKLFFRKNWLRLTW |
| 6792 | LTEsDTEAKYFFRKNWLRLTW |
| 6793 | LTEtPVKTOYFFRKNWLRLTW |
| 6794 | LTEtPVKTQYFFRKNWLRLTW |
| 6795 | MLAEsPSVPRLFFRKNWLRLTW |
| 6796 | MLAEsPSVPRVFFRKNWLRLTW |
| 6797 | MLRsPPRVSKFFRKNWLRLTW |
| 6798 | MMRsPPRVSKFFRKNWLRLTW |
| 6799 | MPRPsIKKAQNSQAARQFFRKNWLRLTW |
| 6800 | MPRQPsAIRMFFRKNWLRLTW |
| 6801 | MPRQPsATRFFFRKNWLRLTW |
| 6802 | MPRQPsATRLFFRKNWLRLTW |
| 6803 | MPRQPsATRMFFRKNWLRLTW |
| 6804 | MPRQPsATRVFFRKNWLRLTW |
| 6805 | MRLsEWLQLFFRKNWLRLTW |
| 6806 | MRLsRELQFFFRKNWLRLTW |
| 6807 | MRLsRELQKFFRKNWLRLTW |
| 6808 | MRLsRELQLFFRKNWLRLTW |
| 6809 | MRLsRELQMFFRKNWLRLTW |
| 6810 | MRLsRELQRFFRKNWLRLTW |
| 6811 | MRLsRELQYFFRKNWLRLTW |
| 6812 | MSDtYRLKYFFRKNWLRLTW |
| 6813 | MSEtYRLKYFFRKNWLRLTW |
| 6814 | MTDtYRLKYFFRKNWLRLTW |
| 6815 | MTEtYRLKYFFRKNWLRLTW |
| 6816 | MTRsPPRVSKFFRKNWLRLTW |
| 6817 | MTRsPPRVSYFFRKNWLRLTW |
| 6818 | NAPPAYEKLsAEFFRKNWLRLTW |
| 6819 | NFKsPVKTIRFFRKNWLRLTW |
| 6820 | NLELSKFRMPQPSSGREsPRHFFRKNWLRLTW |
| 6821 | NLGsRNHVHQLFFRKNWLRLTW |
| 6822 | NLLsPDGKMISVFFRKNWLRLTW |
| 6823 | NLVERKNsKFFRKNWLRLTW |
| 6824 | NLVERKNsLFFRKNWLRLTW |
| 6825 | NMDsPGPMLFFRKNWLRLTW |
| 6826 | NMVERKNsKFFRKNWLRLTW |
| 6827 | NMVERKNsLFFRKNWLRLTW |
| 6828 | NRAMRRVsSVPSRFFRKNWLRLTW |
| 6829 | NRAMRRVsSVPSRAQFFRKNWLRLTW |
| 6830 | NRsWKYNQSISLRFFRKNWLRLTW |
| 6831 | NRsWKYNQSISLRRPFFRKNWLRLTW |
| 6832 | NRYtNRWTFFFRKNWLRLTW |
| 6833 | NRYtNRWTKFFRKNWLRLTW |
| 6834 | NRYtNRWTLFFRKNWLRLTW |
| 6835 | NRYtNRWTMFFRKNWLRLTW |
| 6836 | NRYtNRWTRFFRKNWLRLTW |
| 6837 | NRYtNRWTYFFRKNWLRLTW |
| 6838 | NSDsPLRYFFRKNWLRLTW |
| 6839 | NSEsPLRYFFRKNWLRLTW |
| 6840 | NTDsPLRYFFRKNWLRLTW |
| 6841 | NTEsPLRYFFRKNWLRLTW |
| 6842 | NYVERKNsKFFRKNWLRLTW |
| 6843 | NYVERKNsLFFRKNWLRLTW |
| 6844 | NYVERKNsYFFRKNWLRLTW |
| 6845 | PARsPVTEIFFRKNWLRLTW |
| 6846 | PAYEKLsAEFFRKNWLRLTW |
| 6847 | PAYEKLsAEQSPFFRKNWLRLTW |
| 6848 | PmVTLsLNLFFRKNWLRLTW |
| 6849 | PMVTLsLNLFFRKNWLRLTW |
| 6850 | PNAPPAYEKLsAFFRKNWLRLTW |
| 6851 | PPAYEKLsAFFRKNWLRLTW |
| 6852 | PPAYEKLsAEQSFFRKNWLRLTW |
| 6853 | PPLPEDSIKVIRNMRAAsPPAFFRKNWLRLTW |
| 6854 | PYDPALGsPSRFFRKNWLRLTW |
| 6855 | QAASNFKsPVKTIRFFRKNWLRLTW |
| 6856 | QLDsPQRALYFFRKNWLRLTW |
| 6857 | QLEsPQRALYFFRKNWLRLTW |
| 6858 | QLFsPKKGQKFFRKNWLRLTW |
| 6859 | QMFsPKKGQKFFRKNWLRLTW |
| 6860 | QPQRRsLRLFFRKNWLRLTW |
| 6861 | QPRsPGPDYSFFFRKNWLRLTW |
| 6862 | QPRsPGPDYSLFFRKNWLRLTW |
| 6863 | QPRsPGPDYSMFFRKNWLRLTW |
| 6864 | QPRsPGPDYSVFFRKNWLRLTW |
| 6865 | QPRtPsPLVFFFRKNWLRLTW |
| 6866 | QPRtPsPLVFFFRKNWLRLTW |
| 6867 | QPRtPsPLVLFFRKNWLRLTW |
| 6868 | QPRtPsPLVLFFRKNWLRLTW |
| 6869 | QPRtPsPLVMFFRKNWLRLTW |
| 6870 | QPRtPSPLVMFFRKNWLRLTW |
| 6871 | QPRtPsPLWFFRKNWLRLTW |
| 6872 | QPRtPSPLWFFRKNWLRLTW |
| 6873 | QPSFPsVLPAFFRKNWLRLTW |
| 6874 | QRLsPLSAAYFFRKNWLRLTW |
| 6875 | QSDsPQRALYFFRKNWLRLTW |
| 6876 | QSEsPQRALYFFRKNWLRLTW |
| 6877 | QTDsPQRALYFFRKNWLRLTW |
| 6878 | QTEsPQRALYFFRKNWLRLTW |
| 6879 | QVAMPVKKSPRRSsSDEQGLSYSSLKNVFFRKNWLRLTW |
| 6880 | QVFsPKKGQKFFRKNWLRLTW |
| 6881 | QVFsPKKGQYFFRKNWLRLTW |
| 6882 | RADsPVHMFFRKNWLRLTW |
| 6883 | RAFsFSKTPKFFRKNWLRLTW |
| 6884 | RAFsFSKTPYFFRKNWLRLTW |
| 6885 | RAFsVKFEVFFRKNWLRLTW |
| 6886 | RAHsEPLALFFRKNWLRLTW |
| 6887 | RAHsSPASLFFRKNWLRLTW |
| 6888 | RAHSsPASLFFRKNWLRLTW |
| 6889 | RAKsPISLKFFRKNWLRLTW |
| 6890 | RAKsPISLYFFRKNWLRLTW |
| 6891 | RAPsPSSRFFFRKNWLRLTW |
| 6892 | RAPsPSSRLFFRKNWLRLTW |
| 6893 | RAPsPSSRMFFRKNWLRLTW |
| 6894 | RAPsPSSRVFFRKNWLRLTW |
| 6895 | RARGIsPIVFFFRKNWLRLTW |
| 6896 | RASsDIVsLFFRKNWLRLTW |
| 6897 | RASsDIVSLFFRKNWLRLTW |
| 6898 | RASsLSITVFFRKNWLRLTW |
| 6899 | REAPsPLmIFFRKNWLRLTW |
| 6900 | REAPsPLMIFFRKNWLRLTW |
| 6901 | REAsPAPLAFFRKNWLRLTW |
| 6902 | REAsPRLRVFFRKNWLRLTW |
| 6903 | REAsPSRLSVFFRKNWLRLTW |
| 6904 | REDsTPGKVFLFFRKNWLRLTW |
| 6905 | REIMGtPEYLFFRKNWLRLTW |
| 6906 | REKsPGRmLFFRKNWLRLTW |
| 6907 | REKsPGRMLFFRKNWLRLTW |
| 6908 | REKsPLFQFFFRKNWLRLTW |
| 6909 | REKsPLFQWFFRKNWLRLTW |
| 6910 | REKsPLFQYFFRKNWLRLTW |
| 6911 | RELARKGsLFFRKNWLRLTW |
| 6912 | RELsPLISLFFRKNWLRLTW |
| 6913 | REPsPLPELFFRKNWLRLTW |
| 6914 | RERsPSPSFFFRKNWLRLTW |
| 6915 | RESsPTRRLFFRKNWLRLTW |
| 6916 | REVsPAPAVFFRKNWLRLTW |
| 6917 | REYGsTSSIFFRKNWLRLTW |
| 6918 | RFKtQPVTFFFRKNWLRLTW |
| 6919 | RGDGYGtFFFRKNWLRLTW |
| 6920 | RGDsPKIDLFFRKNWLRLTW |
| 6921 | RIDsKDSASELFFRKNWLRLTW |
| 6922 | RIGsPLSPKFFRKNWLRLTW |
| 6923 | RILsGWTKFFRKNWLRLTW |
| 6924 | RILsGWTYFFRKNWLRLTW |
| 6925 | RILsPSMASKFFRKNWLRLTW |
| 6926 | RILsPSMASYFFRKNWLRLTW |
| 6927 | RINsFEEHVFFRKNWLRLTW |
| 6928 | RIQsKLYRAFFRKNWLRLTW |
| 6929 | RIQyIQSRFFFRKNWLRLTW |
| 6930 | RIQyIQSRFYFFRKNWLRLTW |
| 6931 | RIsHELDSFFRKNWLRLTW |
| 6932 | RITsLIVHVFFRKNWLRLTW |
| 6933 | RIVQyIQSRFFRKNWLRLTW |
| 6934 | RIYQyIQFFRKNWLRLTW |
| 6935 | RIYQyIQSKFFRKNWLRLTW |
| 6936 | RIYQyIQSRFFRKNWLRLTW |
| 6937 | RIYQyIQSRFFFRKNWLRLTW |
| 6938 | RIYQyIQSRFKFFRKNWLRLTW |
| 6939 | RIYQyIQSRFYFFRKNWLRLTW |
| 6940 | RIYQyIQSRKFFRKNWLRLTW |
| 6941 | RIYQyIQSRYFFRKNWLRLTW |
| 6942 | RIYQyIQSYFFRKNWLRLTW |
| 6943 | RIYQyLQSRFFFRKNWLRLTW |
| 6944 | RIYQyLQSRFYFFRKNWLRLTW |
| 6945 | RKLRsLEQLFFRKNWLRLTW |
| 6946 | RKLsVILIKFFRKNWLRLTW |
| 6947 | RKLsVILILFFRKNWLRLTW |
| 6948 | RKLsVILIYFFRKNWLRLTW |
| 6949 | RKPsIVTKYFFRKNWLRLTW |
| 6950 | RKSsIIIRMFFRKNWLRLTW |
| 6951 | RLAsASRALFFRKNWLRLTW |
| 6952 | RLAsFAVRKFFRKNWLRLTW |
| 6953 | RLAsFAVRYFFRKNWLRLTW |
| 6954 | RLAsIELPSMFFRKNWLRLTW |
| 6955 | RLAsIELPSMAVFFRKNWLRLTW |
| 6956 | RLAsIELPSVFFRKNWLRLTW |
| 6957 | RLAsLNAEALFFRKNWLRLTW |
| 6958 | RLAsLNAEAVFFRKNWLRLTW |
| 6959 | RLAsLQSEVFFRKNWLRLTW |
| 6960 | RLAsLSISVFFRKNWLRLTW |
| 6961 | RLAsPLVHKFFRKNWLRLTW |
| 6962 | RLAsPLVHYFFRKNWLRLTW |
| 6963 | RLAsPPPPPKFFRKNWLRLTW |
| 6964 | RLAsPPPPPYFFRKNWLRLTW |
| 6965 | RLAsPTSGVFFRKNWLRLTW |
| 6966 | RLAsPTSGVKFFRKNWLRLTW |
| 6967 | RLAsPTSGVKKFFRKNWLRLTW |
| 6968 | RLAsPTSGVKRFFRKNWLRLTW |
| 6969 | RLAsPTSGVKYFFRKNWLRLTW |
| 6970 | RLAsRPLLLFFRKNWLRLTW |
| 6971 | RLAsSATQVHKFFRKNWLRLTW |
| 6972 | RLAsYLDKVFFRKNWLRLTW |
| 6973 | RLAsYLDRVFFRKNWLRLTW |
| 6974 | RLDsTPGKVFLFFRKNWLRLTW |
| 6975 | RLDsTPGKVFVFFRKNWLRLTW |
| 6976 | RLDsYLRAPFFRKNWLRLTW |
| 6977 | RLDsYVRFFRKNWLRLTW |
| 6978 | RLDsYVRSFFRKNWLRLTW |
| 6979 | RLDsYVRSLFFRKNWLRLTW |
| 6980 | RLDsYVRSVFFRKNWLRLTW |
| 6981 | RLDtGPQSLFFRKNWLRLTW |
| 6982 | RLEsANRRLFFRKNWLRLTW |
| 6983 | RLFsFSKTPKFFRKNWLRLTW |
| 6984 | RLFsKELFFRKNWLRLTW |
| 6985 | RLFsKELRFFRKNWLRLTW |
| 6986 | RLFsKELRCFFRKNWLRLTW |
| 6987 | RLFsKELRVFFRKNWLRLTW |
| 6988 | RLFSLsNPSLFFRKNWLRLTW |
| 6989 | RLFsPTYGLFFRKNWLRLTW |
| 6990 | RLFsPTYGVFFRKNWLRLTW |
| 6991 | RLFsQGQDVFFRKNWLRLTW |
| 6992 | RLFVGsIPKFFRKNWLRLTW |
| 6993 | RLGsFHELLLFFRKNWLRLTW |
| 6994 | RLIsFKAEVFFRKNWLRLTW |
| 6995 | RLIsPYKKKFFRKNWLRLTW |
| 6996 | RLIsQDVKLFFRKNWLRLTW |
| 6997 | RLIsQDVKVFFRKNWLRLTW |
| 6998 | RLKLPsGSKFFRKNWLRLTW |
| 6999 | RLKLPsGSKKFFRKNWLRLTW |
| 7000 | RLKLPsGSKYFFRKNWLRLTW |
| 7001 | RLKsDERPVHIFFRKNWLRLTW |
| 7002 | RLKsPFRKKFFRKNWLRLTW |
| 7003 | RLKsPGsGHVKFFRKNWLRLTW |
| 7004 | RLKsPISLKFFRKNWLRLTW |
| 7005 | RLKsPISLYFFRKNWLRLTW |
| 7006 | RLKsPSPKSEKFFRKNWLRLTW |
| 7007 | RLKsPSPKSERFFRKNWLRLTW |
| 7008 | RLKtPTSQSYKFFRKNWLRLTW |
| 7009 | RLKtPTSQSYRFFRKNWLRLTW |
| 7010 | RLKTtPLRKFFRKNWLRLTW |
| 7011 | RLKTtPLRRFFRKNWLRLTW |
| 7012 | RLLDPsSPLALFFRKNWLRLTW |
| 7013 | RLLDPSsPLALFFRKNWLRLTW |
| 7014 | RLLDRSPsRSAKFFRKNWLRLTW |
| 7015 | RLLDRSPsRSAYFFRKNWLRLTW |
| 7016 | RLLsDGQQHLFFRKNWLRLTW |
| 7017 | RLLsDLEELFFRKNWLRLTW |
| 7018 | RLLsDQTRLFFRKNWLRLTW |
| 7019 | RLLsFQRYLFFRKNWLRLTW |
| 7020 | RLLsGWTKFFRKNWLRLTW |
| 7021 | RLLsGWTYFFRKNWLRLTW |
| 7022 | RLLsHISEAFFRKNWLRLTW |
| 7023 | RLLsHISEVFFRKNWLRLTW |
| 7024 | RLLsPLSSAFFRKNWLRLTW |
| 7025 | RLLsPLSSARLFFRKNWLRLTW |
| 7026 | RLLsPLSSVFFRKNWLRLTW |
| 7027 | RLLsPQQPALFFRKNWLRLTW |
| 7028 | RLLsPRPSLFFRKNWLRLTW |
| 7029 | RLLsPRPSLLFFRKNWLRLTW |
| 7030 | RLLsPSMASKFFRKNWLRLTW |
| 7031 | RLLsSGVSEIFFRKNWLRLTW |
| 7032 | RLLsSGVSEVFFRKNWLRLTW |
| 7033 | RLLsTDAEAVFFRKNWLRLTW |
| 7034 | RLLsVEIVKFFRKNWLRLTW |
| 7035 | RLLsVEIVYFFRKNWLRLTW |
| 7036 | RLLsVHDFDFFFRKNWLRLTW |
| 7037 | RLLsVILIKFFRKNWLRLTW |
| 7038 | RLMsMPVAKFFRKNWLRLTW |
| 7039 | RLMsMPVAYFFRKNWLRLTW |
| 7040 | RLNtSDFQKLFFRKNWLRLTW |
| 7041 | RLPNRIPsLFFRKNWLRLTW |
| 7042 | RLPsFLKKNKFFRKNWLRLTW |
| 7043 | RLPsLVHGYFFRKNWLRLTW |
| 7044 | RLPsSTLKKFFRKNWLRLTW |
| 7045 | RLPsSTLKRFFRKNWLRLTW |
| 7046 | RLPsSTLKYFFRKNWLRLTW |
| 7047 | RLQsLIKNIFFRKNWLRLTW |
| 7048 | RLQsTSERLFFRKNWLRLTW |
| 7049 | RLQsTSERVFFRKNWLRLTW |
| 7050 | RLR(sLss)PTVTLFFRKNWLRLTW |
| 7051 | RLR(sLss)PTVTVFFRKNWLRLTW |
| 7052 | RLRQsPLATKFFRKNWLRLTW |
| 7053 | RLRQsPLATRFFRKNWLRLTW |
| 7054 | RLRQsPLATYFFRKNWLRLTW |
| 7055 | RLRRsPLLKFFRKNWLRLTW |
| 7056 | RLRsAGAAQKFFRKNWLRLTW |
| 7057 | RLRsLSSLREKFFRKNWLRLTW |
| 7058 | RLRsPPPVSKFFRKNWLRLTW |
| 7059 | RLRsYEDMIFFRKNWLRLTW |
| 7060 | RLRTsPITRKFFRKNWLRLTW |
| 7061 | RLRTsPITRRFFRKNWLRLTW |
| 7062 | RLSDtPPLLFFRKNWLRLTW |
| 7063 | RLSsLIRHKFFRKNWLRLTW |
| 7064 | RLSsLRASTSKFFRKNWLRLTW |
| 7065 | RLSsPISKKFFRKNWLRLTW |
| 7066 | RLSsPISKRFFRKNWLRLTW |
| 7067 | RLSsPISKYFFRKNWLRLTW |
| 7068 | RLsSPLHFVFFRKNWLRLTW |
| 7069 | RLSsPLHFVFFRKNWLRLTW |
| 7070 | RLSsPVLHKFFRKNWLRLTW |
| 7071 | RLSsPVLHRFFRKNWLRLTW |
| 7072 | RLSsPVLHYFFRKNWLRLTW |
| 7073 | RLSsRFSSKFFRKNWLRLTW |
| 7074 | RLSsRFSSRFFRKNWLRLTW |
| 7075 | RLSsRFSSYFFRKNWLRLTW |
| 7076 | RLSsRYSQKFFRKNWLRLTW |
| 7077 | RLSsRYSQYFFRKNWLRLTW |
| 7078 | RLSsVKLISKFFRKNWLRLTW |
| 7079 | RLSsVKLISYFFRKNWLRLTW |
| 7080 | RLTFsPTYGVFFRKNWLRLTW |
| 7081 | RLVsLSMRKFFRKNWLRLTW |
| 7082 | RLVsLSMRYFFRKNWLRLTW |
| 7083 | RLYKsPLRHFFRKNWLRLTW |
| 7084 | RLYKsPLRKFFRKNWLRLTW |
| 7085 | RLYQyIQSKFFRKNWLRLTW |
| 7086 | RLYQyIQSRFFRKNWLRLTW |
| 7087 | RLYQyIQSRFKFFRKNWLRLTW |
| 7088 | RLYQyIQSRFYFFRKNWLRLTW |
| 7089 | RLYQyIQSYFFRKNWLRLTW |
| 7090 | RLYQylOSKFFRKNWLRLTW |
| 7091 | RLYQyLQSRFFFRKNWLRLTW |
| 7092 | RLYQyLQSRFKFFRKNWLRLTW |
| 7093 | RLYQyLQSRFYFFRKNWLRLTW |
| 7094 | RLYQyLQSRKFFRKNWLRLTW |
| 7095 | RLYsGPMNKVFFRKNWLRLTW |
| 7096 | RLYsGSRsKFFRKNWLRLTW |
| 7097 | RLYsGSRsRFFRKNWLRLTW |
| 7098 | RLYsGSRsYFFRKNWLRLTW |
| 7099 | RLYsKSRDKFFRKNWLRLTW |
| 7100 | RLYsPDHRQKFFRKNWLRLTW |
| 7101 | RLYsPERSKFFRKNWLRLTW |
| 7102 | RLYsPRNSKFFRKNWLRLTW |
| 7103 | RLYSPYNHKFFRKNWLRLTW |
| 7104 | RLYsPYNHRFFRKNWLRLTW |
| 7105 | RLYsPYNHYFFRKNWLRLTW |
| 7106 | RLYSRsFSKFFRKNWLRLTW |
| 7107 | RLYSRsFSYFFRKNWLRLTW |
| 7108 | RLYsYPRQKFFRKNWLRLTW |
| 7109 | RLYVTTSTRTYsLGFFRKNWLRLTW |
| 7110 | RLYVTTSTRTYsLKFFRKNWLRLTW |
| 7111 | RLYVTTSTRTYsLYFFRKNWLRLTW |
| 7112 | RMAsPPPPPKFFRKNWLRLTW |
| 7113 | RMAsPTSGVFFRKNWLRLTW |
| 7114 | RMAsPTSGVKFFRKNWLRLTW |
| 7115 | RMAsPTSGVKKFFRKNWLRLTW |
| 7116 | RMAsPTSGVKRFFRKNWLRLTW |
| 7117 | RMAsPTSGVKYFFRKNWLRLTW |
| 7118 | RMAsSATQVHKFFRKNWLRLTW |
| 7119 | RMDsTPGKVFLFFRKNWLRLTW |
| 7120 | RMDsTPGKVFVFFRKNWLRLTW |
| 7121 | RMDsYVRSLFFRKNWLRLTW |
| 7122 | RMDsYVRSVFFRKNWLRLTW |
| 7123 | RMFPtPPSLFFRKNWLRLTW |
| 7124 | RMFsFSKTPKFFRKNWLRLTW |
| 7125 | RMFsKELRCFFRKNWLRLTW |
| 7126 | RMFsKELRVFFRKNWLRLTW |
| 7127 | RMFsPMEEKFFRKNWLRLTW |
| 7128 | RMFsPMEEKELLFFRKNWLRLTW |
| 7129 | RMFsPTYGLFFRKNWLRLTW |
| 7130 | RMFsPTYGVFFRKNWLRLTW |
| 7131 | RMIsPYKKKFFRKNWLRLTW |
| 7132 | RMIsQDVKLFFRKNWLRLTW |
| 7133 | RMIsQDVKVFFRKNWLRLTW |
| 7134 | RMIsTGSELFFRKNWLRLTW |
| 7135 | RMKLPsGSKFFRKNWLRLTW |
| 7136 | RMKLPsGSKKFFRKNWLRLTW |
| 7137 | RMKLPsGSKYFFRKNWLRLTW |
| 7138 | RMKsPFRKKFFRKNWLRLTW |
| 7139 | RMKsPGsGHVKFFRKNWLRLTW |
| 7140 | RMKsPSPKSEKFFRKNWLRLTW |
| 7141 | RMKtPTSQSYKFFRKNWLRLTW |
| 7142 | RMKtPTSQSYRFFRKNWLRLTW |
| 7143 | RMKTtPLRKFFRKNWLRLTW |
| 7144 | RMKTtPLRRFFRKNWLRLTW |
| 7145 | RMLDRSPsRSAKFFRKNWLRLTW |
| 7146 | RMLDRSPsRSAYFFRKNWLRLTW |
| 7147 | RMLsHISEAFFRKNWLRLTW |
| 7148 | RMLsHISEVFFRKNWLRLTW |
| 7149 | RMLsLRDQRLFFRKNWLRLTW |
| 7150 | RMLsPLSSAFFRKNWLRLTW |
| 7151 | RMLsPLSSVFFRKNWLRLTW |
| 7152 | RMLsPSMASKFFRKNWLRLTW |
| 7153 | RMLsSGVSEIFFRKNWLRLTW |
| 7154 | RMLsSGVSEVFFRKNWLRLTW |
| 7155 | RMLsVILIKFFRKNWLRLTW |
| 7156 | RMPsFLKKNKFFRKNWLRLTW |
| 7157 | RMPsSTLKKFFRKNWLRLTW |
| 7158 | RMPsSTLKRFFRKNWLRLTW |
| 7159 | RMQsTSERLFFRKNWLRLTW |
| 7160 | RMQsTSERVFFRKNWLRLTW |
| 7161 | RMRQsPLATKFFRKNWLRLTW |
| 7162 | RMRQsPLATRFFRKNWLRLTW |
| 7163 | RMRRsPLLKFFRKNWLRLTW |
| 7164 | RMRsAGAAQKFFRKNWLRLTW |
| 7165 | RMRsLSSLREKFFRKNWLRLTW |
| 7166 | RMRsPPPVSKFFRKNWLRLTW |
| 7167 | RMRTsPITRKFFRKNWLRLTW |
| 7168 | RMRTsPITRRFFRKNWLRLTW |
| 7169 | RMSsLIRHKFFRKNWLRLTW |
| 7170 | RMSsPISKKFFRKNWLRLTW |
| 7171 | RMSsPISKRFFRKNWLRLTW |
| 7172 | RMSsPLHFVFFRKNWLRLTW |
| 7173 | RMSsPVLHKFFRKNWLRLTW |
| 7174 | RMSsRYSQKFFRKNWLRLTW |
| 7175 | RMSsVKLISKFFRKNWLRLTW |
| 7176 | RMSsVKLISYFFRKNWLRLTW |
| 7177 | RMVsLSMRKFFRKNWLRLTW |
| 7178 | RMVsLSMRYFFRKNWLRLTW |
| 7179 | RMYKsPLRHFFRKNWLRLTW |
| 7180 | RMYKsPLRKFFRKNWLRLTW |
| 7181 | RMYQyIQSKFFRKNWLRLTW |
| 7182 | RMYQyIQSRFFRKNWLRLTW |
| 7183 | RMYQyLQSRFFFRKNWLRLTW |
| 7184 | RMYQyLQSRFKFFRKNWLRLTW |
| 7185 | RMYQyLQSRFYFFRKNWLRLTW |
| 7186 | RMYQyLQSRKFFRKNWLRLTW |
| 7187 | RMYsFDDVLFFRKNWLRLTW |
| 7188 | RMYsGSRsKFFRKNWLRLTW |
| 7189 | RMYsGSRsRFFRKNWLRLTW |
| 7190 | RMYsKSRDHFFRKNWLRLTW |
| 7191 | RMYsKSRDKFFRKNWLRLTW |
| 7192 | RMYsKSRDYFFRKNWLRLTW |
| 7193 | RMYsPDHRQKFFRKNWLRLTW |
| 7194 | RMYsPERSKFFRKNWLRLTW |
| 7195 | RMYsPIIYQAFFRKNWLRLTW |
| 7196 | RMYsPIPPSLFFRKNWLRLTW |
| 7197 | RMYsPRNSKFFRKNWLRLTW |
| 7198 | RMYsPYNHKFFRKNWLRLTW |
| 7199 | RMYsPYNHRFFRKNWLRLTW |
| 7200 | RMYsYPRQKFFRKNWLRLTW |
| 7201 | RMYVTTSTRTYsLGFFRKNWLRLTW |
| 7202 | RMYVTTSTRTYsLKFFRKNWLRLTW |
| 7203 | RMYVTTSTRTYsLYFFRKNWLRLTW |
| 7204 | RNLsSPFIFFFRKNWLRLTW |
| 7205 | RPAFFsPSLFFRKNWLRLTW |
| 7206 | RPAKsMDSFFFRKNWLRLTW |
| 7207 | RPAKsMDSLFFRKNWLRLTW |
| 7208 | RPAKsMDSMFFRKNWLRLTW |
| 7209 | RPAKsMDVFFRKNWLRLTW |
| 7210 | RPAsAGAMFFFRKNWLRLTW |
| 7211 | RPAsAGAmLFFRKNWLRLTW |
| 7212 | RPAsAGAMLFFRKNWLRLTW |
| 7213 | RPAsAGAMMFFRKNWLRLTW |
| 7214 | RPAsAGAMVFFRKNWLRLTW |
| 7215 | RPAsARAQPGFFFRKNWLRLTW |
| 7216 | RPAsARAQPGLFFRKNWLRLTW |
| 7217 | RPAsARAQPGMFFRKNWLRLTW |
| 7218 | RPAsARAQPGVFFRKNWLRLTW |
| 7219 | RPAsEARAPGLFFRKNWLRLTW |
| 7220 | RPAsPAAKFFFRKNWLRLTW |
| 7221 | RPAsPAAKLFFRKNWLRLTW |
| 7222 | RPAsPAAKMFFRKNWLRLTW |
| 7223 | RPAsPAAKVFFRKNWLRLTW |
| 7224 | RPAsPEPELFFRKNWLRLTW |
| 7225 | RPAsPGPSLFFRKNWLRLTW |
| 7226 | RPAsPKRAKIFFRKNWLRLTW |
| 7227 | RPAsPKRAKLFFRKNWLRLTW |
| 7228 | RPAsPKRAKXFFRKNWLRLTW |
| 7229 | RPAsPKRAQIFFRKNWLRLTW |
| 7230 | RPAsPKRAQLFFRKNWLRLTW |
| 7231 | RPAsPKRAQXFFRKNWLRLTW |
| 7232 | RPAsPQRAKIFFRKNWLRLTW |
| 7233 | RPAsPQRAKLFFRKNWLRLTW |
| 7234 | RPAsPQRAKXFFRKNWLRLTW |
| 7235 | RPAsPQRAQIFFRKNWLRLTW |
| 7236 | RPAsPQRAQLFFRKNWLRLTW |
| 7237 | RPAsPQRAQXFFRKNWLRLTW |
| 7238 | RPAsPSLQLFFRKNWLRLTW |
| 7239 | RPAsPSLQLLFFRKNWLRLTW |
| 7240 | RPAsPtAIRRIGSVTSRQTFFRKNWLRLTW |
| 7241 | RPAsRFEVLFFRKNWLRLTW |
| 7242 | RPAsYKKKSMLFFRKNWLRLTW |
| 7243 | RPAtGGPGVAFFRKNWLRLTW |
| 7244 | RPAtGGPGVFFFRKNWLRLTW |
| 7245 | RPAtGGPGVLFFRKNWLRLTW |
| 7246 | RPAtGGPGVMFFRKNWLRLTW |
| 7247 | RPAtGGPGWFFRKNWLRLTW |
| 7248 | RPAtPTSQFFFRKNWLRLTW |
| 7249 | RPAtPTSQLFFRKNWLRLTW |
| 7250 | RPAtPTSQMFFRKNWLRLTW |
| 7251 | RPAtPTSQVFFRKNWLRLTW |
| 7252 | RPDsAHKMLFFRKNWLRLTW |
| 7253 | RPDsPTRPTLFFRKNWLRLTW |
| 7254 | RPDsRLGKTEFFFRKNWLRLTW |
| 7255 | RPDsRLGKTELFFRKNWLRLTW |
| 7256 | RPDsRLGKTEMFFRKNWLRLTW |
| 7257 | RPDsRLGKTEVFFRKNWLRLTW |
| 7258 | RPDVAKRLsLFFRKNWLRLTW |
| 7259 | RPEsDSGLKFFFRKNWLRLTW |
| 7260 | RPEsDSGLKLFFRKNWLRLTW |
| 7261 | RPEsDSGLKMFFRKNWLRLTW |
| 7262 | RPEsDSGLKVFFRKNWLRLTW |
| 7263 | RPEsKDRKFFFRKNWLRLTW |
| 7264 | RPEsKDRKLFFRKNWLRLTW |
| 7265 | RPEsKDRKMFFRKNWLRLTW |
| 7266 | RPEsKDRKVFFRKNWLRLTW |
| 7267 | RPFARsHSFFFRKNWLRLTW |
| 7268 | RPFARSHsFFFRKNWLRLTW |
| 7269 | RPFHGISTVsLFFRKNWLRLTW |
| 7270 | RPFsPREAFFFRKNWLRLTW |
| 7271 | RPFsPREALFFRKNWLRLTW |
| 7272 | RPFsPREAMFFRKNWLRLTW |
| 7273 | RPFsPREAVFFRKNWLRLTW |
| 7274 | RPGsLERKFFFRKNWLRLTW |
| 7275 | RPGsLERKLFFRKNWLRLTW |
| 7276 | RPGsLERKMFFRKNWLRLTW |
| 7277 | RPGsLERKVFFRKNWLRLTW |
| 7278 | RPGsRQAGLFFRKNWLRLTW |
| 7279 | RPGsRqAGL FFRKNWLRLTW |
| 7280 | RPHsPEKAFFFRKNWLRLTW |
| 7281 | RPHsPEKALFFRKNWLRLTW |
| 7282 | RPHsPEKAMFFRKNWLRLTW |
| 7283 | RPHsPEKAVFFRKNWLRLTW |
| 7284 | RPHtPTGIYMFFRKNWLRLTW |
| 7285 | RPHtPTPGIYMFFRKNWLRLTW |
| 7286 | RPIsPGLSFFFRKNWLRLTW |
| 7287 | RPIsPGLSLFFRKNWLRLTW |
| 7288 | RPIsPGLSMFFRKNWLRLTW |
| 7289 | RPIsPGLSVFFRKNWLRLTW |
| 7290 | RPIsPGLSYFFRKNWLRLTW |
| 7291 | RPIsPPHTYFFRKNWLRLTW |
| 7292 | RPIsPRIGALFFRKNWLRLTW |
| 7293 | RPItPPRNSAFFRKNWLRLTW |
| 7294 | RPItPPRNSFFFRKNWLRLTW |
| 7295 | RPItPPRNSLFFRKNWLRLTW |
| 7296 | RPItPPRNSMFFRKNWLRLTW |
| 7297 | RPItPPRNSVFFRKNWLRLTW |
| 7298 | RPKLSsPAFFFRKNWLRLTW |
| 7299 | RPKLSsPALFFRKNWLRLTW |
| 7300 | RPKLSsPAMFFRKNWLRLTW |
| 7301 | RPKLSsPAVFFRKNWLRLTW |
| 7302 | RPKPSSsPFFFRKNWLRLTW |
| 7303 | RPKPSSsPLFFRKNWLRLTW |
| 7304 | RPKPSSsPMFFRKNWLRLTW |
| 7305 | RPKPSSsPVFFRKNWLRLTW |
| 7306 | RPKsNIVLFFFRKNWLRLTW |
| 7307 | RPKsNIVLLFFRKNWLRLTW |
| 7308 | RPKsNIVLMFFRKNWLRLTW |
| 7309 | RPKsNIVLVFFRKNWLRLTW |
| 7310 | RPKsPLSKmFFRKNWLRLTW |
| 7311 | RPKsPLSKMFFRKNWLRLTW |
| 7312 | RPKsQVAEFFFRKNWLRLTW |
| 7313 | RPKsQVAELFFRKNWLRLTW |
| 7314 | RPKsQVAEMFFRKNWLRLTW |
| 7315 | RPKsQVAEVFFRKNWLRLTW |
| 7316 | RPKsVDFDSLFFRKNWLRLTW |
| 7317 | RPKtPPWIFFRKNWLRLTW |
| 7318 | RPLsLLLALFFRKNWLRLTW |
| 7319 | RPLsPGGAFFFRKNWLRLTW |
| 7320 | RPLsPGGALFFRKNWLRLTW |
| 7321 | RPLsPGGAMFFRKNWLRLTW |
| 7322 | RPLsPGGAVFFRKNWLRLTW |
| 7323 | RPLsPLLFFFRKNWLRLTW |
| 7324 | RPLsPLLLFFRKNWLRLTW |
| 7325 | RPLsPLLMFFRKNWLRLTW |
| 7326 | RPLsPLLVFFRKNWLRLTW |
| 7327 | RPLsWYVLFFRKNWLRLTW |
| 7328 | RPMsESPHMFFRKNWLRLTW |
| 7329 | RPNsPSPTAFFFRKNWLRLTW |
| 7330 | RPNsPSPTALFFRKNWLRLTW |
| 7331 | RPNsPSPTAMFFRKNWLRLTW |
| 7332 | RPNsPSPTAVFFRKNWLRLTW |
| 7333 | RPPIgTQSSLFFRKNWLRLTW |
| 7334 | RPPPPPDtPFFFRKNWLRLTW |
| 7335 | RPPPPPDtPLFFRKNWLRLTW |
| 7336 | RPPPPPDtPMFFRKNWLRLTW |
| 7337 | RPPPPPDtPPFFRKNWLRLTW |
| 7338 | RPPPPPDtPVFFRKNWLRLTW |
| 7339 | RPPsPGPVFFFRKNWLRLTW |
| 7340 | RPPsPGPVLFFRKNWLRLTW |
| 7341 | RPPsPGPVMFFRKNWLRLTW |
| 7342 | RPPsPGPWFFRKNWLRLTW |
| 7343 | RPPsPSSRFFFRKNWLRLTW |
| 7344 | RPPsPSSRLFFRKNWLRLTW |
| 7345 | RPPsPSSRMFFRKNWLRLTW |
| 7346 | RPPsPSSRVFFRKNWLRLTW |
| 7347 | RPPsSEFLDFFFRKNWLRLTW |
| 7348 | RPPsSEFLDLFFRKNWLRLTW |
| 7349 | RPPsSEFLDMFFRKNWLRLTW |
| 7350 | RPPsSEFLDVFFRKNWLRLTW |
| 7351 | RPQKTQsIIFFRKNWLRLTW |
| 7352 | RPQRAtSNVFFFRKNWLRLTW |
| 7353 | RPQRATsNVFFFRKNWLRLTW |
| 7354 | RPQRAtSNVLFFRKNWLRLTW |
| 7355 | RPQRATsNVLFFRKNWLRLTW |
| 7356 | RPQRAtSNVMFFRKNWLRLTW |
| 7357 | RPQRATsNVMFFRKNWLRLTW |
| 7358 | RPQRAtSNWFFRKNWLRLTW |
| 7359 | RPQRATsNWFFRKNWLRLTW |
| 7360 | RPR(sLss)PTVTLFFRKNWLRLTW |
| 7361 | RPR(sLss)PTVTVFFRKNWLRLTW |
| 7362 | RPRAAtWFFRKNWLRLTW |
| 7363 | RPRAAtWAFFRKNWLRLTW |
| 7364 | RPRAAtWFFRKNWLRLTW |
| 7365 | RPRAAtWAFFRKNWLRLTW |
| 7366 | RPRANsGGVDFFFRKNWLRLTW |
| 7367 | RPRANsGGVDLFFRKNWLRLTW |
| 7368 | RPRANsGGVDMFFRKNWLRLTW |
| 7369 | RPRANsGGVDVFFRKNWLRLTW |
| 7370 | RPRARsVDALFFRKNWLRLTW |
| 7371 | RPRDtRRISLFFRKNWLRLTW |
| 7372 | RPRGsESLLFFRKNWLRLTW |
| 7373 | RPRGsQSLFFFRKNWLRLTW |
| 7374 | RPRGsQSLLFFRKNWLRLTW |
| 7375 | RPRGsQSLMFFRKNWLRLTW |
| 7376 | RPRGsQSLVFFRKNWLRLTW |
| 7377 | RPRIPsPIGFFFRKNWLRLTW |
| 7378 | RPRLSsTNSSRFFFRKNWLRLTW |
| 7379 | RPRPAsSPALFFRKNWLRLTW |
| 7380 | RPRPHsAPSFFFRKNWLRLTW |
| 7381 | RPRPHsAPSLFFRKNWLRLTW |
| 7382 | RPRPHsAPSMFFRKNWLRLTW |
| 7383 | RPRPHsAPSVFFRKNWLRLTW |
| 7384 | RPRPSsAHVGLFFRKNWLRLTW |
| 7385 | RPRPsSVLFFRKNWLRLTW |
| 7386 | RPRPsSVLRTLFFRKNWLRLTW |
| 7387 | RPRPVsPSSFFFRKNWLRLTW |
| 7388 | RPRPVsPSSLFFRKNWLRLTW |
| 7389 | RPRPVsPSSLLFFRKNWLRLTW |
| 7390 | RPRPVsPSSMFFRKNWLRLTW |
| 7391 | RPRPVsPSSVFFRKNWLRLTW |
| 7392 | RPRRsSTQFFFRKNWLRLTW |
| 7393 | RPRRsSTQLFFRKNWLRLTW |
| 7394 | RPRRsSTQMFFRKNWLRLTW |
| 7395 | RPRRsSTQVFFRKNWLRLTW |
| 7396 | RPRsAVEQLFFRKNWLRLTW |
| 7397 | RPRsAVLFFFRKNWLRLTW |
| 7398 | RPRsAVLLFFRKNWLRLTW |
| 7399 | RPRsAVLMFFRKNWLRLTW |
| 7400 | RPRsAVLVFFRKNWLRLTW |
| 7401 | RPRSGsTGSSLFFRKNWLRLTW |
| 7402 | RPRsISVEEFFFRKNWLRLTW |
| 7403 | RPRsISVEELFFRKNWLRLTW |
| 7404 | RPRsISVEEMFFRKNWLRLTW |
| 7405 | RPRsISVEEVFFRKNWLRLTW |
| 7406 | RPRsLEVTFFFRKNWLRLTW |
| 7407 | RPRsLEVTIFFRKNWLRLTW |
| 7408 | RPRsLEVTLFFRKNWLRLTW |
| 7409 | RPRsLEVTMFFRKNWLRLTW |
| 7410 | RPRsLEVTVFFRKNWLRLTW |
| 7411 | RPRSLsSPTVFFRKNWLRLTW |
| 7412 | RPRSLsSPTVTFFFRKNWLRLTW |
| 7413 | RPRSLsSPTVTLFFRKNWLRLTW |
| 7414 | RPRSLsSPTVTMFFRKNWLRLTW |
| 7415 | RPRSLsSPTVTVFFRKNWLRLTW |
| 7416 | RPRsMTVSAFFRKNWLRLTW |
| 7417 | RPRsMVRSFFFRKNWLRLTW |
| 7418 | RPRsPAARFFFRKNWLRLTW |
| 7419 | RPRsPAARLFFRKNWLRLTW |
| 7420 | RPRsPAARMFFRKNWLRLTW |
| 7421 | RPRsPAARVFFRKNWLRLTW |
| 7422 | RPRsPGSNSKVFFRKNWLRLTW |
| 7423 | RPRsPNMQDLFFRKNWLRLTW |
| 7424 | RPRsPPGGPFFRKNWLRLTW |
| 7425 | RPRsPPPRAFFFRKNWLRLTW |
| 7426 | RPRsPPPRALFFRKNWLRLTW |
| 7427 | RPRsPPPRAMFFRKNWLRLTW |
| 7428 | RPRsPPPRAPFFRKNWLRLTW |
| 7429 | RPRsPPPRAVFFRKNWLRLTW |
| 7430 | RPRsPPSSPFFRKNWLRLTW |
| 7431 | RPRsPRENSFFFRKNWLRLTW |
| 7432 | RPRsPRENSIFFRKNWLRLTW |
| 7433 | RPRsPRENSLFFRKNWLRLTW |
| 7434 | RPRsPRENSMFFRKNWLRLTW |
| 7435 | RPRsPRENSVFFRKNWLRLTW |
| 7436 | RPRsPRPPPFFRKNWLRLTW |
| 7437 | RPRsPRQNLIFFRKNWLRLTW |
| 7438 | RPRsPRQNSFFFRKNWLRLTW |
| 7439 | RPRsPRQNSIFFRKNWLRLTW |
| 7440 | RPRsPRQNSMFFRKNWLRLTW |
| 7441 | RPRsPRQNSVFFRKNWLRLTW |
| 7442 | RPRsPSPIFFFRKNWLRLTW |
| 7443 | RPRsPSPILFFRKNWLRLTW |
| 7444 | RPRsPSPIMFFRKNWLRLTW |
| 7445 | RPRsPSPISFFRKNWLRLTW |
| 7446 | RPRSPsPISFFRKNWLRLTW |
| 7447 | RPRsPSPIVFFRKNWLRLTW |
| 7448 | RPRsPTGFFFRKNWLRLTW |
| 7449 | RPRsPTGLFFRKNWLRLTW |
| 7450 | RPRsPTGMFFRKNWLRLTW |
| 7451 | RPRsPTGPFFRKNWLRLTW |
| 7452 | RPRsPTGPsNSFFFRKNWLRLTW |
| 7453 | RPRsPTGPSNSFFFRKNWLRLTW |
| 7454 | RPRsPTGPSNSFLFFRKNWLRLTW |
| 7455 | RPRsPTGPsNSLFFRKNWLRLTW |
| 7456 | RPRsPTGPsNSMFFRKNWLRLTW |
| 7457 | RPRsPTGPsNSVFFRKNWLRLTW |
| 7458 | RPRsPTGVFFRKNWLRLTW |
| 7459 | RPRsPTRSFFFRKNWLRLTW |
| 7460 | RPRsPTRSLFFRKNWLRLTW |
| 7461 | RPRsPTRSMFFRKNWLRLTW |
| 7462 | RPRsPTRSVFFRKNWLRLTW |
| 7463 | RPRsPWGKLFFRKNWLRLTW |
| 7464 | RPRsQYNTKLFFRKNWLRLTW |
| 7465 | RPRSTsQSIVSLFFRKNWLRLTW |
| 7466 | RPRtPLRSLFFRKNWLRLTW |
| 7467 | RPSGRREsFFFRKNWLRLTW |
| 7468 | RPSGRREsLFFRKNWLRLTW |
| 7469 | RPSGRREsMFFRKNWLRLTW |
| 7470 | RPSGRREsVFFRKNWLRLTW |
| 7471 | RPsNPQLFFRKNWLRLTW |
| 7472 | RPSRSsPGFFFRKNWLRLTW |
| 7473 | RPSRSsPGLFFRKNWLRLTW |
| 7474 | RPSRSsPGMFFRKNWLRLTW |
| 7475 | RPSRSsPGVFFRKNWLRLTW |
| 7476 | RPSsGFYELFFRKNWLRLTW |
| 7477 | RPSsLDAEIDSFFFRKNWLRLTW |
| 7478 | RPSsLDAEIDSLFFRKNWLRLTW |
| 7479 | RPSsLDAEIDSMFFRKNWLRLTW |
| 7480 | RPSsLDAEIDSVFFRKNWLRLTW |
| 7481 | RPSsLPDFFFRKNWLRLTW |
| 7482 | RPSsLPDLFFRKNWLRLTW |
| 7483 | RPSsLPDMFFRKNWLRLTW |
| 7484 | RPSsLPDVFFRKNWLRLTW |
| 7485 | RPsSPALYFFFRKNWLRLTW |
| 7486 | RPSsPALYFFFRKNWLRLTW |
| 7487 | RPsSPALYLFFRKNWLRLTW |
| 7488 | RPsSPALYMFFRKNWLRLTW |
| 7489 | RPsSPALYVFFRKNWLRLTW |
| 7490 | RPStPKSDSEFFFRKNWLRLTW |
| 7491 | RPStPKSDSELFFRKNWLRLTW |
| 7492 | RPStPKSDSEMFFRKNWLRLTW |
| 7493 | RPStPKSDSEVFFRKNWLRLTW |
| 7494 | RPTKIGRRsLFFRKNWLRLTW |
| 7495 | RPTsFADELFFRKNWLRLTW |
| 7496 | RPTsPIQIMFFRKNWLRLTW |
| 7497 | RPTsRLNRFFFRKNWLRLTW |
| 7498 | RPTsRLNRLFFRKNWLRLTW |
| 7499 | RPTsRLNRMFFRKNWLRLTW |
| 7500 | RPTsRLNRVFFRKNWLRLTW |
| 7501 | RPVsPFQEFFFRKNWLRLTW |
| 7502 | RPVsPFQELFFRKNWLRLTW |
| 7503 | RPVsPFQEMFFRKNWLRLTW |
| 7504 | RPVsPFQEVFFRKNWLRLTW |
| 7505 | RPVsPGKDFFFRKNWLRLTW |
| 7506 | RPVsPGKDIFFRKNWLRLTW |
| 7507 | RPVsPGKDLFFRKNWLRLTW |
| 7508 | RPVsPGKDMFFRKNWLRLTW |
| 7509 | RPVsPGKDVFFRKNWLRLTW |
| 7510 | RPVSPsSLLFFRKNWLRLTW |
| 7511 | RPVsTDFAQYFFRKNWLRLTW |
| 7512 | RPVtPVSDFFFRKNWLRLTW |
| 7513 | RPVtPVSDLFFRKNWLRLTW |
| 7514 | RPVtPVSDMFFRKNWLRLTW |
| 7515 | RPVtPVSDVFFRKNWLRLTW |
| 7516 | RPWsNSRGLFFRKNWLRLTW |
| 7517 | RPWsPAVSAFFRKNWLRLTW |
| 7518 | RPWsPAVSFFFRKNWLRLTW |
| 7519 | RPWsPAVSLFFRKNWLRLTW |
| 7520 | RPWsPAVSMFFRKNWLRLTW |
| 7521 | RPWsPAVSVFFRKNWLRLTW |
| 7522 | RPYsPPFFSFFFRKNWLRLTW |
| 7523 | RPYsPPFFSLFFRKNWLRLTW |
| 7524 | RPYsPPFFSMFFRKNWLRLTW |
| 7525 | RPYsPPFFSVFFRKNWLRLTW |
| 7526 | RPYSPsQALFFRKNWLRLTW |
| 7527 | RPYsPSQYALFFRKNWLRLTW |
| 7528 | RPYSPsQYALFFRKNWLRLTW |
| 7529 | RPYsQVNVLFFRKNWLRLTW |
| 7530 | RQAsIELPSMFFRKNWLRLTW |
| 7531 | RQAsIELPSMAVFFRKNWLRLTW |
| 7532 | RQAsIELPSVFFRKNWLRLTW |
| 7533 | RQAsLSISVFFRKNWLRLTW |
| 7534 | RQAsPLVHKFFRKNWLRLTW |
| 7535 | RQAsPLVHRFFRKNWLRLTW |
| 7536 | RQAsPLVHYFFRKNWLRLTW |
| 7537 | RQDsTPGKVFLFFRKNWLRLTW |
| 7538 | RQDStPGKVFLFFRKNWLRLTW |
| 7539 | RQDsTPGKVFVFFRKNWLRLTW |
| 7540 | RQIsFKAEVFFRKNWLRLTW |
| 7541 | RQIsQDVKLFFRKNWLRLTW |
| 7542 | RQIsQDVKVFFRKNWLRLTW |
| 7543 | RQKsPLFQFFFRKNWLRLTW |
| 7544 | RQLsALHRAFFRKNWLRLTW |
| 7545 | RQLsLEGSGLGVFFRKNWLRLTW |
| 7546 | RQLsSGVSEIFFRKNWLRLTW |
| 7547 | RQLsSGVSEVFFRKNWLRLTW |
| 7548 | RQSsSRFNLFFRKNWLRLTW |
| 7549 | RRAsFAKSFFFRKNWLRLTW |
| 7550 | RRAsFAKSKFFRKNWLRLTW |
| 7551 | RRAsFAKSLFFRKNWLRLTW |
| 7552 | RRAsFAKSMFFRKNWLRLTW |
| 7553 | RRAsFAKSRFFRKNWLRLTW |
| 7554 | RRAsIITKYFFRKNWLRLTW |
| 7555 | RRAsLSEIGFFFRKNWLRLTW |
| 7556 | RRAsLSEIGKFFRKNWLRLTW |
| 7557 | RRAsLSEIGYFFRKNWLRLTW |
| 7558 | RRAsQEANLFFRKNWLRLTW |
| 7559 | RRASsPFRFFFRKNWLRLTW |
| 7560 | RRASsPFRKFFRKNWLRLTW |
| 7561 | RRASsPFRLFFRKNWLRLTW |
| 7562 | RRASsPFRMFFRKNWLRLTW |
| 7563 | RRASsPFRRFFRKNWLRLTW |
| 7564 | RRAsVFVKFFFRKNWLRLTW |
| 7565 | RRAsVFVKKFFRKNWLRLTW |
| 7566 | RRAsVFVKLFFRKNWLRLTW |
| 7567 | RRAsVFVKMFFRKNWLRLTW |
| 7568 | RRAsVFVKRFFRKNWLRLTW |
| 7569 | RRDsIVAEFFFRKNWLRLTW |
| 7570 | RRDsIVAEKFFRKNWLRLTW |
| 7571 | RRDsIVAELFFRKNWLRLTW |
| 7572 | RRDsIVAERFFRKNWLRLTW |
| 7573 | RRDsIVAEYFFRKNWLRLTW |
| 7574 | RRDsLQKPGLFFRKNWLRLTW |
| 7575 | RRFsFEVTLFFRKNWLRLTW |
| 7576 | RRFsFKFFFRKNWLRLTW |
| 7577 | RRFsFKKFFRKNWLRLTW |
| 7578 | RRFsFKKSFFFRKNWLRLTW |
| 7579 | RRFsFKKSKFFRKNWLRLTW |
| 7580 | RRFsFKKSLFFRKNWLRLTW |
| 7581 | RRFsFKKSMFFRKNWLRLTW |
| 7582 | RRFsFKKSRFFRKNWLRLTW |
| 7583 | RRFsFKLFFRKNWLRLTW |
| 7584 | RRFsFKMFFRKNWLRLTW |
| 7585 | RRFsFKRFFRKNWLRLTW |
| 7586 | RRFsGTAVYFFRKNWLRLTW |
| 7587 | RRFsGTVRFFFRKNWLRLTW |
| 7588 | RRFsGTVRKFFRKNWLRLTW |
| 7589 | RRFsGTVRLFFRKNWLRLTW |
| 7590 | RRFsGTVRMFFRKNWLRLTW |
| 7591 | RRFsGTVRRFFRKNWLRLTW |
| 7592 | RRFsIATLRFFRKNWLRLTW |
| 7593 | RRFsLTTLRFFRKNWLRLTW |
| 7594 | RRFsPDDKYSFFFRKNWLRLTW |
| 7595 | RRFsPDDKYSKFFRKNWLRLTW |
| 7596 | RRFsPDDKYSLFFRKNWLRLTW |
| 7597 | RRFsPDDKYSMFFRKNWLRLTW |
| 7598 | RRFsPPRRFFFRKNWLRLTW |
| 7599 | RRFsPPRRKFFRKNWLRLTW |
| 7600 | RRFsPPRRLFFRKNWLRLTW |
| 7601 | RRFsPPRRmFFRKNWLRLTW |
| 7602 | RRFsPPRRMFFRKNWLRLTW |
| 7603 | RRFsPPRRRFFRKNWLRLTW |
| 7604 | RRFsPPRRYFFRKNWLRLTW |
| 7605 | RRFsRLENRYFFRKNWLRLTW |
| 7606 | RRFsRSDELFFRKNWLRLTW |
| 7607 | RRFsRsPIFFFRKNWLRLTW |
| 7608 | RRFsRSPIFFFRKNWLRLTW |
| 7609 | RRFsRsPIKFFRKNWLRLTW |
| 7610 | RRFsRSPIKFFRKNWLRLTW |
| 7611 | RRFsRsPILFFRKNWLRLTW |
| 7612 | RRFsRSPILFFRKNWLRLTW |
| 7613 | RRFsRSPIMFFRKNWLRLTW |
| 7614 | RRFsRsPIRFFRKNWLRLTW |
| 7615 | RRFsRSPIRFFRKNWLRLTW |
| 7616 | RRFSRsPIRFFRKNWLRLTW |
| 7617 | RRFsRsPIRFFFRKNWLRLTW |
| 7618 | RRFsRSPIRFFFRKNWLRLTW |
| 7619 | RRFsRsPIRKFFRKNWLRLTW |
| 7620 | RRFsRSPIRKFFRKNWLRLTW |
| 7621 | RRFsRsPIRLFFRKNWLRLTW |
| 7622 | RRFsRSPIRLFFRKNWLRLTW |
| 7623 | RRFsRsPIRRFFRKNWLRLTW |
| 7624 | RRFsRSPIRRFFRKNWLRLTW |
| 7625 | RRFsRsPIRYFFRKNWLRLTW |
| 7626 | RRFsRSPIRYFFRKNWLRLTW |
| 7627 | RRFsRsPIYFFRKNWLRLTW |
| 7628 | RRFsRSPIYFFRKNWLRLTW |
| 7629 | RRFsRSPKFFRKNWLRLTW |
| 7630 | RRFSsPPRRMFFRKNWLRLTW |
| 7631 | RRFsVSTLRFFRKNWLRLTW |
| 7632 | RRFsVTTMRFFRKNWLRLTW |
| 7633 | RRFtPPSPAFFFRKNWLRLTW |
| 7634 | RRFtPPSPAKFFRKNWLRLTW |
| 7635 | RRFtPPSPARFFRKNWLRLTW |
| 7636 | RRFtPPSPAYFFRKNWLRLTW |
| 7637 | RRGsFEVTLFFRKNWLRLTW |
| 7638 | RRHsASNLHALFFRKNWLRLTW |
| 7639 | RRIDIsPSTFFFRKNWLRLTW |
| 7640 | RRIDIsPSTKFFRKNWLRLTW |
| 7641 | RRIDIsPSTLRFFRKNWLRLTW |
| 7642 | RRIDIsPSTLRKFFRKNWLRLTW |
| 7643 | RRIDIsPSTRFFRKNWLRLTW |
| 7644 | RRIDIsPSTYFFRKNWLRLTW |
| 7645 | RRIsDPEVFFFRKNWLRLTW |
| 7646 | RRIsDPQVFFFRKNWLRLTW |
| 7647 | RRIsGVDRFFFRKNWLRLTW |
| 7648 | RRIsGVDRKFFRKNWLRLTW |
| 7649 | RRIsGVDRLFFRKNWLRLTW |
| 7650 | RRIsGVDRMFFRKNWLRLTW |
| 7651 | RRIsGVDRRFFRKNWLRLTW |
| 7652 | RRIsGVDRYFFRKNWLRLTW |
| 7653 | RRIsGVDRYFFFRKNWLRLTW |
| 7654 | RRIsGVDRYKFFRKNWLRLTW |
| 7655 | RRIsGVDRYLFFRKNWLRLTW |
| 7656 | RRIsGVDRYRFFRKNWLRLTW |
| 7657 | RRISGVDRYYFFRKNWLRLTW |
| 7658 | RRISPAPQRFFRKNWLRLTW |
| 7659 | RRIsQIQQLFFRKNWLRLTW |
| 7660 | RRKsOVAEFFFRKNWLRLTW |
| 7661 | RRKsOVAEKFFRKNWLRLTW |
| 7662 | RRKsPPPSFFFRKNWLRLTW |
| 7663 | RRKsPPPSKFFRKNWLRLTW |
| 7664 | RRKsPPPSLFFRKNWLRLTW |
| 7665 | RRKsPPPSMFFRKNWLRLTW |
| 7666 | RRKsPPPSRFFRKNWLRLTW |
| 7667 | RRKsQLDSFFFRKNWLRLTW |
| 7668 | RRKsQLDSKFFRKNWLRLTW |
| 7669 | RRKsQLDSLFFRKNWLRLTW |
| 7670 | RRKsQLDSMFFRKNWLRLTW |
| 7671 | RRKsQLDSRFFRKNWLRLTW |
| 7672 | RRKsQLDSYFFRKNWLRLTW |
| 7673 | RRKsQVAEFFFRKNWLRLTW |
| 7674 | RRKsQVAEKFFRKNWLRLTW |
| 7675 | RRKsQVAELFFRKNWLRLTW |
| 7676 | RRKsQVAEMFFRKNWLRLTW |
| 7677 | RRKsQVAERFFRKNWLRLTW |
| 7678 | RRKsQVAEVFFRKNWLRLTW |
| 7679 | RRKsQVAEYFFRKNWLRLTW |
| 7680 | RRLGsPHRFFFRKNWLRLTW |
| 7681 | RRLGsPHRKFFRKNWLRLTW |
| 7682 | RRLGsPHRLFFRKNWLRLTW |
| 7683 | RRLGsPHRMFFRKNWLRLTW |
| 7684 | RRLGsPHRRFFRKNWLRLTW |
| 7685 | RRLsADIRFFFRKNWLRLTW |
| 7686 | RRLsADIRKFFRKNWLRLTW |
| 7687 | RRLsADIRLFFRKNWLRLTW |
| 7688 | RRLsADIRMFFRKNWLRLTW |
| 7689 | RRLsADIRRFFRKNWLRLTW |
| 7690 | RRLsADIRYFFRKNWLRLTW |
| 7691 | RRLsDSPVFFFRKNWLRLTW |
| 7692 | RRLsELLRYFFRKNWLRLTW |
| 7693 | RRLsERETRFFRKNWLRLTW |
| 7694 | RRLsESSALFFRKNWLRLTW |
| 7695 | RRLsFLVSFFFRKNWLRLTW |
| 7696 | RRLsFLVSKFFRKNWLRLTW |
| 7697 | RRLsFLVSLFFRKNWLRLTW |
| 7698 | RRLsFLVSMFFRKNWLRLTW |
| 7699 | RRLsFLVSRFFRKNWLRLTW |
| 7700 | RRLsFLVSYFFRKNWLRLTW |
| 7701 | RRLsGGSHSFFFRKNWLRLTW |
| 7702 | RRLsGGSHSKFFRKNWLRLTW |
| 7703 | RRLsGGSHSLFFRKNWLRLTW |
| 7704 | RRLsGGSHSMFFRKNWLRLTW |
| 7705 | RRLsGGSHSRFFRKNWLRLTW |
| 7706 | RRLsGGSHSYFFRKNWLRLTW |
| 7707 | RRLsGPLHTFFFRKNWLRLTW |
| 7708 | RRLsGPLHTKFFRKNWLRLTW |
| 7709 | RRLsGPLHTLFFRKNWLRLTW |
| 7710 | RRLsGPLHTMFFRKNWLRLTW |
| 7711 | RRLsGPLHTRFFRKNWLRLTW |
| 7712 | RRLsGPLHTVFFRKNWLRLTW |
| 7713 | RRLsGPLHTYFFRKNWLRLTW |
| 7714 | RRLsLFLNVFFRKNWLRLTW |
| 7715 | RRLsNLPTFFFRKNWLRLTW |
| 7716 | RRLsNLPTKFFRKNWLRLTW |
| 7717 | RRLsNLPTRFFRKNWLRLTW |
| 7718 | RRLsNLPTVFFRKNWLRLTW |
| 7719 | RRLsNLPTYFFRKNWLRLTW |
| 7720 | RRLsPAPOFFFRKNWLRLTW |
| 7721 | RRLsPAPQKFFRKNWLRLTW |
| 7722 | RRLsPAPQLFFRKNWLRLTW |
| 7723 | RRLsPAPQMFFRKNWLRLTW |
| 7724 | RRLsPKASQVFFFRKNWLRLTW |
| 7725 | RRLsPKASQVKFFRKNWLRLTW |
| 7726 | RRLsPKASQVLFFRKNWLRLTW |
| 7727 | RRLsPKASQVMFFRKNWLRLTW |
| 7728 | RRLsPKASQVRFFRKNWLRLTW |
| 7729 | RRLsPVPVPFFFRKNWLRLTW |
| 7730 | RRLsPVPVPKFFRKNWLRLTW |
| 7731 | RRLsPVPVPLFFRKNWLRLTW |
| 7732 | RRLsPVPVPMFFRKNWLRLTW |
| 7733 | RRLsPVPVPRFFRKNWLRLTW |
| 7734 | RRLsRELOKFFRKNWLRLTW |
| 7735 | RRLsRELQFFFRKNWLRLTW |
| 7736 | RRLsRELQLFFRKNWLRLTW |
| 7737 | RRLsRELQMFFRKNWLRLTW |
| 7738 | RRLsRELQRFFRKNWLRLTW |
| 7739 | RRLsRKLSLFFRKNWLRLTW |
| 7740 | RRLsVERIFFFRKNWLRLTW |
| 7741 | RRLsVERIKFFRKNWLRLTW |
| 7742 | RRLsVERIMFFRKNWLRLTW |
| 7743 | RRLsVERIRFFRKNWLRLTW |
| 7744 | RRLsYVLFIFFRKNWLRLTW |
| 7745 | RRLTHLsFFFRKNWLRLTW |
| 7746 | RRLTHLsKFFRKNWLRLTW |
| 7747 | RRLTHLsLFFRKNWLRLTW |
| 7748 | RRLTHLsMFFRKNWLRLTW |
| 7749 | RRLTHLsRFFRKNWLRLTW |
| 7750 | RRMsFQKPFFRKNWLRLTW |
| 7751 | RRMsLLSVFFFRKNWLRLTW |
| 7752 | RRMsLLSVKFFRKNWLRLTW |
| 7753 | RRMsLLSVLFFRKNWLRLTW |
| 7754 | RRMsLLSVMFFRKNWLRLTW |
| 7755 | RRMsLLSVRFFRKNWLRLTW |
| 7756 | RRmsLLSWFFRKNWLRLTW |
| 7757 | RRMsLLSWFFRKNWLRLTW |
| 7758 | RRMsLLSVYFFRKNWLRLTW |
| 7759 | RRMsLLSWFFRKNWLRLTW |
| 7760 | RRMsLSVMFFRKNWLRLTW |
| 7761 | RRMsPIKPLFFRKNWLRLTW |
| 7762 | RRMsPKAORFFRKNWLRLTW |
| 7763 | RRMsPKAQFFFRKNWLRLTW |
| 7764 | RRMsPKAQKFFRKNWLRLTW |
| 7765 | RRMsPKAQLFFRKNWLRLTW |
| 7766 | RRMsPKAQMFFRKNWLRLTW |
| 7767 | RRMsPKPFFFRKNWLRLTW |
| 7768 | RRMsPKPKFFRKNWLRLTW |
| 7769 | RRMsPKPMFFRKNWLRLTW |
| 7770 | RRMsPKPRFFRKNWLRLTW |
| 7771 | RRNsAPVSVFFRKNWLRLTW |
| 7772 | RRNsINRNFFFRKNWLRLTW |
| 7773 | RRNsNPVIAEFFFRKNWLRLTW |
| 7774 | RRNsNPVIAEKFFRKNWLRLTW |
| 7775 | RRNsNPVIAELFFRKNWLRLTW |
| 7776 | RRNsNPVIAEMFFRKNWLRLTW |
| 7777 | RRNsNPVIAERFFRKNWLRLTW |
| 7778 | RRNsSERTFFFRKNWLRLTW |
| 7779 | RRNsSERTKFFRKNWLRLTW |
| 7780 | RRNsSERTLFFRKNWLRLTW |
| 7781 | RRNsSERTMFFRKNWLRLTW |
| 7782 | RRNsSERTRFFRKNWLRLTW |
| 7783 | RRNsSERTYFFRKNWLRLTW |
| 7784 | RRNsSIVGFFFRKNWLRLTW |
| 7785 | RRNsSIVGKFFRKNWLRLTW |
| 7786 | RRNsSIVGLFFRKNWLRLTW |
| 7787 | RRNsSIVGMFFRKNWLRLTW |
| 7788 | RRNsSIVGRFFRKNWLRLTW |
| 7789 | RRNsSIVGYFFRKNWLRLTW |
| 7790 | RRNsVFQQGFFFRKNWLRLTW |
| 7791 | RRNsVFQQGKFFRKNWLRLTW |
| 7792 | RRNsVFQQGLFFRKNWLRLTW |
| 7793 | RRNsVFQQGMFFRKNWLRLTW |
| 7794 | RRNsVFQQGRFFRKNWLRLTW |
| 7795 | RRNsVFQQGYFFRKNWLRLTW |
| 7796 | RRPsIAPVLFFRKNWLRLTW |
| 7797 | RRPsLLSEFFFRKNWLRLTW |
| 7798 | RRPsLVHGFFFRKNWLRLTW |
| 7799 | RRPsLVHGKFFRKNWLRLTW |
| 7800 | RRPsLVHGLFFRKNWLRLTW |
| 7801 | RRPsLVHGMFFRKNWLRLTW |
| 7802 | RRPsLVHGRFFRKNWLRLTW |
| 7803 | RRPsLVHGYFFRKNWLRLTW |
| 7804 | RRPsVFERFFFRKNWLRLTW |
| 7805 | RRPsVFERKFFRKNWLRLTW |
| 7806 | RRPsVFERLFFRKNWLRLTW |
| 7807 | RRPsVFERMFFRKNWLRLTW |
| 7808 | RRPsVFERRFFRKNWLRLTW |
| 7809 | RRPsVFERYFFRKNWLRLTW |
| 7810 | RRPsYRKIFFFRKNWLRLTW |
| 7811 | RRPsYRKIKFFRKNWLRLTW |
| 7812 | RRPsYRKILFFRKNWLRLTW |
| 7813 | RRPsYRKIMFFRKNWLRLTW |
| 7814 | RRPsYRKIRFFRKNWLRLTW |
| 7815 | RRPsYRKIYFFRKNWLRLTW |
| 7816 | RRPsYTLGFFFRKNWLRLTW |
| 7817 | RRPsYTLGKFFRKNWLRLTW |
| 7818 | RRPsYTLGLFFRKNWLRLTW |
| 7819 | RRPsYTLGMFFRKNWLRLTW |
| 7820 | RRPsYTLGRFFRKNWLRLTW |
| 7821 | RRPsYTLGVFFRKNWLRLTW |
| 7822 | RRPsYTLGYFFRKNWLRLTW |
| 7823 | RRQsKVEALFFRKNWLRLTW |
| 7824 | RRRsLERLLFFRKNWLRLTW |
| 7825 | RRsFLVSYFFRKNWLRLTW |
| 7826 | RRSFsLEFFRKNWLRLTW |
| 7827 | RRSsFLQFFRKNWLRLTW |
| 7828 | RRssFLQLFFFRKNWLRLTW |
| 7829 | RRssFLQVFFFRKNWLRLTW |
| 7830 | RRSsFLQVFFFRKNWLRLTW |
| 7831 | RRSsFLQVKFFRKNWLRLTW |
| 7832 | RRSsFLQVLFFRKNWLRLTW |
| 7833 | RRssFLQVMFFRKNWLRLTW |
| 7834 | RRSsFLQVMFFRKNWLRLTW |
| 7835 | RRSsFLQVRFFRKNWLRLTW |
| 7836 | RRssFLQWFFRKNWLRLTW |
| 7837 | RRSsFLQVYFFRKNWLRLTW |
| 7838 | RRSsIGLRFFFRKNWLRLTW |
| 7839 | RRSsIGLRKFFRKNWLRLTW |
| 7840 | RRSsIGLRLFFRKNWLRLTW |
| 7841 | RRSsIGLRMFFRKNWLRLTW |
| 7842 | RRSsIGLRRFFRKNWLRLTW |
| 7843 | RRSsIGLRVFFRKNWLRLTW |
| 7844 | RRSsIGLRYFFRKNWLRLTW |
| 7845 | RRsSIQSTFFFRKNWLRLTW |
| 7846 | RRSsIQSTFFFRKNWLRLTW |
| 7847 | RRSsIQSTKFFRKNWLRLTW |
| 7848 | RRSsIQSTLFFRKNWLRLTW |
| 7849 | RRSsIQSTMFFRKNWLRLTW |
| 7850 | RRSsIQSTRFFRKNWLRLTW |
| 7851 | RRSsIQSTYFFRKNWLRLTW |
| 7852 | RRSsLDAEIDSFFFRKNWLRLTW |
| 7853 | RRSsLDAEIDSLFFRKNWLRLTW |
| 7854 | RRSsLDAEIDSMFFRKNWLRLTW |
| 7855 | RRSsLDAEIDSVFFRKNWLRLTW |
| 7856 | RRsSQSWSFFFRKNWLRLTW |
| 7857 | RRSsQSWSFFFRKNWLRLTW |
| 7858 | RRSsQSWSKFFRKNWLRLTW |
| 7859 | RRsSQSWSLFFRKNWLRLTW |
| 7860 | RRSsQSWSLFFRKNWLRLTW |
| 7861 | RRsSQSWSMFFRKNWLRLTW |
| 7862 | RRSsQSWSMFFRKNWLRLTW |
| 7863 | RRSsQSWSRFFRKNWLRLTW |
| 7864 | RRsSQSWSVFFRKNWLRLTW |
| 7865 | RRSsQSWSYFFRKNWLRLTW |
| 7866 | RRSsSVAQVFFRKNWLRLTW |
| 7867 | RRSsTASLVKFFFRKNWLRLTW |
| 7868 | RRSsTASLVKKFFRKNWLRLTW |
| 7869 | RRSsTASLVKLFFRKNWLRLTW |
| 7870 | RRSsTASLVKMFFRKNWLRLTW |
| 7871 | RRSsTASLVKRFFRKNWLRLTW |
| 7872 | RRsSVDLGFFFRKNWLRLTW |
| 7873 | RRSsVDLGFFFRKNWLRLTW |
| 7874 | RRsSVDLGKFFRKNWLRLTW |
| 7875 | RRSsVDLGKFFRKNWLRLTW |
| 7876 | RRsSVDLGLFFRKNWLRLTW |
| 7877 | RRSsVDLGLFFRKNWLRLTW |
| 7878 | RRsSVDLGMFFRKNWLRLTW |
| 7879 | RRSsVDLGMFFRKNWLRLTW |
| 7880 | RRsSVDLGRFFRKNWLRLTW |
| 7881 | RRSsVDLGRFFRKNWLRLTW |
| 7882 | RRsSVDLGYFFRKNWLRLTW |
| 7883 | RRSsVDLGYFFRKNWLRLTW |
| 7884 | RRSsVKVEAFFRKNWLRLTW |
| 7885 | RRSsVKVEFFFRKNWLRLTW |
| 7886 | RRSsVKVEKFFRKNWLRLTW |
| 7887 | RRSsVKVELFFRKNWLRLTW |
| 7888 | RRSsVKVEMFFRKNWLRLTW |
| 7889 | RRSsVKVERFFRKNWLRLTW |
| 7890 | RRSsVKVEYFFRKNWLRLTW |
| 7891 | RRTsPITRFFFRKNWLRLTW |
| 7892 | RRTsPITRKFFRKNWLRLTW |
| 7893 | RRTsPITRLFFRKNWLRLTW |
| 7894 | RRTsPITRMFFRKNWLRLTW |
| 7895 | RRTsPITRRFFRKNWLRLTW |
| 7896 | RRWQRSsFFFRKNWLRLTW |
| 7897 | RRWQRSsKFFRKNWLRLTW |
| 7898 | RRWQRSsLFFRKNWLRLTW |
| 7899 | RRWQRSsMFFRKNWLRLTW |
| 7900 | RRWQRSsRFFRKNWLRLTW |
| 7901 | RRWQRSsYFFRKNWLRLTW |
| 7902 | RRWQRSsLFFRKNWLRLTW |
| 7903 | RRYsGKTEFFFRKNWLRLTW |
| 7904 | RRYsGKTEKFFRKNWLRLTW |
| 7905 | RRYsGKTELFFRKNWLRLTW |
| 7906 | RRYsGKTERFFRKNWLRLTW |
| 7907 | RRYsGKTEYFFRKNWLRLTW |
| 7908 | RRYsGNMEFFFRKNWLRLTW |
| 7909 | RRYsGNMEKFFRKNWLRLTW |
| 7910 | RRYsGNMELFFRKNWLRLTW |
| 7911 | RRYsGNMEMFFRKNWLRLTW |
| 7912 | RRYsGNMERFFRKNWLRLTW |
| 7913 | RRYsKFFDLFFRKNWLRLTW |
| 7914 | RRYsPPIERFFRKNWLRLTW |
| 7915 | RRYsPPIQFFRKNWLRLTW |
| 7916 | RRYsPPIQFFFRKNWLRLTW |
| 7917 | RRYsPPIQKFFRKNWLRLTW |
| 7918 | RRYsPPIQLFFRKNWLRLTW |
| 7919 | RRYsPPIQMFFRKNWLRLTW |
| 7920 | RRYsPPIQRFFRKNWLRLTW |
| 7921 | RRYsPPIQYFFRKNWLRLTW |
| 7922 | RRYsRsPYSFFFRKNWLRLTW |
| 7923 | RRYsRSPYSFFFRKNWLRLTW |
| 7924 | RRYSRsPYSFFFRKNWLRLTW |
| 7925 | RRYsRsPYSKFFRKNWLRLTW |
| 7926 | RRYsRSPYSKFFRKNWLRLTW |
| 7927 | RRYSRsPYSKFFRKNWLRLTW |
| 7928 | RRYsRsPYSLFFRKNWLRLTW |
| 7929 | RRYsRSPYSLFFRKNWLRLTW |
| 7930 | RRYSRsPYSLFFRKNWLRLTW |
| 7931 | RRYsRsPYSMFFRKNWLRLTW |
| 7932 | RRYsRSPYSMFFRKNWLRLTW |
| 7933 | RRYSRsPYSMFFRKNWLRLTW |
| 7934 | RRYsRsPYSRFFRKNWLRLTW |
| 7935 | RRYsRSPYSRFFRKNWLRLTW |
| 7936 | RRYSRsPYSRFFRKNWLRLTW |
| 7937 | RRYtNRWTKFFRKNWLRLTW |
| 7938 | RRYtNRWTLFFRKNWLRLTW |
| 7939 | RRYtNRWTMFFRKNWLRLTW |
| 7940 | RRYtNRWTRFFRKNWLRLTW |
| 7941 | RSAsFSRKVFFRKNWLRLTW |
| 7942 | RSAsPDDDLGSSNFFRKNWLRLTW |
| 7943 | RSAsSATQVHKFFRKNWLRLTW |
| 7944 | RSAsSATQVHYFFRKNWLRLTW |
| 7945 | RSDPSKsPGSLRYFFRKNWLRLTW |
| 7946 | RSDsPKIDLFFRKNWLRLTW |
| 7947 | RSDsPKIDYFFRKNWLRLTW |
| 7948 | RSDsRAQAVFFRKNWLRLTW |
| 7949 | RSDsRAQAYFFRKNWLRLTW |
| 7950 | RSDsVGENLFFRKNWLRLTW |
| 7951 | RSDsVGENYFFRKNWLRLTW |
| 7952 | RSDsYVELFFRKNWLRLTW |
| 7953 | RSDsYVELSQYFFRKNWLRLTW |
| 7954 | RSEPSKsPGSLRYFFRKNWLRLTW |
| 7955 | RSEsKDRKFFFRKNWLRLTW |
| 7956 | RSEsKDRKLFFRKNWLRLTW |
| 7957 | RSEsKDRKMFFRKNWLRLTW |
| 7958 | RSEsKDRKVFFRKNWLRLTW |
| 7959 | RSEsPKIDLFFRKNWLRLTW |
| 7960 | RSEsPKIDYFFRKNWLRLTW |
| 7961 | RSEsPPAELFFRKNWLRLTW |
| 7962 | RSEsRAQAVFFRKNWLRLTW |
| 7963 | RSEsRAQAYFFRKNWLRLTW |
| 7964 | RSEsVGENLFFRKNWLRLTW |
| 7965 | RSEsVGENYFFRKNWLRLTW |
| 7966 | RSEsYVELSQYFFRKNWLRLTW |
| 7967 | RSFsPTMKVFFRKNWLRLTW |
| 7968 | RSGsLERKFFFRKNWLRLTW |
| 7969 | RSGsLERKLFFRKNWLRLTW |
| 7970 | RSGsLERKMFFRKNWLRLTW |
| 7971 | RSGsLERKVFFRKNWLRLTW |
| 7972 | RSHSsPASLFFRKNWLRLTW |
| 7973 | RSIsVGENLFFRKNWLRLTW |
| 7974 | RSLsESYELFFRKNWLRLTW |
| 7975 | RSLsPGGAAFFRKNWLRLTW |
| 7976 | RSLsPGGAFFFRKNWLRLTW |
| 7977 | RSLsPGGALFFRKNWLRLTW |
| 7978 | RSLsPGGAMFFRKNWLRLTW |
| 7979 | RSLsPGGAVFFRKNWLRLTW |
| 7980 | RSLsPLLFFFRKNWLRLTW |
| 7981 | RSLsPLLLFFRKNWLRLTW |
| 7982 | RSLsPLLMFFRKNWLRLTW |
| 7983 | RSLsPLLVFFRKNWLRLTW |
| 7984 | RSLsQELVGVFFRKNWLRLTW |
| 7985 | RSLsVEIVKFFRKNWLRLTW |
| 7986 | RSLsVEIVYFFRKNWLRLTW |
| 7987 | RSMsMPVAHFFRKNWLRLTW |
| 7988 | RSMsMPVAKFFRKNWLRLTW |
| 7989 | RsPEDEYELLMPHRISSHFFRKNWLRLTW |
| 7990 | RSRRsPLLKFFRKNWLRLTW |
| 7991 | RSRRsPLLYFFRKNWLRLTW |
| 7992 | RSRsPLELFFRKNWLRLTW |
| 7993 | RSRsPPPVSKFFRKNWLRLTW |
| 7994 | RSRsPPPVSYFFRKNWLRLTW |
| 7995 | RSRsPRPAFFFRKNWLRLTW |
| 7996 | RSRsPRPAIFFRKNWLRLTW |
| 7997 | RSRsPRPALFFRKNWLRLTW |
| 7998 | RSRsPRPAMFFRKNWLRLTW |
| 7999 | RSRsPRPAVFFRKNWLRLTW |
| 8000 | RSRsPRPAXFFRKNWLRLTW |
| 8001 | RSRTsPITRRFFRKNWLRLTW |
| 8002 | RSRTsPITRYFFRKNWLRLTW |
| 8003 | RSSsLIRHKFFRKNWLRLTW |
| 8004 | RSSsLIRHYFFRKNWLRLTW |
| 8005 | RSVsLSMRKFFRKNWLRLTW |
| 8006 | RSVsLSMRYFFRKNWLRLTW |
| 8007 | RsWKYNQSISLRRPFFRKNWLRLTW |
| 8008 | RSYsGSRsKFFRKNWLRLTW |
| 8009 | RSYsGSRsRFFRKNWLRLTW |
| 8010 | RSYsGSRsYFFRKNWLRLTW |
| 8011 | RSYsPDHRQKFFRKNWLRLTW |
| 8012 | RSYsPDHRQYFFRKNWLRLTW |
| 8013 | RSYsPERSKFFRKNWLRLTW |
| 8014 | RSYsPERSYFFRKNWLRLTW |
| 8015 | RSYsPRNSRFFRKNWLRLTW |
| 8016 | RSYsPRNSYFFRKNWLRLTW |
| 8017 | RSYSRsFSKFFRKNWLRLTW |
| 8018 | RSYsRSFSRFFRKNWLRLTW |
| 8019 | RSYSRsFSRFFRKNWLRLTW |
| 8020 | RSYSRsFSYFFRKNWLRLTW |
| 8021 | RSYsYPRQKFFRKNWLRLTW |
| 8022 | RSYsYPRQYFFRKNWLRLTW |
| 8023 | RSYVTTSTRTYsLGFFRKNWLRLTW |
| 8024 | RTAsFAVRKFFRKNWLRLTW |
| 8025 | RTAsFAVRYFFRKNWLRLTW |
| 8026 | RTAsLIIKVFFRKNWLRLTW |
| 8027 | RTAsPPPPPKFFRKNWLRLTW |
| 8028 | RTDPSKsPGSLRYFFRKNWLRLTW |
| 8029 | RTDsPKIDLFFRKNWLRLTW |
| 8030 | RTDsPKIDYFFRKNWLRLTW |
| 8031 | RTDsRAQAVFFRKNWLRLTW |
| 8032 | RTDsRAQAYFFRKNWLRLTW |
| 8033 | RTDsYVELSQYFFRKNWLRLTW |
| 8034 | RTEPSKsPGSLRYFFRKNWLRLTW |
| 8035 | RTEsDSGLKFFFRKNWLRLTW |
| 8036 | RTEsDSGLKKFFRKNWLRLTW |
| 8037 | RTEsDSGLKLFFRKNWLRLTW |
| 8038 | RTEsDSGLKMFFRKNWLRLTW |
| 8039 | RTEsDSGLKVFFRKNWLRLTW |
| 8040 | RTEsPKIDLFFRKNWLRLTW |
| 8041 | RTEsPKIDYFFRKNWLRLTW |
| 8042 | RTEsRAQAVFFRKNWLRLTW |
| 8043 | RTEsRAQAYFFRKNWLRLTW |
| 8044 | RTEsYVELSQYFFRKNWLRLTW |
| 8045 | RTFsLDTILFFRKNWLRLTW |
| 8046 | RTFsPTYGFFFRKNWLRLTW |
| 8047 | RTFsPTYGLFFRKNWLRLTW |
| 8048 | RTFsPTYGMFFRKNWLRLTW |
| 8049 | RTFsPTYGVFFRKNWLRLTW |
| 8050 | RTHsLLLLLFFRKNWLRLTW |
| 8051 | RTLsHISEAFFRKNWLRLTW |
| 8052 | RTLsHISEVFFRKNWLRLTW |
| 8053 | RTLsPEIITVFFRKNWLRLTW |
| 8054 | RTMsEAALVRKFFRKNWLRLTW |
| 8055 | RTNsPGFQKFFRKNWLRLTW |
| 8056 | RTPsDVKELFFRKNWLRLTW |
| 8057 | RTPsFLKKNKFFRKNWLRLTW |
| 8058 | RTPsFLKKNYFFRKNWLRLTW |
| 8059 | RTRsLSSLREKFFRKNWLRLTW |
| 8060 | RTRsLSSLREYFFRKNWLRLTW |
| 8061 | RTRsPSPTFFFRKNWLRLTW |
| 8062 | RTRsPSPTLFFRKNWLRLTW |
| 8063 | RTRsPSPTMFFRKNWLRLTW |
| 8064 | RTRsPSPTVFFRKNWLRLTW |
| 8065 | RTSsFALNLFFRKNWLRLTW |
| 8066 | RTSsFTEQLFFRKNWLRLTW |
| 8067 | RTSsFTFQNFFRKNWLRLTW |
| 8068 | RTSSFtFQNFFRKNWLRLTW |
| 8069 | RTSsPLFNKFFRKNWLRLTW |
| 8070 | RTYKsPLRHFFRKNWLRLTW |
| 8071 | RTYKsPLRKFFRKNWLRLTW |
| 8072 | RTYKsPLRYFFRKNWLRLTW |
| 8073 | RTYsGPMNKFFRKNWLRLTW |
| 8074 | RTYsGPMNKVFFRKNWLRLTW |
| 8075 | RTYsHGTYRFFRKNWLRLTW |
| 8076 | RVAsFAVRKFFRKNWLRLTW |
| 8077 | RVAsFAVRYFFRKNWLRLTW |
| 8078 | RVAsPLVHKFFRKNWLRLTW |
| 8079 | RVAsPLVHYFFRKNWLRLTW |
| 8080 | RVAsPPPPPKFFRKNWLRLTW |
| 8081 | RVAsPPPPPYFFRKNWLRLTW |
| 8082 | RVAsPTSGVFFRKNWLRLTW |
| 8083 | RVAsPTSGVKFFRKNWLRLTW |
| 8084 | RVAsPTSGVKKFFRKNWLRLTW |
| 8085 | RVAsPTSGVKRFFRKNWLRLTW |
| 8086 | RVAsPTSGVYFFRKNWLRLTW |
| 8087 | RVDsPSHGLFFRKNWLRLTW |
| 8088 | RVGsLVLNLFFRKNWLRLTW |
| 8089 | RVIsGVLQLFFRKNWLRLTW |
| 8090 | RVKLPsGSKKFFRKNWLRLTW |
| 8091 | RVKsPGsGHVKFFRKNWLRLTW |
| 8092 | RVKsPGsGHVYFFRKNWLRLTW |
| 8093 | RVKsPISLKFFRKNWLRLTW |
| 8094 | RVKsPSPKSERFFRKNWLRLTW |
| 8095 | RVKsPSPKSEYFFRKNWLRLTW |
| 8096 | RVKtPTSQSYKFFRKNWLRLTW |
| 8097 | RVKtPTSQSYRFFRKNWLRLTW |
| 8098 | RVKtPTSQSYYFFRKNWLRLTW |
| 8099 | RVKTtPLRRFFRKNWLRLTW |
| 8100 | RVKTtPLRYFFRKNWLRLTW |
| 8101 | RVLDRSPsRSAKFFRKNWLRLTW |
| 8102 | RVLDRSPsRSAYFFRKNWLRLTW |
| 8103 | RVLHsPPAVFFRKNWLRLTW |
| 8104 | RVLsGWTKFFRKNWLRLTW |
| 8105 | RVLsPLIIKFFRKNWLRLTW |
| 8106 | RVPsLLVLLFFRKNWLRLTW |
| 8107 | RVPsSTLKKFFRKNWLRLTW |
| 8108 | RVPsSTLKYFFRKNWLRLTW |
| 8109 | RVRKLPsTTLFFRKNWLRLTW |
| 8110 | RVRQsPLATKFFRKNWLRLTW |
| 8111 | RVRQsPLATRFFRKNWLRLTW |
| 8112 | RVRQsPLATYFFRKNWLRLTW |
| 8113 | RVRRsSFLNAKFFRKNWLRLTW |
| 8114 | RVRsLSSLREKFFRKNWLRLTW |
| 8115 | RVRsLSSLREYFFRKNWLRLTW |
| 8116 | RVRsPTRSFFFRKNWLRLTW |
| 8117 | RVRsPTRSLFFRKNWLRLTW |
| 8118 | RVRsPTRSMFFRKNWLRLTW |
| 8119 | RVRsPTRSPFFRKNWLRLTW |
| 8120 | RVRsPTRSVFFRKNWLRLTW |
| 8121 | RVSsPISKKFFRKNWLRLTW |
| 8122 | RVSsPISKYFFRKNWLRLTW |
| 8123 | RVSsRFSSKFFRKNWLRLTW |
| 8124 | RVSsRFSSRFFRKNWLRLTW |
| 8125 | RVSsRFSSYFFRKNWLRLTW |
| 8126 | RVSsVKLISRFFRKNWLRLTW |
| 8127 | RVSsVKLISYFFRKNWLRLTW |
| 8128 | RVTsAEIKLFFRKNWLRLTW |
| 8129 | RWsLSMRKFFRKNWLRLTW |
| 8130 | RWsLSMRYFFRKNWLRLTW |
| 8131 | RVWEDRPSsAFFRKNWLRLTW |
| 8132 | RVWsPPRVHKVFFRKNWLRLTW |
| 8133 | RVYQyIQSRFFRKNWLRLTW |
| 8134 | RVYQyIQSRFKFFRKNWLRLTW |
| 8135 | RVYQyIQSRFYFFRKNWLRLTW |
| 8136 | RVYQyIQSRKFFRKNWLRLTW |
| 8137 | RVYQyIQSRYFFRKNWLRLTW |
| 8138 | RVYsPYNHKFFRKNWLRLTW |
| 8139 | RVYsPYNHRFFRKNWLRLTW |
| 8140 | RVYsPYNHYFFRKNWLRLTW |
| 8141 | RVYSRsFSKFFRKNWLRLTW |
| 8142 | RVYSRsFSYFFRKNWLRLTW |
| 8143 | RYPsNLQLFFFRKNWLRLTW |
| 8144 | RYQtQPVTLFFRKNWLRLTW |
| 8145 | SAARESHPHGVKRSAsPDDDLGFFRKNWLRLTW |
| 8146 | SARGsPTRPNPPVRFFRKNWLRLTW |
| 8147 | SARRtPVSYFFRKNWLRLTW |
| 8148 | sDDEKMPDLEFFRKNWLRLTW |
| 8149 | sDFHAERAAREKFFRKNWLRLTW |
| 8150 | SDmPRAHsFFFRKNWLRLTW |
| 8151 | SDMPRAHsFFFRKNWLRLTW |
| 8152 | SEFKAMDsIFFRKNWLRLTW |
| 8153 | SEGsLHRKFFFRKNWLRLTW |
| 8154 | SEGsLHRKWFFRKNWLRLTW |
| 8155 | SEGsLHRKYFFRKNWLRLTW |
| 8156 | SELsPGRSVFFRKNWLRLTW |
| 8157 | SFDsGSVRLFFRKNWLRLTW |
| 8158 | SGGAQsPLRYLHVLFFRKNWLRLTW |
| 8159 | sGGDDDWTHLSSKEVDPSTFFRKNWLRLTW |
| 8160 | sGGDDDWTHLSSKEVDPSTGFFRKNWLRLTW |
| 8161 | sGGDDDWTHLSSKEVDPSTGEFFRKNWLRLTW |
| 8162 | sGGDDDWTHLSSKEVDPSTGELFFRKNWLRLTW |
| 8163 | sGGDDDWTHLSSKEVDPSTGELQFFRKNWLRLTW |
| 8164 | SGPKPLFRRMsSLVGPTQFFRKNWLRLTW |
| 8165 | SIDsPQKLFFRKNWLRLTW |
| 8166 | SIDsPQKYFFRKNWLRLTW |
| 8167 | SILsFVSGLFFRKNWLRLTW |
| 8168 | SIMsFHIDLFFRKNWLRLTW |
| 8169 | SImsPEIQLFFRKNWLRLTW |
| 8170 | SIMsPEIQLFFRKNWLRLTW |
| 8171 | SIPtVSGQIFFRKNWLRLTW |
| 8172 | SISsMEVNVFFRKNWLRLTW |
| 8173 | SISStPPAVFFRKNWLRLTW |
| 8174 | SKEDKNGHDGDTHQEDDGEKsDFFRKNWLRLTW |
| 8175 | SKRGyIGLFFRKNWLRLTW |
| 8176 | SKtVATFILFFRKNWLRLTW |
| 8177 | SLAsLTEKIFFRKNWLRLTW |
| 8178 | SLDSEDYsLFFRKNWLRLTW |
| 8179 | SLDsLGDVFLFFRKNWLRLTW |
| 8180 | SLDsPSYVLYFFRKNWLRLTW |
| 8181 | SLEsPSYVLYFFRKNWLRLTW |
| 8182 | SLFGGsVKLFFRKNWLRLTW |
| 8183 | SLFKRLYsLFFRKNWLRLTW |
| 8184 | SLFsGDEENAFFRKNWLRLTW |
| 8185 | SLFsGSYSSLFFRKNWLRLTW |
| 8186 | SLFsPQNTLFFRKNWLRLTW |
| 8187 | SLFsPRRNKFFRKNWLRLTW |
| 8188 | SLFsPRRNYFFRKNWLRLTW |
| 8189 | SLFsSEESNLFFRKNWLRLTW |
| 8190 | SLFsSEESNLGAFFRKNWLRLTW |
| 8191 | SLHDIQLsLFFRKNWLRLTW |
| 8192 | SLKsPVTVKFFRKNWLRLTW |
| 8193 | SLLAsPGHISVFFRKNWLRLTW |
| 8194 | SLLHTSRsLFFRKNWLRLTW |
| 8195 | SLLNKSsPVKFFRKNWLRLTW |
| 8196 | SLLNKSsPVKKFFRKNWLRLTW |
| 8197 | SLLNKSsPVKYFFRKNWLRLTW |
| 8198 | SLLsLHVDLFFRKNWLRLTW |
| 8199 | SLLTsPPKAFFRKNWLRLTW |
| 8200 | SLLTsPPKVFFRKNWLRLTW |
| 8201 | SLMsGTLESLFFRKNWLRLTW |
| 8202 | SLMsPGRRKFFRKNWLRLTW |
| 8203 | SLMsPGRRYFFRKNWLRLTW |
| 8204 | SLQPRSHsVFFRKNWLRLTW |
| 8205 | SLQsLETSVFFRKNWLRLTW |
| 8206 | SLRRsVLMKFFRKNWLRLTW |
| 8207 | SLRRsVLMYFFRKNWLRLTW |
| 8208 | SLSsLLVKLFFRKNWLRLTW |
| 8209 | SLtRSPPRVFFRKNWLRLTW |
| 8210 | SLTRsPPRVFFRKNWLRLTW |
| 8211 | SLVDGyFRLFFRKNWLRLTW |
| 8212 | SLYDRPAsYFFRKNWLRLTW |
| 8213 | SLYsPVKKKFFRKNWLRLTW |
| 8214 | SMFsPRRNKFFRKNWLRLTW |
| 8215 | SMKsPVTVKFFRKNWLRLTW |
| 8216 | SMLNKSsPVKFFRKNWLRLTW |
| 8217 | SMLNKSsPVKKFFRKNWLRLTW |
| 8218 | SMLsQEIQTLFFRKNWLRLTW |
| 8219 | SMLTsPPKAFFRKNWLRLTW |
| 8220 | SMLTsPPKVFFRKNWLRLTW |
| 8221 | SMMsPGRRKFFRKNWLRLTW |
| 8222 | SMQPRSHsVFFRKNWLRLTW |
| 8223 | SMRRsVLMKFFRKNWLRLTW |
| 8224 | SMSsLSREVFFRKNWLRLTW |
| 8225 | SMtRSPPRVFFRKNWLRLTW |
| 8226 | SMTRsPPRVFFRKNWLRLTW |
| 8227 | SMYsPVKKKFFRKNWLRLTW |
| 8228 | SNFKsPVKTIRFFRKNWLRLTW |
| 8229 | SPAASISRLsGEQVDGKGFFRKNWLRLTW |
| 8230 | SPAsPKISFFFRKNWLRLTW |
| 8231 | SPAsPKISLFFRKNWLRLTW |
| 8232 | SPAsPKISMFFRKNWLRLTW |
| 8233 | SPAsPKISVFFRKNWLRLTW |
| 8234 | SPDsSQSSLFFRKNWLRLTW |
| 8235 | sPEDEYELLMPHRISSHFFRKNWLRLTW |
| 8236 | SPEDEYELLMPHRIsSHFFRKNWLRLTW |
| 8237 | SPEKAGRRsSFFFRKNWLRLTW |
| 8238 | SPEKAGRRsSLFFRKNWLRLTW |
| 8239 | SPEKAGRRsSMFFRKNWLRLTW |
| 8240 | SPEKAGRRsSVFFRKNWLRLTW |
| 8241 | sPERPFLAILGGAKVADKFFRKNWLRLTW |
| 8242 | sPERPFLAILGGAKVADKIQFFRKNWLRLTW |
| 8243 | SPFKRQLsFFFRKNWLRLTW |
| 8244 | SPFKRQLsLFFRKNWLRLTW |
| 8245 | SPFKRQLsMFFRKNWLRLTW |
| 8246 | SPFKRQLsVFFRKNWLRLTW |
| 8247 | SPFLsKRSLFFRKNWLRLTW |
| 8248 | SPGLARKRsFFFRKNWLRLTW |
| 8249 | SPGLARKRsLFFRKNWLRLTW |
| 8250 | SPGLARKRsMFFRKNWLRLTW |
| 8251 | SPGLARKRsVFFRKNWLRLTW |
| 8252 | SPGsPRPAFFFRKNWLRLTW |
| 8253 | SPGsPRPALFFRKNWLRLTW |
| 8254 | SPGsPRPAMFFRKNWLRLTW |
| 8255 | SPGsPRPAVFFRKNWLRLTW |
| 8256 | SPKsPGLKAFFRKNWLRLTW |
| 8257 | SPKsPGLKFFFRKNWLRLTW |
| 8258 | SPKsPGLKLFFRKNWLRLTW |
| 8259 | SPKsPGLKMFFRKNWLRLTW |
| 8260 | SPKsPGLKVFFRKNWLRLTW |
| 8261 | SPKsPTAAFFFRKNWLRLTW |
| 8262 | SPKsPTAALFFRKNWLRLTW |
| 8263 | SPKsPTAAMFFRKNWLRLTW |
| 8264 | SPKsPTAAVFFRKNWLRLTW |
| 8265 | SPLTKSIsLFFRKNWLRLTW |
| 8266 | sPPFPVPVYTRQAPKQVIKFFRKNWLRLTW |
| 8267 | SPRAPVsPLKFFFRKNWLRLTW |
| 8268 | SPRERsPALFFRKNWLRLTW |
| 8269 | SPRGEAsSLFFRKNWLRLTW |
| 8270 | SPRGEASsLFFRKNWLRLTW |
| 8271 | SPRPPNsPSIFFRKNWLRLTW |
| 8272 | SPRRsLGLALFFRKNWLRLTW |
| 8273 | SPRRsRSISFFFRKNWLRLTW |
| 8274 | SPRRsRSISFFFRKNWLRLTW |
| 8275 | SPRRsRSIsLFFRKNWLRLTW |
| 8276 | SPRRsRSISLFFRKNWLRLTW |
| 8277 | SPRRsRSIsMFFRKNWLRLTW |
| 8278 | SPRRsRSISMFFRKNWLRLTW |
| 8279 | SPRRsRSIsVFFRKNWLRLTW |
| 8280 | SPRRsRSISVFFRKNWLRLTW |
| 8281 | SPRsITSTFFFRKNWLRLTW |
| 8282 | SPRsITSTLFFRKNWLRLTW |
| 8283 | SPRsITSTMFFRKNWLRLTW |
| 8284 | SPRsITSTPFFRKNWLRLTW |
| 8285 | SPRsITSTVFFRKNWLRLTW |
| 8286 | SPRsPDRTLFFRKNWLRLTW |
| 8287 | SPRsPGKPFFFRKNWLRLTW |
| 8288 | SPRsPGKPLFFRKNWLRLTW |
| 8289 | SPRsPGKPMFFRKNWLRLTW |
| 8290 | SPRsPGKPVFFRKNWLRLTW |
| 8291 | SPRsPGRSFFFRKNWLRLTW |
| 8292 | SPRsPGRSIFFRKNWLRLTW |
| 8293 | SPRsPGRSLFFRKNWLRLTW |
| 8294 | SPRsPGRSMFFRKNWLRLTW |
| 8295 | SPRsPGRSVFFRKNWLRLTW |
| 8296 | SPRsPGRSXFFRKNWLRLTW |
| 8297 | SPRsPSGLRFFRKNWLRLTW |
| 8298 | SPRsPSTTYFFFRKNWLRLTW |
| 8299 | SPRsPSTTYLFFRKNWLRLTW |
| 8300 | SPRSPsTTYLFFRKNWLRLTW |
| 8301 | SPRsPSTTYMFFRKNWLRLTW |
| 8302 | SPRsPSTTYVFFRKNWLRLTW |
| 8303 | SPRsSQLVFFRKNWLRLTW |
| 8304 | SPRtPVsPVKFFFRKNWLRLTW |
| 8305 | SPRTPVsPVKFFFRKNWLRLTW |
| 8306 | SPRtPVsPVKLFFRKNWLRLTW |
| 8307 | SPRTPVsPVKLFFRKNWLRLTW |
| 8308 | SPRtPVsPVKMFFRKNWLRLTW |
| 8309 | SPRTPVsPVKMFFRKNWLRLTW |
| 8310 | SPRtPVsPVKVFFRKNWLRLTW |
| 8311 | SPRTPVsPVKVFFRKNWLRLTW |
| 8312 | SPSsPSVRRQFFFRKNWLRLTW |
| 8313 | SPSsPSVRRQLFFRKNWLRLTW |
| 8314 | SPSsPSVRRQMFFRKNWLRLTW |
| 8315 | SPSsPSVRRQVFFRKNWLRLTW |
| 8316 | SPSTSRSGGsSRFFFRKNWLRLTW |
| 8317 | SPSTSRSGGsSRLFFRKNWLRLTW |
| 8318 | SPSTSRSGGsSRMFFRKNWLRLTW |
| 8319 | SPSTSRSGGsSRVFFRKNWLRLTW |
| 8320 | sPTRPNPPVRNLHFFRKNWLRLTW |
| 8321 | SPVsPMKELFFRKNWLRLTW |
| 8322 | SPVsTRPLEPFFRKNWLRLTW |
| 8323 | SPVStRPLEPFFRKNWLRLTW |
| 8324 | SPWHQsFFFRKNWLRLTW |
| 8325 | SPWHQsLFFRKNWLRLTW |
| 8326 | SPWHQsMFFRKNWLRLTW |
| 8327 | SPWHQsVFFRKNWLRLTW |
| 8328 | SQIsPKSWGVFFRKNWLRLTW |
| 8329 | SRDKHsEYFFRKNWLRLTW |
| 8330 | SREKHsEIFFRKNWLRLTW |
| 8331 | SREKHsElFFRKNWLRLTW |
| 8332 | SRFNRRVsVFFRKNWLRLTW |
| 8333 | SRLTHLsFFFRKNWLRLTW |
| 8334 | SRLTHLsKFFRKNWLRLTW |
| 8335 | SRLTHLsLFFRKNWLRLTW |
| 8336 | SRLTHLsMFFRKNWLRLTW |
| 8337 | SRLTHLsRFFRKNWLRLTW |
| 8338 | SRLTHLsYFFRKNWLRLTW |
| 8339 | SRMsPKAQFFFRKNWLRLTW |
| 8340 | SRMsPKAQKFFRKNWLRLTW |
| 8341 | SRMsPKAQLFFRKNWLRLTW |
| 8342 | SRMsPKAQMFFRKNWLRLTW |
| 8343 | SRMsPKAQRFFRKNWLRLTW |
| 8344 | SRMsPKAQYFFRKNWLRLTW |
| 8345 | SRsSRSPYSRFFRKNWLRLTW |
| 8346 | SRSsSVLsLFFRKNWLRLTW |
| 8347 | SRSSsVLSLFFRKNWLRLTW |
| 8348 | SRSSSVLsLFFRKNWLRLTW |
| 8349 | SRTsPITRFFFRKNWLRLTW |
| 8350 | SRTsPITRKFFRKNWLRLTW |
| 8351 | SRTsPITRLFFRKNWLRLTW |
| 8352 | SRTsPITRMFFRKNWLRLTW |
| 8353 | SRTsPITRRFFRKNWLRLTW |
| 8354 | SRTsPITRYFFRKNWLRLTW |
| 8355 | SRWsGSHQFFFRKNWLRLTW |
| 8356 | SRWsGSHQKFFRKNWLRLTW |
| 8357 | SRWsGSHQRFFRKNWLRLTW |
| 8358 | SRWsGSHQYFFRKNWLRLTW |
| 8359 | SRYsRsPYSFFFRKNWLRLTW |
| 8360 | SRYsRSPYSFFFRKNWLRLTW |
| 8361 | SRYSRsPYSFFFRKNWLRLTW |
| 8362 | SRYsRsPYSKFFRKNWLRLTW |
| 8363 | SRYsRSPYSFFFRKNWLRLTW |
| 8364 | SRYSRsPYSFFFRKNWLRLTW |
| 8365 | SRYsRsPYSLFFRKNWLRLTW |
| 8366 | SRYsRSPYSLFFRKNWLRLTW |
| 8367 | SRYSRsPYSLFFRKNWLRLTW |
| 8368 | SRYsRsPYSMFFRKNWLRLTW |
| 8369 | SRYsRSPYSMFFRKNWLRLTW |
| 8370 | SRYSRsPYSMFFRKNWLRLTW |
| 8371 | SRYsRsPYSRFFRKNWLRLTW |
| 8372 | SRYsRSPYSRFFRKNWLRLTW |
| 8373 | SRYSRsPYSRFFRKNWLRLTW |
| 8374 | SRYsRsPYSYFFRKNWLRLTW |
| 8375 | SRYsRSPYSYFFRKNWLRLTW |
| 8376 | SRYSRsPYSYFFRKNWLRLTW |
| 8377 | SRYsRtsPYSRFFRKNWLRLTW |
| 8378 | SSDIsPTRLFFRKNWLRLTW |
| 8379 | SSDIsPTRYFFRKNWLRLTW |
| 8380 | SSDKHsEYFFRKNWLRLTW |
| 8381 | SSDPASQLsYFFRKNWLRLTW |
| 8382 | SSDsETLRYFFRKNWLRLTW |
| 8383 | SSDsPQKLFFRKNWLRLTW |
| 8384 | SSDsPQKYFFRKNWLRLTW |
| 8385 | SSDsPSYVLYFFRKNWLRLTW |
| 8386 | SSDsPTNHFFFFRKNWLRLTW |
| 8387 | SSEIsPTRYFFRKNWLRLTW |
| 8388 | SSEKHsEYFFRKNWLRLTW |
| 8389 | SSEPASQLsYFFRKNWLRLTW |
| 8390 | SSEsETLRYFFRKNWLRLTW |
| 8391 | SSEsPQKLFFRKNWLRLTW |
| 8392 | SSEsPQKYFFRKNWLRLTW |
| 8393 | SSEsPSYVLYFFRKNWLRLTW |
| 8394 | SSEsPTNHFYFFRKNWLRLTW |
| 8395 | SSNGKMASRRsEEKEAGFFRKNWLRLTW |
| 8396 | SSNGKMASRRsEEKEAGEIFFRKNWLRLTW |
| 8397 | SsPIMRKKVSLFFRKNWLRLTW |
| 8398 | sSPPFPVPVYTRQAPKQVIKFFRKNWLRLTW |
| 8399 | SSsPTHAKSAHVFFRKNWLRLTW |
| 8400 | SSsWRILGSKQSEHRPFFRKNWLRLTW |
| 8401 | STDIsPTRLFFRKNWLRLTW |
| 8402 | STDIsPTRYFFRKNWLRLTW |
| 8403 | STDKHsEYFFRKNWLRLTW |
| 8404 | STDPASQLsYFFRKNWLRLTW |
| 8405 | STDsETLRYFFRKNWLRLTW |
| 8406 | STDsPQKYFFRKNWLRLTW |
| 8407 | STDsPSYVLYFFRKNWLRLTW |
| 8408 | STDsPTNHFYFFRKNWLRLTW |
| 8409 | STEIsPTRLFFRKNWLRLTW |
| 8410 | STEIsPTRYFFRKNWLRLTW |
| 8411 | STEKHsEYFFRKNWLRLTW |
| 8412 | STEPASQLsYFFRKNWLRLTW |
| 8413 | STEsETLRYFFRKNWLRLTW |
| 8414 | STEsPQKYFFRKNWLRLTW |
| 8415 | STEsPSYVLYFFRKNWLRLTW |
| 8416 | STEsPTNHFYFFRKNWLRLTW |
| 8417 | STIQNsPTKKFFRKNWLRLTW |
| 8418 | sTMSLNIITVFFRKNWLRLTW |
| 8419 | STMsLNIITVFFRKNWLRLTW |
| 8420 | SVDIsPIRLFFRKNWLRLTW |
| 8421 | SVDIsPTRLFFRKNWLRLTW |
| 8422 | SVDIsPTRYFFRKNWLRLTW |
| 8423 | SVFsPSFGLFFRKNWLRLTW |
| 8424 | SVGsDYYIQLFFRKNWLRLTW |
| 8425 | SVKPRRTsLFFRKNWLRLTW |
| 8426 | SVKsPVTVKFFRKNWLRLTW |
| 8427 | SVKsPVTVYFFRKNWLRLTW |
| 8428 | SVLsPSFQLFFRKNWLRLTW |
| 8429 | SVMDsPKKLFFRKNWLRLTW |
| 8430 | SVRRsVLMKFFRKNWLRLTW |
| 8431 | SVRRsVLMYFFRKNWLRLTW |
| 8432 | SVRsLSLSLFFRKNWLRLTW |
| 8433 | SVYsGDFGNLEVFFRKNWLRLTW |
| 8434 | SVYsPVKKKFFRKNWLRLTW |
| 8435 | SVYsPVKKYFFRKNWLRLTW |
| 8436 | sYIEHIFEIFFRKNWLRLTW |
| 8437 | SYPsPVATSYFFRKNWLRLTW |
| 8438 | sYQKVIELFFFRKNWLRLTW |
| 8439 | TDKYsKMMFFRKNWLRLTW |
| 8440 | TEAsPESMLFFRKNWLRLTW |
| 8441 | THKGEIRGASTPFQFRAssPFFRKNWLRLTW |
| 8442 | TIGEKKEPsDKSVDSFFRKNWLRLTW |
| 8443 | TKDKYMASRGQKAKsMEGFFRKNWLRLTW |
| 8444 | TKsVKALSSLHGDDFFRKNWLRLTW |
| 8445 | TKsVKALSSLHGDDQFFRKNWLRLTW |
| 8446 | TKsVKALSSLHGDDQDFFRKNWLRLTW |
| 8447 | TLAsPSVFKSTFFRKNWLRLTW |
| 8448 | TLAsPSVFKSVFFRKNWLRLTW |
| 8449 | TLLAsPMLKFFRKNWLRLTW |
| 8450 | TLMERTVsLFFRKNWLRLTW |
| 8451 | TLSsPPPGLFFRKNWLRLTW |
| 8452 | TMAsPGKDNYFFRKNWLRLTW |
| 8453 | TMAsPSVFKSTFFRKNWLRLTW |
| 8454 | TMAsPSVFKSVFFRKNWLRLTW |
| 8455 | TMDsPGKDNYFFRKNWLRLTW |
| 8456 | TMEsPGKDNYFFRKNWLRLTW |
| 8457 | TMMsPSQFLFFRKNWLRLTW |
| 8458 | TPAQPQRRsFFFRKNWLRLTW |
| 8459 | TPAQPQRRsLFFRKNWLRLTW |
| 8460 | TPAQPQRRsMFFRKNWLRLTW |
| 8461 | TPAQPQRRsVFFRKNWLRLTW |
| 8462 | TPDPSKFFSQLsSEHGGDVFFRKNWLRLTW |
| 8463 | tPDPSKFFSQLSSEHGGDVQFFRKNWLRLTW |
| 8464 | TPIsPGRASGFFFRKNWLRLTW |
| 8465 | TPIsPGRASGLFFRKNWLRLTW |
| 8466 | TPIsPGRASGMFFRKNWLRLTW |
| 8467 | TPIsPGRASGVFFRKNWLRLTW |
| 8468 | TPMKKHLsLFFRKNWLRLTW |
| 8469 | TPRsPPLGFFFRKNWLRLTW |
| 8470 | TPRsPPLGLFFRKNWLRLTW |
| 8471 | TPRsPPLGLFFFRKNWLRLTW |
| 8472 | TPRsPPLGLIFFRKNWLRLTW |
| 8473 | TPRsPPLGLLFFRKNWLRLTW |
| 8474 | TPRsPPLGLMFFRKNWLRLTW |
| 8475 | TPRsPPLGLVFFRKNWLRLTW |
| 8476 | TPRsPPLGMFFRKNWLRLTW |
| 8477 | TPRsPPLGVFFRKNWLRLTW |
| 8478 | TQSSGKsSVFFRKNWLRLTW |
| 8479 | TRKtPESFLFFRKNWLRLTW |
| 8480 | TRLsPAKIVLFFFRKNWLRLTW |
| 8481 | TRLsPAKIVLKFFRKNWLRLTW |
| 8482 | TRLsPAKIVLRFFRKNWLRLTW |
| 8483 | TRLsPAKIVLYFFRKNWLRLTW |
| 8484 | TSAsPGKDNYFFRKNWLRLTW |
| 8485 | TSDsPGKDNYFFRKNWLRLTW |
| 8486 | TSDtPDYLLKYFFRKNWLRLTW |
| 8487 | TSEsPGKDNYFFRKNWLRLTW |
| 8488 | TSEtPDYLLKYFFRKNWLRLTW |
| 8489 | TTAsPGKDNYFFRKNWLRLTW |
| 8490 | TTDsPGKDNYFFRKNWLRLTW |
| 8491 | TTDtPDYLLKYFFRKNWLRLTW |
| 8492 | TTEsPGKDNYFFRKNWLRLTW |
| 8493 | TTEtPDYLLKYFFRKNWLRLTW |
| 8494 | TTKsVKALSSLHGFFRKNWLRLTW |
| 8495 | TTKsVKALSSLHGDDFFRKNWLRLTW |
| 8496 | TTKsVKALSSLHGDDQFFRKNWLRLTW |
| 8497 | TTKsVKALSSLHGDDQDFFRKNWLRLTW |
| 8498 | TTKsVKALSSLHGDDQDSFFRKNWLRLTW |
| 8499 | TTKsVKALSSLHGDDQDsEDFFRKNWLRLTW |
| 8500 | TTKSVKALSSLHGDDQDsEDFFRKNWLRLTW |
| 8501 | TTKsVKALSSLHGDDQDsEDEFFRKNWLRLTW |
| 8502 | TTKSVKALSSLHGDDQDsEDEFFRKNWLRLTW |
| 8503 | TVFsPTLPAAFFRKNWLRLTW |
| 8504 | TVMsNSSVIHLFFRKNWLRLTW |
| 8505 | VAKRLsLFFRKNWLRLTW |
| 8506 | VAMPVKKSPRRSsSDEQGLSYSSLKNVFFRKNWLRLTW |
| 8507 | VIDsQELSKVFFRKNWLRLTW |
| 8508 | VLDsPASKKFFRKNWLRLTW |
| 8509 | VLFPEsPARAFFRKNWLRLTW |
| 8510 | VLFRtPLASVFFRKNWLRLTW |
| 8511 | VLFsSPPQMFFRKNWLRLTW |
| 8512 | VLFSsPPQMFFRKNWLRLTW |
| 8513 | VLIENVAsLFFRKNWLRLTW |
| 8514 | VLIGsPKKVFFRKNWLRLTW |
| 8515 | VLIGsPKKYFFRKNWLRLTW |
| 8516 | VLKGsRSSELFFRKNWLRLTW |
| 8517 | VLKGsRSSEVFFRKNWLRLTW |
| 8518 | VLKSRKssVTEEFFRKNWLRLTW |
| 8519 | VLKVMIGsPKFFRKNWLRLTW |
| 8520 | VLKVMIGsPKKFFRKNWLRLTW |
| 8521 | VLKVMIGsPKKKFFRKNWLRLTW |
| 8522 | VLLsPVPELFFRKNWLRLTW |
| 8523 | VLLsPVPEVFFRKNWLRLTW |
| 8524 | VLMK(sPs)PALFFRKNWLRLTW |
| 8525 | VLMK(sPs)PAVFFRKNWLRLTW |
| 8526 | VLQtPPYVKFFRKNWLRLTW |
| 8527 | VLQtPPYVKKFFRKNWLRLTW |
| 8528 | VLQtPPYVKYFFRKNWLRLTW |
| 8529 | VLSDVIPsIFFRKNWLRLTW |
| 8530 | VLSSLtPAKVFFRKNWLRLTW |
| 8531 | VLWDTPsIFFRKNWLRLTW |
| 8532 | VLYsPQMALFFRKNWLRLTW |
| 8533 | VMFRtPLASVFFRKNWLRLTW |
| 8534 | VMIGsKKVFFRKNWLRLTW |
| 8535 | VMIGsPKKVFFRKNWLRLTW |
| 8536 | VMIGsPKKYFFRKNWLRLTW |
| 8537 | VMKVMIGsPKFFRKNWLRLTW |
| 8538 | VMKVMIGsPKKFFRKNWLRLTW |
| 8539 | VMKVMIGsPKKKFFRKNWLRLTW |
| 8540 | VMKVMIGsPKKYFFRKNWLRLTW |
| 8541 | VMLsPVPELFFRKNWLRLTW |
| 8542 | VMLsPVPEVFFRKNWLRLTW |
| 8543 | VMQtPPYVKFFRKNWLRLTW |
| 8544 | VMQtPPYVKKFFRKNWLRLTW |
| 8545 | VPHHGFEDWsQIRFFRKNWLRLTW |
| 8546 | VPKSGRSSsLFFRKNWLRLTW |
| 8547 | VPKsPAFALFFRKNWLRLTW |
| 8548 | VPLIRKKsLFFRKNWLRLTW |
| 8549 | VPNAPPAYEKLsAEQSPPPYFFRKNWLRLTW |
| 8550 | VPREVLRLsFFFRKNWLRLTW |
| 8551 | VPREVLRLsLFFRKNWLRLTW |
| 8552 | VPREVLRLsMFFRKNWLRLTW |
| 8553 | VPREVLRLsVFFRKNWLRLTW |
| 8554 | VPRPERRsSLFFRKNWLRLTW |
| 8555 | VPRsPKHAHSSSFFFRKNWLRLTW |
| 8556 | VPRsPKHAHSSSLFFRKNWLRLTW |
| 8557 | VPRsPKHAHSSSMFFRKNWLRLTW |
| 8558 | VPRsPKHAHSSSVFFRKNWLRLTW |
| 8559 | VPStPKSSLFFRKNWLRLTW |
| 8560 | VPTsPKSSLFFRKNWLRLTW |
| 8561 | VPVsPGQQLFFRKNWLRLTW |
| 8562 | VRAsKDLAQFFRKNWLRLTW |
| 8563 | VRQsVTSFPDADAFHHQFFRKNWLRLTW |
| 8564 | VSKVMIGsPKKVFFRKNWLRLTW |
| 8565 | VSKVMIGsPKKYFFRKNWLRLTW |
| 8566 | VTQtPPYVKKFFRKNWLRLTW |
| 8567 | VTQtPPYVKYFFRKNWLRLTW |
| 8568 | WDsPGQEVLFFRKNWLRLTW |
| 8569 | VYTyIQSRFFFRKNWLRLTW |
| 8570 | WTHLsSKEVDPSFFRKNWLRLTW |
| 8571 | WTHLsSKEVDPSTGFFRKNWLRLTW |
| 8572 | YARsVHEEFFFRKNWLRLTW |
| 8573 | YAVPRRGsLFFRKNWLRLTW |
| 8574 | YAYDGKDyIFFRKNWLRLTW |
| 8575 | YEGsPIKVFFRKNWLRLTW |
| 8576 | YEKLsAEQSPPPFFRKNWLRLTW |
| 8577 | YFsPFRPYFFRKNWLRLTW |
| 8578 | yIQSRFFFRKNWLRLTW |
| 8579 | YLAsLEKKLFFRKNWLRLTW |
| 8580 | YLDsGIHSGFFRKNWLRLTW |
| 8581 | YLDsGIHsGAFFRKNWLRLTW |
| 8582 | YLDsGIHSGAFFRKNWLRLTW |
| 8583 | YLDsGIHsGVFFRKNWLRLTW |
| 8584 | YLDsGIHSGVFFRKNWLRLTW |
| 8585 | yLGLDVPVFFRKNWLRLTW |
| 8586 | YLGsISTLVTLFFRKNWLRLTW |
| 8587 | YLIHsPMSLFFRKNWLRLTW |
| 8588 | YLLsPLNTLFFRKNWLRLTW |
| 8589 | YLLsPTKLPSIFFRKNWLRLTW |
| 8590 | YLLsPTKLPSVFFRKNWLRLTW |
| 8591 | yLQSRYYRAFFRKNWLRLTW |
| 8592 | YLQsRYYRAFFRKNWLRLTW |
| 8593 | YLSDsDTEAKLFFRKNWLRLTW |
| 8594 | YMDsGIHsGAFFRKNWLRLTW |
| 8595 | YMDsGIHSGAFFRKNWLRLTW |
| 8596 | YMDsGIHsGVFFRKNWLRLTW |
| 8597 | YMDsGIHSGVFFRKNWLRLTW |
| 8598 | YPDPHsPFAVFFRKNWLRLTW |
| 8599 | YPGGRRsSLFFRKNWLRLTW |
| 8600 | YPLsPAKVNQYFFRKNWLRLTW |
| 8601 | YPLsPTKISEYFFRKNWLRLTW |
| 8602 | YPLsPTKISQYFFRKNWLRLTW |
| 8603 | YPRsEDEVEGVMFFRKNWLRLTW |
| 8604 | YPRsFDEVEGFFFRKNWLRLTW |
| 8605 | YPRsFDEVEGLFFRKNWLRLTW |
| 8606 | YPRsFDEVEGMFFRKNWLRLTW |
| 8607 | YPRsFDEVEGVFFRKNWLRLTW |
| 8608 | YPRsFDEVEGVFFFRKNWLRLTW |
| 8609 | YPRsFDEVEGVLFFRKNWLRLTW |
| 8610 | YPRsFDEVEGVMFFRKNWLRLTW |
| 8611 | YPRsFDEVEGWFFRKNWLRLTW |
| 8612 | YPSFRRsSLFFRKNWLRLTW |
| 8613 | YPSsPRKALFFRKNWLRLTW |
| 8614 | YPSsPRKFFFRKNWLRLTW |
| 8615 | YPSsPRKLFFRKNWLRLTW |
| 8616 | YPSsPRKMFFRKNWLRLTW |
| 8617 | YPSsPRKVFFRKNWLRLTW |
| 8618 | YPYEFsPVKMFFRKNWLRLTW |
| 8619 | YQLsPTKLPSIFFRKNWLRLTW |
| 8620 | YQLsPTKLPSVFFRKNWLRLTW |
| 8621 | YQRPFsPSAYFFRKNWLRLTW |
| 8622 | YQRsFDEVEGFFFRKNWLRLTW |
| 8623 | YQRsFDEVEGLFFRKNWLRLTW |
| 8624 | YQRsFDEVEGMFFRKNWLRLTW |
| 8625 | YQRsFDEVEGVFFRKNWLRLTW |
| 8626 | YQRsFDEVEGVFFFRKNWLRLTW |
| 8627 | YQRsFDEVEGVLFFRKNWLRLTW |
| 8628 | YQRsFDEVEGVMFFRKNWLRLTW |
| 8629 | YQRsFDEVEGWFFRKNWLRLTW |
| 8630 | YRYsPQSFLFFRKNWLRLTW |
| 8631 | YTAGtPYKVFFRKNWLRLTW |
| 8632 | YYTAGSSsPTHAKSAHVFFRKNWLRLTW |
| 8810 | RLLsAAENFLFFRKNWLRLTW |

Lowercase s, t, and y indicate phosphorylated serine, phosphorylated threonine, and phosphorylated tyrosine, respectively.
Lowercase c indicates that the cysteine is present in a cysteine-cysteine disulfide bond.
Lowercase m indicates oxidized methionine.
(AcS) indicates an N-terminally acetylated serine.
(sLss) indicates that at least one serine residue in the amino acid sequence SLSS is phosphorylated.
(sPs) indicates that at least one serine residue in the amino acid sequence SPS is phosphorylated.

TABLE 6
Amino acid sequences of exemplary
antigenic polypeptides

| SEQ | |
|---|---|
| ID | |
| NO | Amino Acid Sequence |
| 8633 | ALTtsAHSVFFRKNLLRLTG |
| 8634 | ALTtSAHSVFFRKNLLRLTG |
| 8635 | ALTTsAHSVFFRKNLLRLTG |
| 8636 | APP(sts)AAALFFRKNLLRLTG |
| 8637 | APPsTSAAALFFRKNLLRLTG |
| 8638 | APPsTsAAALFFRKNLLRLTG |
| 8639 | APPStSAAALFFRKNLLRLTG |
| 8640 | APPSTsAAALFFRKNLLRLTG |
| 8641 | APPstSAAALFFRKNLLRLTG |
| 8642 | APPStsAAALFFRKNLLRLTG |
| 8643 | APP<s>TSAAALFFRKNLLRLTG |
| 8644 | APPS<t>SAAALFFRKNLLRLTG |
| 8645 | APPST<s>AAALFFRKNLLRLTG |
| 8646 | APPS<t>sAAALFFRKNLLRLTG |
| 8647 | APP<s><t>SAAALFFRKNLLRLTG |
| 8648 | APP<s>T<s>AAALFFRKNLLRLTG |
| 8649 | APPS<t><s>AAALFFRKNLLRLTG |
| 8650 | APRG<n>VISLFFRKNLLRLIG |
| 8651 | APRtNGVAMFFRKNLLRLTG |
| 8652 | APTsAAALFFRKNLLRLTG |
| 8653 | APTsASNVMFFRKNLLRLTG |
| 8654 | APTSAsNVMFFRKNLLRLTG |
| 8655 | APVsASASVFFRKNLLRLTG |
| 8656 | APVsSKSSLFFRKNLLRLTG |
| 8657 | EP(sst)VVSLFFRKNLLRLTG |
| 8658 | EPsSTVVSLFFRKNLLRLTG |
| 8659 | EPSsTVVSLFFRKNLLRLTG |
| 8660 | EPSStVVSLFFRKNLLRLTG |
| 8661 | GLSsLAEEAAFFRKNLLRLTG |
| 8662 | HP(sss)AAVLFFRKNLLRLTG⁽ⁱ⁾ |
| 8663 | HP(sst)ASTALFFRKNLLRLTG |
| 8664 | HPMsTASQVFFRKNLLRLTG |
| 8665 | HPssTAAVLFFRKNLLRLTG |
| 8666 | HPsStAAVLFFRKNLLRLTG |
| 8667 | HPSstAAVLFFRKNLLRLTG |
| 8668 | HPsSTASTALFFRKNLLRLTG |
| 8669 | HPSsTASTALFFRKNLLRLTG |
| 8670 | HPSStASTALFFRKNLLRLTG |
| 8671 | HPTtVASYFFRKNLLRLTG |
| 8672 | IPIsLHTSLFFRKNLLRLTG |
| 8673 | IPTsSVLSLFFRKNLLRLTG |
| 8674 | IPVsKPLSLFFRKNLLRLTG |
| 8675 | IPV<s>KPLSLFFRKNLLRLTG |
| 8676 | IPVsSHNSLFFRKNLLRLTG |
| 8677 | IPVssHNSLFFRKNLLRLTG |
| 8678 | IPV<s>SHNSLFFRKNLLRLTG |
| 8679 | IPV[s]SHNSLFFRKNLLRLTG |
| 8680 | KPPtSQSSVLFFRKNLLRLTG |
| 8681 | KPP<t>SQSSVLFFRKNLLRLTG |
| 8682 | KPPTsQSSVLFFRKNLLRLTG |
| 8683 | KPPT<s>QSSVLFFRKNLLRLTG |
| 8684 | KPPV<s>FFSLFFRKNLLRLIG |
| 8685 | KPTLY<n>VSLFFRKNLLRLIG |
| 8686 | LPRN(st)MMFFRKNLLRLTG |
| 8687 | LPRNstMMFFRKNLLRLTG |
| 8688 | LPTsLPSSLFFRKNLLRLTG |
| 8689 | MPVRPT<t>NTFFFRKNLLRLTG |
| 8690 | (diMe)MPVRPT<t>NTFFFRKNLL |
| | RLTG |
| 8691 | MPVtSSSFFFFRKNLLRLTG |
| 8692 | NPVsLPSLFFRKNLLRLTG |
| 8693 | PPS<t>SAAALFFRKNLLRLTG |
| 8694 | PPST<s>AAALFFRKNLLRLIG |
| 8695 | RPP(sss)QQLFFRKNLLRLTG |
| 8696 | RPPItQSSLFFRKNLLRLTG |
| 8697 | (Me)RPPItQSSLFFRKNLLRLTG |
| 8698 | (diME)RPPItQSSLFFRKNLLRLT |
| | G |
| 8699 | (diME)RPPI[t]QSSLFFRKNLLR |
| | LTG |
| 8700 | RPPQ<s>SSVSLFFRKNLLRLTG |
| 8701 | RPPsSSQQLFFRKNLLRLTG |
| 8702 | RPPSsSQQLFFRKNLLRLTG |
| 8703 | RPPSSsQQLFFRKNLLRLTG |
| 8704 | RPPVtKASSFFFRKNLLRLTG |
| 8705 | RPVtASITTMFFRKNLLRLTG |
| 8706 | TPASsRAQTLFFRKNLLRLTG |
| 8707 | TPAsSSSALFFRKNLLRLTG |
| 8708 | TPIsQAQKLFFRKNLLRLTG |
| 8709 | TPVsSANMMFFRKNLLRLTG |
| 8710 | VLTsNVQTIFFRKNLLRLTG |
| 8711 | VPAsSTSTLFFRKNLLRLTG |
| 8712 | VPAtHGQVTYFFRKNLLRLTG |
| 8713 | VPtTSSSLFFRKNLLRLTG |
| 8714 | VPTtSSSLFFRKNLLRLTG |
| 8715 | VPTTsSSLFFRKNLLRLTG |
| 8716 | VPVsGTQGLFFRKNLLRLTG |
| 8717 | VPVsNQSSLFFRKNLLRLTG |
| 8718 | VPVsSASELFFRKNLLRLTG |
| 8719 | VPVsVGPSLFFRKNLLRLTG |

Lowercase s and t indicate O-GlcNAcylated serine and O-GlcNAcylated threonine, respectively.
(sts), (sss), (ts), (sst), and (st) indicates at least one of the serine or threonine residues is modified with O-GlcNAc.
⁽ⁱ⁾indicates that two GlcNAc moeities were detected, but could not be assigned to specific amino acids.
(Me) indicates methylation of the following arginine.
(diMe) indicates asymmetric di-methylation of the following arginine.
<n> indicates hexose-GlcNAcylated asparagine.
<s> indicates hexose-GlcNAcylated serine.
<t> indicates hexose-GlcNAcylated threonine.
[s] indicates acetyl-GlcNAcylated serine.
[t] indicates acetyl-GlcNAcylated threonine.

TABLE 7
Amino acid sequences of exemplary
antigenic polypeptides

| SEQ | |
|---|---|
| ID | |
| NO | Amino Acid Sequence |
| 8720 | ALTtsAHSVFFRKNWLRLTW |
| 8721 | ALTtSAHSVFFRKNWLRLTW |
| 8722 | ALTTsAHSVFFRKNWLRLTW |
| 8723 | APP(sts)AAALFFRKNWLRLTW |
| 8724 | APPsTSAAALFFRKNWLRLTW |
| 8725 | APPsTsAAALFFRKNWLRLTW |
| 8726 | APPStSAAALFFRKNWLRLTW |
| 8727 | APPSTsAAALFFRKNWLRLTW |
| 8728 | APPstSAAALFFRKNWLRLTW |
| 8729 | APPStsAAALFFRKNWLRLTW |
| 8730 | APP<s>TSAAALFFRKNWLRLTW |
| 8731 | APPS<t>SAAALFFRKNWLRLTW |
| 8732 | APPST<s>AAALFFRKNWLRLTW |
| 8733 | APPS<t>sAAALFFRKNWLRLTW |
| 8734 | APP<s><t>SAAALFFRKNWLRLTW |
| 8735 | APP<s>T<s>AAALFFRKNWLRLTW |
| 8736 | APPS<t><s>AAALFFRKNWLRLTW |
| 8737 | APRG<n>VISLFFRKNWLRLTW |
| 8738 | APRtNGVAMFFRKNWLRLTW |
| 8739 | APTsAAALFFRKNWLRLTW |
| 8740 | APTsASNVMFFRKNWLRLTW |
| 8741 | APTSAsNVMFFRKNWLRLTW |
| 8742 | APVsASASVFFRKNWLRLTW |
| 8743 | APVsSKSSLFFRKNWLRLTW |
| 8744 | EP(sst)VVSLFFRKNWLRLTW |
| 8745 | EPsSTVVSLFFRKNWLRLTW |
| 8746 | EPSsTVVSLFFRKNWLRLTW |
| 8747 | EPSStVVSLFFRKNWLRLTW |
| 8748 | GLSsLAEEAAFFRKNWLRLTW |
| 8749 | HP(sss)AAVLFFRKNWLRLTW⁽ⁱ⁾ |
| 8750 | HP(sst)ASTALFFRKNWLRLTW |
| 8751 | HPMsTASQVFFRKNWLRLTW |
| 8752 | HPssTAAVLFFRKNWLRLTW |
| 8753 | HPsStAAVLFFRKNWLRLTW |
| 8754 | HPSstAAVLFFRKNWLRLTW |
| 8755 | HPsSTASTALFFRKNWLRLTW |
| 8756 | HPSsTASTALFFRKNWLRLTW |
| 8757 | HPSStASTALFFRKNWLRLTW |
| 8758 | HPTtVASYFFRKNWLRLTW |
| 8759 | IPIsLHTSLFFRKNWLRLTW |
| 8760 | IPTsSVLSLFFRKNWLRLTW |
| 8761 | IPVsKPLSLFFRKNWLRLTW |
| 8762 | IPV<s>KPLSLFFRKNWLRLTW |
| 8763 | IPVsSHNSLFFRKNWLRLTW |
| 8764 | IPVssHNSLFFRKNWLRLTW |
| 8765 | IPV<s>SHNSLFFRKNWLRLTW |
| 8766 | IPV[s]SHNSLFFRKNWLRLTW |
| 8767 | KPPtSQSSVLFFRKNWLRLTW |
| 8768 | KPP<t>SQSSVLFFRKNWLRLTW |
| 8769 | KPPTsQSSVLFFRKNWLRLTW |
| 8770 | KPPT<s>QSSVLFFRKNWLRLTW |
| 8771 | KPPV<s>FFSLFFRKNWLRLTW |
| 8772 | KPTLY<n>VSLFFRKNWLRLTW |
| 8773 | LPRN(st)MMFFRKNWLRLTW |
| 8774 | LPRNstMMFFRKNWLRLTW |
| 8775 | LPTsLPSSLFFRKNWLRLTW |
| 8776 | MPVRPT<t>NIFFFRKNWLRLTW |
| 8777 | (diMe)MPVRPT<t>NIFFFRKNW |
| | LRLTW |
| 8778 | MPVtSSSFFFFRKNWLRLTW |
| 8779 | NPVsLPSLFFRKNWLRLTW |
| 8780 | PPS<t>SAAALFFRKNWLRLTW |
| 8781 | PPST<s>AAALFFRKNWLRLTW |
| 8782 | RPP(sss)QQLFFRKNWLRLTW |
| 8783 | RPPItQSSLFFRKNWLRLTW |
| 8784 | (Me)RPPItQSSLFFRKNWLRLTW |
| 8785 | (diME)RPPItQSSLFFRKNWLRLT |
| | W |
| 8786 | (diME)RPPI[t]QSSLFFRKNWLR |
| | LTW |
| 8787 | RPPQ<s>SSVSLFFRKNWLRLTW |
| 8788 | RPPsSSQQLFFRKNWLRLTW |
| 8789 | RPPSsSQQLFFRKNWLRLTW |
| 8790 | RPPSSsQQLFFRKNWLRLTW |
| 8791 | RPPVtKASSFFFRKNWLRLTW |
| 8792 | RPVtASITTMFFRKNWLRLTW |
| 8793 | TPASsRAQTLFFRKNWLRLTW |
| 8794 | TPAsSSSALFFRKNWLRLTW |
| 8795 | TPIsQAQKLFFRKNWLRLTW |
| 8796 | TPVsSANMMFFRKNWLRLTW |
| 8797 | VLTsNVQTIFFRKNWLRLTW |
| 8798 | VPAsSTSTLFFRKNWLRLTW |
| 8799 | VPAtHGQVTYFFRKNWLRLTW |
| 8800 | VPtTSSSLFFRKNWLRLTW |
| 8801 | VPTtSSSLFFRKNWLRLTW |
| 8802 | VPTTsSSLFFRKNWLRLTW |
| 8803 | VPVsGTQGLFFRKNWLRLTW |
| 8804 | VPVsNQSSLFFRKNWLRLTW |
| 8805 | VPVsSASELFFRKNWLRLTW |
| 8806 | VPVsVGPSLFFRKNWLRLTW |

Lowercase s and t indicate O-GlcNAcylated serine and O-GlcNAcylated threonine, respectively.
(sts), (sss), (ts), (sst), and (st) indicates at least one of the serine or threonine residues is modified with O-GlcNAc.
⁽ⁱ⁾indicates that two GlcNAc moeities were detected, but could not be assigned to specific amino acids.
(Me) indicates methylation of the following arginine.
(diMe) indicates asymmetric di-methylation of the following arginine.
<n> indicates hexose-GlcNAcylated asparagine.
<s> indicates hexose-GlcNAcylated serine.
<t> indicates hexose-GlcNAcylated threonine.
[s] indicates acetyl-GlcNAcylated serine.
[t] indicates acetyl-GlcNAcylated threonine.

In certain embodiments, the instant disclosure provides: an antigenic polypeptide comprising an MHC-binding peptide comprising an amino acid sequence selected from the group consisting of SEQ ID NOs: 98-3000 and 8808; and an HSP-binding peptide comprising the amino acid sequence of X₁X₂X₃X₄X₅X₆X₇ (SEQ ID NO: 1), wherein X₁ is omitted, N, F, or Q; X₂ is W, L, or F; X₃ is L or I; X₄ is R, L, or K; X₅ is L, W, or I; X₆ is T, L, F, K, R, or W; and X₇ is W, G, K, or F.

In certain embodiments, the HSP-binding peptide comprises the amino acid sequence of:
(a) X₁LX₂LTX₃ (SEQ ID NO: 2), wherein X₁ is W or F; X₂ is R or K; and X₃ is W, F, or G;
(b) NX₁LX₂LTX₃ (SEQ ID NO: 3), wherein X₁ is W or F; X₂ is R or K; and X₃ is W, F, or G;
(c) WLX₁LTX₂ (SEQ ID NO: 4), wherein X₁ is R or K; and X₂ is W or G;
(d) NWLX₁LTX₂ (SEQ ID NO: 5), wherein X₁ is R or K; and X₂ is W or G; or
(e) NWX₁X₂X₃X₄X₅ (SEQ ID NO: 6), wherein X₁ is L or I; X₂ is L, R, or K; X₃ is L or I; X₄ is T, L, F, K, R, or W; and X₅ is W or K.

In certain embodiments, the instant disclosure provides: an antigenic polypeptide comprising an WIC-binding peptide comprising an amino acid sequence selected from the group consisting of SEQ ID NOs: 98-3000 and 8808, optionally wherein the amino acid sequence of the WIC-binding peptide consists of an amino acid sequence selected from the group consisting of SEQ ID NOs: 98-3000 and 8808; and an HSP-binding peptide comprising an amino acid sequence selected from the group consisting of SEQ ID NOs: 1-42, optionally wherein the amino acid sequence of the HSP-binding peptide consists of an amino acid sequence selected from the group consisting of SEQ ID NOs: 1-42.

In certain embodiments, the C-terminus of the WIC-binding peptide is linked (either directly or indirectly) to the N-terminus of the HSP-binding peptide. Accordingly, in certain embodiments, the antigenic polypeptide comprises an MHC-binding peptide comprising an amino acid sequence selected from the group consisting of SEQ ID NOs: 98-3000 and 8808, and an HSP-binding peptide comprising an amino acid sequence selected from the group consisting of SEQ ID NOs: 1-42, wherein the C-terminus of the MHC-binding peptide is linked (either directly or indirectly) to the N-terminus of the HSP-binding peptide.

In certain embodiments, the N-terminus of the MHC-binding peptide is linked (either directly or indirectly) to the C-terminus of the HSP-binding peptide. Accordingly, in certain embodiments, the antigenic polypeptide comprises an MHC-binding peptide comprising an amino acid sequence selected from the group consisting of SEQ ID NOs: 98-3000 and 8808, and an HSP-binding peptide comprising an amino acid sequence selected from the group consisting of SEQ ID NOs: 1-42, wherein the N-terminus of the WIC-binding peptide is linked (either directly or indirectly) to the C-terminus of the HSP-binding peptide.

In certain embodiments, the MHC-binding peptide is 8 to 50 amino acids in length, optionally 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

In certain embodiments, the HSP-binding peptide is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In certain embodiments, the HSP-binding peptide is less than 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

In certain embodiments, the HSP-binding peptide is linked to the MHC-binding peptide via a chemical linker. Any chemical linkers can be employed to link the HSP-binding peptide and the WIC-binding peptide. Exemplary chemical linkers include moieties generated from chemical crosslinking (see, e.g., Wong, 1991, Chemistry of Protein Conjugation and Cross-Linking, CRC Press, incorporated herein by reference in its entirety), UV crosslinking, and click chemistry reactions (see, e.g., U.S. Patent Publication 20130266512, which is incorporated by reference herein in its entirety).

In certain embodiments, the HSP-binding peptide is linked to the MHC-binding peptide via a peptide linker (e.g., a peptide linker as disclosed herein). In certain embodiments, the peptide linker comprises the amino acid sequence of SEQ ID NO: 43 or FR. In certain embodiments, the amino acid sequence of the peptide linker consists of the amino acid sequence of SEQ ID NO: 43 or FR.

In certain embodiments, the C-terminus of the MHC-binding peptide is linked by the peptide linker of SEQ ID NO: 43 or FR to the N-terminus of the HSP-binding peptide. Accordingly, in certain embodiments, the antigenic polypeptide comprises from N-terminus to C-terminus: an MHC-binding peptide comprising an amino acid sequence selected from the group consisting of SEQ ID NOs: 98-3000 and 8808; the peptide linker of SEQ ID NO: 43 or FR; and an HSP-binding peptide comprising an amino acid sequence selected from the group consisting of SEQ ID NOs: 1-42. In certain embodiments, the amino acid sequence of the WIC-binding peptide consists of an amino acid sequence selected from the group consisting of SEQ ID NOs: 98-3000 and 8808, and the amino acid sequence of the HSP-binding peptide consists of an amino acid sequence selected from the group consisting of SEQ ID NOs: 1-42.

In certain embodiments, the antigenic polypeptide comprises an amino acid sequence selected from the group consisting of SEQ ID NOs: 3001-8806, 8809, and 8810. In certain embodiments, the amino acid sequence of the antigenic polypeptide consists of an amino acid sequence selected from the group consisting of SEQ ID NOs: 3001-8806, 8809, and 8810. In certain embodiments, the antigenic polypeptide consists of an amino acid sequence selected from the group consisting of SEQ ID NOs: 3001-8806, 8809, and 8810.

In certain embodiments, the N-terminus of the MHC-binding peptide is linked by the peptide linker of SEQ ID NO: 43 or FR to the C-terminus of the HSP-binding peptide. Accordingly, in certain embodiments the antigenic polypeptide comprises from N-terminus to C-terminus: an HSP-binding peptide comprising an amino acid sequence selected from the group consisting of SEQ ID NOs: 1-42; the peptide linker of SEQ ID NO: 43 or FR; and an WIC-binding peptide comprising an amino acid sequence selected from the group consisting of SEQ ID NOs: 98-3000 and 8808. In certain embodiments, the amino acid sequence of the MHC-binding peptide consists of an amino acid sequence selected from the group consisting of SEQ ID NOs: 98-3000 and 8808, and the amino acid sequence of the HSP-binding peptide consists of an amino acid sequence selected from the group consisting of SEQ ID NOs: 1-42.

In certain embodiments, the antigenic polypeptide comprises an MHC-binding peptide comprising an amino acid sequence selected from the group consisting of SEQ ID NOs: 98-3000 and 8808, and wherein the N-terminus of the MHC-binding peptide is linked to the C-terminus of an amino acid sequence selected from the group consisting of SEQ ID NOs: 74-97. In certain embodiments, the amino acid sequence of the MHC-binding peptide consists of an amino acid sequence selected from the group consisting of SEQ ID NOs: 98-3000 and 8808.

In certain embodiments, the antigenic polypeptide comprises an MHC-binding peptide comprising an amino acid sequence selected from the group consisting of SEQ ID NOs: 98-3000 and 8808, and wherein the C-terminus of the WIC-binding peptide is linked to the N-terminus of an amino acid sequence selected from the group consisting of SEQ ID NOs: 50-67. In certain embodiments, the amino acid sequence of the MHC-binding peptide consists of an amino acid sequence selected from the group consisting of SEQ ID NOs: 98-3000 and 8808.

In certain embodiments, the antigenic peptides disclosed herein are 8 to 100 amino acids, (e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids) in length. In certain embodiments, an antigenic peptide is 8 to 50 amino acids in length.

In certain embodiments, the antigenic peptides disclosed herein are less than 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length.

In certain embodiments, the amino acid sequence of the antigenic polypeptides disclosed herein does not comprise more than 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 contiguous amino acids of a protein (e.g., a naturally occurring protein) that comprises an amino acid sequence selected from the group consisting of SEQ ID NO: 98-3000 and 8808.

In certain embodiments, the instant disclosure provides a polypeptide comprising an amino acid sequence selected from the group consisting of SEQ ID NOs: 3001-8806, 8809, and 8810. In certain embodiments, the polypeptide is 15 to 100 amino acids in length, optionally, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length. In certain embodiments, the amino acid sequence of the antigenic polypeptide consists of an amino acid sequence selected from the group consisting of SEQ ID NOs: 3001-8806, 8809, and 8810.

The antigenic polypeptide disclosed herein can comprise one or more MHC-binding peptides. In certain embodiments, the antigenic peptide comprises one MHC-binding peptides. In certain embodiments, the antigenic polypeptide comprises two or more (e.g., 3, 4, 5, 6, 7, 8, 9, 10, or more) MHC-binding peptides. The two or more MHC-binding peptides can be linked via a chemical linker or a peptide linker, wherein the peptide linker optionally comprises an amino acid sequence that can be recognized and/or cleaved by a protease.

The skilled worker will appreciate that the antigenic polypeptides disclosed herein also encompass derivatives of antigenic polypeptides that are modified during or after synthesis. Such modifications include, but are not limited to: glycosylation, acetylation, methylation, phosphorylation (e.g., phosphorylation of Tyr, Ser, Thr, Arg, Lys, or His on a side chain hydroxyl or amine), formylation, or amidation (e.g., amidation of a C-terminal carboxyl group); derivatization using reactive chemical groups (e.g., derivatization of: free NH₂, COOH, or OH groups); specific chemical cleavage (e.g., by cyanogen bromide, hydroxylamine, BNPS-Skatole, acid, NaBH₄, or alkali hydrolysis); enzymatic cleavage (e.g., by trypsin, chymotrypsin, papain, V8 protease; oxidation; reduction; etc. Methods for effecting the foregoing modification to antigenic polypeptides are well known in the art.

In certain embodiments, the antigenic polypeptide comprises one or more modified amino acid residues (e.g., in the MHC-binding peptide portion of the antigenic polypeptide). In certain embodiments, the antigenic polypeptide comprises a phosphorylated residue (e.g., a Tyr, Ser, Thr, Arg, Lys, or His that has been phosphorylated on a side chain hydroxyl or amine). In certain embodiments, the antigenic polypeptide comprises a phosphomimetic residue (e.g., a mimetic of a Tyr, Ser, Thr, Arg, Lys, or His amino acid that has been phosphorylated on a side chain hydroxyl or amine). Non-limiting examples of phosphomimetic groups include O-boranophospho, borono, O-dithiophospho, phosphoramide, H-phosphonate, alkylphosphonate, phosphorothioate, phosphodithioate and phosphorofluoridate, any of which may be derivatized on Tyr, Thr, Ser, Arg, Lys, or His residues. In certain embodiments, an Asp or Glu residue is used as a phosphomimetic in place of a phospho-Tyr, phospho-Thr, phospho-Ser, phospho-Arg, phospho-Lys and/or phospho-His residue in a peptide. In certain embodiments, the phosphomimetic residue is a non-hydrolyzable analogue of a phosphorylated residue. Accordingly, in certain embodiments, the antigenic polypeptide comprises a phosphopeptide selected from the group consisting of SEQ ID NOs: 98-3000 and 8808, wherein a phosphorylated amino acid residue of the phosphopeptide is replaced by a non-hydrolyzable mimetic of the phosphorylated amino acid residue.

The skilled worker will further appreciate that, in certain embodiments, the antigenic polypeptides disclosed herein can comprise one or more natural and/or non-natural amino acids (e.g., D-amino acids), and amino acid analogues and derivatives (e.g., disubstituted amino acids, N-alkyl amino acids, lactic acid, 4-hydroxyproline, γ-carboxyglutamate, ε-N,N,N-trimethyllysine, ε-N-acetyllysine, 0-phosphoserine, N-acetylserine, N-formylmethionine, 3-methylhistidine, 5-hydroxylysine, σ-N-methylarginine). In certain embodiments, the antigenic polypeptides disclosed herein comprise one or more retro-inverso peptides. A “retro-inverso peptide” refers to a peptide with a reversal of the peptide sequence in two or more positions and inversion of the stereochemistry from L to D configuration in chiral amino acids. Thus, a retro-inverso peptide has reversed termini, reversed direction of peptide bonds, and reversed peptide sequence from N-to-C-terminus, while approximately maintaining the topology of the side chains as in the native peptide sequence. Synthesis of retro-inverso peptide analogues are described in Bonelli, F. et al., Int J Pept Protein Res. 24(6):553-6 (1984); Verdini, A and Viscomi, G. C, J. Chem. Soc. Perkin Trans. 1:697-701 (1985); and U.S. Pat. No. 6,261,569, which are incorporated herein in their entirety by reference.

### 6.2.1 Production of Antigenic Polypeptides by Chemical Synthesis

Antigenic polypeptides disclosed herein can be synthesized by standard chemical methods including the use of a peptide synthesizer. Conventional peptide synthesis or other synthetic protocols well known in the art can be used.

In certain embodiments, the polypeptide disclosed herein consists of amino acid residues (natural or non-natural) linked by peptide bonds. Such polypeptides can be synthesized, for example, by solid-phase peptide synthesis using procedures similar to those described by Merrifield, 1963, J. Am. Chem. Soc., 85:2149, incorporated herein by reference in its entirety. During synthesis, N-α-protected amino acids having protected side chains are added stepwise to a growing polypeptide chain linked by its C-terminal end to an insoluble polymeric support i.e., polystyrene beads. The polypeptides are synthesized by linking an amino group of an N-α-deprotected amino acid to an α-carboxyl group of an N-α-protected amino acid that has been activated by reacting it with a reagent such as dicyclohexylcarbodiimide or 2-(6-Chloro-1-H-benzotriazole-1-yl)-1,1,3,3-tetramethylaminium hexafluorophosphate. The attachment of a free amino group to the activated carboxyl leads to peptide bond formation. The most commonly used N-α-protecting groups include Boc which is acid labile and Fmoc which is base labile. Details of appropriate chemistries, resins, protecting groups, protected amino acids and reagents are well known in the art (See, Atherton, et al., 1989, Solid Phase Peptide Synthesis: A Practical Approach, IRL Press, and Bodanszky, 1993, Peptide Chemistry, A Practical Textbook, 2nd Ed., Springer-Verlag, each of which is incorporated herein by reference in its entirety).

In addition, analogs and derivatives of polypeptides can be chemically synthesized as described supra. If desired, nonclassical amino acids or chemical amino acid analogs can be introduced as a substitution or addition into the peptide sequence. Non-classical amino acids include, but are not limited to, the D-isomers of the common amino acids, α-amino isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, C-α-methyl amino acids, and N-α-methyl amino acids.

Polypeptides phosphorylated on the side chains of Tyr, Ser, Thr, Arg, Lys, and His can be synthesized in Fmoc solid phase synthesis using the appropriate side chain protected Fmoc-phospho amino acid. In this way, polypeptides with a combination of phosphorylated and non-phosphorylated Tyr, Ser, Thr, Arg, Lys, and His residues can be synthesized. For example, the method of Staerkaer et al can be applied (1991, Tetrahedron Letters 32: 5389-5392). Other procedures (some for specific amino acids) are detailed in De Bont et al. (1987, Tray. Chim Pays Bas 106: 641, 642), Bannwarth and Trezeciak (1987, Helv. Chim. Acta 70: 175-186), Perich and Johns (1988, Tetrahedron Letters 29: 2369-2372), Kitas et al. (1990, J. Org. Chem. 55:4181-4187), Valerio et al. (1989, Int. J. Peptide Protein Res. 33:428-438), Perich et al. (1991, Tetrahedron Letters 32:4033-4034), Pennington (1994, Meth. Molec. Biol. 35:195-2), and Perich (1997, Methods Enzymol. 289:245-266, Chan et al. (2000, White, Fmoc Solid Phase Peptide Synthesis: A Practical Approach, Oxford University Press), Graham et al. (1999, Org. Lett., Vol. 1, No. 5), Russell et al., (2008 Org. Biomol. Chem. (2008, Sep. 21; 6(18):3270-5), each of which is incorporated herein by reference in its entirety).

A phosphorylated polypeptide can also be produced by first culturing a cell transformed with a nucleic acid that encodes the amino acid sequence of the polypeptide. After producing such a polypeptide by cell culture, the hydroxyl groups of the appropriate amino acid are substituted by phosphate groups using organic synthesis or enzymatic methods with phosphorylation enzymes. For example, in the case of serine-specific phosphorylation, serine kinases can be used.

Phosphopeptide mimetics can also be synthesized, wherein a phosphorylated amino acid residue in a polypeptide is replaced with a phosphomimetic group. Non-limiting examples of phosphomimetic groups include O-boranophospho, borono, O-dithiophospho, phosphoramide, H-phosphonate, alkylphosphonate, phosphorothioate, phosphodithioate and phosphorofluoridate, any of which may be derivatized on Tyr, Thr, Ser, Arg, Lys, or His residues. In certain embodiments, an Asp or Glu residue is used as a phosphomimetic. Asp or Glu residues can also function as phosphomimetic groups, and be used in place of a phospho-Tyr, phospho-Thr, phospho-Ser, phospho-Arg, phospho-Lys and/or phospho-His residue in a peptide.

Purification of the resulting peptide is accomplished using conventional procedures, such as preparative HPLC using reverse-phase, gel permeation, partition and/or ion exchange chromatography. The choice of appropriate matrices and buffers are well known in the art and so are not described in detail herein.

### 6.2.2 Production of Antigenic Polypeptides Using Recombinant DNA Technology

Polypeptides disclosed herein can also be prepared by recombinant DNA methods known in the art. A nucleic acid sequence encoding a polypeptide can be obtained by back translation of the amino acid sequence and synthesized by standard chemical methods, such as the use of an oligonucleotide synthesizer. Alternatively, coding information for polypeptides can be obtained from DNA templates using specifically designed oligonucleotide primers and PCR methodologies. Variations and fragments of the polypeptides can be made by substitutions, insertions or deletions that provide for functionally equivalent molecules. Due to the degeneracy of nucleotide coding sequences, DNA sequences which encode the same or a variant of a polypeptide may be used in the practice of the present invention. These include, but are not limited to, nucleotide sequences which are altered by the substitution of different codons that encode a functionally equivalent amino acid residue within the sequence, thus producing a silent or conservative change. The nucleic acid encoding a polypeptide can be inserted into an expression vector for propagation and expression in host cells.

As the coding sequence for peptides of the length contemplated herein can be synthesized by chemical techniques, for example, the phosphotriester method of Matteucci et al., J. Am. Chem. Soc. 103:3185 (1981) (incorporated herein by reference in its entirety), modification can be made simply by substituting the appropriate base(s) for those encoding the native peptide sequence. The coding sequence can then be provided with appropriate linkers and ligated into expression vectors commonly available in the art, and the vectors used to transform suitable hosts to produce the desired peptide or fusion protein. A number of such vectors and suitable host systems are now available. For expression of the peptide or fusion proteins, the coding sequence will be provided with operably linked start and stop codons, promoter and terminator regions and usually a replication system to provide an expression vector for expression in the desired cellular host.

An expression construct refers to a nucleotide sequence encoding a polypeptide operably linked with one or more regulatory regions which enables expression of the peptide in an appropriate host cell. “Operably-linked” refers to an association in which the regulatory regions and the peptide sequence to be expressed are joined and positioned in such a way as to permit transcription, and ultimately, translation.

The regulatory regions necessary for transcription of the peptide can be provided by the expression vector. A translation initiation codon (ATG) may also be provided if the peptide gene sequence lacking its cognate initiation codon is to be expressed. In a compatible host-construct system, cellular transcriptional factors, such as RNA polymerase, will bind to the regulatory regions on the expression construct to effect transcription of the peptide sequence in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites.

In order to attach DNA sequences with regulatory functions, such as promoters, to the peptide gene sequence or to insert the peptide gene sequence into the cloning site of a vector, linkers or adapters providing the appropriate compatible restriction sites may be ligated to the ends of the cDNAs by techniques well known in the art (Wu et al., 1987, Methods in Enzymol 152:343-349, incorporated herein by reference in its entirety). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA by use of PCR with primers containing the desired restriction enzyme site.

An expression construct comprising a polypeptide coding sequence operably linked with regulatory regions can be directly introduced into appropriate host cells for expression and production of the peptide without further cloning. The expression constructs can also contain DNA sequences that facilitate integration of the DNA sequence into the genome of the host cell, e.g., via homologous recombination. In this instance, it is not necessary to use an expression vector comprising a replication origin suitable for appropriate host cells in order to propagate and express the peptide in the host cells.

A variety of expression vectors may be used including plasmids, cosmids, phage, phagemids or modified viruses. Typically, such expression vectors comprise a functional origin of replication for propagation of the vector in an appropriate host cell, one or more restriction endonuclease sites for insertion of the peptide gene sequence, and one or more selection markers. Expression vectors may be constructed to carry nucleotide sequences for one or more of the polypeptides disclosed herein. The expression vector must be used with a compatible host cell which may be derived from a prokaryotic or eukaryotic organism including but not limited to bacteria, yeasts, insects, mammals and humans. Such host cells can be transformed to express one or more polypeptides disclosed herein, such as by transformation of the host cell with a single expression vector containing a plurality of nucleotide sequences encoding any of the polypeptides disclosed herein, or by transformation of the host cell with multiple expression vectors encoding different polypeptides disclosed herein.

In bacterial systems, a number of expression vectors may be advantageously selected to produce polypeptides. For example, when a large quantity of such a protein is to be produced, such as for the generation of pharmaceutical compositions, vectors that direct the expression of high levels of fusion protein products that are readily purified may be desirable. Such vectors include the E. coli expression vector pUR278 (Ruther et al., 1983, EMBO J. 2, 1791, incorporated herein by reference in its entirety), in which the peptide coding sequence may be ligated individually into the vector in frame with the lac Z coding region so that a fusion protein is produced; pIN vectors (Inouye and Inouye, 1985, Nucleic Acids Res. 13, 3101-3109; Van Heeke and Schuster, 1989, J. Biol. Chem 264, 5503-5509, each of which is incorporated herein by reference in its entirety); and the like. pGEX vectors may also be used to express these peptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the polypeptide can be released from the GST moiety.

Alternatively, for long term, high yield production of properly processed peptide complexes, stable expression in mammalian cells is preferred. Cell lines that stably express peptide complexes may be engineered by using a vector that contains a selectable marker. By way of example, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while the peptide is expressed continuously.

The recombinant cells may be cultured under standard conditions of temperature, incubation time, optical density and media composition. However, conditions for growth of recombinant cells may be different from those for expression of the polypeptides. Modified culture conditions and media may also be used to enhance production of the peptides. For example, recombinant cells containing peptides with their cognate promoters may be exposed to heat or other environmental stress, or chemical stress. Any techniques known in the art may be applied to establish the optimal conditions for producing peptide complexes.

In one embodiment disclosed herein, a codon encoding methionine is added at the 5′ end of the nucleotide sequence encoding a polypeptide to provide a signal for initiation of translation of the peptide. This methionine may remain attached to the polypeptide, or the methionine may be removed by the addition of an enzyme or enzymes that can catalyze the cleavage of methionine from the peptide. For example, in both prokaryotes and eukaryotes, N-terminal methionine is removed by a methionine aminopeptidase (MAP) (Tsunasawa et al., 1985, J. Biol. Chem. 260, 5382-5391, incorporated herein by reference in its entirety). Methionine aminopeptidases have been isolated and cloned from several organisms, including E. coli, yeast, and rat.

The peptide may be recovered from the bacterial, mammalian, or other host cell types, or from the culture medium, by known methods (see, for example, Current Protocols in Immunology, vol. 2, chapter 8, Coligan et al. (ed.), John Wiley & Sons, Inc.; Pathogenic and Clinical Microbiology: A Laboratory Manual by Rowland et al., Little Brown & Co., June 1994, incorporated herein by reference in its entirety).

Both of the foregoing methods can be used for synthesizing a polypeptide disclosed herein. For example, a peptide comprising the amino acid sequence of the HSP-binding peptide can be synthesized chemically, and joined to an antigenic peptide, optionally produced by recombinant DNA technology, via a peptide bond.

Included within the scope disclosed herein are derivatives or analogs of the polypeptides disclosed herein that are modified during or after translation, e.g., by glycosylation, acetylation, phosphorylation, amidation (e.g., of the C-terminal carboxyl group), or derivatization by known protecting/blocking groups, or proteolytic cleavage. Any of numerous chemical modifications may be carried out by known techniques, including but not limited to, reagents useful for protection or modification of free NH₂— groups, free COOH— groups, OH— groups, side groups of Trp-, Tyr-, Phe-, His-, Arg-, or Lys-; specific chemical cleavage by cyanogen bromide, hydroxylamine, BNPS-Skatole, acid, or alkali hydrolysis; enzymatic cleavage by trypsin, chymotrypsin, papain, V8 protease, NaBH₄; acetylation, formylation, oxidation, reduction; metabolic synthesis in the presence of tunicamycin; etc.

### 6.3 Compositions Comprising Antigenic Polypeptides

In another aspect, the instant disclosure provides a composition (e.g., a pharmaceutical composition, a vaccine, or a unit dosage form thereof) comprising one or more antigenic polypeptide as disclosed herein. In certain embodiments, the composition comprises a plurality of the antigenic polypeptides disclosed herein. For example, in certain embodiments, the composition comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 different antigenic polypeptides as disclosed herein.

6.3.1 Compositions Comprising Antigenic Polypeptides in Complex with Stress Proteins

In certain embodiments, the instant disclosure provides a composition (e.g., a pharmaceutical composition) comprising one or more antigenic polypeptides as disclosed herein and a purified stress protein. In certain embodiments, at least a portion of the purified stress protein binds to the antigenic polypeptide in the composition. Such compositions are useful as vaccines for the treatment of a cancer.

Stress proteins, which are also referred to interchangeably herein as heat shock proteins (HSPs), useful in the practice of the instant invention can be selected from among any cellular protein that is capable of binding other proteins or peptides and capable of releasing the bound proteins or peptides in the presence of adenosine triphosphate (ATP) or under acidic conditions. The intracellular concentration of such protein may increase when a cell is exposed to a stressful stimulus. In addition to those heat shock proteins that are induced by stress, the HSP60, HSP70, HSP90, HSP100, sHSPs, and PDI families also include proteins that are related to stress-induced HSPs in sequence similarity, for example, having greater than 35% amino acid identity, but whose expression levels are not altered by stress. Therefore, stress protein or heat shock protein embraces other proteins, mutants, analogs, and variants thereof having at least 35% (e.g., at least 40, 45, 50, 55, 60, 65, 70, 75, 80, 85, 90, 95, or 99%) amino acid identity with members of these families whose expression levels in a cell are enhanced in response to a stressful stimulus. Accordingly, in certain embodiments, the stress protein is a member of the hsp60, hsp70, or hsp90 family of stress proteins (e.g., Hsc70, human Hsc70), or a mutant, analog, or variant thereof. In certain embodiments, the stress protein is selected from the group consisting of hsc70, hsp70, hsp90, hsp110, grp170, gp96, calreticulin, a mutant thereof, and combinations of two or more thereof. In certain embodiments, the stress protein is Hsc70 (e.g., human Hsc70). In certain embodiments, the stress protein comprises the amino acid sequence of SEQ ID NO: 8807. In certain embodiments, the amino acid sequence of the stress protein consists of the amino acid sequence of SEQ ID NO: 8807. In certain embodiments, the stress protein is Hsp70 (e.g., human Hsp70). In certain embodiments, the stress protein (e.g., human hsc70) is a recombinant protein.

Amino acid sequences and nucleotide sequences of naturally occurring HSPs are generally available in sequence databases, such as GenBank. For example, Homo sapiens heat shock protein HSP70 (Heat Shock 70 kDa Protein 1A) has the following identifiers HGNC: 5232; Entrez Gene: 3303; Ensembl: ENSG00000204389; OMIM: 140550; UniProtKB: P08107 and NCBI Reference Sequence: NM_005345.5. Computer programs, such as Entrez, can be used to browse the database, and retrieve any amino acid sequence and genetic sequence data of interest by accession number. These databases can also be searched to identify sequences with various degrees of similarities to a query sequence using programs, such as FASTA and BLAST, which rank the similar sequences by alignment scores and statistics. Nucleotide sequences of non-limiting examples of HSPs that can be used for preparation of the HSP peptide-binding fragments disclosed herein are as follows: human Hsp70, Genbank Accession No. NM_005345, Sargent et al., 1989, Proc. Natl. Acad. Sci. U.S.A., 86:1968-1972; human Hsc70: Genbank Accession Nos. P11142, Y00371; human Hsp90, Genbank Accession No. X15183, Yamazaki et al., Nucl. Acids Res. 17:7108; human gp96: Genbank Accession No. X15187, Maki et al., 1990, Proc. Natl. Acad Sci., 87: 5658-5562; human BiP: Genbank Accession No. M19645; Ting et al., 1988, DNA 7: 275-286; human Hsp27, Genbank Accession No. M24743; Hickey et al., 1986, Nucleic Acids Res. 14:4127-45; mouse Hsp70: Genbank Accession No. M35021, Hunt et al., 1990, Gene, 87:199-204; mouse gp96: Genbank Accession No. M16370, Srivastava et al., 1987, Proc. Natl. Acad. Sci., 85:3807-3811; and mouse BiP: Genbank Accession No. U16277, Haas et al., 1988, Proc. Natl. Acad. Sci. U.S.A., 85: 2250-2254 (each of these references is incorporated herein by reference in its entirety).

In addition to the major stress protein families described above, an endoplasmic reticulum resident protein, calreticulin, has also been identified as yet another heat shock protein useful for eliciting an immune response when complexed to antigenic molecules (Basu and Srivastava, 1999, J. Exp. Med. 189:797-202; incorporated herein by reference in its entirety). Other stress proteins that can be used in the invention include grp78 (or BiP), protein disulfide isomerase (PDI), hsp110, and grp170 (Lin et al., 1993, Mol. Biol. Cell, 4:1109-1119; Wang et al., 2001, J. Immunol., 165:490-497, each of which is incorporated herein by reference in its entirety). Many members of these families were found subsequently to be induced in response to other stressful stimuli including nutrient deprivation, metabolic disruption, oxygen radicals, hypoxia and infection with intracellular pathogens (see Welch, May 1993, Scientific American 56-64; Young, 1990, Annu. Rev. Immunol. 8:401-420; Craig, 1993, Science 260:1902-1903; Gething, et al., 1992, Nature 355:33-45; and Lindquist, et al., 1988, Annu. Rev. Genetics 22:631-677, each of which is incorporated herein by reference in its entirety). It is contemplated that HSPs/stress proteins belonging to all of these families can be used in the practice disclosed herein. In certain embodiments, a stress protein encompasses any chaperone protein that facilitates peptide-WIC presentation. Suitable chaperone proteins include, but are not limited to, ER chaperones and tapasin (e.g., human tapasin).

The major stress proteins can accumulate to very high levels in stressed cells, but they occur at low to moderate levels in cells that have not been stressed. For example, the highly inducible mammalian hsp70 is hardly detectable at normal temperatures but becomes one of the most actively synthesized proteins in the cell upon heat shock (Welch, et al., 1985, J. Cell. Biol. 101:1198-1211, incorporated herein by reference in its entirety). In contrast, hsp90 and hsp60 proteins are abundant at normal temperatures in most, but not all, mammalian cells and are further induced by heat (Lai, et al., 1984, Mol. Cell. Biol. 4:2802-10; van Bergen en Henegouwen, et al., 1987, Genes Dev. 1:525-31, each of which is incorporated herein by reference in its entirety).

In various embodiments, nucleotide sequences encoding heat shock protein within a family or variants of a heat shock protein can be identified and obtained by hybridization with a probe comprising nucleotide sequence encoding an HSP under conditions of low to medium stringency. By way of example, procedures using such conditions of low stringency are as follows (see also Shilo and Weinberg, 1981, Proc. Natl. Acad. Sci. USA 78:6789-6792). Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for signal detection. If necessary, filters are washed for a third time at 65-68° C. before signal detection. Other conditions of low stringency which may be used are well known in the art (e.g., as used for cross-species hybridizations).

Where stress proteins are used, peptide-binding fragments of stress proteins and functionally active derivatives, analogs, and variants thereof can also be used. Accordingly, in certain embodiments, the stress protein is a full-length HSP. In certain embodiments, the stress protein is a polypeptide comprising a domain of an HSP (e.g., a member of the Hsp60, Hsp70, or Hsp90 family, such as Hsc70, particularly human Hsc70), wherein the domain is capable of being noncovalently associated with a peptide (e.g., an HSP-binding peptide as described herein) to form a complex and optionally eliciting an immune response, and wherein the stress protein is not a full-length HSP.

In certain embodiments, the stress protein is a polypeptide that is capable of being noncovalently associated with a peptide (e.g., an HSP-binding peptide as described herein) to form a complex and optionally eliciting an immune response, wherein the stress protein shares a high degree of sequence similarity with a wild-type HSP (e.g., a member of the Hsp60, Hsp70, or Hsp90 family, such as Hsc70, particularly human Hsc70). To determine a region of identity between two amino acid sequences or nucleic acid sequences, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877 (each of which is incorporated herein by reference in its entirety). Such an algorithm is incorporated into the NBLAST and XBLAST programs of Altschul, et al., 1990, J. Mol. Biol. 215:403-410 (incorporated herein by reference in its entirety). BLAST nucleotide searches can be performed with the NBLAST program, e.g., score=100, wordlength=12 to obtain nucleotide sequences homologous to a nucleic acid molecule disclosed herein. BLAST protein searches can be performed with the XBLAST program, e.g., score=50, wordlength=3 to obtain amino acid sequences homologous to a protein molecule disclosed herein. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Altschul et al., 1997, supra). When utilizing BLAST, Gapped BLAST, and PSI-Blast programs, the default parameters of the respective programs (e.g., XBLAST and NBLAST) can be used. Another example of a mathematical algorithm utilized for the comparison of sequences is the algorithm of Myers and Miller, 1988, CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In certain embodiments, isolated peptide-binding domains of a stress protein (e.g., Hsp70 or Hsc70) are employed. These peptide-binding domains can be identified by computer modeling of the three-dimensional structure of the peptide-binding site of a stress protein (e.g., Hsp70 and Hsc70). See for example, the peptide-binding fragments of HSPs disclosed in United States patent publication US 2001/0034042 (incorporated herein by reference in its entirety).

In certain embodiments, the stress protein is a mutated stress protein which has an affinity for a target polypeptide that is greater than a native stress protein. Such mutated stress proteins can be useful when the target polypeptide is phosphorylated or is a phosphopeptide mimetic (such as non-hydrolyzable analogs) or has some other post-translational modification.

The stress proteins can be prepared by purification from tissues, or by recombinant DNA techniques. HSPs can be purified from tissues in the presence of ATP or under acidic conditions (pH 1 to pH 6.9), for subsequent in vitro complexing to one or more polypeptides. See Peng, et al., 1997, J. Immunol. Methods, 204:13-21; Li and Srivastava, 1993, EMBO J. 12:3143-3151 (each of these references is incorporated herein by reference in its entirety). “Purified” stress proteins are substantially free of materials that are associated with the proteins in a cell, in a cell extract, in a cell culture medium, or in an individual. In certain embodiments, the stress protein purified from a tissue is a mixture of different HSPs, for example, hsp70 and hsc70.

Using the defined amino acid or cDNA sequences of a given HSP or a peptide-binding domain thereof, one can make a genetic construct which is transfected into and expressed in a host cell. The recombinant host cells may contain one or more copies of a nucleic acid sequence comprising a sequence that encodes an HSP or a peptide-binding fragment, operably linked with regulatory region(s) that drives the expression of the HSP nucleic acid sequence in the host cell. Recombinant DNA techniques can be readily utilized to generate recombinant HSP genes or fragments of HSP genes, and standard techniques can be used to express such HSP gene fragments. Any nucleic acid sequence encoding an HSP peptide-binding domain, including cDNA and genomic DNA, can be used to prepare the HSPs or peptide-binding fragments disclosed herein. The nucleic acid sequence can be wild-type or a codon-optimized variant that encodes the same amino acid sequence. An HSP gene fragment containing the peptide-binding domain can be inserted into an appropriate cloning vector and introduced into host cells so that many copies of the gene sequence are generated. A large number of vector-host systems known in the art may be used such as, but not limited to, bacteriophages such as lambda derivatives, or plasmids such as pBR322, pUC plasmid derivatives, the Bluescript vectors (Stratagene) or the pET series of vectors (Novagen). Any technique for mutagenesis known in the art can be used to modify individual nucleotides in a DNA sequence, for purpose of making amino acid substitution(s) in the expressed peptide sequence, or for creating/deleting restriction sites to facilitate further manipulations.

The stress proteins may be expressed as fusion proteins to facilitate recovery and purification from the cells in which they are expressed. For example, the stress proteins may contain a signal sequence leader peptide to direct its translocation across the endoplasmic reticulum membrane for secretion into culture medium. Further, the stress protein may contain an affinity label fused to any portion of the protein not involved in binding to a target polypeptide, for example, the carboxyl terminus. The affinity label can be used to facilitate purification of the protein, by binding to an affinity partner molecule. A variety of affinity labels known in the art may be used, non-limiting examples of which include the immunoglobulin constant regions, polyhistidine sequence (Petty, 1996, Metal-chelate affinity chromatography, in Current Protocols in Molecular Biology, Vol. 2, Ed. Ausubel et al., Greene Publish. Assoc. & Wiley Interscience, incorporated herein by reference in its entirety), glutathione S-transferase (GST; Smith, 1993, Methods Mol. Cell Bio. 4:220-229, incorporated herein by reference in its entirety), the E. coli maltose binding protein (Guan et al., 1987, Gene 67:21-30, incorporated herein by reference in its entirety), and various cellulose binding domains (U.S. Pat. Nos. 5,496,934; 5,202,247; 5,137,819; Tomme et al., 1994, Protein Eng. 7:117-123, each of which is incorporated herein by reference in its entirety).

Such recombinant stress proteins can be assayed for peptide binding activity (see, e.g., Klappa et al., 1998, EMBO J., 17:927-935, incorporated herein by reference in its entirety) for their ability to elicit an immune response. In certain embodiments, the recombinant stress protein produced in the host cell is of the same species as the intended recipient of the immunogenic composition (e.g., human).

The stress protein may be bound to the polypeptide(s) non-covalently or covalently. In certain embodiments, the stress protein is non-covalently bound to the polypeptide. Methods of preparing such complexes are set forth infra.

The molar ratio of total polypeptide(s) to total stress protein(s) can be any ratio from about 0.01:1 to about 100:1, including but not limited to about 0.01:1, 0.02:1, 0.05:1. 0.1:1. 0.2:1, 0.5:1, 1:1, 1.5:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 49:1, up to 100:1. In certain embodiments, the composition comprises a plurality of complexes each comprising a polypeptide disclosed herein and a stress protein, wherein the molar ratio of the polypeptide to the stress protein in each complex is at least about 1:1 (e.g., about 1.5:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 49:1, up to 100:1).

In certain embodiments, the molar ratio of total polypeptide(s) to total stress protein(s) is about 0.5:1 to 5:1. In certain embodiments, the molar ratio of total polypeptide(s) to total stress protein(s) is about 1:1 to 2:1. In certain embodiments, the molar ratio of total polypeptide(s) to total stress protein(s) is about 1:1, 1.25:1, 1.5:1, 2:1, 2.5:1, 3:1, 3.5:1, 4:1, 4.5:1, or 5:1. Such ratios, particularly the ratios close to 1:1, are advantageous in that the composition does not comprise a great excess of free peptide(s) that is not bound to a stress protein. Since many antigenic peptides comprising WIC-binding peptides tend to comprise hydrophobic regions, an excess amount of free peptide(s) may tend to aggregate during preparation and storage of the composition. Substantial complexation with a stress protein at a molar ratio of total polypeptide(s) to total stress protein(s) close to 1:1 (e.g., 1:1, 1.25:1, 1.5:1, or 2:1) is enabled by a high binding affinity of the polypeptide to the stress protein. Accordingly, in certain embodiments, the polypeptide binds to an HSP (e.g., Hsc70, Hsp70, Hsp90, Hsp110, Grp170, Gp96, or Calreticulin) with a K_{d} lower than 10⁻³ M, 10⁻⁴ M, 10⁻⁵ M, 10⁻⁶ M, 10⁻⁷ M, 10⁻⁸ M, or 10⁻⁹ M. In certain embodiments, the polypeptide binds to Hsc70 (e.g., human Hsc70) with a K_{d} of 10⁻³ M, 10⁻⁴ M, 10⁻⁵ M, 10⁻⁶ M, 10⁻⁷ M, 10⁻⁸M, 10⁻⁹M, or lower.

In certain embodiments, at least 40%, 45%, 50%, 55%, 60%, 65%, 70%, 75%, 80%, 85%, 90%, or 95% of the stress protein binds to the polypeptide in the composition. In certain embodiments, substantially all of the stress protein binds to the polypeptide in the composition.

Any number of different polypeptides can be included in a single composition as disclosed herein. In certain embodiments, the compositions comprise no more than 100 different polypeptides, e.g., 2-50, 2-30, 2-20, 5-20, 5-15, 5-10, or 10-15 different polypeptides.

In certain embodiments, each of the antigenic polypeptides comprises the same HSP-binding peptide and a different antigenic peptide. In certain embodiments, the composition comprises a single stress protein, wherein the stress protein is capable of binding to the HSP-binding peptide.

Pharmaceutical compositions comprising the complexes of stress proteins and antigenic polypeptides disclosed herein can be formulated to contain one or more pharmaceutically acceptable carriers or excipients including bulking agents, stabilizing agents, buffering agents, sodium chloride, calcium salts, surfactants, antioxidants, chelating agents, other excipients, and combinations thereof.

Bulking agents are preferred in the preparation of lyophilized formulations of the composition. Such bulking agents form the crystalline portion of the lyophilized product and may be selected from the group consisting of mannitol, glycine, alanine, and hydroxyethyl starch (HES).

Stabilizing agents may be selected from the group consisting of sucrose, trehalose, raffinose, and arginine. These agents are preferably present in amounts between 1-4%. Sodium chloride can be included in the present formulations preferably in an amount of 100-300 mM, or if used without the aforementioned bulking agents, can be included in the formulations in an amount of between 300-500 mM NaCl. Calcium salts include calcium chloride, calcium gluconate, calcium glubionate, or calcium gluceptate.

Buffering agents can be any physiologically acceptable chemical entity or combination of chemical entities which have a capacity to act as buffers, including but not limited to histidine, potassium phosphate, TRIS [tris-(hydroxymethyl)-aminomethane], BIS-Tris Propane (1,3-bis-[tris-(hydroxymethyl)methylamino]-propane), PIPES [piperazine-N,N′-bis-(2-ethanesulfonic acid)], MOPS [3-(N-morpholino)ethanesulfonic acid], HEPES (N-2-hydroxyethyl-piperazine-N′-2-ethanesulfonic acid), IVIES [2-(N-morpholino)ethanesulfonic acid], and ACES (N-2-acetamido-2-aminoethanesulfonic acid). Typically, the buffering agent is included in a concentration of 10-50 mM. Specific examples of base buffers include (i) PBS; (ii) 10 mM KPO₄, 150 mM NaCl; (iii) 10 mM HEPES, 150 mM NaCl; (iv) 10 mM imidazole, 150 mM NaCl; and (v) 20 mM sodium citrate. Excipients that can be used include (i) glycerol (10%, 20%); (ii) Tween 50 (0.05%, 0.005%); (iii) 9% sucrose; (iv) 20% sorbitol; (v) 10 mM lysine; or (vi) 0.01 mM dextran sulfate.

Surfactants, if present, are preferably in a concentration of 0.1% or less, and may be chosen from the group including but not limited to polysorbate 20, polysorbate 80, pluronic polyols, and BRIJ 35 (polyoxyethylene 23 laurel ether). Antioxidants, if used, must be compatible for use with a pharmaceutical preparation, and are preferably water soluble. Suitable antioxidants include homocysteine, glutathione, lipoic acid, 6-hydroxy-2,5,7,8-tetramethylchroman-2-carboxylic acid (Trolox), methionine, sodium thiosulfate, platinum, glycine-glycine-histidine (tripeptide), and butylatedhydroxytoluene (BHT). Chelating agents should preferably bind metals such as copper and iron with greater affinity than calcium, if a calcium salt is being used in the composition. An exemplary chelator is deferoxamine.

Many formulations known in the art can be used. For example, U.S. Pat. No. 5,763,401 describes a therapeutic formulation, comprising 15-60 mM sucrose, up to 50 mM NaCl, up to 5 mM calcium chloride, 65-400 mM glycine, and up to 50 mM histidine. In some embodiments, the therapeutic formulation is a solution of 9% sucrose in potassium phosphate buffer.

U.S. Pat. No. 5,733,873 (incorporated herein by reference in its entirety) discloses formulations which include between 0.01-1 mg/ml of a surfactant. This patent discloses formulations having the following ranges of excipients: polysorbate 20 or 80 in an amount of at least 0.01 mg/ml, preferably 0.02-1.0 mg/ml; at least 0.1 M NaCl; at least 0.5 mM calcium salt; and at least 1 mM histidine. More particularly, the following specific formulations are also disclosed: (1) 14.7-50-65 mM histidine, 0.31-0.6 M NaCl, 4 mM calcium chloride, 0.001-0.02-0.025% polysorbate 80, with or without 0.1% PEG 4000 or 19.9 mM sucrose; and (2) 20 mg/ml mannitol, 2.67 mg/ml histidine, 18 mg/ml NaCl, 3.7 mM calcium chloride, and 0.23 mg/ml polysorbate 80.

The use of low or high concentrations of sodium chloride has been described, for example U.S. Pat. No. 4,877,608 (incorporated herein by reference in its entirety) teaches formulations with relatively low concentrations of sodium chloride, such as formulations comprising 0.5 mM-15 mM NaCl, 5 mM calcium chloride, 0.2 mM-5 mM histidine, 0.01-10 mM lysine hydrochloride and up to 10% maltose, 10% sucrose, or 5% mannitol.

U.S. Pat. No. 5,605,884 (incorporated herein by reference in its entirety) teaches the use of formulations with relatively high concentrations of sodium chloride. These formulations include 0.35 M-1.2 M NaCl, 1.5-40 mM calcium chloride, 1 mM-50 mM histidine, and up to 10% sugar such as mannitol, sucrose, or maltose. A formulation comprising 0.45 M NaCl, 2.3 mM calcium chloride, and 1.4 mM histidine is exemplified.

International Patent Application WO 96/22107 (incorporated herein by reference in its entirety) describes formulations which include the sugar trehalose, for example formulations comprising: (1) 0.1 M NaCl, 15 mM calcium chloride, 15 mM histidine, and 1.27 M (48%) trehalose; or (2) 0.011% calcium chloride, 0.12% histidine, 0.002% TRIS, 0.002% Tween 80, 0.004% PEG 3350, 7.5% trehalose; and either 0.13% or 1.03% NaCl.

U.S. Pat. No. 5,328,694 (incorporated herein by reference in its entirety) describes a formulation which includes 100-650 mM disaccharide and 100 mM-1.0 M amino acid, for example (1) 0.9 M sucrose, 0.25 M glycine, 0.25 M lysine, and 3 mM calcium chloride; and (2) 0.7 M sucrose, 0.5 M glycine, and 5 mM calcium chloride. Pharmaceutical compositions can be optionally prepared as lyophilized product, which may then be formulated for oral administration or reconstituted to a liquid form for parenteral administration.

In certain embodiments, the composition stimulates a T-cell response against a cell expressing or displaying a polypeptide comprising one or more of the WIC-binding peptides in a subject to whom the composition is administered. The cell expressing the polypeptide may be a cell comprising a polynucleotide encoding the polypeptide, wherein the polynucleotide is in the genome of the cell, in an episomal vector, or in the genome of a virus that has infected the cell. The cell displaying the polypeptide may not comprise a polynucleotide encoding the polypeptide, and may be produced by contacting the cell with the polypeptide or a derivative thereof.

In certain embodiments, the composition induces in vitro activation of T cells in peripheral blood mononuclear cells (PBMCs) isolated from a subject. The in vitro activation of T cells includes, without limitation, in vitro proliferation of T cells, production of cytokines (e.g., IFNγ) from T cells, and increased surface expression of activation markers (e.g., CD25, CD45RO) on T cells.

### 6.3.2 Preparation of Complexes of Antigenic Polypeptides and Stress Proteins

In another aspect, the instant disclosure provides a method of making complexes of antigenic polypeptides and stress proteins (e.g., for the purposes of making a vaccine), the method comprising mixing one or more antigenic polypeptides as disclosed herein with a purified stress protein in vitro under suitable conditions such that the purified stress protein binds to at least one of the antigenic polypeptides. The method is also referred to as a complexing reaction herein. In certain embodiments, two or more purified stress proteins are employed, wherein each purified stress protein binds to at least one of the antigenic polypeptides. In certain embodiments, at least a portion of the purified stress protein binds to the antigenic polypeptide in the composition.

The stress protein may be bound to the polypeptide non-covalently or covalently. In certain embodiments, the stress protein is non-covalently bound to the polypeptide. In various embodiments, the complexes formed in vitro are optionally purified. Purified complexes of stress proteins and polypeptides are substantially free of materials that are associated with such complexes in a cell, or in a cell extract. Where purified stress proteins and purified polypeptides are used in an in vitro complexing reaction, the term “purified complex(es)” does not exclude a composition that also comprises free stress proteins and conjugates or peptides not in complexes.

Any stress proteins described supra may be employed in the method disclosed herein. In certain embodiments, the stress protein is selected from the group consisting of Hsc70, Hsp70, Hsp90, Hsp110, Grp170, Gp96, Calreticulin, a mutant thereof, and combinations of two or more thereof. In one embodiment, the stress protein is an Hsc70, e.g., a human Hsc70. In another embodiment, the stress protein is an Hsp70, e.g., a human Hsp70. In certain embodiments, the stress protein (e.g., human Hsc70 or human Hsp70) is a recombinant protein.

Prior to complexing, HSPs can be pretreated with ATP or exposed to acidic conditions to remove any peptides that may be non-covalently associated with the HSP of interest. Acidic conditions are any pH levels below pH 7, including the ranges pH 1-pH 2, pH 2-pH 3, pH 3-pH 4, pH 4-pH 5, pH 5-pH 6, and pH 6-pH 6.9. When the ATP procedure is used, excess ATP is removed from the preparation by the addition of apyranase as described by Levy, et al., 1991, Cell 67:265-274 (incorporated herein by reference in its entirety). When acidic conditions are used, the buffer is readjusted to neutral pH by the addition of pH modifying reagents.

In certain embodiments, prior to complexation with purified stress proteins, the polypeptides may be reconstituted from powder in 100% DMSO. Equimolar amounts of the peptides may then be pooled in a solution of 75% DMSO diluted in sterile water.

In certain embodiments, prior to complexation with purified stress proteins, the polypeptides may be reconstituted in neutral water.

In certain embodiments, prior to complexation with purified stress proteins, the polypeptides may be reconstituted in acidic water containing HCl or another acid.

In certain embodiments, prior to complexation with purified stress proteins, the polypeptides may be reconstituted in basic water containing NaOH, or NH₄OH, or another base.

In certain embodiments, prior to complexation with purified stress proteins, the solubility of each polypeptide in water may be tested. If a polypeptide is soluble in neutral water, neutral water may be used as a solvent for the polypeptide. If the polypeptide is not soluble in neutral water, solubility in acidic water containing HCl, or another acid, e.g., acetic acid, phosphoric acid, or sulfuric acid may be tested. If the polypeptide is soluble in acidic water containing HCl (or another acid), acidic water containing HCl (or another acid) may be used as the solvent for the polypeptide. If the polypeptide is not soluble in acidic water containing HCl (or another acid), solubility in basic water containing NaOH may be tested. If the polypeptide is soluble in basic water containing NaOH, basic water containing NaOH may be used as the solvent for the polypeptide. If the polypeptide is not soluble in basic water containing NaOH, the polypeptide may be dissolved in DMSO. If the polypeptide is not soluble in DMSO the polypeptide may be excluded. The dissolved polypeptides may then be mixed to make a pool of polypeptides. The dissolved polypeptides may be mixed at equal volume. The dissolved polypeptides may be mixed in equimolar amounts.

The molar ratio of total polypeptide(s) to total stress protein(s) can be any ratio from 0.01:1 to 100:1, including but not limited to 0.01:1, 0.02:1, 0.05:1. 0.1:1. 0.2:1, 0.5:1, 1:1, 1.5:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 49:1, up to 100:1. In certain embodiments, the composition to be prepared comprises a plurality of complexes each comprising a polypeptide disclosed herein and a stress protein, and the complexing reaction comprises mixing the polypeptides with the stress proteins, wherein the molar ratio of the polypeptide to the stress protein in each complex is at least 1:1 (e.g., about 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 49:1, up to 100:1).

In certain embodiments, the molar ratio of total polypeptide(s) to total stress protein(s) is about 0.5:1 to 5:1. In certain embodiments, the molar ratio of total polypeptide(s) to total stress protein(s) is about 1:1, 1.25:1, 1.5:1, 2:1, 2.5:1, 3:1, 3.5:1, 4:1, 4.5:1, or 5:1. In certain embodiments, the molar ratio of total polypeptide(s) to total stress protein(s) is about 1:1, 1.25:1, or 1.5:1. Such ratios, particularly the ratios close to 1:1, are advantageous in that the composition does not comprise a great excess of free peptide(s) that is not bound to a stress protein. Since many antigenic peptides comprising WIC-binding peptides tend to comprise hydrophobic regions, an excess amount of free peptide(s) may tend to aggregate during preparation and storage of the composition. Substantial complexation with a stress protein at a molar ratio of total polypeptide(s) to total stress protein(s) close to 1:1 (e.g., 1:1, 1.25:1, 1.5:1, or 2:1) is enabled by a high binding affinity of the polypeptide to the stress protein. Accordingly, in certain embodiments, the polypeptide used in the complexing reaction binds to an HSP (e.g., Hsc70, Hsp70, Hsp90, Hsp110, Grp170, Gp96, or Calreticulin) with a K_{d} lower than 10⁻³ M, 10⁻⁴ M, 10⁻⁵ M, 10⁻⁶ M, 10⁻⁷ M, 10⁻⁸ M, or 10⁻⁹ M. In certain embodiments, the polypeptide binds to Hsc70 (e.g., human Hsc70) with a K_{d} of 10⁻³ M, 10⁻⁴ M, 10⁻⁵ M, 10⁻⁶ M, 10⁻⁷ M, 10⁻⁸ M, 10⁻⁹ M, or lower.

The method disclosed herein can be used to prepare a composition (e.g., a pharmaceutical composition) in bulk (e.g., greater than or equal to 30 mg, 50 mg, 100 mg, 200 mg, 300 mg, 500 mg, or 1 g of total peptide and protein). The prepared composition can then be transferred to single-use or multi-use containers, or apportioned to unit dosage forms. Alternatively, the method disclosed herein can be used to prepare a composition (e.g., a pharmaceutical composition) in a small amount (e.g., less than or equal to 300 μg, 1 mg, 3 mg, 10 mg, 30 mg, or 100 mg of total peptide and protein). In certain embodiments, the composition is prepared for single use, optionally in a unit dosage form.

In certain embodiments, the total amount of the polypeptide(s) and stress protein in the composition is about 10 μg to 600 μg (e.g., about 50 μg, 100 μg, 200 μg, 300 μg, 400 μg, or 500 μg, optionally about 120 μg, 240 μg, or 480 μg). In certain embodiments, the total amount of the polypeptide(s) and stress protein in the composition is about 300 μg. Amounts of the stress protein(s) and polypeptide(s) in a unit dosage form are disclosed infra.

An exemplary protocol for noncovalent complexing of a population of polypeptides to a stress protein in vitro is provided herein. The population of polypeptides can comprise a mixture of the different polypeptide species disclosed herein. Then, the mixture is incubated with the purified and/or pretreated stress protein for from 15 minutes to 3 hours (e.g., 1 hour) at from 4° to 50° C. (e.g., 37° C.) in a suitable binding buffer, such as phosphate buffered saline pH 7.4 optionally supplemented with 0.01% Polysorbate 20; a buffer comprising 9% sucrose in potassium phosphate buffer; a buffer comprising 2.7 mM Sodium Phosphate Dibasic, 1.5 mM Potassium Phosphate Monobasic, 150 mM NaCl, pH 7.2; a buffer containing 20 mM sodium phosphate, pH 7.2-7.5, 350-500 mM NaCl, 3 mM MgCl₂ and 1 mM phenyl methyl sulfonyl fluoride (PMSF); and the buffer optionally comprising 1 mM ADP. Any buffer may be used that is compatible with the stress protein. The preparations are then optionally purified by centrifugation through a Centricon 10 assembly (Millipore; Billerica, Mass.) to remove any unbound peptide. The non-covalent association of the proteins/peptides with the HSPs can be assayed by High Performance Liquid Chromatography (HPLC), Mass Spectrometry (MS), mixed lymphocyte target cell assay (MLTC), or enzyme-linked immunospot (ELISPOT) assay (Taguchi T, et al., J Immunol Methods 1990; 128: 65-73, incorporated herein by reference in its entirety). Once the complexes have been isolated and diluted, they can be optionally characterized further in animal models using the administration protocols and excipients described herein (see, e.g., Example 2 infra).

Complexes of stress proteins and antigenic polypeptides from separate covalent and/or non-covalent complexing reactions can be prepared to form a composition before administration to a subject. In certain embodiments, the composition is prepared within 1, 2, 3, 4, 5, 6, or 7 days before administration to a subject. In certain embodiments, the composition is prepared within 1, 2, 3, 4, 5, 6, 7, or 8 weeks before administration to a subject. In certain embodiments, the composition is prepared within 1, 2, 3, 4, 5, 6, 7, 8, 9, 10, 11, or 12 months before administration to a subject. The composition can optionally be stored at about 4° C., −20° C., or −80° C. after preparation and before use.

In certain embodiments, the complexes prepared by the method disclosed herein are mixed with an adjuvant at bedside just prior to administration to a patient. In certain embodiments, the adjuvant comprises a saponin or an immunostimulatory nucleic acid. In certain embodiments, the adjuvant comprises QS-21. In certain embodiments, the dose of QS-21 is 10 μg, 25 μg, 50 μg, 75 μg, 100 μg, 125 μg, 150 μg, 175 μg, or 200 μg. In certain embodiments, the dose of QS-21 is about 100 μg. In certain embodiments, the adjuvant comprises a TLR agonist. In certain embodiments, the TLR agonist is an agonist of TLR4. In certain embodiments, the TLR agonist is an agonist of TLR7 and/or TLR8. In certain embodiments, the TLR agonist is an agonist of TLR9. In certain embodiments, the TLR agonist is an agonist of TLR5.

As an alternative to making non-covalent complexes of stress proteins and polypeptides, the polypeptides can be covalently attached to stress proteins, e.g., by chemical crosslinking or UV crosslinking. Any chemical crosslinking or UV crosslinking methods known in the art (see, e.g., Wong, 1991, Chemistry of Protein Conjugation and Cross-Linking, CRC Press, incorporated herein by reference in its entirety) can be employed. For example, glutaraldehyde crosslinking (see, e.g., Barrios et al., 1992, Eur. J. Immunol. 22: 1365-1372, incorporated herein by reference in its entirety) may be used. In an exemplary protocol, 1-2 mg of HSP-peptide complex is cross-linked in the presence of 0.002% glutaraldehyde for 2 hours. Glutaraldehyde is removed by dialysis against phosphate buffered saline (PBS) overnight (Lussow et al., 1991, Eur. J. Immunol. 21: 2297-2302, incorporated herein by reference in its entirety).

### 6.3.3 Vaccines

In another aspect, the instant disclosure provides a vaccine comprising the polypeptide compositions (e.g., antigenic polypeptide compositions) disclosed herein. The vaccine may be prepared by any method that results in a stable, sterile, preferably injectable formulation.

In certain embodiments, the vaccine comprises one or more compositions disclosed herein and one or more adjuvants. A variety of adjuvants may be employed, including, for example, systemic adjuvants and mucosal adjuvants. A systemic adjuvant is an adjuvant that can be delivered parenterally. Systemic adjuvants include adjuvants that create a depot effect, adjuvants that stimulate the immune system, and adjuvants that do both.

An adjuvant that creates a depot effect is an adjuvant that causes the antigen to be slowly released in the body, thus prolonging the exposure of immune cells to the antigen. This class of adjuvants includes alum (e.g., aluminum hydroxide, aluminum phosphate); or emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720, AirLiquide, Paris, France); MF-59 (a squalene-in-water emulsion stabilized with Span 85 and Tween 80; Chiron Corporation, Emeryville, Calif.; and PROVAX (an oil-in-water emulsion containing a stabilizing detergent and a micelle-forming agent; IDEC, Pharmaceuticals Corporation, San Diego, Calif.).

Other adjuvants stimulate the immune system, for instance, cause an immune cell to produce and secrete cytokines or IgG. This class of adjuvants includes immunostimulatory nucleic acids, such as CpG oligonucleotides; saponins purified from the bark of the Q. saponaria tree, such as QS-21; poly[di(carboxylatophenoxy)phosphazene (PCPP polymer; Virus Research Institute, USA); RNA mimetics such as polyinosinic:polycytidylic acid (poly I:C) or poly I:C stabilized with poly-lysine (poly-ICLC [Hiltonol®; Oncovir, Inc.]; derivatives of lipopolysaccharides (LPS) such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.).

Other systemic adjuvants are adjuvants that create a depot effect and stimulate the immune system. These compounds have both of the above-identified functions of systemic adjuvants. This class of adjuvants includes but is not limited to ISCOMs (Immunostimulating complexes which contain mixed saponins, lipids and form virus-sized particles with pores that can hold antigen; CSL, Melbourne, Australia); AS01 which is a liposome based formulation containing MPL and QS-21 (GlaxoSmithKline, Belgium); AS02 (GlaxoSmithKline, which is an oil-in-water emulsion containing MPL and QS-21: GlaxoSmithKline, Rixensart, Belgium); AS04 (GlaxoSmithKline, which contains alum and MPL; GSK, Belgium); AS15 which is a liposome based formulation containing CpG oligonucleotides, MPL and QS-21 (GlaxoSmithKline, Belgium); non-ionic block copolymers that form micelles such as CRL 1005 (these contain a linear chain of hydrophobic polyoxypropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

The mucosal adjuvants useful according to the invention are adjuvants that are capable of inducing a mucosal immune response in a subject when administered to a mucosal surface in conjunction with complexes disclosed herein. Mucosal adjuvants include CpG nucleic acids (e.g. PCT published patent application WO 99/61056, incorporated herein by reference in its entirety), bacterial toxins: e.g., Cholera toxin (CT), CT derivatives including but not limited to CT B subunit (CTB); CTD53 (Val to Asp); CTK97 (Val to Lys); CTK104 (Tyr to Lys); CTD53/K63 (Val to Asp, Ser to Lys); CTH54 (Arg to His); CTN107 (His to Asn); CTE114 (Ser to Glu); CTE112K (Glu to Lys); CTS61F (Ser to Phe); CTS 106 (Pro to Lys); and CTK63 (Ser to Lys), Zonula occludens toxin (zot), Escherichia coli heat-labile enterotoxin, Labile Toxin (LT), LT derivatives including but not limited to LT B subunit (LTB); LT7K (Arg to Lys); LT61F (Ser to Phe); LT112K (Glu to Lys); LT118E (Gly to Glu); LT146E (Arg to Glu); LT192G (Arg to Gly); LTK63 (Ser to Lys); and LTR72 (Ala to Arg), Pertussis toxin, PT. including PT-9K/129G; Toxin derivatives (see below); Lipid A derivatives (e.g., monophosphoryl lipid A, MPL); Muramyl Dipeptide (MDP) derivatives; bacterial outer membrane proteins (e.g., outer surface protein A (OspA) lipoprotein of Borrelia burgdorferi, outer membrane protein of Neisseria meningitidis); oil-in-water emulsions (e.g., MF59; aluminum salts (Isaka et al., 1998, 1999); and Saponins (e.g., QS-21, e.g., QS-21 Stimulon®, Antigenics LLC, Lexington, Mass.), ISCOMs, MF-59 (a squalene-in-water emulsion stabilized with Span 85 and Tween 80; Chiron Corporation, Emeryville, Calif.); the Seppic ISA series of Montanide adjuvants (e.g., Montanide ISA 720; AirLiquide, Paris, France); PROVAX (an oil-in-water emulsion containing a stabilizing detergent and a micelle-forming agent; DEC Pharmaceuticals Corporation, San Diego, Calif.); Syntext Adjuvant Formulation (SAF; Syntex Chemicals, Inc., Boulder, Colo.); poly [di(carboxylatophenoxy)]phosphazene (PCPP polymer; Virus Research Institute, USA) and Leishmania elongation factor (Corixa Corporation, Seattle, Wash.).

In certain embodiments, the adjuvant added to the compositions disclosed herein comprises a saponin and/or an immunostimulatory nucleic acid. In certain embodiments, the adjuvant added to the composition comprises or further comprises QS-21.

In certain embodiments, the adjuvant added to the compositions disclosed herein comprises a Toll-like receptor (TLR) agonist. In certain embodiments, the TLR agonist is an agonist of TLR4. In certain embodiments, the TLR agonist is an agonist of TLR7 and/or TLR8. In certain embodiments, the TLR agonist is an agonist of TLR9. In certain embodiments, the TLR agonist is an agonist of TLR5.

The compositions disclosed herein described herein may be combined with an adjuvant in several ways. For example, different polypeptides may be mixed together first to form a mixture and then complexed with stress protein(s) and/or adjuvant(s) to form a composition. As another example, different polypeptides may be complexed individually with a stress protein and/or adjuvant(s), and the resulting batches of complexes may then be mixed to form a composition.

The adjuvant can be administered prior to, during, or following administration of the compositions comprising complexes of stress protein and polypeptides. Administration of the adjuvant and the compositions can be at the same or different administration sites.

### 6.3.4 Unit Dosage Forms

In another aspect, the instant disclosure provides a unit dosage form of a composition (e.g., pharmaceutical composition or vaccine) disclosed herein.

The amounts and concentrations of the polypeptides (e.g., antigenic polypeptides), stress proteins, and/or adjuvants at which the efficacy of a vaccine disclosed herein is effective may vary depending on the chemical nature and the potency of the polypeptides, stress proteins, and/or adjuvants. Typically, the starting amounts and concentrations in the vaccine are the ones conventionally used for eliciting the desired immune response, using the conventional routes of administration, e.g., intramuscular injection. The amounts and concentrations of the polypeptides (e.g., antigenic polypeptides), conjugates, stress proteins, and/or adjuvants can then be adjusted, e.g., by dilution using a diluent, so that an effective immune response is achieved as assessed using standard methods known in the art (e.g., determined by the antibody or T-cell response to the vaccine relative to a control formulation).

In certain embodiments, the total amount of the polypeptides and stress protein in the composition is about 10 μg to 600 μg (e.g., about 50 μg, 100 μg, 200 μg, 300 μg, 400 μg, or 500 μg, optionally about 120 μg, 240 μg, or 480 μg). In certain embodiments, the total amount of the polypeptides and stress protein in the composition is about 300 μg. In certain embodiments, the amount of the stress protein in the composition is about 250 μg to 290 μg.

In certain embodiments, the amount of the stress protein in the composition is about 10 μg to 600 μg (e.g., about 50 μg, 100 μg, 200 μg, 300 μg, 400 μg, or 500 μg, optionally about 120 μg, 240 μg, or 480 μg). In certain embodiments, the amount of the stress protein in the composition is about 300 μg. The amount of the polypeptide is calculated based on a designated molar ratio and the molecular weight of the polypeptides.

In certain embodiments, the total molar amount of the polypeptides in the unit dosage form of the composition is about 0.1 to 10 nmol (e.g., about 0.1 nmol, 0.5 nmol, 1 nmol, 2 nmol, 3 nmol, 4 nmol, 5 nmol, 6 nmol, 7 nmol, 8 nmol, 9 nmol, or 10 nmol). In certain embodiments, the total molar amount of the polypeptides in the unit dosage form of the composition is about 4 nmol. In certain embodiments, the total molar amount of the polypeptides in the unit dosage form of the composition is about 5 nmol.

The molar ratio of total polypeptides to total stress proteins can be any ratio from about 0.01:1 to about 100:1, including but not limited to about 0.01:1, 0.02:1, 0.05:1. 0.1:1. 0.2:1, 0.5:1, 1:1, 1.5:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 49:1, up to 100:1. In certain embodiments, the composition comprises a plurality of complexes each comprising a polypeptide and a stress protein, wherein the molar ratio of the polypeptide to the stress protein in each complex is at least about 1:1 (e.g., about 1.5:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 49:1, up to 100:1). In certain embodiments, the molar ratio of total polypeptide(s) to total stress protein(s) is about 0.5:1 to 5:1.

In certain embodiments, the molar ratio of total polypeptide(s) to total stress protein(s) is about 1:1 to 2:1. In certain embodiments, the molar ratio of total polypeptide(s) to total stress protein(s) is about 1:1, 1.25:1, or 1.5:1. Such ratios, particularly the ratios close to 1:1, are advantageous in that the composition does not comprise a great excess of free peptide(s) that is not bound to a stress protein. Since many antigenic peptides comprising WIC-binding peptides tend to comprise hydrophobic regions, an excess amount of free peptide(s) may tend to aggregate during preparation and storage of the composition. Substantial complexation with a stress protein at a molar ratio of total polypeptide(s) to total stress protein(s) close to 1:1 (e.g., 1:1, 1.25:1, 1.5:1, or 2:1) is enabled by a high binding affinity of the polypeptide to the stress protein. Accordingly, in certain embodiments, the polypeptide binds to an HSP (e.g., Hsc70, Hsp70, Hsp90, Hsp110, Grp170, Gp96, or Calreticulin) with a K_{d} lower than 10⁻³ M, 10⁻⁴ M, 10⁻⁵ M, 10⁻⁶ M, 10⁻⁷ M, 10⁻⁸ M, or 10⁻⁹ M. In certain embodiments, the polypeptide binds to Hsc70 (e.g., human Hsc70) with a K_{d} of 10⁻³ M, 10⁻⁴ M, 10⁻⁵ M, 10⁻⁶ M, 10⁻⁷ M, 10⁻⁸ M, 10⁻⁹M, or lower.

Methods of calculating the amounts of components in the unit dosage form are provided. For example, in certain embodiments, the polypeptides have an average molecular weight of about 3 kD, and the molecular weight of Hsc70 is about 71 kD. Assuming in one embodiment that the total amount of the polypeptides and stress protein in the composition is 300 μg, and the molar ratio of the polypeptides to hsc70 is 1.5:1. The molar amount of Hsc70 can be calculated as 300 μg divided by 71 kD+1.5×3 kD, resulting in about 4.0 nmol, and the mass amount of Hsc70 can be calculated by multiplying the molar amount with 71 kD, resulting in about 280 kD. The total molar amount of the polypeptides can be calculated as 1.5×4.0 nmol, resulting in 6.0 nmol. If 10 different polypeptides are employed, the molar amount of each polypeptide is 0.60 nmol. Assuming in another embodiment that a 300 μg dose of Hsc70 is intended to be included in a unit dosage form, and the molar ratio of polypeptides to Hsc70 is 1.5:1. The total molar amount of the polypeptides can be calculated as 300 μg divided by 71 kD then times 1.5, resulting in 6.3 nmol. If 10 different polypeptides are employed, the molar amount of each polypeptide is 0.63 nmol. In cases where one or more of the variables are different from those in the examples, the quantities of the stress proteins and of the polypeptides are scaled accordingly.

It is further appreciated that the unit dosage form can optionally comprise one or more adjuvants as disclosed supra. In certain embodiments, the adjuvant comprises a saponin and/or an immunostimulatory nucleic acid. In certain embodiments, the adjuvant comprises or further comprises QS-21. In certain embodiments, the amount of QS-21 in the unit dosage form of composition is 10 μg, 25 μg, 50 μg, 75 μg, 100 μg, 125 μg, 150 μg, 175 μg, or 200 μg. In certain embodiments, the amount of QS-21 in the unit dosage form of composition is 100 μg. In certain embodiments, the adjuvant comprises a Toll-like receptor (TLR) agonist. In certain embodiments, the TLR agonist is an agonist of TLR4. In certain embodiments, the TLR agonist is an agonist of TLR7 and/or TLR8. In certain embodiments, the TLR agonist is an agonist of TLR9. In certain embodiments, the TLR agonist is an agonist of TLR5.

### 6.4 Methods of Use

The compositions (e.g., pharmaceutical compositions and vaccines, and unit dosage forms thereof) disclosed herein are particularly useful for inducing a cellular immune response. In certain embodiments, stress proteins can deliver antigenic polypeptides through the cross-presentation pathway in antigen presenting cells (APCs) (e.g., macrophages and dendritic cells (DCs) via membrane receptors (mainly CD91) or by binding to Toll-like receptors, thereby leading to activation of CD8⁺ and CD4⁺ T cells. Internalization of a stress protein/antigenic polypeptide complex results in functional maturation of the APCs with chemokine and cytokine production leading to activation of natural killer cells (NK), monocytes and Th1 and Th-2-mediated immune responses.

In one aspect, the instant disclosure provides a method of inducing a cellular immune response to an antigenic peptide in a subject, the method comprising administering to the subject an effective amount of a composition as disclosed herein. In another aspect, the instant disclosure provides a method of treating a disease (e.g., cancer) in a subject, the method comprising administering to the subject an effective amount of a composition as disclosed herein. The compositions disclosed herein can also be used in preparing a medicament or vaccine for the treatment of a subject.

In various embodiments, such subjects can be an animal, e.g., a mammal, a non-human primate, and a human. The term “animal” includes companion animals, such as cats and dogs; zoo animals; wild animals, including deer, foxes and raccoons; farm animals, livestock and fowl, including horses, cattle, sheep, pigs, turkeys, ducks, and chickens, and laboratory animals, such as rodents, rabbits, and guinea pigs. In certain embodiments, the subject has cancer.

### 6.4.1 Treatment of Cancer

The compositions disclosed herein can be used alone or in combination with other therapies for the treatment of cancer. One or more of the MHC-binding peptides disclosed herein can be present in the subject's cancer cells. In certain embodiments, one or more of the MHC-binding peptides are common to or frequently found in the type and/or stage of the cancer. In certain embodiments, one or more MHC-binding peptides are found in greater than 5% of cancers. In certain embodiments, one or more of the WIC-binding peptides are specific to the cancer of the subject.

Cancers that can be treated using the compositions disclosed herein include, without limitation, a solid tumor, a hematological cancer (e.g., leukemia, lymphoma, myeloma, e.g., multiple myeloma), and a metastatic lesion. In one embodiment, the cancer is a solid tumor. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma), and pancreas, as well as adenocarcinomas which include malignancies such as colon cancers, rectal cancer, renal-cell carcinoma, liver cancer, lung cancer (e.g., non-small cell lung cancer or small cell lung cancer), cancer of the small intestine and cancer of the esophagus. The cancer may be at an early, intermediate, late stage or metastatic cancer. In certain embodiments, the cancer is associated with elevated PD-1 activity (e.g., elevated PD-1 expression).

In one embodiment, the cancer is chosen from a lung cancer (e.g., lung adenocarcinoma or a non-small cell lung cancer (NSCLC) (e.g., a NSCLC with squamous and/or non-squamous histology, or a NSCLC adenocarcinoma)), a melanoma (e.g., an advanced melanoma), a renal cancer (e.g., a renal cell carcinoma), a liver cancer (e.g., hepatocellular carcinoma or intrahepatic cholangiocellular carcinoma), a myeloma (e.g., a multiple myeloma), a prostate cancer, a breast cancer (e.g., a breast cancer that does not express one, two or all of estrogen receptor, progesterone receptor, or Her2/neu, e.g., a triple negative breast cancer), an ovarian cancer, a colorectal cancer, a pancreatic cancer, a head and neck cancer (e.g., head and neck squamous cell carcinoma (HNSCC), anal cancer, gastro-esophageal cancer (e.g., esophageal squamous cell carcinoma), mesothelioma, nasopharyngeal cancer, thyroid cancer, cervical cancer, epithelial cancer, peritoneal cancer, or a lymphoproliferative disease (e.g., a post-transplant lymphoproliferative disease). In one embodiment, the cancer is NSCLC. In one embodiment, the cancer is a renal cell carcinoma. In one embodiment, the cancer is an ovarian cancer, optionally wherein the ovarian cancer is associated with human papillomavirus (HPV) infection. In a specific embodiment, the ovarian cancer is a platinum-refractory ovarian cancer.

In one embodiment, the cancer is a hematological cancer, for example, a leukemia, a lymphoma, or a myeloma. In one embodiment, the cancer is a leukemia, for example, acute lymphoblastic leukemia (ALL), acute myelogenous leukemia (AML), acute myeloblastic leukemia (AML), chronic lymphocytic leukemia (CLL), chronic myelogenous leukemia (CML), chronic myeloid leukemia (CML), chronic myelomonocytic leukemia (CMML), chronic lymphocytic leukemia (CLL), or hairy cell leukemia. In one embodiment, the cancer is a lymphoma, for example, B cell lymphoma, diffuse large B-cell lymphoma (DLBCL), activated B-cell like (ABC) diffuse large B cell lymphoma, germinal center B cell (GCB) diffuse large B cell lymphoma, mantle cell lymphoma, Hodgkin lymphoma, non-Hodgkin lymphoma, relapsed non-Hodgkin lymphoma, refractory non-Hodgkin lymphoma, recurrent follicular non-Hodgkin lymphoma, Burkitt lymphoma, small lymphocytic lymphoma, follicular lymphoma, lymphoplasmacytic lymphoma, or extranodal marginal zone lymphoma. In one embodiment the cancer is a myeloma, for example, multiple myeloma.

In another embodiment, the cancer is chosen from a carcinoma (e.g., advanced or metastatic carcinoma), melanoma or a lung carcinoma, e.g., a non-small cell lung carcinoma.

In one embodiment, the cancer is a lung cancer, e.g., a lung adenocarcinoma, non-small cell lung cancer or small cell lung cancer.

In one embodiment, the cancer is a melanoma, e.g., an advanced melanoma. In one embodiment, the cancer is an advanced or unresectable melanoma that does not respond to other therapies. In other embodiments, the cancer is a melanoma with a BRAF mutation (e.g., a BRAF V600 mutation). In yet other embodiments, the compositions disclosed herein is administered after treatment with an anti-CTLA-4 antibody (e.g., ipilimumab) with or without a BRAF inhibitor (e.g., vemurafenib or dabrafenib).

In another embodiment, the cancer is a hepatocarcinoma, e.g., an advanced hepatocarcinoma, with or without a viral infection, e.g., a chronic viral hepatitis.

In another embodiment, the cancer is a prostate cancer, e.g., an advanced prostate cancer.

In yet another embodiment, the cancer is a myeloma, e.g., multiple myeloma.

In yet another embodiment, the cancer is a renal cancer, e.g., a renal cell carcinoma (RCC) (e.g., a metastatic RCC, clear cell renal cell carcinoma (CCRCC) or kidney papillary cell carcinoma).

In yet another embodiment, the cancer is chosen from a lung cancer, a melanoma, a renal cancer, a breast cancer, a colorectal cancer, a leukemia, or a metastatic lesion of the cancer.

In a particular embodiment, the cancer is AML. In another particular embodiment, the cancer is colorectal cancer.

The compositions disclosed herein may be administered when a cancer is detected, or prior to or during an episode of recurrence.

Administration can begin at the first sign of cancer or recurrence, followed by boosting doses until at least symptoms are substantially abated and for a period thereafter.

In some embodiments, the compositions can be administered to a subject with cancer who has undergone tumor resection surgery that results in an insufficient amount of resected tumor tissue (e.g., less than 7 g, less than 6 g, less than 5 g, less than 4 g, less than 3 g, less than 2 g, or less than 1 g of resected tumor tissue) for production of a therapeutically effective amount of an autologous cancer vaccine comprising a representative set of antigens collected from the resected tumor tissue. See, for example, cancer vaccines described in Expert Opin. Biol. Ther. 2009 Febuary; 9(2):179-86; incorporated herein by reference.

The compositions disclosed herein can also be used for immunization against recurrence of cancers. Prophylactic administration of a composition to an individual can confer protection against a future recurrence of a cancer.

### 6.4.2 Combination Therapy

Combination therapy refers to the use of compositions disclosed herein, as a first modality, with a second modality to treat cancer. Accordingly, in certain embodiments, the instant disclosure provides a method of inducing a cellular immune response to an antigenic peptide in a subject as disclosed herein, or a method of treating a disease in a subject as disclosed herein, the method comprising administering to the subject an effective amount of (a) a composition as disclosed herein and (b) a second modality.

In one embodiment, the second modality is a non-HSP modality, e.g., a modality that does not comprise HSP as a component. This approach is commonly termed combination therapy, adjunctive therapy or conjunctive therapy (the terms are used interchangeably). With combination therapy, additive potency or additive therapeutic effect can be observed. Synergistic outcomes are sought where the therapeutic efficacy is greater than additive. The use of combination therapy can also provide better therapeutic profiles than the administration of either the first or the second modality alone.

The additive or synergistic effect may allow for a reduction in the dosage and/or dosing frequency of either or both modalities to mitigate adverse effects. In certain embodiments, the second modality administered alone is not clinically adequate to treat the subject (e.g., the subject is non-responsive or refractory to the single modality), such that the subject needs an additional modality. In certain embodiments, the subject has responded to the second modality, yet suffers from side effects, relapses, develops resistance, etc., such that the subject needs an additional modality. Methods disclosed herein comprising administration of the compositions disclosed herein to such subjects to improve the therapeutic effectiveness of the second modality. The effectiveness of a treatment modality can be assayed in vivo or in vitro using methods known in the art.

In one embodiment, a lesser amount of the second modality is required to produce a therapeutic benefit in a subject. In specific embodiments, a reduction of about 10%, 20%, 30%, 40% and 50% of the amount of second modality can be achieved. The amount of the second modality, including amounts in a range that does not produce any observable therapeutic benefits, can be determined by dose-response experiments conducted in animal models by methods well known in the art.

In certain embodiments, the second modality comprises a TCR, e.g., a soluble TCR or a cell expressing a TCR. In certain embodiments, the second modality comprises a cell expressing a chimeric antigen receptor (CAR). In certain embodiments, the cell expressing the TCR or CAR is a T cell. In a particular embodiment, the TCR or CAR binds to (e.g., specifically binds to) at least one WIC-binding epitope in the composition disclosed herein.

In certain embodiments, the second modality comprises a TCR mimic antibody. In certain embodiments, the TCR mimic antibody is an antibody that specifically binds to a peptide-WIC complex. Non-limiting examples of TCR mimic antibodies are disclosed in U.S. Pat. No. 9,074,000, U.S. Publication Nos. US 2009/0304679 A1 and US 2014/0134191 A1, all of which are incorporated herein by reference in their entireties. In a particular embodiment, the TCR mimic antibody binds to (e.g., specifically binds to) at least one WIC-binding epitope in the composition disclosed herein.

In certain embodiments, the second modality comprises a checkpoint targeting agent. In certain embodiments, the checkpoint targeting agent is selected from the group consisting of an antagonist anti-CTLA-4 antibody, an antagonist anti-PD-L1 antibody, an antagonist anti-PD-L2 antibody, an antagonist anti-PD-1 antibody, an antagonist anti-TIM-3 antibody, an antagonist anti-LAG-3 antibody, an antagonist anti-CEACAM1 antibody, an agonist anti-CD137 antibody, an antagonist anti-TIGIT antibody, an antagonist anti-VISTA antibody, an agonist anti-GITR antibody, and an agonist anti-OX40 antibody.

In certain embodiments, an anti-PD-1 antibody is used as the second modality in methods disclosed herein. In certain embodiments, the anti-PD-1 antibody is nivolumab, also known as BMS-936558 or MDX1106, developed by Bristol-Myers Squibb. In certain embodiments, the anti-PD-1 antibody is pembrolizumab, also known as lambrolizumab or MK-3475, developed by Merck & Co. In certain embodiments, the anti-PD-1 antibody is pidilizumab, also known as CT-011, developed by CureTech. In certain embodiments, the anti-PD-1 antibody is MEDI0680, also known as AMP-514, developed by Medimmune. In certain embodiments, the anti-PD-1 antibody is PDR001 developed by Novartis Pharmaceuticals. In certain embodiments, the anti-PD-1 antibody is REGN2810 developed by Regeneron Pharmaceuticals. In certain embodiments, the anti-PD-1 antibody is PF-06801591 developed by Pfizer. In certain embodiments, the anti-PD-1 antibody is BGB-A317 developed by BeiGene. In certain embodiments, the anti-PD-1 antibody is TSR-042 developed by AnaptysBio and Tesaro. In certain embodiments, the anti-PD-1 antibody is SHR-1210 developed by Hengrui.

Further non-limiting examples of anti-PD-1 antibodies that may be used in treatment methods disclosed herein are disclosed in the following patents and patent applications, all of which are herein incorporated by reference in their entireties for all purposes: U.S. Pat. Nos. 6,808,710; 7,332,582; 7,488,802; 8,008,449; 8,114,845; 8,168,757; 8,354,509; 8,686,119; 8,735,553; 8,747,847; 8,779,105; 8,927,697; 8,993,731; 9,102,727; 9,205,148; U.S. Publication No. US 2013/0202623 A1; U.S. Publication No. US 2013/0291136 A1; U.S. Publication No. US 2014/0044738 A1; U.S. Publication No. US 2014/0356363 A1; U.S. Publication No. US 2016/0075783 A1; and PCT Publication No. WO 2013/033091 A1; PCT Publication No. WO 2015/036394 A1; PCT Publication No. WO 2014/179664 A2; PCT Publication No. WO 2014/209804 A1; PCT Publication No. WO 2014/206107 A1; PCT Publication No. WO 2015/058573 A1; PCT Publication No. WO 2015/085847 A1; PCT Publication No. WO 2015/200119 A1; PCT Publication No. WO 2016/015685 A1; and PCT Publication No. WO 2016/020856 A1.

In certain embodiments, an anti-PD-L1 antibody is used as the second modality in methods disclosed herein. In certain embodiments, the anti-PD-L1 antibody is atezolizumab developed by Genentech. In certain embodiments, the anti-PD-L1 antibody is durvalumab developed by AstraZeneca, Celgene and Medimmune. In certain embodiments, the anti-PD-L1 antibody is avelumab, also known as MSB0010718C, developed by Merck Serono and Pfizer. In certain embodiments, the anti-PD-L1 antibody is MDX-1105 developed by Bristol-Myers Squibb. In certain embodiments, the anti-PD-L1 antibody is AMP-224 developed by Amplimmune and GSK.

Non-limiting examples of anti-PD-L1 antibodies that may be used in treatment methods disclosed herein are disclosed in the following patents and patent applications, all of which are herein incorporated by reference in their entireties for all purposes: U.S. Pat. Nos. 7,943,743; 8,168,179; 8,217,149; 8,552,154; 8,779,108; 8,981,063; 9,175,082; U.S. Publication No. US 2010/0203056 A1; U.S. Publication No. US 2003/0232323 A1; U.S. Publication No. US 2013/0323249 A1; U.S. Publication No. US 2014/0341917 A1; U.S. Publication No. US 2014/0044738 A1; U.S. Publication No. US 2015/0203580 A1; U.S. Publication No. US 2015/0225483 A1; U.S. Publication No. US 2015/0346208 A1; U.S. Publication No. US 2015/0355184 A1; and PCT Publication No. WO 2014/100079 A1; PCT Publication No. WO 2014/022758 A1; PCT Publication No. WO 2014/055897 A2; PCT Publication No. WO 2015/061668 A1; PCT Publication No. WO 2015/109124 A1; PCT Publication No. WO 2015/195163 A1; PCT Publication No. WO 2016/000619 A1; and PCT Publication No. WO 2016/030350 A1.

In certain embodiments, a compound that targets an immunomodulatory enzyme(s) such as IDO (indoleamine-(2,3)-dioxygenase) and/or TDO (tryptophan 2,3-dioxygenase) is used as the second modality in methods disclosed herein. Therefore, in one embodiment, the compound targets an immunomodulatory enzyme(s), such as an inhibitor of indoleamine-(2,3)-dioxygenase (IDO). In certain embodiments, such compound is selected from the group consisting of epacadostat (Incyte Corp; see, e.g., WO 2010/005958 which is herein incorporated by reference in its entirety), F001287 (Flexus Biosciences/Bristol-Myers Squibb), indoximod (NewLink Genetics), and NLG919 (NewLink Genetics). In one embodiment, the compound is epacadostat. In another embodiment, the compound is F001287. In another embodiment, the compound is indoximod. In another embodiment, the compound is NLG919. In a specific embodiment, an anti-TIM-3 (e.g., human TIM-3) antibody disclosed herein is administered to a subject in combination with an IDO inhibitor for treating cancer. The IDO inhibitor as described herein for use in treating cancer is present in a solid dosage form of a pharmaceutical composition such as a tablet, a pill or a capsule, wherein the pharmaceutical composition includes an IDO inhibitor and a pharmaceutically acceptable excipient. As such, the antibody as described herein and the IDO inhibitor as described herein can be administered separately, sequentially or concurrently as separate dosage forms. In one embodiment, the antibody is administered parenterally, and the IDO inhibitor is administered orally. In particular embodiments, the inhibitor is selected from the group consisting of epacadostat (Incyte Corporation), F001287 (Flexus Biosciences/Bristol-Myers Squibb), indoximod (NewLink Genetics), and NLG919 (NewLink Genetics). Epacadostat has been described in PCT Publication No. WO 2010/005958, which is herein incorporated by reference in its entirety for all purposes. In one embodiment, the inhibitor is epacadostat. In another embodiment, the inhibitor is F001287. In another embodiment, the inhibitor is indoximod. In another embodiment, the inhibitor is NLG919.

In certain embodiments, the second modality comprises a different vaccine (e.g., a peptide vaccine, a DNA vaccine, or an RNA vaccine) for treating cancer. In certain embodiments, the vaccine is a heat shock protein-based tumor vaccine or a heat shock protein-based pathogen vaccine (e.g., a vaccine as described in WO 2016/183486, which is incorporated herein by reference in its entirety). In a specific embodiment, the second modality comprises a stress protein-based vaccine. For example, in certain embodiments, the second modality comprises a composition as disclosed herein that is different from the first modality. In certain embodiments, the second modality comprises a composition analogous to those disclosed herein except for having a different sequence of the HSP-binding peptide. In certain embodiments, the stress protein-based vaccine is derived from a tumor preparation, such that the immunity elicited by the vaccine is specifically directed against the unique antigenic peptide repertoire expressed by the cancer of each subject.

In certain embodiments, the second modality comprises one or more adjuvants, such as the ones disclosed supra that may be included in the vaccine formulation disclosed herein. In certain embodiments, the second modality comprises a saponin, an immunostimulatory nucleic acid, and/or QS-21. In certain embodiments, the second modality comprises a Toll-like receptor (TLR) agonist. In certain embodiments, the TLR agonist is an agonist of TLR4. In certain embodiments, the TLR agonist is an agonist of TLR7 and/or TLR8. In certain embodiments, the TLR agonist is an agonist of TLR9. In certain embodiments, the TLR agonist is an agonist of TLR5.

In certain embodiments, the second modality comprises one or more of the agents selected from the group consisting of lenalidomide, dexamethasone, interleukin-2, recombinant interferon alfa-2b, and peginterferon alfa-2b.

In certain embodiments, where the pharmaceutical composition is used for treating a subject having cancer, the second modality comprises a chemotherapeutic or a radiotherapeutic. In certain embodiments, the chemotherapeutic agent is a hypomethylating agent (e.g., azacitidine).

The composition disclosed herein can be administered separately, sequentially, or concurrently from the second modality (e.g., chemotherapeutic, radiotherapeutic, checkpoint targeting agent, IDO inhibitor, vaccine, adjuvant, soluble TCR, cell expressing a TCR, cell expressing a CAR, and/or TCR mimic antibody), by the same or different delivery routes.

### 6.4.3 Dosage Regimen

The dosage of the compositions disclosed herein, and the dosage of any additional treatment modality if combination therapy is to be administered, depends to a large extent on the weight and general state of health of the subject being treated, as well as the frequency of treatment and the route of administration. Amounts effective for this use will also depend on the stage and severity of the disease and the judgment of the prescribing physician, but generally range for the initial immunization (that is, for therapeutic administration) from about 1.0 μg to about 1000 μg (1 mg) (including, for example, 10, 20, 25, 30, 40, 50, 60, 70, 80, 90, 100, 150, 200, 240, 250, 300, 350, 400, 450, 500, 550, 600, 650, 700, 750, 800, 850, 900, 950, or 1000 μg) of any one of the compositions disclosed herein for a 70 kg patient, followed by boosting dosages of from about 1.0 μg to about 1000 μg of the composition (including, for example, 10, 20, 25, 30, 40, 50, 60, 70, 80, 90, 100, 150, 200, 250, 300, 350, 400, 450, 500, 550, 600, 650, 700, 750, 800, 850, 900, 950, or 1000 μg) pursuant to a boosting regimen over weeks to months depending upon the patient's response and condition by measuring specific CTL activity in the patient's blood. Regimens for continuing therapy, including site, dose and frequency may be guided by the initial response and clinical judgment. Dosage ranges and regimens for adjuvants are known to those in the art, see, e.g., Vogel and Powell, 1995, A Compendium of Vaccine Adjuvants and Excipients; M. F. Powell, M. J. Newman (eds.), Plenum Press, New York, pages 141-228.

Preferred adjuvants include QS-21, e.g., QS-21 Stimulon®, and CpG oligonucleotides. Exemplary dosage ranges for QS-21 are 1 μg to 200 μg per administration. In other embodiments, dosages for QS-21 can be 10, 25, and 50 μg per administration. In certain embodiments, the adjuvant comprises a Toll-like receptor (TLR) agonist. In certain embodiments, the TLR agonist is an agonist of TLR4. In certain embodiments, the TLR agonist is an agonist of TLR7 and/or TLR8. In certain embodiments, the TLR agonist is an agonist of TLR9. In certain embodiments, the TLR agonist is an agonist of TLR5.

In certain embodiments, the administered amount of compositions depends on the route of administration and the type of HSPs in the compositions. For example, the amount of HSP in the compositions can range, for example, from 5 to 1000 μg (1 mg) per administration. In certain embodiments, the administered amount of compositions comprising Hsc70-, Hsp70- and/or Gp96-polypeptide complexes is, for example, 5, 10, 20, 25, 30, 40, 50, 60, 70, 80, 90, 100, 200, 250, 300, 400, 500, 600, 700, 750, 800, 900, or 1000 μg. In certain embodiments, the administered amount of the composition is in the range of about 10 to 600 μg per administration and about 5 to 100 μg if the composition is administered intradermally. In certain embodiments, the administered amount of the composition is about 5 μg to 600 μg, about 5 μg to 300 μg, about 5 μg to 150 μg, or about 5 μg to 60 μg. In certain embodiments, the administered amount of the composition is less than 100 μg. In certain embodiments, the administered amount of the composition is about 5 μg, 25 μg, 50 μg, 120 μg, 240 μg, or 480 μg. In certain embodiments, the compositions comprising complexes of stress proteins and polypeptides are purified.

In one embodiment of a therapeutic regimen, a dosage substantially equivalent to that observed to be effective in smaller non-human animals (e.g., mice or guinea pigs) is effective for human administration, optionally subject to a correction factor not exceeding a fifty-fold increase, based on the relative lymph node sizes in such mammals and in humans. Specifically, interspecies dose-response equivalence for stress proteins (or HSPs) noncovalently bound to or mixed with antigenic molecules for a human dose is estimated as the product of the therapeutic dosage observed in mice and a single scaling ratio, not exceeding a fifty-fold increase. In certain embodiment, the dosages of the composition can be much smaller than the dosage estimated by extrapolation.

The doses recited above can be given once or repeatedly, such as daily, every other day, weekly, biweekly, or monthly, for a period up to a year or over a year. Doses are preferably given once every 28 days for a period of about 52 weeks or more.

In one embodiment, the compositions are administered to a subject at reasonably the same time as an additional treatment modality or modalities. This method provides that the two administrations are performed within a time frame of less than one minute to about five minutes, or up to about sixty minutes from each other, for example, at the same doctor's visit.

In another embodiment, the compositions and an additional treatment modality or modalities are administered concurrently.

In yet another embodiment the compositions and an additional treatment modality or modalities are administered in a sequence and within a time interval such that the complexes disclosed herein, and the additional treatment modality or modalities can act together to provide an increased benefit than if they were administered alone.

In another embodiment, the compositions and an additional treatment modality or modalities are administered sufficiently close in time so as to provide the desired therapeutic or prophylactic outcome. Each can be administered simultaneously or separately, in any appropriate form and by any suitable route. In one embodiment, the complexes disclosed herein, and the additional treatment modality or modalities are administered by different routes of administration. In an alternate embodiment, each is administered by the same route of administration. The compositions can be administered at the same or different sites, e.g. arm and leg. When administered simultaneously, the compositions and an additional treatment modality or modalities may or may not be administered in admixture or at the same site of administration by the same route of administration.

In various embodiments, the compositions and an additional treatment modality or modalities are administered less than 1 hour apart, at about 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In other embodiments, the compositions and a vaccine composition are administered 2 to 4 days apart, 4 to 6 days apart, 1 week a part, 1 to 2 weeks apart, 2 to 4 weeks apart, one month apart, 1 to 2 months apart, or 2 or more months apart. In preferred embodiments, the compositions and an additional treatment modality or modalities are administered in a time frame where both are still active. One skilled in the art would be able to determine such a time frame by determining the half-life of each administered component.

In certain embodiments, the compositions are administered to the subject weekly for at least four weeks. In certain embodiments, after the four weekly doses, at least 2 (e.g., 2, 3, 4, 5, 6, 7, 8, 9, 10, 11, 12, 13, 14, 15, 16, 17, 18, 19, or 20) further doses of the compositions are administered biweekly to the subject. In certain embodiments, the compositions administered as a booster three months after the final weekly or biweekly dose. The booster administered every three months can be administered for the life of the subject (e.g., at least 1, 2, 3, 4, 5, 6, 7, 8, 9, 10, 11, 12, 13, 14, 15, 16, 17, 18, 19, 20, 25, 30, 40, 50, or more years). In certain embodiments, the total number of doses of the compositions administered to the subject is 1, 2, 3, 4, 5, 6, 7, 8, 9, 10, 11, 12, 13, 14, 15, 16, 17, 18, 19, or 20.

In one embodiment, the compositions and an additional treatment modality or modalities are administered within the same patient visit. In certain embodiments, the compositions are administered prior to the administration of an additional treatment modality or modalities. In an alternate specific embodiment, the compositions are administered subsequent to the administration of an additional treatment modality or modalities.

In certain embodiments, the compositions and an additional treatment modality or modalities are cyclically administered to a subject. Cycling therapy involves the administration of the compositions for a period of time, followed by the administration of a modality for a period of time and repeating this sequential administration. Cycling therapy can reduce the development of resistance to one or more of the therapies, avoid or reduce the side effects of one of the therapies, and/or improve the efficacy of the treatment. In such embodiments, the disclosure contemplates the alternating administration of the compositions followed by the administration of a modality 4 to 6 days later, preferable 2 to 4 days, later, more preferably 1 to 2 days later, wherein such a cycle may be repeated as many times as desired. In certain embodiments, the compositions and the modality are alternately administered in a cycle of less than 3 weeks, once every two weeks, once every 10 days or once every week. In certain embodiments, the compositions are administered to a subject within a time frame of one hour to twenty-four hours after the administration of a modality. The time frame can be extended further to a few days or more if a slow- or continuous-release type of modality delivery system is used.

### 6.4.4 Routes of Administration

The compositions disclosed herein may be administered using any desired route of administration. Many methods may be used to introduce the compositions described above, including but not limited to, oral, intradermal, intramuscular, intraperitoneal, intravenous, subcutaneous, mucosal, intranasal, intra-tumoral, and intra-lymph node routes. Non-mucosal routes of administration include, but are not limited to, intradermal and topical administration. Mucosal routes of administration include, but are not limited to, oral, rectal and nasal administration. Advantages of intradermal administration include use of lower doses and rapid absorption, respectively. Advantages of subcutaneous or intramuscular administration include suitability for some insoluble suspensions and oily suspensions, respectively. Preparations for mucosal administrations are suitable in various formulations as described below.

Solubility and the site of the administration are factors which should be considered when choosing the route of administration of the compositions. The mode of administration can be varied between multiple routes of administration, including those listed above.

If the compositions are water-soluble, then it may be formulated in an appropriate buffer, for example, phosphate buffered saline or other physiologically compatible solutions, preferably sterile. Alternatively, if a composition has poor solubility in aqueous solvents, then it may be formulated with a non-ionic surfactant such as Tween, or polyethylene glycol. Thus, the compositions may be formulated for administration by inhalation or insufflation (either through the mouth or the nose) or oral, buccal, parenteral, or rectal administration.

For oral administration, the composition may be in liquid form, for example, solutions, syrups or suspensions, or may be presented as a drug product for reconstitution with water or other suitable vehicle before use. Such a liquid preparation may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The compositions may take the form of, for example, tablets or capsules prepared by conventional means with pharmaceutically acceptable excipients such as binding agents (e.g., pre-gelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

The compositions for oral administration may be suitably formulated to be released in a controlled and/or timed manner.

For buccal administration, the compositions may take the form of tablets or lozenges formulated in conventional manner.

The preparation may be formulated for parenteral administration by injection, e.g., by bolus injection or continuous infusion. Formulations for injection may be presented in unit dosage form, e.g., in ampoules or in multi-dose containers, with an added preservative. The preparation may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The preparation may also be formulated in a rectal preparation such as a suppository or retention enema, e.g., containing conventional suppository bases such as cocoa butter or other glycerides.

In addition to the formulations described above, the preparation may also be formulated as a depot preparation. Such long acting formulations may be administered by implantation (for example, subcutaneously or intramuscularly) or by intramuscular injection. Thus, for example, the preparation may be formulated with suitable polymeric or hydrophobic materials (for example, as emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

For administration by inhalation, the compositions are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

### 6.4.5 Patient (Subject) Evaluation

Patients treated with the compositions disclosed herein may be tested for an anti-tumor immune response. In this regard, peripheral blood from patients may be obtained and assayed for markers of anti-tumor immunity. Using standard laboratory procedures, leukocytes may be obtained from the peripheral blood and assayed for frequency of different immune cell phenotypes, HLA subtype, and function of anti-tumor immune cells.

The majority of effector immune cells in the anti-tumor response is CD8⁺ T cells and thus is HLA class I restricted. Using immunotherapeutic strategies in other tumor types, expansion of CD8+ cells that recognize HLA class I restricted antigens is found in a majority of patients. However, other cell types are involved in the anti-tumor immune response, including, for example, CD4+ T cells, and macrophages and dendritic cells, which may act as antigen-presenting cells. Populations of T cells (CD4+, CD8+, and Treg cells), macrophages, and antigen presenting cells may be determined using flow cytometry. HLA typing may be performed using routine methods in the art, such as methods described in Boegel et al. Genome Medicine 2012, 4:102 (seq2HLA), or using a TruSight® HLA sequencing panel (Illumina, Inc.). The HLA subtype of CD8+ T cells may be determined by a complement-dependent microcytotoxicity test.

To determine if there is an increase in anti-tumor T cell response, an enzyme linked immunospot assay may be performed to quantify the IFNγ-producing peripheral blood mononuclear cells (PBMC). This technique provides an assay for antigen recognition and immune cell function. In some embodiments, subjects who respond clinically to the vaccine may have an increase in tumor-specific T cells and/or IFNγ-producing PBMCs. In some embodiments, immune cell frequency is evaluated using flow cytometry. In some embodiments, antigen recognition and immune cell function is evaluated using enzyme linked immunospot assays.

In some embodiments, a panel of assays may be performed to characterize the immune response generated to the composition alone or given in combination with standard of care (e.g., maximal surgical resection, radiotherapy, and concomitant and adjuvant chemotherapy with temozolomide for glioblastoma multiforme). In some embodiments, the panel of assays includes one or more of the following tests: whole blood cell count, absolute lymphocyte count, monocyte count, percentage of CD4⁺CD3⁺ T cells, percentage of CD8⁺CD3⁺ T cells, percentage of CD4⁺CD25⁺FoxP3⁺ regulatory T cells and other phenotyping of PBL surface markers, intracellular cytokine staining to detect proinflammatory cytokines at the protein level, qPCR to detect cytokines at the mRNA level and CFSE dilution to assay T cell proliferation.

In evaluating a subject, a number of other tests may be performed to determine the overall health of the subject. For example, blood samples may be collected from subjects and analyzed for hematology, coagulation times and serum biochemistry. Hematology for CBC may include red blood cell count, platelets, hematocrit, hemoglobin, white blood cell (WBC) count, plus WBC differential to be provided with absolute counts for neutrophils, eosinophils, basophils, lymphocytes, and monocytes. Serum biochemistry may include albumin, alkaline phosphatase, aspartate amino transferase, alanine amino transferase, total bilirubin, BUN, glucose, creatinine, potassium and sodium. Protime (PT) and partial thromboplastin time (PTT) may also be tested. One or more of the following tests may also be conducted: anti-thyroid (anti-microsomal or thyroglobulin) antibody tests, assessment for anti-nuclear antibody, and rheumatoid factor. Urinalysis may be performed to evaluated protein, RBC, and WBC levels in urine. Also, a blood draw to determine histocompatibility leukocyte antigen (HLA) status may be performed.

In some embodiments, radiologic tumor evaluations are performed one or more times throughout a treatment to evaluate tumor size and status. For example, tumor evaluation scans may be performed within 30 days prior to surgery, within 48 hours after surgery (e.g., to evaluate percentage resection), 1 week (maximum 14 days) prior to the first vaccination (e.g., as a baseline evaluation), and approximately every 8 weeks thereafter for a particular duration. MRI or CT imaging may be used. Typically, the same imaging modality used for the baseline assessment is used for each tumor evaluation visit.

### 6.5 Kits

Kits are also provided for carrying out the prophylactic and therapeutic methods disclosed herein. The kits may optionally further comprise instructions on how to use the various components of the kits.

In certain embodiments, the kit comprises a first container containing a composition (e.g., composition comprising stress protein(s) and antigenic polypeptide(s) disclosed herein, and a second container containing one or more adjuvants. The adjuvant can be any adjuvant disclosed herein, e.g., a saponin, an immunostimulatory nucleic acid, or QS-21 (e.g., QS-21 Stimulon®). In certain embodiments, the kit further comprises a third container containing an additional treatment modality. The kit can further comprise an instruction on the indication, dosage regimen, and route of administration of the composition, adjuvant, and additional treatment modality, e.g., as disclosed in herein.

Alternatively, the kit can comprise the stress protein(s) and antigenic polypeptide(s) of a composition disclosed herein in separate containers. In certain embodiments, the kit comprises a first container containing one or more antigenic polypeptides disclosed herein, and a second container containing a purified stress protein capable of binding to the polypeptide.

The first container can contain any number of different polypeptides. For example, in certain embodiments, the first container contains no more than 100 different polypeptides, e.g., 2-50, 2-30, 2-20, 5-20, 5-15, 5-10, or 10-15 different polypeptides. In certain embodiments, each of the different polypeptides comprises the same HSP-binding peptide and a different antigenic peptide. In certain embodiments, the total amount of the polypeptide(s) in the first container is a suitable amount for a unit dosage. In certain embodiments, the total amount of the polypeptide(s) in the first container is about 0.1 to 20 nmol (e.g., 3, 4, 5, or 6 nmol).

The second container can contain any stress protein disclosed herein. In certain embodiments, the stress protein is selected from the group consisting of Hsc70, Hsp70, Hsp90, Hsp110, Grp170, Gp96, or Calreticulin, and a mutant or fusion protein thereof. In certain embodiments, the stress protein is Hsc70 (e.g., human Hsc70). In certain embodiments, the stress protein is a recombinant protein. In certain embodiments, the total amount of the stress protein(s) in the second container is about 10 μg to 600 μg (e.g., 120 μg, 240 μg, or 480 μg). In certain embodiments, the total amount of the stress protein(s) in the second container is about 50 μg, 100 μg, 200 μg, 300 μg, 400 μg, or 500 μg. In certain embodiments, the amount of the stress protein in the composition is about 300 μg. In certain embodiments, the total molar amount of the stress protein(s) in the second container is calculated based on the total molar amount of the polypeptide(s) in the first container, such that the molar ratio of the polypeptide(s) to the stress protein(s) is about 0.5:1 to 5:1 (e.g., about 1:1, 1.25:1, 1.5:1, 2:1, 2.5:1, 3:1, 3.5:1, 4:1, 4.5:1, or 5:1). In certain embodiments, the total amount of the stress protein(s) in the second container is an amount for multiple administrations (e.g., less than or equal to 1 mg, 3 mg, 10 mg, 30 mg, or 100 mg).

In certain embodiments, the kit further comprises an instruction for preparing a composition from the polypeptide(s) in the first container and the stress protein(s) in the second container (e.g., an instruction for the complexing reaction as disclosed herein).

In certain embodiments, the kit further comprises a third container containing one or more adjuvants. The adjuvant can be any adjuvant disclosed herein, e.g., a saponin, an immunostimulatory nucleic acid, or QS-21 (e.g., QS-21 Stimulon®). In certain embodiments, the kit further comprises a fourth container containing an additional treatment modality. The kit can further comprise an instruction on the indication, dosage regimen, and route of administration of the composition prepared from the polypeptide(s) and stress protein(s), the adjuvant, and the additional treatment modality, e.g., as disclosed herein.

In certain embodiments, the composition, polypeptide(s), stress protein(s), adjuvant(s), and additional treatment modality in the containers are present in pre-determined amounts effective to treat cancers. If desired, the compositions can be presented in a pack or dispenser device which may contain one or more unit dosage forms of the compositions. The pack may, for example, comprise metal or plastic foil, such as a blister pack. The pack or dispenser device may be accompanied by instructions for administration

### 7. EXAMPLES

The examples in this section are offered by way of illustration, and not by way of limitation.

### 7.1 Example 1: Phosphopeptide Synthesis

The antigenic peptides set forth in SEQ ID NOs: 119, 123, 125, 133, 134, 221, 247, 281, 296, 355, 407, 410, 417, 419, 421, 455, 456, 457, 546, 563, 611, 641, 642, 650, 654, 657, 669, 670, 701, 703, 791, 809, 821, 824, 859, 869, 911, 954, 974, 979, 1016, 1028, 1032, 1049, 1143, 1148, 1149, 1167, 1176, 1179, 1199, 1217, 1218, 1220, 1228, 1232, 1240, 1253, 1254, 1260, 1266, 1267, 1274, 1298, 1300, 1314, 1343, 1350, 1468, 1486, 1493, 1534, 1536, 1539, 1550, 1552, 1561, 1571, 1574, 1598, 1621, 1651, 1658, 1669, 1670, 1683, 1694, 1720, 1735, 1763, 1767, 1779, 1787, 1804, 1811, 1812, 1814, 1818, 1822, 1825, 1867, 1918, 1927, 1981, 2078, 2111, 2255, 2265, 2328, 2331, 2332, 2355, 2363, 2364, 2386, 2400, 2451, 2453, 2454, 2463, 2465, 2467, 2470, 2471, 2472, 2485, 2486, 2505, 2507, 2512, 2525, 2557, 2570, 2580, 2706, 2740, 2747, 2751, 2753, 2784, 2793, 2803, 2813, 2816, 2863, 2872, 2911, 3124, 3199, 3324, 3360, 3553, 3560, 3572, 4052, 4157, 4253, 4477, 4572, 4682, 4690, 4714, 4715, 5231, 5266, 5303, 5654, 5656, 5719, 5766, 5842, 6015, 6074, 6175, 6176, 6330, 6369, 6630, 6747, 6751, 6768, 6868, 6936, 6947, 6973, 6993, 7019, 7069, 7258, 7269, 7271, 7290, 7377, 7388, 7389, 7402, 7413, 7454, 7482, 7498, 7523, 7530, 7531, 7586, 7637, 7646, 7700, 7797, 7830, 8047, 8082, 8119, 8231, 8244, 8470, 8472, 8808, and 8810 were synthesized using standard Fmoc solid-phase chemical synthesis with pre-loaded polystyrene Wang (PS-Wang) resin in a Symphony®X automatic synthesizer (Gyros Protein Technologies Inc®). A sample of the first amino acid loaded resin from the C-terminus was placed in a dry reaction vessel and was charged to each of the 24 reaction/pre-activation vessels. The synthesizer was programmed to run the complete synthesis cycle using O-(1H-6-Chloro benzotriazole-1-yl)-1,1,3,3-tetramethyluronium hexafluorophosphate/N-methylmorpholine HCTU/NMM activation chemistry. The phosphate group was incorporated using N-α-Fmoc-O-benzyl-L-phosphoserine, N-α-Fmoc-O-benzyl-L-phosphothreonine and N-α-Fmoc-O-benzyl-L-phosphotyrosine for serine, threonine and tyrosine respectively. A 5-fold excess of amino acid, 5-fold excess of activating reagent (HCTU) and 10-fold excess of N-methyl morpholine was used for the peptide coupling reaction. The coupling reaction was performed for 10 min with double coupling cycle for any incomplete coupling throughout the synthesis. These steps were repeated until the desired sequence was obtained.

At the end of the peptide synthesis, the resin was washed with dichloromethane (DCM) and dried. Upon completion of phosphopeptide assembly, the resin was transferred to a cleavage vessel for cleavage of the peptide from the resin. The cleavage reagent (TFA:DTT:Water:TIS at 88:5:5:2 (v/w/v/v)) was mixed with the resin and stirred for 4 hours at 25° C. Crude peptides were isolated from the resin by filtration and evaporated with N₂ gas, followed by precipitation with chilled diethyl ether and storage at 20° C. for 12 hours.

The precipitated peptides were centrifuged and washed twice with diethyl ether, dried, dissolved in a 1:1 (v/v) mixture of acetonitrile and water, and lyophilized to produce a crude dry powder. The crude peptides were analyzed by reverse phase HPLC with a Luna® C18 analytical column (Phenomenex®, Inc) using a water (0.1% TFA)-acetonitrile (0.1% TFA) gradient. Peptides were further purified by prep-HPLC with a preparative Luna® C18 column (Phenomenex®, Inc) using a water (0.1% TFA)-acetonitrile (0.1% TFA) gradient. Purified fractions were analyzed using analytical HPLC and pure fractions were pooled for subsequent lyophilization. Peptide purity was tested using an analytical Luna® C18-column (Phenomenex®, Inc) and identity confirmed either by LC/MS (6550 Q-TOF, Agilent Technologies®) or MSQ Plus™ (Thermo Electron®, North America).

### 7.2 Example 2: HLA Binding

In this example, the HLA binding affinity of selected phosphopeptides (synthesized according to the method set forth in Example 1) was determined.

Phosphopeptides were synthesized according to the methods described in Example 1.

An AlphaScreen® assay was used to evaluate the binding of peptides to HLA molecules. Donor beads conjugated with streptavidin, and acceptor beads conjugated with the anti-human HLA class I antibody W6/32, were used to assess peptide binding. Biotinylated HLAs (A*02:01, B*07:02, C*07:01, or C*07:02) were mixed with a fixed excess of β2m and the mixtures added to each well of a 384-well microplate. Serial dilutions of the synthesized phosphopeptides were added to the wells, and the resultant HLA/β2/peptide mixtures were incubated overnight at 18° C. W6/32 conjugated acceptor beads were subsequently added to the wells, and the mixture was incubated for 1 hour at 21° C. Streptavidin conjugated donor beads were then added to the wells, and the mixture was incubated for a further 1 hour at 21° C.

The microplate was read using the PerkinElmer® plate reader, and data were plotted using the Michaelis-Menten equation to determine the K_{d} for each phosphopeptide.

Table 8 lists the K_{d} of each of the selected phosphopeptides to the indicated HLAs (A*02:01, B*07:02, C*07:01, or C*07:02). NT means that binding was not tested. NB means no binding was detected. LB stands for low binding and indicates that while some binding was observed, it was below the level that would allow accurate calculation of a K_{d}.

TABLE 8
HLA binding characteristics of
selected phosphopeptides

| | SEQ | Kd | Kd | Kd | Kd |
|---|---|---|---|---|---|
| | ID | in nM | in nM | in nM | in nM |
| Peptide | NO: | A*02:01 | B*07:01 | C*07:01 | C*07:02 |
| RTLsHISEA | 2332 | 157 | NB | 380 | LB |
| RVAsPTSGV | 2363 | 241.6 | 227 | LB | NB |
| SIMsPEIQL | 2451 | 83.96 | NB | LB | LB |
| SISsMEVNV | 2453 | 305.9 | NB | NB | NB |
| SISStPPAV | 2454 | 168.8 | NB | NB | NB |
| SLFGGsVKL | 2463 | 53.89 | NB | LB | LB |
| SLFsGDEENA | 2465 | 277.8 | NB | NB | NB |
| SLFsPQNTL | 2467 | 115.8 | LB | LB | LB |
| SLFsSEESNL | 2470 | 466.2 | NB | NB | NB |
| SLFsSEESNLGA | 2471 | 148.7 | NB | NB | NB |
| SLHDIQLsL | 2472 | 31.86 | 642 | 962 | 413 |
| SLQPRSHsV | 2485 | 779.8 | LB | NB | NB |
| SLQsLETSV | 2486 | 120.8 | NB | NB | NB |
| SMSsLSREV | 2505 | 926.6 | NB | NB | NB |
| SMTRsPPRV | 2507 | 701.3 | NB | NB | NB |
| SVKPRRTsL | 2706 | NB | LB | NB | NB |
| TVFsPTLPAA | 2784 | 46.94 | NB | NB | NB |
| VLFSsPPQM | 2793 | 226.7 | NB | LB | NB |
| VLLsPVPEL | 2803 | 149.5 | NB | LB | NB |
| VLYsPQMAL | 2813 | 40.45 | LB | NB | LB |
| VMIGsPKKV | 2816 | LB | NB | NB | NB |
| yLQSRYYRA | 2872 | 255.4 | LB | NB | LB |
| RQAsIELPSMAV | 1812 | 14.8 | NB | NB | NB |
| RIYQyIQSR | 1217 | NB | NT | NT | NT |
| RPRsPTGPSNSFL | 1735 | NB | 360.2 | NT | NT |
| RPRIPsPIGF | 1658 | NB | 84.37 | NT | NT |
| LPRPAsPAL | 1049 | NB | 222.73 | NT | NT |
| RPAFFsPSL | 1486 | NB | 397.5 | NT | NT |
| RPKtPPVVI | 1598 | NB | LB | NT | NT |
| KIKsFEVVF | 642 | LB | NB | NT | NT |
| YRYsPQSFL | 2911 | NB | NB | NT | NT |
| RPFsPREAL | 1552 | NT | 162.72 | NT | NT |
| GPRSASLLsL | 457 | NT | 47.07 | NT | NT |
| SPFKRQLsL | 2525 | NT | 64.68 | NT | NT |
| SPAsPKISL | 2512 | NT | 274.8 | NT | NT |
| RPDVAKRLsL | 1539 | NT | 216.1 | NT | NT |
| RPRPVsPSSLL | 1670 | NT | 186.4 | NT | NT |
| KRFsGTVRL | 911 | NT | NB | NT | NT |
| KAFsPVRSV | 611 | NT | NB | NT | NT |
| RLLsFQRYL | 1300 | 49.72 | NB | NT | NT |
| RLSsPLHFV | 1350 | 90.61 | NT | NT | NT |
| TPRsPPLGL | 2751 | NB | 75.55 | NT | LB |
| TPRsPPLGLI | 2753 | NB | 90.98 | NB | LB |
| QPRtPSPLVL | 1149 | NT | 198.75 | NT | NT |
| GPRSAsLLSL | 456 | NB | 34.09 | NT | NT |
| RPYsPPFFSL | 1804 | NT | 156.7 | NT | NT |
| LPAsPRARL | 1028 | NT | 83.46 | NT | NT |
| RPSsLPDL | 1763 | NB | 369.4 | NT | NT |
| RPRsISVEEF | 1683 | NT | 86.14 | NT | NT |
| RRGsFEVTL | 1918 | NT | NB | 16.58 | 15.87 |
| RRLsFLVSY | 1981 | NT | NT | 1574 | 112.8 |
| RIYQyIQSRF | 1218 | NT | NT | 476.8 | 377.2 |
| RRPsLLSEF | 2078 | NT | NT | 804.1 | 35.69 |
| RRSsFLQVF | 2111 | NT | NB | LB | 18.98 |
| RRIsDPQVF | 1927 | NT | NB | 11.41 | 19.94 |
| RRFsGTAVY | 1867 | NT | NB | 2176 | 16.69 |
| FRRsPTKSSLDY | 355 | NT | NT | NT | 98.63 |
| QPRtPsPLVL | 1148 | NB | LB | NB | NB |
| RQAsIELPSM | 1811 | 20.37 | NB | NB | NB |
| KLIDRTEsL | 670 | 260 | LB | NB | NB |
| GPRSAsLLsL | 455 | 500 | 50 | NB | NB |
| RPTsRLNRL | 1779 | NB | 117.35 | NB | NB |
| RPRPVsPSSL | 1669 | NB | 218 | NB | NB |
| AVRPTRLsL | 221 | NB | 117 | NT | NT |
| GLLGsPVRA | 421 | 431 | NB | NT | NT |
| EPRsPSHSM | 296 | NB | 198.6 | NT | NT |
| RVRsPTRSP | 2400 | NB | 551 | NT | NT |
| RLLsAAENFL | 8808 | 114 | NT | NT | NT |

‘s’, ‘t’ and ‘y’ indicate phosphorylated serine, threonine and tyrosine, respectively.

The invention is not to be limited in scope by the specific embodiments described herein. Indeed, various modifications disclosed herein in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. An antigenic polypeptide comprising:
an WIC-binding peptide comprising an amino acid sequence selected from the group consisting of SEQ ID NOs: 98-3000 and 8808; and
an HSP-binding peptide comprising the amino acid sequence of X₁X₂X₃X₄X₅X₆X₇ (SEQ ID NO: 1), wherein X₁ is omitted, N, F, or Q; X₂ is W, L, or F; X₃ is L or I; X₄ is R, L, or K; X₅ is L, W, or I; X₆ is T, L, F, K, R, or W; and X₇ is W, G, K, or F.

2. (canceled)

3. The antigenic polypeptide of claim 1, wherein the HSP-binding peptide comprises the amino acid sequence of:
(a) X₁LX₂LTX₃ (SEQ ID NO: 2), wherein X₁ is W or F; X₂ is R or K; and X₃ is W, F, or G;
(b) NX₁LX₂LTX₃ (SEQ ID NO: 3), wherein X₁ is W or F; X₂ is R or K; and X₃ is W, F, or G;
(c) WLX₁LTX₂ (SEQ ID NO: 4), wherein X₁ is R or K; and X₂ is W or G;
(d) NWLX₁LTX₂ (SEQ ID NO: 5), wherein X₁ is R or K; and X₂ is W or G; or
(e) NWX₁X₂X₃X₄X₅ (SEQ ID NO: 6), wherein X₁ is L or I; X₂ is L, R, or K; X₃ is L or I; X₄ is T, L, F, K, R, or W; and X₅ is W or K;
optionally wherein the amino acid sequence of the HSP-binding peptide comprises an amino acid sequence selected from the group consisting of SEQ ID NOs: 7-42.

4.-40. (canceled)

41. The antigenic polypeptide of claim 1, wherein
the MHC-binding peptide is 8 to 50 amino acids in length, optionally 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length;
the C-terminus of the MHC-binding peptide is linked to the N-terminus of the HSP-binding peptide, or the N-terminus of the MHC-binding peptide is linked to the C-terminus of the HSP-binding peptide; and/or
the HSP-binding peptide is linked to the MHC-binding peptide via a chemical or peptide linker, optionally wherein the peptide linker comprises the amino acid sequence FR or the amino acid sequence of SEQ ID NO: 43.

42.-47. (canceled)

48. The antigenic polypeptide of claim 41, wherein the N-terminus of the MHC-binding peptide is linked to the C-terminus of:
(a) the amino acid sequence of X₁X₂X₃X₄X₅X₆X₇FFRK (SEQ ID NO: 68), wherein X₁ is omitted, N, F, or Q; X₂ is W, L, or F; X₃ is L or I; X₄ is R, L, or K; X₅ is L, W, or I; X₆ is T, L, F, K, R, or W; and X₇ is W, G, K, or F;
(b) the amino acid sequence of X₁LX₂LTX₃FFRK (SEQ ID NO: 69), wherein X₁ is W or F; X₂ is R or K; and X₃ is W, F, or G;
(c) the amino acid sequence of NX₁LX₂LTX₃FFRK (SEQ ID NO: 70), wherein X₁ is W or F; X₂ is R or K; and X₃ is W, F, or G;
(d) the amino acid sequence of WLX₁LTX₂FFRK (SEQ ID NO: 71), wherein X₁ is R or K; and X₂ is W or G;
(e) the amino acid sequence of NWLX₁LTX₂FFRK (SEQ ID NO: 72), wherein X₁ is R or K; and X₂ is W or G;
(f) the amino acid sequence of NWX₁X₂X₃X₄X₅FFRK (SEQ ID NO: 73), wherein X₁ is L or I; X₂ is L, R, or K; X₃ is L or I; X₄ is T, L, F, K, R, or W; and X₅ is W or K; or
(g) an amino acid sequence selected from the group consisting of SEQ ID NOs: 74-97.

49.-72. (canceled)

73. The isolated polypeptide of claim 41, wherein the C-terminus of the MHC-binding peptide is linked to the N-terminus of:
(a) the amino acid sequence of FFRKX₁X₂X₃X₄X₅X₆X₇ (SEQ ID NO: 44), wherein X₁ is omitted, N, F, or Q; X₂ is W, L, or F; X₃ is L or I; X₄ is R, L, or K; X₅ is L, W, or I; X₆ is T, L, F, K, R, or W; and X₇ is W, G, K, or F;
(b) the amino acid sequence of FFRKX₁LX₂LTX₃ (SEQ ID NO: 45), wherein X₁ is W or F; X₂ is R or K; and X₃ is W, F, or G;
(c) the amino acid sequence of FFRKNX₁LX₂LTX₃ (SEQ ID NO: 46), wherein X₁ is W or F; X₂ is R or K; and X₃ is W, F, or G;
(d) the amino acid sequence of FFRKWLX₁LTX₂ (SEQ ID NO: 47), wherein X₁ is R or K; and X₂ is W or G;
(e) the amino acid sequence of FFRKNWLX₁LTX₂ (SEQ ID NO: 48), wherein X₁ is R or K; and X₂ is W or G;
(f) the amino acid sequence of FFRKNWX₁X₂X₃X₄X₅ (SEQ ID NO: 49), wherein X₁ is L or I; X₂ is L, R, or K; X₃ is L or I; X₄ is T, L, F, K, R, or W; and X₅ is W or K; or
(g) an amino acid sequence selected from the group consisting of SEQ ID NOs: 50-67.

74.-91. (canceled)

92. The antigenic polypeptide of claim 1, comprising an amino acid sequence selected from the group consisting of SEQ NOs: 3001-8806, 8809, and 8810.

93. The antigenic polypeptide of claim 1, wherein the antigenic polypeptide is 15 to 100 amino acids in length, optionally 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length.

94. (canceled)

95. The antigenic polypeptide of claim 1, wherein the antigenic polypeptide is:
chemically synthesized; and/or
comprises a phosphopeptide selected from the group consisting of SEQ ID NOs: 98-3000 and 8808, wherein a phosphorylated amino acid residue of the phosphopeptide is replaced by a non-hydrolyzable mimetic of the phosphorylated amino acid residue.

96. (canceled)

97. A composition comprising at least one of the antigenic polypeptides of claim 1, optionally wherein
the composition comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 different antigenic polypeptides, and/or
the composition comprises an adjuvant, optionally wherein the adjuvant comprises a saponin or an immunostimulatory nucleic acid, optionally wherein the adjuvant comprises QS-21, a TLR agonist, a TLR4 agonist, a TLR5 agonist, a TLR7 agonist, a TLR8 agonist, and/or a TLR9 agonist.

98. A composition comprising a complex of the antigenic polypeptide of claim 1 and a purified stress protein, optionally wherein:
the stress protein is selected from the group consisting of Hsc70, Hsp70, Hsp90, Hsp110, Grp170, Gp96, Calreticulin, and a mutant or fusion protein thereof, optionally wherein the Hsc70 comprises a human Hsc70, optionally wherein the Hsc70 comprises the amino acid sequence of SEQ ID NO: 8807;
the stress protein is a recombinant protein; and/or
each of the different polypeptides comprise the same HSP-binding peptide and a different WIC-binding peptide.

99.-115. (canceled)

116. A method of inducing a cellular immune response to an antigenic polypeptide in a subject, the method comprising administering to the subject an effective amount of an antigenic polypeptide of claim 1.

117. (canceled)

118. A method of treating a disease in a subject, the method comprising administering to the subject an effective amount of an antigenic polypeptide of claim 1.

119.-128. (canceled)

129. A kit comprising a first container containing an antigenic polypeptide of claim 1 and a second container containing a purified stress protein capable of binding to the antigenic polypeptide, optionally wherein the kit comprises a third container containing an adjuvant.

130.-142. (canceled)

143. A method of making a vaccine, the method comprising mixing one or more antigenic polypeptides of claim 1 with a purified stress protein under suitable conditions such that the purified stress protein binds to at least one of the polypeptides.

144.-150. (canceled)
